SEQUENCE LISTING (9 sequences)

SEQ ID NO: 1 - DNA, 131384 bp, Unknown
  Bacteriophage P100
aaggttgtgctaaattaatacagtagaactatgtggaatggaggaaatattatgaacaagccagacaaaaatttctttatgaaaatgattatcgttattctatcaggattacttgtgttatttgttataaacctagcatctatctactttgggtactctcctcacgttgtagctacgtactttatgacagggggctttattgcgtatatcgtgataaacttatacgaccgaattgttttccaaaaaggttctcctagggaagaacgagagaaacaagagagacaaaagatagcatctagacaaaaagctttagaaagcgtagtaactcaacgactaggtatccctatagaggagctagatttagaagtactcattcagatggaaaaggatttagcagacaaaactgctaaaggtaataaaaatgaaaaaactgagtagagacgagataaagaaaagacgaaaatatttcattaacgaagcaaaaagagttacacatatacgagcaggagaccgtagaatagcaacaggttcaagaatatgtaccttatgtggtagaagtctatcatctcagaaacggtctacaggagagacctttgcctcagtaaaccacatccattgttactttaatgaacttcttatcgttaacttatgtgaagatgcaacttcttgttatagaagacatgaaaggagttagatagtatgaatttagacaacatagaaagaaacttaagcaagaaaaaaactacaggagatgccgtaaaggatgagttcaatgacatggctatgaagctactagcaggctttaaagcagatgtttcggcaggagataaaaaattagaagatgtagcagacatggctaggctgttacaaacctttatacaagttaacaaagaagatagtggtagcggaggaacatccacttctttaccagagcttagtagcttgcaacaagaagcaataaccaatcagataaagacagagagcgtagtttcaggtgataaagagcaagaactagtagacctacaggacttagctaacatgacagaagaggacgtacaacggatgataatggataaagaaaaagcaatgaacatggaaaatgaggagacgttttaatggatggcaacactcttttaaagatggcgcaagaaacttttcaaacagaaaatgtaactacagagcagttaaactatgttctggatatgctaactccatcaacatatttactaagaaaccacaaagtaagaaatcataacattacgttctctattgcaaacaagtctactgaaaaggcgcaggcacatcgaccttggcaacctaagattataaatgactcccacccgaatgtagctgttattaagtcaagacaattagggttaaaattagcccctctataaagtaatttataaagaaaactctgttaaacgggcataggaaaataaccaatatcctgataagagaccctaaatcctaagtaggacagtgggaatcccgtgctaaatcaggcgcaagcttgtaaatgcctaacgactaaatttctaggtagctaaccaaatggggttagtgagaactagataagaaaatatattctcttacgataccaactgaaaaactctatcagatgttaaaaaattcttagaagaaaatataaagtaaagcagagcaacctaaaccaaaaaattaggttgatgatatagtctagtcccctaataaatatcgggaaaccgagggtataacgtaagtgagatgggtgtagggagtatgattcattttgcagatacacacagctatgcagctgttaaatgtttatacacgtttcctacaggtgaacagatgcgtaaatttgtacagtcacggttagaccctgtacttgagtctagttactatagttctattctagacaaagataacaacagtctgaactacaaaagaatacgaaatagtcacctttactttagaacatctagtaagcccggagctgtagagggtgtagatattgactacttatccatggatgaatatgacagagttcctgttttctcagaggcttccgctttagagtctatggctagttcaccctttaaaattgttagaagatggtctacaccatcagctcccggtatgggaatacaccgattattcgaaaactcagaccaacattggtacttgcataaatgtgaaaaatgtaaccactggaaccaactagactacgaggactacgacagtagctctgtagaagcaggaggaaatatactatgtgtaaatccacaaggtgttgataccttagctaaaacagttgtagaaggttcgttccaattcgtatgtaaaaaatgtggagccccattagaccgttggtataatggtgaatgggtagcaaagcaccctgatagaacaaaaaatggagacggtattagaggatatatgatttctcagatgaatgctgtttggataagtgctgatgacttaaaacgtaaagaactaaattcactttcaaaacaagcattttttaactatacactagggtacccattcgaagatgctaaactagcagtttatagtgaagatattatagaaaatccaagcccaagagtaaaggttccaatgcacagcagaggagattacaagtacatttctgttggtatagactggggaaatacacactgggtttctgttcatggtatgacggaaagaggagaaatagacttgataagactgtttagtgttgagaagagtcgaggagtaggtaatattgagtctgatttagataagattatcctagaagtatctatgtacaaccctgatatgattgtagctgacgtaggggatagcgggaactacgtggacaaattagtaaaacatttcggagaagaccgagtatttggttgtatttataagtccagccctaaatctacaggtcagttgaaaccgcagtggaatgaagcagggaatgtagttactgtagacaagttaatgcagaacaaaagatatattatagaaatgaagacaaagaaagttaacttctatagctttatagaccctatgctaaaactattggtagaccattggagaaacgttgtaatacaagacgaagaggacgaaaaagatggaactttctaccaagttattggcagaagaggagacgaccatttagcgcaggcatcagtgtacagctttattggtttagacagactagctgatatgtatttgagaggctctaagtatgagttcaactctacttttgtaacaagcaactaccaagatgtttcgtcaacaccaccagatatattcagtaaatattaaattaatttagttagggggctatgctccctttcttatttgtgttataatatagacaaggaggttttactgtgacaaatgaagagaagtttgacaaagtaatgtctatcgttaaagaagacaactctaccgtcctacctgactccgcttctattagtaaaggaattttaagagagttgacacaggaaagtttacaagactacggagaggtcattgaaaagaatgattttttaagcagggagtttaaacatattgtttccctctacggttttgacgacttttactcactatatgcctatgctgatttttgcgatactatgagtaatctagctattaaaggcgggaacaaagatttatctaagttaaaaaaggtaaaaagaaccgttatgagaaacggaaaacctacagagatgactttttatgagtcaactaccgaagaagagaataaagacaagaaagaagaaagtaataccaagcgccaaggagtaagcacagtatctgcaaaagacttacttgcagagctactaaagtctaaaggggaaccagaaaaagtaactcagttaataaaaataattcttaaattccctgatacacctgctaaccccaaagtatctgattatttttaccagttaaaagacgactcgggagaagtagtcgctgtggcaggttacagtataggagcccaatacgtctctcttgatttttataccagtaatggctctattagcggagtagcatcaagagcgttctttgaattactacggatagcaatagaaaaagacttaggagctgaattaaatgacgaggataacgagctagctcaagtactattttcaaaatttaattttcagctagatgagggaaaacagagagtgtctgctagaaacttaaaaaaacactttaaggggtaactatatgttgccattctattcaatatatgctataataatagcatgtgttatatacacagttatctttattttagtagtacgcagaatgtatcgtaaagcagacaaacgagagaaggaaaaagaagacattcttaacactatgacagaaataacagaggaaataaaaacatttattggaggagaaaataacaatgacaattgagaacacgaagactttagaacaaacactaaatactgtagccgaggaaaaagattatgacttattcctatcaacgttggcgggggtagttaaatcactatacaaagagtaccaatttcttagacaagacccagcacccgaggataaaacatcaggaaacattttgattttccaaattttccgaggagaccctgacggttatgctaaagattttgatacactgtttaaaattgcagtagacagaaatttagagcacagaaaaatatcccaaaaattctttgaatgggtagagaaaggtaagttcttagagttgggtgataacatcgttattagcagggatggtacaggagtaagctctattgttgagtctgttgaacttaaagtttttattacttttatgactaaaagttatgccgaagaacgagaagattttttagctaaagagaaggaaaagcaagaagaggctaaaaagaaagaagaagagcgactagctctaaaagaggaatcgtataaatctttaggagaggctacagaaatgctaaatactctcatggaagagcaaggtgtatctcctgaggagcttgctttagctttagaacaggggaaggagagttatcgtaatgcacgataatgaatttgaagtacctgagagtgttattaatgcttctgctacaccttttgaggcgcctgatagaacaaaagatacctaccaaagttcattaaagaactggttggacagagtaaaatcagtcggagatgcaggaatgtcacgagagtttgttattcgtattccagcaaaggcactagaggagaacaacccagaagctaccttatttttaaaacagtgtaacgaaataaaaagaaaccctaagtgcaaccttcctattaaaattgaaaaaacagtagagaagtacaaagacaagaaacacttaatgttagacttggtgttcacttatagtaataaaacagatattgatgcagtaaatagtgtaataaaataacaaacaggttcctctttacgtctgctatattagatagtagaaaacgtaaggaggaatttttatatggtaaaatataccgtagagaacaaaattattgcaggattacctaaaggtaaactaaaaggggctaactttgttattgctcatgaaactgcaaatagcaagtctactattgacaatgaagtaagctacatgactaggaactggaagaacgcatttgtaactcactttgtaggtggcggaggtagagtcgttcaggttgctaatgtaaactatgtttcttggggagcaggtcagtatgctaactcttattcctatgcgcaggtagagttgtgccgtacaagtaatgcaactacatttaagaaagactatgaagtgtactgtcaattactagtagacctagctaaaaaagcaggtatccctattacacttgactctggtagtaaaactagtgataaaggtattaaatcccataaatgggttgctgataagctaggaggaacaacacaccaagacccatatgcttacttaagctcatggggtattagtaaagcacaatttgctagtgacttggctaaagtatctggcggaggaaacacaggaacagcgccagctaaaccaagcacaccagcacctaaaccaagcacaccatctactaacctagacaaacttggcttagtagactacatgaacgctaagaaaatggactctagctacagtaacagagctaagttagctaaacagtatggtattgctaactattcaggaacagctagccagaacactacactccttagtaaaattaaaggaggagcacctaaaccaagcacaccagcacctaaacctagtacatctacagctaagaaaatttatttcccaccaaataaaggaaactggtctgtgtatccaacaaataaagcacccgttaaggctaatgctattggtgctattaaccctactaaattcggaggattgacttacactatccaaaaagatagaggaaacggtgtatacgaaatccaaacagaccaattcggcagagttcaagtctatggtgcacctagtacaggagcagttatcaaaaaataaataatggtaaactagaggagacttttatagtctcttcttttcttttgtaacagtagtgaaataaaacttaacccctttgtaatattatggaattacttctgtaacacatctatgttatatttagttggtaatttaaaaacgcatatgcggaatatagaaaggaaaccagctaatgaaaaaaggaagtaagattgcaattgctactgtagtagctgtacttattgtcgttacagttagcctagggtaccatacagttaagttgaatgagacaaatgaggcaaagacagaagaggtagttaagctaaaggagaatatctctagtgtatctaaagatttagataatttaggtaaagaacatgaaaagaaagtctcagaatccgaagctcttaagaaagaccttgacaataaaaagaaagagttagaggataagaacaagaaagaagagcaacttaaaaaagaaaaagaagaactacaaaaacaagtaagctataaaaagagcttaaagaaaaaggcagtagcagaaaacattccaaatgcaactagtagtaataaaagtgctaaaacggcagaaagcaaaggaaatggaagaagttttaacgtagaagttacttactatgctattggtgacggttttactcccggtgtacataccgcaaatggtactaacatcaaaaacacaataacaacatcagaaggacatcgaataattgctgtagacccatcagtaatccctatgaactctattgttaaagtaactacaggtaacggagagagttttgtagctaaagcttgtgatacaggtggagctatcaaaggtaacattattgatattttagtagaacctagcgcagatatttatcaactagggagaacaacaggtactgtagaaattattggataatttaaagaaataagagtctgcaaggtataacaaccttgtgggcttttttattatgctatattatatatgacggattatgaagtttgaggaggtatattaacgtggttataacacagatagtgatgattatagcgattattattactacatttcttattatttatggtatatactcttctaggagaaaaacgtctaagtcgtttagactagtactcagatatgctataatggtagtaggtcttcttattgtctactatctatttcaagtttcctctattgacattcaacaggattttttacagcacttaaaaacaggaggtaacgggatagaagaagtttttttcacaaatttctttcttatcatggtaggagcagtaggtacacttgtagtacaactattccaaaatagaaagaaggattaatgtgtcatatacaaaagactccttgtggttggagacaaaaaaggttgctagagataatgggtttttaggtaactggatagagatggttgactactatgagagtataggaggaagccatgtattcgtttatgcggttatggaaggtcagaagtaccgaattttaggcgttcttagcacggatgaggtattactactaaaaagagatttaacgctctcaaaagccctatatgaagatgttatgaatagcaaaaagatgtttttctataaagaggacagtgaaaagaaaaaaataaaattgacagatgaaataagtaagttagaagataaagatagtatcatagtatatatgtaggtaggtgaaaaataaattgggactatttgaaagtataagattagcgggggtaaataagagcgatgcagtaaaacacattgaggttgatgataactactccattgcaattcaacaaagggaacaggaacaaatatctaaagcaatgaataacaaagaagtagcttattctcaaccagtaattggctctatgtctgctaacccggggtttaaaacaaaacctagtattcgtaataaccaagatttacacggtgttcttaaaaagtttggtggaaacatcatcctgaacgcaataatcaatacaaggtcaaaccaagtttctatgtattgtaaaccagctagacactctgagaagggtgttggttttgaggttagattaaaagatttagataagaagcctacttctcatgatgaagcaacaattaaaagaatagaaagctttatcgaaaaaacaggagtagataacgatattaaccgagatagcttttcttcctttgttaaaaaaatagtacgagacacttatatgtacgaccaagtaaactttgaaaaagtatttaacaggaaccaaagcatggttcggtttgtagctaaagacccaacaactatattttttgcgacaacagcagatgggaaaattcctgataacggtaatcggtttgtacaggttatagaccagaaaatagtagctacgtttaacgcaagagagatggcatttgccgtaaggaacccacggtctgatatttatgctacaggatacgggtaccctgagttagagatagcacttaagcagtttatagcacatgagaatacagaagcgttcaacgatagattttttagtcacggtgggacaacacgaggtattctacaaataaaagcagcacaacaacagtcgcaacatgctttagagatatttaaaagagaatggaagaactcgttaagtggtatcaacggttcatggcaaatccctgtagtatctgccgaagacgttaagtttgtaaatatgacaccttctgcaagagatatggagtttgaaaagtggttaaattatctcattaatgttatctccgcactgtatggcatcgacccagcagaaataaacatacctaacaacggaggagccacaggttcaaaaggtggtagcctaaacgaaggaaactctgccgaaaaaaatcaggcatccaagaacaagggcttacaaccattattaggatttattgaagactttataaataaacatattgtagctgaatttggagacaagtacacattccaatttgtaggcggagacataaaatcagaactagagtcggttaagatactagcagaaaaagcaaaagttgctatgacagtaaatgaagttcgtaaagaactaaacttaccgggagatgtaattggaggggacattccattaaacggagtaatcgttcagcgtataggacaacttatgcaacaagagcagtttgagcatgagaaacaacagtctaatttacaaatgttacaagaacagacaggtaacagagtaagtactgatgtagaagacattcctgatgggaaagatacaacaggagacataggaaaagacggacaaagaaaagataaagataacgcaaacgctggtaaacaaggcatgaaaggtgataaacctaacgattggcagacctaatattttgtagttctgccttttaggtaactaactagtagaggtatatgctatattaataacagaatgtgttataatagatatgagaggtgatttcttaattgaataacgtcgtagagaaagagatgaaaatctttgtacctattgatattgaaggttctttagaaaaatcccataagggagatgacaaggactggtatgttaaaggttacgcatctacacctgctctagacttacagggagaaatggttaaccctgctggaatagacattggttattttgtagataatggttacattaactttgagcatagacaagaaccagactatgtagttggagttcctacaagtaactgttatgtagacttccaaaaaggtctgttcgttgaagcaagactgtttaaagacaacgaacacgcccaatctatgtggaagttagctaacagcattagtaagacaggtattgatagacaacttggtttttctatcgaaggtgcaataagaaagagaaacacagatgacagccgaattattgaagaggttgtaatcaaaaatgtagcagtcacaatgaacccagcaaacccagaagctacgtgggaaacatttatgaaatcatggactactggacatggaacaacacctgatacgcaagtaaatgcaggagctttaagaagagagagcttagctaacagcatatcagtactagcatcagcatacaaaatttctgaccctattgaatttgacaacttatggaaagatgtaagtaagtttttagatgaaaaaggagagtataacgagaatgaggcaatagttatgttacaattagctaaaggcgtttctcgtgtaagtgcatgtgagtctttaatgaatagacgaaaggggaaacaaaatgaataacagtgaagaaaagttcgaagatgttttaaacgaatttggtaatacagtagaaaaagataaagaagaaatttctaagtctaccgaagaaaaagaggaggttacagatattaataaagataaaaaatcagaagaagaaactgctgaggaagaagtaactgaggctcccgaagtagacgttgcagaagacaaagaagtgtccactgagactcccgaagaagagtctactagtaagtcagctaaggaagaaaagaaagaaaaagatagtgaagaagacaaggacgaagacgagaacaaaaaaggtaagaaagacaaaaaagacaaaaaagaagaagacagcaaagaagaaaaaacggagaagtctgatgaagaagattccgaagaggctgtaaaaaatactaactctgatgcagatattgcaggagcattgaatgctgttgctaagtcttatgagaatgttaaagaaagccaaagagagcaaaatacaactgtagaacgcttagagaagaatatcggtagcttagtcagtaaattagacagcttggtagatttaatgagtgaacgtgtatctaagagtgtagtagaagatattgaaactcttgaaggagctagtgaaaaagctgttggctatgtagcaaaaaacgtagatagcagtgtagtagaagttattgaaggagaagaaacttctaagagtgtagatgcagaagtgagtcctagagatgagtttctagctaataagagtgctttcctagagtcttttgaatatgcaagtcgtaacggtgctgagcgtagagatttaactgatgttcgtcacgctcatgggaacattacaaatgatattgctagtaaagcagattataaccgagttaacgactttatgaaaaaatggagtaagtaataaaggtaactgtatagagtattttttgtaaacagtatgctatattataggaacatgggaaacatgttacgttatgagagagcttgcccgttttaggactcctcccctagggcaggttatctcactaacaaaaaaataaaactatgaaactgaaaggtgataaatataaatgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacagcttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagacttagcagcacacgaatacaaagttgttgtaagttctgacgatgcagagtctattgcaagtgaagtggctacagctacagttactgcaaaagatgacggcgttaaactagaaatcgagttagctccaatgtacagctcccgtccacaattcgtttcaatctatagaaaaggtgcagaaacaggtttattctacctaatcgctcgtgtacctgctagcaaagcagagaacaacgtaatcactttctatgacttaaacgactctattcctgaaacagtagacgtattcgttggtgaaatgtctgctaacgtagtacacttgtttgaattactaccaatgatgagattacctctagctcaaattaacgcatctgttacatttgcagttttatggtatggagcattagctctaagagcacctaagaaatgggtacgtattagaaacgttaaatatattcctgtaaaaaacgttcatagcaactaataattataggataattgaataaaaacagtatagagagcagataaatactgctctctattttactaataaggaggatttaaattgctaaaaaatacaaacttagctaattataaaaaagtgaatacacgatttggaaatcttagttttgatgataaaggtatttctaatgacctaacggaagagcagcaaaaagaattaggtaagcttagaggattcgaatatattaagacagaacagaaaacgaaagaagaacctaagaaagaagaacctaagaaagaaagtacagaaaatgaattagacagcttcttagctaaagaaccttcaatcaaagaattaaaagaatttgcgagtaaaaaaggcattaaaattgaaaaaactaagaaaaatgatataattgaagaactaaagagagggtaatgtacaatgtatggaggttatgaaggacaagattcttacgaatacccttactcacacgggaaccctaagcatgtagagccagaaaaagttgacgaatatgttctttctgattatggctggactgcggaaacaattaaagcatacatgtatggtgttcgtgtagtagaccctgaaacaggagaggaaatgggagacaccttctacaatcatattatagaggttgccgttgataaggcagagaaagaactagatatagctattctcccaagacgggagtcagaacaccacgattataaccaaacagaatttagtagttatatgtatacccatgttcataaaaaacctatcttacaagtggatgacttaaaactagaaatgaatggtagggcaatatatagataccctgctaactggtggaaagtttattgcttagctggacatatagaactgtacccgacagccttgatgcagacttctcaaagtatgacatatgactctgtatttggaggatacccgcagttggcaggaacagccccttctataggtagaacatatgcccctcaaatgattcatgtagaatatatagcaggtatgctaccaaggaaaagagcaggtgttagtcgtgactgggaagtacctgctgcattagaacagttagtaactaaatatgctttagtagaaatattccaagtttggggtagacttatcatcggagcaggtattgctagtagaacactagtttacgacggagtttcagagtctattgatacaactcagtctgccatgtatacaggttcagcagcagatattgagcttataaataaagacatagacagtttagtagctcaactgcgttctcactttggaataaacatgattggactataagagaggagaatgtttaaatggcagagcctactagattttcaagtgatactcctattaaaaacctaacaacacaaaaagtagagtacaacatatcacagcatagaaagtttattaaagaccgtagtatccatacgttatgggaaagagcttatttatgcccttgtatgaacaagtcctcgggttctgctgactcttcttgtcaaagatgccatggtaggggatgggcgtatttaccagctaagcccgcaaatatgattattcaaggacaggaaaaaggggttactaacacagacctaggactatatgactcaggaacggctatcggtacaaatgattttgatgctagaatggcttttagggacagaataacagtacctgatgttaaaataggtcaatcccttatctactctataaccgaaagcagacaaaaacgaggtatatttttaacatatgacgttgatactatagactttataacaagtatggaaagagaactaatagaaggtaaagactacacctttgataaacacagtaataaactattccctgatattagtttgataggtgaagttatatctttaaacgtatctgttactctaaggtatattgttattgacttactaaaagaaagtcgctatcagtacaccacattaaatagaacagaccctaagtttgagcagttgcctcagaagttattactaaaaagagaagacactattgtattacctgaaccatttgtacttgatgctgatacagagactagagtagagttagttaatgaccctaaaaggaaaccaagacaacaaggcgggttctacgaagggatgtttgactagtgaaacagagtacacagagaccatctctattcaataatccaaagcaggtaatgagaaatatggcaagagctatagaatcagatgttattgcaacaggagctcaagaggcatctcgtatacccaatgtagagattaaaattatgcctaagtatttagaagtaactgaaaagagaatggaaaaaaatggtgtaattgaccttaaaccttattttgctaaaagtaataaaaagaaatggaatcaagaaggtaactggtacttagtaatccctataagattaaagacaagagatatgtctcgaaaattatatgatgacttaaggaaacagtcttccccaccagttggaagttcaacaacaattgtatcagatttcctatatgaccgcagaagaacgagccctagtgtcccttctataaactataatccaaagtcaaataatgttacaaaaaaacgtattagtaacagtagaacaacatatacagtataccgtacagtaactgctaagtcacctgctaatagttggattgttaacagggacagggttaatgaagaggatatgagtaaaacgatgttagacagaatagacaggctaatgaaatggaagcttaaaaacttgtagaggaggagtacaatgcctattccaagccttgattcttatgtagcaagaagagtaagagagagccttaaaattatgctttcagagtgctatattatagaggaagctttgagtgacatggatgtcgaagcaaaaagtaactttatgaagacatattgtggagataatcctaaaaaagaaatagaagtgatatacgagttccctcaggcaaaaacatcagtagatgctacttatgtaattcagctaggaggaggttccgaagatactaaaagcatcggcggtattgaaggtacttacgagtataaagaggattctttaagaaaagagacagtagttgctactagagaaggaaccgagttatttctagaaacaaaagagctaattggaagtatagagggaatacctaatttatccttttcagaaagtgataataaacgaatagaaggtaaacgattatacttctcttatgaaggtaatgcagatattgagggagtttccttcgatattatctatacaagtaaagtagacctagagagcgaccccgttggactaaaaaaagggtttacttctagagacaatatcactgttgtaggtttaagcactaacatggatactgcaaggtgcctagatgctatcttaaaagttatccttattatcatgagagaaaacttagaagagaagagttactactccctacaaacaactcagttttccgatatgtctcctgtaattatgggagaggaaaaacctgtatttggtagacctgtaacaatgagttacactatatcatatactgtagactttaatctagtcaaccaaataaataaaattatagtacaggagcgtaataagtgatggcagaaaaaaaagaggtaactaaagaacctattgtagacgaagtaaaagtaacaaagaagaaccttaaggaacctacaacagaagaaaataaagtagtgaagaaaaaacttaaagaagaacaaacagatactgtagagaaagttactacaggatatgttcatatagatgtattccttggaacagccaagccgtggtacggtttatcagaaatgcaagtacaagggtttaaatcttttatgcaagggaaacaataccagttcggtgaacaggcttttgttccttatttagaaaaatatctaggaagggaagtaaaataaaatatggcaatcgaaatttacccaagaaaaccagtttctagacctcatacagaaatttctgtagatacaagcggtattggtggttctagctctggaagtgaaaaaatactttgtttagtaggttcagctacaggaggaaaacctaatgctgtttacaaagtaagaaactattctcaagcaaaatctgttttccgttcaggagaacttttagatgcaattgaaagagcatggaaccccggtgaaggaacaggtgcaggagacattttagctatgcgtgttgaggaagcaaaagaagctacctttgaagctgagggggttaaggttagctctactatttatggagctgatgccaacgacatccaagtggcactcgaagataacaccatcacaggtacaaaacgcttaagcattgtatttgcgaaagaaagagtcaaccaagtttatgacaacttaggtagcatcttctctatcaagtataaaggtaccgaagcttctgcaacatttacagtagctgtagacccagtaacatttaaagctacaaagctaactcttaaagcaggggataaaacagttaaggaatacgatttaggttcaggagcgtatgcagaaactaatgtacttattagtgatattaacaacctacctgactttgaagctaagtttttcccaatcggagataaaaacttaacaacagacaacttcgatgcacaaatcgatgtagatattaaaacaaaagaagcttatgtaaaagctgttggcggagacattgagaaacaaacagcatataatggttacgtagactttgaatttgaccgctcaaaagaaattgctaatttcccactaactaagctaactggtggagacaacggtacaattcctgagtcttgggctgataaattcagttactttgcaaacgagggtggctactacctagttccattaacatctaaacaagctgttcatgcagaggcacttcaatttgttagagattgctcttataacggtaatccaatgcgggtattcgtaggtggaggaatcggagagtcaatggagcagttgttcacaagagctatcggcttacagaatgaacgagcaggtctaatcgggttctcaggaacagtgaagatggacgatggacgttctctgaaaatgccgggatatatgtttgcagcacaagttgcaggacttacatgtggactagagataggagaagcaatcactttcaaaaacattgcgattgagacattagatacaatttacgaaggttctcaactagaccaattaaacgaaagtggtattattaccgcagaatttgtacgtaatagagcagttaccaatttccgtattgtagacgatgtaactacatttaatgataaaacagaccctgttaagtcagaaattggtgtaggagaagctaatgacttcttagtttctgagttgaagataagcctagacaacgagtatattggaactaaaattattgacacctctgcttctttagttaagaacttcgttcaatctttcttagatagaaagaaattagctaaagaaattcaagactacagcccagaggaagttcaagttgtaattgagggcgacgttgctcgtatctcactaacagtattcccaatccgcagtatgaagaaaattgaggtatctctagtataccgtcaacaaatccttactgcctaatctaatacaggggagggcttagctctccctatgtaaataaaaaataaaacaggagttgaaacataaaatggcaagtgttactaatcagacggttcatacaggtaatacggtctaccttatgataaaaggtaaaatcgtaggaagatgtcagtctgcatcaggtgaacgttcttatggaacaactggtgtatacgaaatcggttctatcatgccacaagaacacgtttacttaaaatacgaaggtagtttaacagtagacagactgcgcatgagaaaagaagatttagctaagctaggtattacagcattaggagaagacattcttaaaagagatgttatcgacattgttatgatggataatactactaaagaagtaatcattgcttatcgcggatgttctgctgagacatacaatgaagaaatcaaagctaatgagattgtttctgaaagtgcaagattcttgtacctaagtgcagcaaacgtaaaataatactaataaaaggctaccttaatctggtagtcctttttttatagtttactaaccttagcaaatatggtataataaagtaagagaacgaaaaagtattactagtttgttatattagataatgagagagtaaacaaggaggagacaaattacatgtcagaagaacaaaaagtaaatgaagaacaaatagaagaaagtaaagagaagatgttgacgaaagaagacatcgaccgtatcaactatgaacaacaaacgaagcaaaagaaaacacttgaccgtattattaaaggggtaaatgatgtttggacacatgagtatgactttaaggagttagatttaaagttcaccattagattacgagcacctaatgctttagaacaagcaacaattcaagctaacagagaagcttatttaaaaggaacaggtgcattagttagtgactacatttatacaatctatcatacattagccactataagaacagttggtgtagatattccaaaggaactagcagaagacgataacatttacaatactgctattttataccaaattggacgtgattttgccgaatggctagacacgtttcgctactgagataaaagatttaggtggaataaaagctttagtaaaaactccctatgcaagaaacatgtggggaataatgaaagagttcaatgttttacctacagacgagaggttcagagcgcttactgaggagcaaataaacttcatctactataacaaagagagagactacaaagaggcagagagagcttctagaggcgtgtctatggagtctgaatttgaagactataactcggatgactggtgggaagcttctcatgaggacttccaagcaattcaggaaggtcatgatgaagatgacatcgctcaacaagtagagaacatgactagtatcgaagaccaaaaacgcctaagagacagattccagtctcatgaggagtacgaagagtttataagaaatggtggtaaagatactagacaggaaatgattgatgaacacatcaataacaaactgaaagaactgtttgcagatgccgaagagttaaataaacaaggcttgagtaagtggggagaagtatcagaaatgacaactatccaagaagctgtagcagacgagttagaagagctaacatcagatagtgttaaggaagcaatcagtatgttcgaagatgacgaagttcaggtaataccgggtcaagatgaagacgatgattttgaaatttaatactaaaaagaaagggtagagctgtgtactctactcttttttactaaggaagtgaaagtaattggaaaacactaactatcgcttttcggtagaagccttaacggcagatgcagagaaaaagttagagcgtatcgctagaataatggacgacattgagggaacaaataggaaaggtgcaagtaatttcttcaccagctctcaaaaagatatagacaacaacataagagctatgagagaatatgtttcattagttaatgaagttgagaaggaaatgaaaaacgcccagtcttctatgtctcgttctggaaataacgaaggtgttcaaaaattaataagagaccagcaaagactaagggaagaagcggaaaaaacacggaaaacattccaagatactacacaaaataaaacaggtagtaacgcttatcttaaagacctgttaagagaccaaaaagcatacaacgatgaaatagacgaaggtactcgtagattaaaagagttgaaagacttacaaagagatgtagccagaacagatagcagactggctgccagaacaaggcaagctacttcatcaggtaatatgaactcacaacaagcaggaacagctaggtctgacttagcaaataaaaccgcatatatgagtcaattacaagccaaccaacaacgaagagatgagttaagaaaagggctagagtccgaccaagcaaaaaaaagaaacttaaatgctaactttagaggtaacgaccaagaaagacagaatgagttgtcagcgttaaaccataatattgagactcgtagaaaagaaatagaagctttaaaagagctcgatagtcaaataaataagacagtaagtaatatggaaagctacagtaaccaattaaataatgtaaatgtaaaaccagaccgtaactcgtggcaaggggtcgcttatgaaagagcaccatctatcgctatggctgcattaggtggagcaggtgcaatatttggcggtctatatgctaaaggtagctctataaaagaccagattagacctgacactatctctatagggcaacgaacaggtaactatgatttccgttctatcagaaaacagaacgaagaagtaggtatggataggtctcttggttacaacgcagtagacatgattgatttccaaaacacctatttgtcttctgctgggtttaaaaaccaaaaagacttagatgcaggaactagacaaatggcagaagggtctagagctgtacccgtagataaagattctctaaatcagtttatgggtgggctaatgacgaatggagccgtatcaggagcagaccaagtaaaagcggtacaacaaggttttattggagcagtaaaagcttctggaatggaaggtagggaaaaagaccaactaaaagctttagaagctatgagtacccaaacctttgcaggaagaaatggttctaatgctgagctaaataacctgttagctatccagtcactgttttcttcatcaggtgaccgttctatgcaaggaacagcaggagcagaagccttatctagtctagatgcaggaatgaaacaatctgcatataacccacaagcaagattactatttggtatggggagtgagtaccaaggagtaggtggtatgtatcaagttcaaggacaaatggaaaaaggtctcggagatacagacaatttaaatagactacttaaattctccgcaggtgcaggaagaggtaaagaagaacaaaaaggttacttcaagaacatggctgagaatgtatttggtactacattaacaaatgaccaagtcgatgcaatgtataaacaagtaggctcagatggaacaatagataaagacaaaatgaaaaacatccaaagtattattgatggaggaaaagacggatacgataagaacaaaaaagggtatgaagactctaaggaagctacagaaaacgcctctaaagcagtaactgataagcaggctacagcgatgaacgacatgggggacatagttagaaaactaaacagtgttctcggtggtatgcccgcaccattgtatggtttggcagccgcagcagttgcagcagcaggagccttagctatgtctggcggaatgagtattctatctggtggtattaaaggaaaactaggtaagaaatttggaggcggaataggtaaagcaggtagcagtacagtagcttccgcaggagcaggcgcaggagcctcttctacagctagtaaagtatttaccaataaaacatggggagcaggagcagaaggagctacaaaggcttcctcggggctctcatggggtagtaaagcaatggaaggcggaatttctgcaactaaaggattaggtggagcaggaaaactagcccagtatggaaaaggagccttaaaaggtctcgggccgcttggtcttctaataggcggaggagcctcaatcgccaatattgcaggtgcagaaaacaaaaccgaagccatcggagaggaagcgggtggctggggaggcgccatgggtggtgcagcaggcggtgcagccatcggagccatgtttggtggagtaggtgctataccgggagcccttattggaggggtactgggaggtattggaggaagcaaagtaggtaagtgggctggcggaaaaataaagagtggctgggattctctatggggcggagacaaagtagatgctgctgagataaacggcacaggaggagctaaaaaagctcaaacagagaaaaaaaccaacatgaacggtgcttctatggctaactctggtgtaggaactatgctaggaaatacagtaatgcccggtgtaggaggaattatcggaggagctataggaggttcaggtatcctagaacaactttctaagctaactggtattggaagaaacgcacaagaagaaacaagttcaggaaaacaagatgaaaaagacacagtaggtaagaaagctacagcagagaaagagagacaaagtaatacaagagaagaagcagcaaacctttctttttacagtaaacttcttgacagagccgaagcattattgatgcaagctagaaatcaaaatggattattcggaaaagaaggcttaggaactactgctacaggtagcggtggaggagatgctctcaattcaggtcttaacttcttaacagatggtaaaaagtggactaacaaagatttaacaaagcatgacttaggttcaactattcaaggtattactgctgatgagttagacgaatggattaactcgaaagcacctgagggttctgcaatgagagggatgggttcaactttcctaaaagcagcacaagaatcaggattagacccaagatatttagtatctcatgcagccacagaaactggttggggaacatcgaacatcctaaaagaaaaaggtaactggtttggtattggggcatatgataactcacctatggcaagtgcctacaaatacggggacaaagaaactggtgtagtagaaggagctaaatggattgctgaaaactactacaataaaggtcaaacaacactagagagtatgcgacataacggaggacaacacgagtatgctaccgaccctaactgggataaaacgatagctagcataatggggggctcagagaaatacgtgcaaggtaaaggtgcttctttaacaacttctgctaatataactgttaacatgaatggagactcatcgaaagataagaagacagcaggaaaagcagtaggagatgccgttgctaaatcgtataaagaacaatcagagttcttctctaaacaatacaagagaacataatgttctcttgttattacatataagataaggaggataaaattatatatgactacagttaaaagaatgcctgaatttgatttaaagtttgtaacagaaaagaatgactatcttataagatatgatgcaaggaacccttcctcagatacattagctgagaaagtaataagtgtaactactaaaaatgcaatgtctgacgacagcgccgttttttctattgtagtagctggtgatatggaatgggataaaatattagactctaatgatgtagtaatactaaaaatatacccaaacttaagagtaatgatgcctgacaacgttgttgttttagtgggtctcatatccgaagttcgaagagaaggcgactactctaataactctattatttatcgtataacagggcaatcttttgctaaatccttcatgcagttccaattaggggtaattcaagaggtatctgtagtaattaccgatattgggtggttgcctgactctaaggcagatggtgttgagtttacaggaaaaacagcagcagagataggtaagtctatcacagataggtttaaaaaatatatgaaatacaattttaacagagaatatacaatggaaaacttcttagactactctttcagcagttggaaagattacgaaaaattagcagaccctaccccatttattaactatgagggtagcctaaaacaacttctagatgatgttacagcaaagccatttaatgagttattctttgagtccacaagtgatgagaagtgtaaaatgattatgagaagaacaccttttaataaagaagactgggacaagttaccatcttataaaatatctacagaagcagtaatatcagattctttagctaaaggggacacagaagcctactctatttttaacgtcactagtggaaatatggcaggagcaacatcagtagacttaaattcattccctcaatatcaccaagctttagtagataaatatggatacaaaaagttagaggtagacaacaggtatttatttgagtcctcaacagatggctctagtaccacagaaaaggcagatgtaggaagtaaggaaaaaacaaaaacagtaattacttacagcaagtttaactcttttatgcgtagttatacttccgaccaagttcgtatgaatcaaagtagtattgcaaaaagtttagtggacagctacgacaaattgacaacttctcaagccaaccaactgcttgctaaatattctgctgtaggtgcgataagtgaggcagattttaaaaaaattgtaggagacatagcggagggagacaacacaggaacagctactttagattttgatagtgtaaactcttggttctctctaaattatagttctttgtctgaggtttctacaaatagagatgctacaataaaagcttttgtaaaaaactttgctaacacagatgaagaccaagcaactaagatagtagcactatatatatcaagtcaaggagtcatgacaaaagaaaaatttgatgcaatcataaaggagtctactagttctagcactaaagacccagataacacaacaggcaactctagttcagcactacagtacttctctaaaacgatatataactggtacagtgaaaacgctaatttttatgcaggggatataaaggttattggtagtcctgtatataggttaggaagtaaactccttgtagaagataaacaacaaggtgatgagtgggagttttatattgaaagtgtctctcatgaatatagctatacagcaggttatactacaacactcggagtcactaggggattgaacaataaagggaaagatagatttactcatttatggggaaaaagttcggactttaaaggcgggttacttggagaaaaaacaagtgctgaacttatacaagaagcaggttctacctcgtcaggttccgatggaagtggcgatgtaagcgcaccagatgtacaaggtagtgatgttgccgtagccgctttaagatacgggctagctcataaaaagccagagaaaaaatcagtatattcttttggtggaggtagaggtagtagtaacccaatggagggtaaggaaccttacgcaatggactgttcttcttttgtatggtggtgttataaagcctgtggtgttactttagcaggagcacaaacacaagcgatattaggggacgataggtttaacacagtaagctcaagaggttctaagagtaaagaaatatttaaaaaaatgcaagtaggcgaccttgtttacttttacgataacaacactcatattggtatgtatgcaggagaaggaaagttcttaggatgtaatggagatggaagttgggacaccaacggaggagtacagttgaaaccaatggactctggttattggtggacacagtttcaaggacatgtaatacgttttgtttaaaaaggaggaatgactagatgagtgatgtagaatcaggagcaccacgaacacaggctagtttaggtagtgagtgggaacgagtatacaaagcaggagaaaaagtagacacaatcgtattagcccagataataaaagtaaactataaatataacaccgcagaggttatgatactaaaaaataaagaagtgatgtctaagtcgtatatttctaaaggtaagttctcagctccactacctcaagattttgctggaagaactctagaaggacttccatatggacaaattaaaccaattcaagaaggagctattgttcttatagggttcctaaatagcaagaaggacaacccaattattttagcagtctacggggactcagatgtaaataataagctagctagaactccttatgaaagtgcagacccattagaccctaagctagctagaataaataaccagttttttaacttgtacccttccttgacgtatgataatatagacggggacggaaatagaatatccacttaccccggcaagacattttctattatgacaagtttaataaacgagtcttccaacatttcagacagcaataatgggactgaatatgcagacttacccgaatcatactacacagactacaaactgatagagccgagaagccctttagcaccatcaatgctatttaaacaccaaggaataagaacactagacgggaaggaagacaatcatttagtaactgcctttataaatacagatggaacaacaaggctgtctttcctaaataaggttatgaactggcgttcctattatgaggctgatactgagggaggtcaaaagttcagacttcaaagagacagtaaggtaccaaaccaaggaagcatattttcagagtttgggttaacaaaaaacggagacttctttattgtaaatggaaaccataagtttgaagtaagagagaacggtatttttattaatgactctgctttagcagacgacttaggggaagttttagaagaaattcaaaatagaatactagggataaacgaggagattgctaatatcatccgtatgttggatgagattgatgttgagaacctaggaaagttatttgaccgtatgacaaaagtagaaacagacattattagcttaaggaatgattataaaggaatagacagtagagtgaacgatatgtcagaagaccttaacagtctgattaaatcctttgataactttaaacagacaatgtctgcctacaccgaggctaatactataagggttactgctattgagaccgctactgcaaataatacagtatctataggaaacattaataatagtattactgcgattgagcagaagaacacagcccaagatacgaaaataagtagcctagagagtaaagttacgtcactagagacaagtaatgctagcttagtaacaaaagtaggggagttagaatctgatgttactttcctaaaagcagaaaatatagatttaactaatgcactagcccaattagcagaaagagttactgctttagaaaatagctgatagtagtactatattataaaggagacgaggtggaaaaatgccacaagcagacggaaaaggtggagtattaagacgagttgctttccaaataggagagaataaatggtatcgttttgcgataaaccccggtagttactcttttacaggaggacaaagggttgctgtgtttaaaacgaagtccttaaacgtagtagaagactttggtagagatattcctactataactttctcgggaacgacaggtcttagaaaagactctaacggtaaaacaggagcagataggttaaaagagcttaaaaaatatattgaagattatgcagactcatcaggaaacggaaatagacctaatacagaccttacttttcataattttacagacgatgaaagctatattgtacatttatcacctgaggcatttaagatagaaagaagcgcagaaaaacctctgttatatgactacacaataactttagtagttttaagggatgcttctcaaccagaccaaggagacattgtagacccagacttaggtaataaacaacctagtgtaggggacaagacaaaggataatgacgatgtttatggggatgtaggaggcgcagacaagacaccagaagctaatattactggaagagtccctctagaagtaggaggtaacgggggaggggcagtaaacccccgaacaacaggtaaagcatactcctacggtgtagatgaattaaaaaaacaaattggttatggggagggttaacaatatggaggtatatcagtcatcagatttaatgaggtttattacagggttaaaagttaacccagataacactattgtagtaaacatcttagaccagcaatcaccatttatttctaggttttacactcctaagcttattctaagttctttatcagtaaagcttattcctgaattgcaaggagtaaacattatacaaacaaacaaagcattaagtaaaaatacagttacagctaaaatatcagcaagtaggttaaaaatagaggctccttacatgtatctaatcgcacagtctatagtcatggaatgctttgctctaatctacagtatagaagaaaagcaagatatgctaaggtacattgaagtaggagaagttaaacaagttagagataacataaattatttagctgactacttagctgacaaaccagaatacagcagtattgttacggacttacatgatatggaaattgctgtagggtatattgagaaccaaataccaattatacagagggagataagattaaatggctcgttataaaaaacacgtagtacaatatggcgatactatgcagtctatctctcagctagagactggtagtgttggaaactgggtagatatagtagaatataataagttaaagtatccttatattgtacaaacaaatgaggaaagaaaagaaaatcttgagagtacagttactattggggatgttattatcatacctgtagcatcagacctaacagaaatagacccttatgctctaacaaaccaagataagaacttaataatgagtttagctctaggtagggatttaagtatgacctccaatcaagattactataataaaagaggaactagtgacgagatactagaagttgtagctaacggcaagggagacttagcgactgttgacggagtagaaaacctaaagcaggcactaatagctagactattaactcctaaaggagccttgatgctacatccagaatatggctctaacttacacaagatgtttaaaaaagctacacatgaacaagcatcactaatagatattgaaataacaagaacattgttactagacggtagagtagaaacagtagaaagtctcggatacaaagtagaagaggacgtatatgtaggaaaattcctagttactatagagtctttacagacagcgtttgagttcgttgttcaaggtgatactacaggttctattattttgctagataattgaggaggaaaacaggttgagattaaaaagaatttcagaaattttatctcgtttaatagataagattatgattaacacatcagagctaaatgatttttctgtagggtctactatcttgtctatactagaggcagtttctatggagttggagcagtactacatcctatctagagagaacattgtgtggggtattcagcaaggggtatttcaggctttcgacttttttaagagagagccaaaaagagcttatggagatgtcctaattgagtttcatagcgcagttcaaagtgatgtatatattccaaaaggaaccacttttagttcaacaattaacggatacgaccagcaattcgagacagtcatagactactacgttcctaaagattccatattagctcgtgtagaagtgtattgtaaagtagcgggggaaataggtaatgtcccagcaggaactattaatttgattatgaatagtgtaagtaacgttaagagtgtcataaatgagagtaacttccaaacaggaactgatgaagagtccttagaatcagtaaaaaataggtttcatgctttcgtagagagcagagggagagctacagtccctgctataaactatggtacccgtttagtagaggaagtatcaggcgtatatgtaaaggaatatgtagggtatgtaattgtctatgcacacgatagaaacggaaacctttcagctaacatccaagataacattaaaatagcattagaagactatagaccagcaggtataaaattagatgtaaaacccgtaaacaaaaggttgctacctgtcgaggttactgtaactattactaacaaaaacagaataggggatacactacaggcacacattcaaggagtgataacagactacttaaatagtatgcaagtatctaacaacttaattttatcagagcttattcaaataattatgagtatagacgactcccttatttatgactgtaagattgaaaactttacagaaaatgttataataaatagtgaggatattattagagcaggagaagtaaaggttaacttaaaataacatagttaacaaatgagaggagctgacaactacccttgccaaactttttaaaaaatatacaccctttattaaaaagaaaaaaagatgacaaccaagtaagtagcgtaaactacgctgttttacaagcgctcgaaacagagctgagtgaagtagaacaggaaaccttagagagtaaagtacagtcatcccttggaacagctacaggagactacctagatatttggggagactggtttaatgtataccgtagagaagatgaaggagacgaacgttacagagataggattattcggcatttgttattaaagagaggtacaataccagctataatcgaggcaattcgtgattttctagaagactatagcgcagatattactatctatgagccgtttaaaaatatcttttacacaaataaatcagacttaaacggtgaagactacttgatggggtactactatcgttttgctatcattgatgttacaattggtagacggtttcctttagaagtaattgacattataaacgcatttaagccagcaggtgtaaaattctatctgacttatgacggtagtggagaaattgatgaagaaaacagtatagttgaactaccaccttatagtatagaaattgaaacatatcaagaggtagatattctaaacgggttaaatacaacacttcacgggcacttcactttaggagagtctaatattgtagagggtgttatccttgagttgtttcgaacaaatagaagtccactaaactctaatgatgtactatctggctcttttaatgcaggtagacgagtatacaatctagcaacaattggagacaactcctataaacctacgatgagttctcttattggggaaataaaaaattcgttcgaggaattacccgaagacttctacgtagaaactaaccgcttagatgataggtttgctagcatagacatggcaaataatacacaagatactgcattctatttcaacatggatttaggaacttatatcaatactaactacgcaggatatttagctgagttagcaggaagccagattataaatagagcacactatgtatccttagctaaagagccaaagttaaccgtggcgtttaaagctaaaatatccccagcaaacccagtaaattacaatatagaaggttatgacttagtttctaaaaaatgggttgttcttgagacaggagctatagcggttgaaaagaaaatatcacagatacctttaggttcattagaagactatcttaatagtaactatatatgttttttccgaattgtagtgaaacctaaacgggaaacctactcctttgaattagactatttcgaattaggatttttaaacaactttaaagaaggggagtatacaattaagccttttacaggtattgtagaaacttatgcagagttgttcggtagagttatagaagttcctatgtatcggataataaattcaggaacagaaaatattatttctaaacaaggtcatatacctgttagatatattagagatactttagagggtggtagtactacaaacactgctaacctatggatagaaattcaggctaaaaataaagacaatatcaatgttgcgcttaataagcctgtatatagaccacaagctgaacctacaaatggtaatattcttctatatccacagttccttaatattggagctacttacatgaatggtaagtggagaggaaaactaactagtgaggtaaacatggcatatactgtagacggtgtaagaacttacggaggaaacatgaacgcagatggaacctatacagtctatatcgggaacaaagtaacctctactagccaaaaagtagttgtggaggtatatgcagacactgcaagaacgaagttaatagacaaagctaatgttcctattacaaatgcaggggacggaagccctatagaggctatcacagacggagtaattagtggctctaatatattctatattgcaccaacaagcgccaaatactcagtaactgtagacttagaagctacctttgtagacttatcagaggtaaggatatggcactacccaacacgaacgtttaagtctgttttgacagaagtatcagcagatggtgtaaactggactactatatataactccgctgttgatgggacttatctagaaactagtgaaggaaaagcgtttcctgtaccagagatggacttatatggattgaaagctactcctctaacaagtgtactagcaggttctgaaagacaaattattacattcctagactacaaaatagaggactttgagaacccagctaatactattgaaaacctaaacaatgattactttaacgcagtgtggcaagagattgaccgacttgaagagtttgttctaggcaagttcactattttagctgatgttggctctgacggagtactagtaaatgactggggtcttattgaggctaaaatacctacaggtgctacagcataccaagaagcggacagtagaagtgaattgtcttccgtgtacactgtggagctcacagcgggctctcacgaggttattatggatgttgctaataaagttgttactacagaggcagtaaccctaaaaacggattacagggtgtctaacgagcttaccttggaagggctacctacaaaaatacctgtgagatacattcgagactccttgtggggaggaaacagtataaatacagcaaaccactgggtagagctacaggctagagacttgaaaggggacaatgttgcccttaataaaaaagtggtagagaacaacggagttagcttactatctcctgaaaatctaatgttagactcgaagacagagattactagtacaacaaacaatggttatacttttgcttctgatatagcttatatatttgataattatggagtagataaagagtatactattagttttgacatcaagtccactacggtaggcacagttactgtttatagtaggtacaaagatgaagctaaatattctataggaaacaaaactgttcctgtaacgactgcttacacaagacagcaagttgtagtaacacctactagaaacacagcagaacaagctaaatatgcagtcagctctattctaggttttcttaatagtacaggagcaatatctacagttaaggatataaaaatatcagtaggaaacatctcgaatatgatttggacacctgcacctaaagactacagtattcttactgatggagtaacaacaagtgctaacggattcttgagtggagttaactccttaactattgacttaggtagccctagatatgatattgacaacatcactatgtggcactattggcaaggggctgaacctaggtactacaaatatgtaaaactagaagtatcagcagatggtgtgaactggactacactgtttgatagtgatgtaagtggaacttattacgagactagtggaggtaagcaggttagcgtacctgctataaaagaataccttaagagtactaatgctatatttgcacctagcgtagcaggaacaggtacttttgagagagtaaccgaagcaaacggagtaagttctataaaagtgacacctgatactgataagacagctagaatgtacaagtttttcggaacttctacagaccctaacgctaattcgagagaccttctagaatcagggctaactacaacgaagaaatacacagtagcagtatccctaaaacctcaaaataatagttcggttatcttagcggtaaaagatggaagtaacactaacatagagtcgggctccttaaatattgcaaaagatgtgtggacaagagtttcttacaatgtaacgttacctgcaacagctattgataggctagctttatgtttgtacaatgctaacacaggagaatatattaattataaagattggagcattaaagaaacgatgtttccataaagtaaaaattatggtataatagagatagggcacaaagtagtgccttatctttgctatattagtaatgagagtacgaatacaataccgattttaaaataaggaaatgaggtaaataaatggcaatagcaacatataatgcgcatgtatatgcagcactaaacctaaaaagtaaggtcgatacaacatttatggcaattggtaaaacaacagcgtggactgatgagactaaccctccagaaccagacccaaatgctacagggttaacagaagttatcggttacaagaagctaaaaactatgtctctatgtagaccacaaagaacaggtgaaacaccaacactacctacagtttcttacggaaataaaacatgggttcttgtacctgatgcacaagcttatacagaaggagctaaatggctttattgtgaggcagagttcgtaggggatgaactaccagtaggaacatacagacaagtaggtgtatttacagacttagcgccaaaatctggtgtaacaaaaccgaatttattaccaagtgaagtagctaatgtaggtgtactacagttttttgagaacaagcagttccaaaacagaacaccacaagttacagctagagaaagattcgtagcggaattataagaaagggtgaataagtttggcagatttcaattttagttcagcaccatatcataataggttcaatgccttgaatgattggacattagcacttttccaaccagacagaccccttcaacagtccgagttaaacgaaatgcaatctattttgcattattacctaaaatctgttggggatgctgttttttctgatgggaacatgcaaacaggtatggagtttgtcttggataactcaggaaacttaactgttggtgatggtcaagtttatgttgagggcaaagtaagaaacttcaaaaaacaaactataaaattaaataagataggtagagagtttgtaggagtaaaagtagaacagtctattattaccgcagaccaagacagtagtcttctagaccaaacagcaggagttcctagtgctttttctaaaggagctgacagactaaaagaaactgttgttttaacgttagacgaccctcagtcagcaactatctaccagtttgataacggaaaactttttatctcctctgataacccagagatgacaaaaataaacgaaattctagctgaaagaacatacgatgaatcaggctcttatcgtgttaacggttttaacatgtggacagaacctcatccgacagacactagtaaaattcagttagttattgacacaggtagagcttatgtgttaggatacaaggtagataaaccaacatctacacgtatacctttagataaagctagagatactcgtagtgtagccaatgaagggcaatactacgatacagcagttaaccgtattaagctaggaaacagtcctgtagaaacagtaacacgagttacaggtcaagttcaagtacctaaggagcaagtgagcagaggttctcaatcaggaggcaccgacttcttgagtagaacttccgtattccagatagaccgtgtgtggacagaaaacccaacatcaggagaagttattaaggaatacactcaagggacagacttccagttaataaatggacaagcggtttcatgggcacctaacggagcagaacctacggcaggagcaacttactacgtgtcttaccgttataacaagtctatggtactgaatacagactacaagataactattgaaggtacaggggatacaaaaacttggtatattgattttagtgatatgacaggaagtaaacctatccaacaaagtttagtaaacgttgattacaactactacttggctcgtaaagacctaatagtacttgatagatacggagaagttacagtacacaaaggacaaccagataacttaagattagcggaagcacctgcacaggtagacccctcaacacttcaaattggggtagttactatttaccctgaaagtgagaccgctagtgctatgtcattctctgttactcgtctttctatggctgatttacaaaaaatgagaacacgtattgataatattgagtataacgaagcagtaaatgctttagataatcctgcaatggcaggagaaaacccagtgacattacgttctgtattctctgacggttttatttcactagacaaatacgatagcacgcatccagatgctattatagcttttagttttgaggatgcagaaattaccctaccttaccaagaagttaacaaggtagttccaaaaatgttagagaacgcatcagaagcttatgtttggggacgtttagtatcagcaccatttacagaagagaaaacaatttctcaaccttttgcatcagaagctatgaacgtaaacccatataatgtgtttaataaacaaggggttattactttaacaccttctgcggataactggattgaggaagaacgagtaactgtaactcaggacgaagttaagtcttatacggtaaaccgttggtggctacatggaggtaactactattctgatgaagctagatacttgcataacaacctagcactagatgcagggcagtcatggagtgggactccttctcagaacaaacaagtaacaggaacaacattaacagcaggtggacaacaaacacttgagtctatgatagagtttatgcgacaaattgatatagtggtagaaggtaaaaactacttgcctaacgctaacaacttatacgcactattcgatggtgttcgggtacctattacaccagcttctggttaccgaaaaggtacagacacaggaacattaatggctaatgcagatgggacagtaaaaggaactatcactattccagcaggtatccgttgtggtgtgagagaagtaactttacggaatgcagataacttaggtatttctacattcactgcacaaggtagaaagaaaaccgtgcaagacatcattatcagaactcacgtaacagctaacttgtatgacccgttagctcagtcattccaattcacagatgaccgagttataactagcttaggattacattttgctagtaaagatgcaggagaaaacaacgttatcgttcagattagaggtatgagtgacggaggacaaccaaataaaactatttatgcagaaagggtattaaccccagctcaagtaaatgtgtctgctgacagtagtaaggaaactaaagtcacttttgatgacccaattatgtgtaaagcaggtcaagagtactgtatcgttattgtaacagactctgatagttacactatgtggttaggtactagaggtaaacctagaatagactcacctagcgacttaattacgtcaaacccttatgtacaaggtgtactatttagttcttctaatgcttctacatggacagcacaccaagattctgatttgaagtttaatgtatatactgctaagtttaacgaacaagctgtactagaatttgacacaatgaaaaatgttaaagttgatagagtagtactaatgagtacgtacctaactcctaataacacaggatgtatctgggatatgaagattgttctagacaatgaacctacaggagttacagtagcagacaaagaatggcaaccaattggtaactaccaagatttggatgtagagcaattagctagagaagtcaagttaagagctacttttgttgctaacaactacatttcaccattgttatcattggacgacatcatgtttgcaggtttcttaactgctttaaaaggtagttatgttagtagaaatattaatatgacagactctccttataatacagttaagattagttacgaagctttcctaccagctaacactacagtaaaagcacagtttagtactgatgcaggtattacatggaaagacttgacgtctattcctgtagtaacacaacaaacagccgatttctacaagtatgaatacgaagagaaagtcatccaatcaggcacatataaaggatttaaggttcggctgaacttagcttcacaaaacagtttcttaagacctcgtgctcggagactaatgtgtttaatgaaagatgaataggggaacttttgttcccttatttttaactaaaaaggggcggtaaacatgccagagtcatatagagacccccattcaggggctttaaagtttattcctacgaaacaggagaaaataattaaagatgtgactgaaaaactaagagaagacgaagaaaaaattcgaaagaaaatagaagagttagatattaaactagcacaggcagacagcaggttgcaagaaataactaacttgtatgaagaattaaaagaactaaaggagggtaactaaatttgtctagatacgaccatagtactgtagagtacacagacaaaattaaggacttaacagattcggtaaaccgagtaggaaactacttatcaggagacaatagcccttatgacgatgttcagaaattaaaagcaattacacaaaatattaagctaactaaagatacaggtttagctaaaagcattacagcaggaacaactgcgttgaggtcagtagtcgaagtaggggtttactatattaatagcacagaagctctagccctaactgataaacccccagagctaacaggagcctttatactagtaaactacccgacaacagcctctacttctgttaagcaagaagtacacatgtttgctacaggaacaacaggtagctacgtagggtaccgatggatttctgcttctagtgtcagctcatggtggacttatgaaaacacactaggttcacaagctaaagcagataaagcattagcggacgggaaaacatatactgatagctcagtaaactccgctttgcaagcaattcaaaactcagcacaaatgtacaagttaacagcggatgatggtaagcctattgatgcttctgctatggctactccaccaactagtgtagcttctctaacgaagacaggaatatattactttactgccgcttttggaaatacaatgccagataccccttgcacagggcaaccattctggttggttgttttacagcatgtaacagataacagtatatcacaatctgtgacagccaacactgtagcagtagagagggtagtcgcagataggattattactactctaggggtaccgagtaagtgggaatacagagctaaagcctctaatttcttctttagtgcttccaattcgtcacgtataacactagtatcgacccaacagaatattataacacctaataaatttattaataaccccggtagtttaccactatctgcgataaaccccgcaataactattccagaagatgggatgtaccaagtgatggcggccctaaacattaacggtattttagaaaaagtgtacctagtaagtgagctaactctactaataaatgacgtagtacacccagctacatttggaatggtaaaaacagcagataatgctaacgggcagtactctttagcagggaatggcatatatcaactaaaaaaaggggacaagttaaaactaagaagttactgtaacaatacagggaataacccttacttggatgtagacaaactttatatatctgttggtaagattgcagacatttcattatttaaataaacttagtatgctgagaaaggaagtcacaataatggcagaagaaaaattttcattagacggaaggcagtatgtaacagcagtcctatatatgtaccctaacctagaccctcagtacgactttggagttgtatcatccgaagggaaggtagaggcttttgaatggaacgtaaaaaatataacaaagccttctattgcacagttagaccaagcatataaagactatgtaaaatctatcggaagtccaagtaacccaccagccctagaaccagaggtctcaaaagaagcctcaataggtaagattctaactgaaaaatttagtgcgttagaggaagttaatgctaatatgttattggaaaatttagagcttcaagctagcctagataaaacaaaacaacaaaattcagaactagtatttgagctagtagacaggggagtcttatgagtgtaaattggtatgagttagttttagattactacacaaaaggttattacacaccaagtcaggtaaaaagatttgctgaggtaggaaaactaactttcgagcaagccgaagaaattatagctacaaaatcatagagtatgtttaaactatataggtaattaagactaggactaatatcctagtctttttatgttataataaagatatagtaaatatatttggaggagtacattaatgagaattgttgtagatataatgaatacacgagttttattcgataaagaagaagaggcaattaaaaaaagatttcttagggatatgcaccaaaaattaggggtaaaagtagatgggttcttctttagcccagcatataaatcaggtcattgggacggtatccaagacttctatgactttaaaacaaatacgttccctacagggctgttgccacaagtgcaagagatactaggagctatgcaaaatgatgccgtatttcaatttgagattattgatgacagaccagacccgtttttaacagtagaggacttagatagtgagataactcttataggagatgacggaggagatatacagcttagagattatcagtatgactctgtaaagtctatcattgaaaactatagtgggattatcaatattgcaactaatggaggaaaaacggagattgcttctggtatcattcagcagattttaccgcatttagagtctggggagagaatagccttttttactcactcctcatctatatttaaccaatcaattgaccgtatagaaaaaagactaggtattaaagtaggtaggctaggaaatggtaaaaaggacatcagacaagttacttttgtaatgatacctacgttaacagcctccctaaaagaccccgaagttggtttgaaattaacgcataaggaacgagttattaagaagttagctaaagaggtagttcctcgtttcactatgcaaaaaaatcatagaaagcttcttaatatttatattgagaacaacagaccgaaaaataaagtagaagaagatataaaagctcgactagaagacttgttatattcctgtggaacagactccgaagttctaatgggactaaaaggttttaacgcagaatatgaaaaaattattgagaaaaaaaataaaaaactatatgacaagaacaaacaagctagagaatttctatcatctgtatctgttatgattgcggatgaggcgcaccataccagttcagaaacttggtacaactctttgatgtcttgtgagaatgctatgtacagggtggctttgacaggttctatagacaaagaaaataaaatgttatggcagagaatgcaaggagtattcggtcaaattacggctagaacaagtaatgagaccttaattacacttgggcactctgctaaaccaacgattaaaatattccctatcaaggctcctacagatattgaaaatgtagataactatgctactgcttatgaaaaaggaatagtagaaaatgattatcgtaatttacttattgcaaaattagcaaagatgtggtatgataaagacaaaggtgttcttattatagtaaatagaatagagcacggtaacagattatctgaacttcttgagtctctcgaagtgtcgcactacttcattcatggagacttagacatagatttaagagaacaacaactccaaaatatgagagatgggaagcttaaagtgatgatttctacatctattgtagatgagggagtagacatctcaggtattgatgcgcttattttaggtgcaggagggaagtctttaagacagacacttcaacgtgttggcagggctttacgtaagaagaagcaaggagaaaacacttgccaagtatttgactttaaagatatgactcatagatttttactaagtcactcagaccaacgaagacagatttacgtagatgaaaactttgaaattcaagacatttagggggaagaacaagaatggatttagtatatgtaggatgtcagatggaaataggaatgaggaaaggtttacgagactttctagtggatgttgctaaaacagctcagcaatggggagaaagcgtggctatggttactcaaaaacatagcctagctgagcaaacaggagtaaacattagaacagtctcacgttatatagctgaacttgaaaacaaaggtattattaaaacagaagcaaaaagaggaagaaacggtggactagttatcatgtttaataaaaatatggttacttttaaattaacagacaaccctataacctctgattcacctgatgcggtagaacttagagaccgtatgttccctaaaaagaatgaagacccgaataaagagaaaagacctcgtagaacaaaagaacaaatggcagaggaaaaagtactaaaaaatcgtaagagaagtcttaataatgagttaaacgatatgttagacgatacattattccctacagaagagatatttagtaaaacagaggagcctgagaggtactttaaagcttatttactgtctcgaatggttaatgcttatgcgtatatttatcctacagagagagagctagacatgctaagcagaggggtgcaggtagaagcagagaaggcacgaaaaaatagagtacgagcttactcttttaactcactacctgctagattttttgggacatctcagtttacttgctacttaaaactacaagacttattagaaaaagagaacattaacccattatcttatttaacagttcaatttgactacttagactacagattagataacagtcttggaggaactccactagcatatagaggtatccctgaggcaaactgcttaataggtgagactgcctttagtcgttacacatctgctaaggacttctacactgttatgcgaggtaaaaacatgttacgcaccagtacagcagatgttgcctttaaaggtggaaaataccctattgtggaggcgttattacacgcttacaatgtaggtaagcaagatattactgcactaaaccaacaaatagaggatatgaagacacaagctagtgtgttccataaatccgaagccctagcagacttttatgaaacaacaaaactattagttgaagaaagtgctttagaaacaaatgaaaaagacgagctatacaaatttgttactgaacaagttgttttacactctcgtaagaatgcaatgagtcttagccaacagttgctaagcttcccacttcaaattcaacgtgtaagacagacagccgaattaaaaggattagacaaaaaggactactatacttttgtaggcaacttagagaaacaagtgaaagtaagtagaaacacgtttaactcgtttactgaaaaaggtaagcttttacacgagtctctagaggcaaacagtactttttatagtaccattcgtatgctaggagactaccgaggactaaccgtaaacatcccgttactcggagaagctatcaagaaattaggagaggaacatattcctctagattcttatggaatgttagatataacacaggtttacgacaagtatttaacagacctgcaaatgcaagaagatgaagagaactatgaaacacctacaagttttattgttaagtaaggaggaggagatatacgttgagttccattattcaaaagcaagttatataccgagctattagagaccctttatttggtaaggatgtgttagcacagttacctagtaatgtatttgacgagaatgagcactacaaaaatctagtaattattattaaaaagcattaccagacaagtaacaaggcgcttgatgaaagtacgttgctaactattacggaggaacatttagttagacagaaaaagagcttagaagtacaggaaagctttaatgagacaattcatgatttgtataaaattgatgaactaggtatagacgatgaggctgtcggagaaagcattaaacgattcgtcaaaaaaaatctggcagctcacttgataaaggagactttgtctaagggtgaagggctagacaacgaagaaacattagaaaaactatacacaggtatcaaagaaatctctattttagatattaaccgtagtgaaaacactttatttagtttctttgatgatgtagaagccaagaaaaagatgctacaaagtatccaccaagataagttctctacaggattcacagacttagatgttattgctgagggtggtattggacgtggagagctcggtctagttattgcccctacaggtggtggtaaaactatgtgggcagtaaaccaagcaaaaaactatgttgctcaaggtttaaacgttttgtatgtagtgttagaggaacgattagaacgtatgattttacgttttgaacaaaatatgatgggtatccctaaatctcgacttatgccagataatgaacttgacgaagaagctttcgacaaattacaaacgatttatgatattaaacgtaaacaaggattaggtaatctttacattgaaaagagaatgcctcaggaagttagtcctacaatgttagagcaaattatcgttgatgctagtgttcgtaaagggaaacatttagatgtggttattattgactatcctgacttaatgaaaaaccctcatgaaagagataatagtgagtcccgtgcaggaggaaaactatacgaagaaatacgtaagttagctcaaacctataagtttgctggttgggtactctctcaggttaaccgcggaggatattcagaggatgtacttactgctaaaagtatcgagggttctaaacagaaactaaatgctgtggaaattgctatgaccttaaaccaaaaacctattgagtttgaaaaaggttttattagagcatatgccgacaagattcgtaataataacggtaaatcttataaccgtatgcttagctttaaggttgttccagaaacaatgactatcagaaatataacacctgatgaaagagtagaacacgatatgattttaaataacctcgaagaaaaagaaggtcgtgtatttaaaaatacgaaaaaggctacacctgacccccaagcagttgtagacgaagtaaaagcactaaatagtagattataaggaggagatagtttttggataaaattatattttttactgattttcatgcccatatttttaccgaattttctaaaccagatgtggagtatgaaacagacagatttagagaacaaatggaagtcttacaacaggtatttgacttagctagggagaataaggcagatattgtttttggaggagacttattccataaacgaggagctgtagatactagagtactaaaccatgtttttcgtgtatttgaaagaaacacggatattgtagttcacctactgagaggaaatcatgatagctttacaaactcgatgaacacagagagtagtttggagacacttagaggtttacctaatgtggaagtttacagcaacccttctattgtagagactgaaaaatataagttatatatgctaccttatggggatgaagtgcaagagatgaaagactggattaacagtttagacattgaagcaggtaaaagtaatatcttagttgctcatataggggtagagggagcgttaacaggtaaaggttcccacagactagaaggtgcatttacaacaggagatttaagaacaagcgactttgatgttattgctcttgggcattatcacaagagacagctactactagatgttcctaatatattttatggggggaacaccattcagaacactttttctgatgaagggcaagagaagggtgtatggctaattgactgttcagatgttaaaaatacaacaagtacttttattcctattagtagccgtaagtttattactattgacggagataacattcctgatgatatagaagaacttattgaacaaggcaactttattaggtttattggaagtaaagaacaggctaaaaaaattgacttatatggagaagaaagtacaggtattagagtagagctacgaaaagagtatacaactgatgttcgactagggattgatgaaaagtcctctcccgaagctatttctaaagcctatgcagagaaatttagtccctctgcaacaagtgaaatagtagagtgtataaaagaggcacttgtgagagaaaaataagcctaaagctgaggatattcctcagcttttcttattgacttttttagtaaagtatagtatactaaagttacaaataaaaagaaggaggaaaagaatgttcacttttaaaaaagtaaagataaaaaacttcttatctatcaaagacatggagcttaatttagacaaacaaggtctagttctgatagaaggaaagaataagacaaatgaagcctttaagtctaatggtgcaggtaagacaagtatgattgatgctattacttacgctatctttggtaagacagtaggtgggcttaaatcggattcagttgttaataacaaagagaagaagaacactgctgtaattttagacttcgaagttgacaagaataaataccgaattgaacgctatagaaaagacaaaaaagagggtaatatagtaaagctctaccaaggtaaaaaacaatattacaaagagtacagtggacaacacagataaagagattttatctctattcggtattgattttaatacgtatgtaaatgctattatgtacggtcaaggagacatgcctattttctcccaagcaacggataaaggtaaaaaggagatactagagagaattactagtattgacatctatcagaaggcacaagaaatcgctaaagagaaagttaaagagaagcaagctgagcaacaagagatagaaaatgaacttgtaaaatatgactatgagcttgtagctattgacaacttagaagagcaagaaaaagaaaactataaagcagtccagcaaacaatagaaggtatccaagcaaccctatcagtagctagtacagagctagaaaactttatgacagaggcttaccctaagaaggcagaattagagaatagtattgcacagtttatagtccctcaagtagataatacaaaagttaacgagctaagagctgacttaacggaggcagaacgtaaattattacaaattaacagtattaaagaaggttttgacagagacttacgaaagctagctaaagactatgaaaacctttctagtagcaagatttgtcctacatgtggaacagacctcggggtggatagtgaagcacacattgcagaaacaaaagcagatattgaaaaacaaatgagtgttcttggtgcaaaaacccataaaattttagtagcgttaccttctgtaaatacaactattgagagcaaaaaggagctcgtacaacaagagatagctaaaaatgaggagatacaaagaaattacaatcagctagtgtcaacagtcaggggatatgaacaagaggtacaacaactagaaaacacaaaaaatagtttagaagagaaagtggcttcacaacaaacatatttatcacaggtacaaagtatgcctaagccaaaaagtagagacaaggatagagaagaagtacacaaaaaaatagaagcaactaagttgcaagtgctagacaaacaaaaagagactgagaagtatgaggaagcagtaaaagccttctcaaatgctggtattcgttctatggttcttgacttagtaactccttttttaaatgagaaagctaataaatatttaacagtgttgtcaggttcagatattgaaattaattttactacacaagttaaaaactctaaaggggaactaaaagataagtttgatgtagaaataatcaatggtagtggaggctctaactataaagctaactctgctggtgagaagaaaagaatcgaccttgcaatttcttttgcaatccaagatttagttatgagtaaggcagaactatctacaaatattgcattgtatgacgaatgttttgacggtctggacagcataggttgtgagaacgttattactttgttaaaagaaagattagaaacagtaagcagtatatttgttataacgcacaatgagaacttaaaacctttatttgagaaggtaatcactgtgcagaaagaggatggggttactaccctaataaactaaggatttggagagctataaaatgaaagtaaaaattatgacagaagaaaaaggggtactcacctcagaagtaagcaagaggtcaactaagaaagaggctatctactccgcaaatgtttttcctattagtagcttgtacaagtggtacccctttaatactaactacgcctatgtagtagacaagcatggggtatacagactcactgaactagatgactgtattcatacatcgtataacttggtaagtgaagaaaaagctaagttagacccgacaaagtggcacaataaaaccgtaacacaaagcaacttattactttgtagactagcaaataagctagggataaaaagcattaaagatgattttaaccaacaagatgtagaaaacggatttgaggttatcagtacagagttagcctatatcaaagaacaagcgacagcacgttgctacggggataaagatgtctttgaaagaagtgaactactcaagaaaagtaaagaagaaagagaacaagcatactttgagtacttaactgtagaggaggtaaacttccaatataagttaggagagctatacgctatgcttgtacttatgcggaaggtggtaaactactaatgtttttagactacattacaagtgagctaggaacacataaagatatagggcatgaattacggttctgttgccctttttgtggagaagacgactacaaattttatgtgaaggtatcaggggacaaaacagaaggtctttggcattgtaaaaaatgtaatgaaaaaggtaaccctgttacttttgttatgaagtatcttaacattaattttaaagatgccttagatatactagaggtgtacgactatgagatagacgaaaacagcctatctagcagagacgtaggggtaacagaggaagagtttctgcttatactattaaagaaatctagtgagcctgaggaagtagaaattagtgaggaagtgactctcacgccacctaggctacctaccaattttaaaagactagcagacaacatgaataaccccgaggcattaccttttctaaactacgcacactcccgaggtataactttagaccaactattcctacacaacataggatatgttatagaggggcaagtagaaaaagtagatggtaatatgatgacacttagaaatcaccttgtttttctaacacatgggaatgatggacaataccaatattggaacacccgagccataacacctatggggataaaagcttttaatgctcctagcaaacaggacgagtactctaaaagaacaactgtttttaacctaaatattgctaagaatacgccttatgttattgttcacgaaggggtaattgatgccttaacaatgggagagtcgggagtagctacttttggtaaacagataacaagagaacagatagagttactattaaataatgttagagaagaccagcctatttatttattcctagatacagaggcttatgaagttactactaagatagctagcagtctttatgaaaagcataaagaaacctacatcgttatccatggagatgaggatgccaatgatatgggatatgaaaaatcttgggactgtttaagaaataattcagtaagagctaatgaggaaggtataagtatttttttagtatcttgcattcagtaataaagtatgatataatagagcagacaagtaagaaggaggaattttattgagtaataaagtaaatgtaaaagttaagttagagggaggagctactatgccctcacaagggtatgataatgatgtagccttcgatttatacacatcagaggatgctattatttgccctgatgcagtgggagctacactaattcctacaggattaagaacagagtttgaccccactaagtttgggctattcatcagtcctcgttcaagtattatgaacttacctttaaccctagctaacaacacaggaattgtagaaggaacatatcgtggaggtattggtattcctttaaaaaatacgttgtctatgacaggtttttctgattttgcactaacatggaacccagacaagaaggtagtagaacgtatccctgtagagaagttacctacaggtaaacttagaagtgcggaaaaaatgttttacacagaccaagcattattgtatgataacacactaccagaagaactagcagaccaattgtttgttactttggtgccgactgggacattctttttaccaaaaggcttccgtattatgcaagcttaccttctacctaaatatcatctagacctagaaaaaacagacaaactatcagacagccctagaggggataaagggtttggcagttcaggacatggaggaaagtgattcctttagaccctattatccttattgccgactctataggcataggttattcaaaaggaaatataacactgtctttaaaactacgggataagtatacgggtaccgaggtagaaaaaatagctttattagccgttggtagcttctcagaaaaagtaacgcttgttagtagtgagctagtagataatgtgtataaaataaaagttaagatggaggaattatgaaagattttactcagtattttaaagaagtacaagatgcacaaaaaagagaggactctgaaactgtagaaagacttgcagtagaatatggagataaaactattacgtataaagaacttagacatacgttaggttccgtcttgttgccactaattattggaacaatggaaatcaataagcagttggcagacacctctttagacatcgttatcaatatgttagaaaaagagaatgtattatcagaggataccttaattgatttaaggcaactaattactagcatgaaacaatcagtagaaggaggagaaggagaaaatgagtaaagaacaaaagcagttaagtaaagaggatttaactttatgggttaactttgtagtaactggtaaggttgaaaaagggacggagaaacagctaaatcgtatttctaaacgagaggtatctgtaggagatgtaaccaccctaatccgtgctcttacaagtcatcatgatgcgtacattagttctcttattgaagctaattcagttcttagagtggtaatgcgagatgagttaaatatctcagaagctcaaatggacaaggcgttaaatacctataagcaacaagtaggtacagcactccaaaagacacatgatgctatcatggaaaaagcacaagaaaatgtggagttcacagaagaagaactagaggtacagaaaaaactagaagaatatgatactaaaaataaaaaagaggaataagaatggctagtacaggaagaggaagtcaagtaaaaggttctaaatttgaaagagagaccgctaaagctttatctgtttggtggggagctacctttaatagaacaccgggttctggcgggctaaaatgggcggaagaaaacaatgtaggtggcgatatagtagcccctccctccgcaggtttccctttcattgttgaatgtaaaaaacatgaagggtggacactagacaatgtattccttaatacaggagagcctaagtcttggtggaaacaagtagtatcagatgccacaagaacgaataagatacccttgttaatgttctctagaaacagggctaaaagctttgttatgctaccttatgatgacttgctatataacgtattacgtgataaaagaagtacagtagctagaactacagtgtcatttaagaatatcagggaagaaagtgtgttctatgatgtaatggtgttcactatagaagagttaactagcttctctcctgattttttaagagaacactatagtaagtatagttgggatttcttttacaaagaagtggacgagaactctgtagaagatttagaaacaggtatagataaaatgttagacaacctagaaggttaaaataaagaaaaacactaagtaggtcttttgtggtgacatatacataagcctacttttagtgtattgtgctatactagagtagaacggaaaaacaaaatatgtttggaggaatatcgttgacaataatagtaaaagacaacggaagtagaaaactaccttttgaggaagacagactaaacaggtacctagataacataaaaaaagagtttcctaagttaaatatagagagttatcgaaaaaaggcaatacactatattttaagcaaggacgagtattctgtagacgaaatggtagactacctaataagagaagcagaatctcgggtagacgttaccacacctgaatgggagttttttgctactagactatatttacaaagattgtataagaaagctagtaaaaacaggttttacgatgtaattatgaagtatggttcttacgtgggtttgcatgagagtctagcacaaagaggaatatattccgtagatattttagagaagtattcaagagaagagcttatggaagctgaaacgtttattgacagtagcaaagaccttctctttacttacaatggtttgtacctactagctactcgctatctagcaacagatgaggataagaaagtatttgagttaccacaagaacgatggttaactattgctttatttttgatgcaagatgaaaaagaaaatagaatgaagttagtagaagaagcttattgggcgttaagtaacttgtatatgacagtagcaacccccacattagcaaatgcaggtaaagtaggcgggcaactatccagttgttttatagatacggtagatgatagtttgcaaggtatttacgatagcaatacagatgttgctagagtttctaagcaaggcggaggcgtaggagtctatataggcaatgtaaggtcgtcaggagcacctattagaggagttctcggtgctagtgggggaacggtaccttggattaaacaattaaacaatacggctgttagtgtagaccagctaggagcaagaaaaggcgctataactgtaacgttagatgttttccacaaagatatagaagcctttctggatttaagactaaacaatggagaccaacgtttacgggctcacgacatttttacttctgtttctattcctgatttatttatggaagctgtagagaatagaggggaatggtatttatttgacccttacgaggttaagcagaaaaaaggttggtacctacaagactactatgataaagaaaaaggtagtggtttattccgagaaaaatatgaagaactcattcaagatggcacgattagtaagaagagagtgaaagctcttgatatgatgaaacgtatcatgattagtcagctagagacaggaacaccattcatgttttatcgagatactgtaaatagagctaaccccaactcccatgctggaatgatttatgctagtaatttgtgttaattctatggcacctttgggtagcaatactcatcgaaaacttttcttaaacgggcatagctgaatacgatgaataagctggtaagagagcctaagtccttgagtgaggatagaggtaatcccgtgctaaattataataaaatctttattgtataaatgcctaacgactatcgaaaagctactttttagtagaacttagtagagtacactcaagtgagtggaaacagaaaagactcccatgtggagtatgatatagtcttttctttacagaaatgtaaagcagttagtacactaacgggtagggaattaacgacccttactgaaagcaagcaagaaatcgcacaaaatatgtcagctacggtagttacagaggaaattatagatggagataaaattgttgtaacaaaagaagcaggagacttcgtagtttgtaacttgtcttctattaacttaggttctgtattttccgaagaagacgttatcgaccgactagtaaccattcaagttaggatgctagacaatgtaattgagttaaacaaactacctgtaccacaagctctaattactaaccagaagtatagggcagtgggattaggaacttttgggtggcaccacctactagctaaaaaaggtattcattgggacagtgaagaggctactaattttgcagatagcctatatgaaagaattgcttttcacactatcgaagcaagtatgaaactagccgaggaaaaaggagcttacccatactttaaagacagttcgtgggatacaggggagtacttctctagtagaaactatactagtgatacttgggttgacctagctaaaaaagtgaaagagaaaggtattagaaatggttacttaatggcagtagcacctaatatgtccacagcgcagatagcaggctctacagcctctattgaccctatctatgactctttctattatgaagagaaaaaagactacagacgacctgtgatagcccctgagttgaacttgaacacttacgctttctataaaagtgcttacaaagtaaaccagattcaaagtgttaagcagaatggtaaaagacaaagacacatagaccaagctatcagttttaacttctatgtacctagtggcattaaagcaagtaaactacttgagttgcacactacagcgtggagtgaaggtctaaaaacaacttactatgttcgttctaatgacattgatgtggaagaatgtgagtggtgctctagctaacagctattgacaacatgacccttttatgatatgcttatagcataaagtaagagggtttttccttgttgcagagaggtgtgagaaataattcaaggtatttgacaaacgatttaatgtgtggtatacttgccctaaatgtacaggaaaactactaaaaatagaggaggagaactaattgttaaaaaaaataaaaatattagaaccactaaaccctaacaaatcaacagcgattattaatggagaggctagtggtatcgtaaactggaacgatattcggtatccgtctttctatcgagcttacaaagagttaactacaaattactggttgcctgatgaggtagatatgaagagcgatgccaaacagtacaaccacttatccgaagaagagaagtatgcgtttgactccattatcggacttttagctacacttgactcaccccaaacacgttttatttataacattgcagagtatgtcagtgatgcctccgttcatgctattacagctatcattgcacaacaagaagctattcataatgagagttattcttatgtacttgcttctattactaacttacaagagcagaatcgagtatttgaggaagctagaacacaccctacgattattaaacgtaatgcacctattatgaagtcttatgacgatttcatgaataacaaaacacctgagaatatggtaaaagctttagtacagtcatctatcttagagggtattaatttctacagcggttttgcttacttctacaacctcgttcgtcaaaacaagatgacggggacaggtaaaatcattagcttcattaatcgtgatgaattagcccattcgaaatttatttctgagctaattagagctattgtaggggaaaaccctgaactacaaacagatgaactaaatgactacgtgtacgcttcttttagacatgccgtagagctagaacaagcgtggtctagtgaggtactacaaggcattgaaggaattgatgtggaagaaatgttagactatgtaaaatatcgtgctaataaaatgttagggatgttaggtcttcaaccaatgtatcaaggtcacacagaaaattctatgacatggattaaagcttacgccgataactttacagaaacaaaaaccgactttttcgaaatgcgtaattctagttacaagaaaacaaatttagacaatgggttcgatgacttatgagggtagggattttatattcttcattgagtggtaacactagtgaactagctcaaggaattaaagataacttggaggaactagcagtagaggtgaaatcttttcatattgaaagagaagaccaaatgactaatgaagtaatcccttatctatctaattgtgacctgattatggtaggtacatggacaacagattacggagtcatccctgaggagatggaagaggctttagaaaagtatgactatacttgggtagataagcctgttgctgtatttggaacaggagaaacccagtggggtatggaatacttctgtggagctaatgacaaaatagccaaaaagtaccactctccttttgatacacttaaaatagagcagtttattatcacagaagagcaagaaaataagctattaagttgggttactgatatagttaagcactggagggagtagctatgtacgagctagaaaaacttctaggtaaagataaacatggaaatcagataagaattggagatagactaagtttacaactagatgatggggagtaccgagagtgcgtagtagcggttagaaaagttaatactatcgtgcttaaccctgtagctttaactagtaaaagtgaaactgtaagtgtaacaggtgtattctttgtagttactgacccacatacaggagaagaactacctttactacctaaagtagatagtaacggtgtggtagacactcaaaaagcagaaataatcttataattttatagaaagaaggaaaacaagtgcattcagtagaagtagaaaagaaagaacagttagcggacattgtatcacaagacaaggttgtattaaagttttataaggataactgtagcccttgtaagatagtaggtatgtctattgaggaagctagtagtaatataaaagaggatattactatcgctaatgttaagttagaaactttaggagaggaagtcttctttgatttcggcatcaaaggcacacctacactgttgttagtagaaaaaggaatagagaaaaagagacatagtgggtttatttcccctgatgactccaagagtttgttaaataagagaggcttaggcttctctttttttgttgacatttacaagtttatatggtatagtaacagtaacagaggggaggtaaaaaaaatgacaaaaaagaatattaaaataacgagtaagacagaggtaaaaaatagctctgttgaggcaggagacatcttctacattgaaggggtgtctaaggggcttactaatttgtctggtttgtatatagtgacatttgattttcatgcaacagaagagtcgctgtctaggtacgctctagtttctttggagacagggatgttaaacaccatttccatcaaccagttttctctagaagaaaaaatagggtgtatagtgaaggtgaataatatagaaatagtagcaaatactaaggaggtataactatgaaagtaggagaaaaagtagtaattaaaggtgaagtagttcacgtaaatggttctttggtaaaagtagagacaaaaaatagttctttttgggtaaacaaagaagacctaaagaaagataagagtagtggtaagtaggtatggcacacttaattgaaaagctaagactatataaaaaaccagaagaggtcgtaagaggaacatcagtaaaagcacagcatatttatgttgcaaatgatgaagcaaagtcattttcaggagccgaaaaatgggcgggaacgtctgcctataccttgacaagtaagaacgaatttacaaatgtaactattcactctctagacattagaggagagggaggcagagcttaccaagttacactaaatcacgataataatacttatttatgtgacttaagggaaactcaattaatggaaactattcgtaacgtaggaattgaggctggcggaaagttaattggaacttacagctttattgctgacggctcacagaaagttctagtaaggataggttcagacgcgtatgcaaaagcaagtgaacaagcggagactaaaaataaggcaaagagaattcccaagaaggagctagaagtaggaggagtatacgcacctaaggctacttatttaggaacaaatgactccctagacttatatataggggacttgtatagaaatgaaattgtagacgataacccttggtaccgagggtactacgaacgaagaatagaattagggaagcgtaagaagttctctgtaagcttaaatgtagcattacatgagggacaggtatgtgttccaacatacagagaagctgaaatcatacctgtatttctaacagacgatggggttacttcactaaatattagcaaccagctagggcgtttatccccaagaatggacttaaaatcaagccctaatttctttggaaaggtaggggaggctcctgaaattaaaacaacagcagagttcttagaatgtttaagacgtggcttagaagctcgcattgaagctttacctagtaaaatagagggcaatattttaaagggaaacttaaggttacaaaacgttatccctgaaatggagactatactcaagttaataggactcaatactacgattgaaaagcctaaatactccgctagttttgttaacaaaataaaaaatataaaatatatagttatagcagaacgtaaccaaaaagggacgttaaccgcatcggaaagtacagacttccatcagtttagtcgtgcttttgatgacttaatgtccaatattttagaggttattatagtagaggaggaaaataattgacgttatatatgaatggtaaaaaagtacatgagagtactttccccaatggggagattaatatcatgcctgatgaaaatttatgggtacttgaggaggagaaagcagagattacttggcagtactcaggaaacggtgcagaagacttgctagctgtcagactactagtgaacagtataagagagcacaacaagaaacataatagcattaatttaacagttctttatatgccatacagcagaatggatagacttcaaggaaatcagccctttagcttactagagtattttaaaatgttccctcaagttgacaaactagaaatagtggaacctcattcagacgttacagggagtattgctagaaaacttattgacagtggaacttactactcagagcggtttgctacagtagagatgctaccagaagtgttagcagaagagttgtctaatacttggtgggaacctgtattagtctatcctgataaaggagcaaaagacagatatgacaaattagcaactgattctgattattcattcagaagagctactgaaaacagacgagtactatttggaaataaagtacgggactttgatacaggtagaattaaaagtttagagctagtaggggaaccacctactagaaaaggatggacagctattattgtcgatgatttaacttcttatggtggaacttttgtaaaaacagctacagaggctataaagttaggagcaagccgagtagtactactagtagctcatatggaaggtagcgccactaagggagaattatttgaatctattgataaggtgtatactacgaacagtatggaagctagctacacaccagaagagctaaaagaagtagaaagatataaaaaactagggttactagacgtaactaatattataaaataaggaggaaaatatgcgtaaattaaatgaaatttctatgttaatgagtgattgctataaatttggtcacatgtctttatacccgaaaggtatcacacaggtgtatagtacacttgttcctcgggataatacttacttttttggaggtcgtacagataaaatggttgtctttggataccagatgtttgtacaacgtttcctgttagaacattttaataaaaactttttcgagttacctattgagactgtaaaagaggactaccactatgtagttagtaacgctttaggggagaagaatgctcggacagaacatatcgaagcactgcacaagctaggatacttacctataaaagtaagagcccttccagagggtaccttagttcctatgcgtgtacctgtgttaaccattgaaaatacacaccctgactttgcttggttaactaatttcttagaaacagtattactatctcaaaccttcttacctgcaacagtagccactactgctttagaagctaagaaaattatgactaagtatggagaagaaacttgtgataacttagaccatattatgttccaaggtcatgatttttcggagagagggcaacactctaatgaggcttccttgttagcaggtatgggacatttagtaaactttgcaggttcagatactattcaagctagtgtgatggctcacaactactacagtgctgatttagagaaggaactcgttcttaagtcggtgacagcatcagaacactccgttatgcaaagctacggtacagatgaagtagagacattctcaacactaatagacaataaccctgatggtatcttgtctctagttagtgatacgtatgactattacggtgtacttacagaagttcttccaaaacttaaagataaaattatgaatcgaaatggtaaactagtaattagacctgatagtggaaaccctgtagacattattgctggacgtgactatatagagtttggaactaattggtttaactatcatataactaaagagaacttagtaaaagcagtaaccactcgatacgagcagtcatgctcaaacaaaccctcaataggtcaaagagtcgatattgtagactacctaaatctatttgtagatagcgactatatttataataatgatattgttgataaagagaaaaaattagtatcagggtattataaagtaacattaagtgccgttttccttggtgaagacctgtgtaacccttataaagtagacaaacttgaactagaagattaccaaccaacccctgaggataaaggttctctacaactactatgggaaacctttggtggaagtgtaaatgagaaagggtacaaagttttagacgagcatattggtttactgtacggggagggtattgacttagaaatgttagagaagatttttactcgtatgggtgaaaaagggtttgcttctagtaacgtagtatttggtcttggtgcttatatgtactccttctctctttccagagacagttttgcccaagcaataaagagccaaatggtagttattaatggggaagaaaaattaatctttaagaatcctaagacagatactaatcatattaagaaatctcttaaaggaagagtgtgtgttacaaaaaaagaaggaacatatgaagttacagatgctttaacagctaaagaagaagccaaccttgaaaacttaaacgagctgaatgtaatttttgaagatggtaagttagcctatcaagactccttccatactattagagagcgtttagacagagaagcaaaaaaatattagaaaaacgaaagagcctttagtgggctcttttttgttgacaattaagtaaaagtgtggtaagatactagtataaatcaataaaggagatgttttatttgctagttaaagaaagaaaattacgtacctcaaaaaatagtatgctagattttttaaaggagggggagtttgtaagagccttcggtgacttatggtatagaaaagataagaaactatataatgttacacttaaagaaatatttgacatttatctggagcttgacgtttctatgctagaggctactatgctagcttttagcactaaagaaggattctcacctaaaaagtatgcgttagaagatttaaaggaagaatatggtactaatatagtaatcgactttactcttcctgagaataaagaggactttgtagtagataatacaaaaagtctgcctctattatgggacacttacagaagttatatgcctaaaacccaagcagagtttcactacaaaaaacctatttctaagaaagtgttgaaagaaacttgggggcacgtcctagacacaggctcactaagtgaagttagaaagaaaatggttttaggtacaactgctagcattcgaggtctacgtaattcggtaagttcgtcaagaataaaaactagcggagaactaaccgttacctctgctaaaatggattttaaaacaggagtacttacttccgttgttaagcgtgcaatggtagacgaatacgaactatctgcgaacgaatatacagtaaactttaatctttataatgctatggagcttattaatgacatatcggactataaagtattagacaaaggacacagagaccattacgattctaacatgatgtgggagattgttacaaagagtataacccatatatacaaaaatattgaggataagtacaccctcttaaggctagtatcctctcttacctcaggtgtgcaaggaagatactgtagaagtgttcgttatgagatagaaggctcagagttggaacactcagaagtacctatgcctttagtacgtgaagtagtagctaatctaaatttaggtgaaaatatacttgacagagcaggaaactggttttaccaagacgtagtaggcagagaaagtgttgcatctacattaaaaacaaacaattatcaagagttagtagagcgtgcaggttctttagtagcagtttttcctcttttcgacaggattgcttcttatatgaacaaccaaactaaaaaagagcagaagatttttgagagagctattgaacgacattggctaggtggatacaactaccttgagcgcagtaatttaaaaattattggtaatgcactagtacccaatgaagtattaagagacctactcacttataatgcagtacaactcgtcattgagaagtttacacgctaccaacacaatgtggttgcttacgattacttaccagataaagggtatgaacaggagattgaaaaaatacttccaagccttcaaaaagaaagtaagcggattggagaaaaaattcctaagtacattgaggatacattccagaacataaaggaaaatatggtagaggatgtaaaaaatgtacaaaataagattacacctctaaagaaaccatgggaatatattatccatgttaaccaaggtatctggaaaaaagtaatggaattagggaattttgagaaagagtcctatattattatctggaaagctttgtacgaaaatacggaagagaatgtaccatttcttaaatataaatccaagatagaaaacaagttactattcgaaagctctgttttagcaaaccctgctatacaatcatcatttactgaggcatttcgagctattggcgggtatcttgagagactaggaagaacacctgatgagtttatcaaagtcattaactacttgtctacagactgtacattaagacagagaatgagtcttagaaacgcaacagtccaatatagagactatattaattcctatgtagcattaaactcattagacttgaacgaggctaaaagattcgatttgtgtcctttctccttgaagttggctcatgatattgcaacatctgattaccaaactatgcaggattctagagcggatgagtttatggtagcacgatacaaagttatgcctgacgagttcagcttaaacaacagaaaattctctattgataaagaggaatatgttttagtacaacctaaaggagtagacgacctaaaagcggaaggaagttctttaaaccactgtgtcggaacatatgcagctagaattattaaaggggataacttgatttatatgttacgtaagaaagacaacatagaagagagctatcttacaattgaaatccttgtccacgatgactcccgagtatcattaggtcatctacagggagagtcttatcgcagtaagattccaacaaaaataacagataaattaaaggaatttgtggttaaagtaaatgaggctatgttagctcaaagagaaggcagaaattttactaaagacactaaatccgaaactttagttagagccagttaacttgtatttaaactcttaaattatggtataataaaggtagaggctagctaataatatagctagccttataattatactgaaagaggaaattaaatgaaaaaagataatactcggaaaaaaaggttaaacatttataacaccgaccgatattttaatatgaaccttatgaaaagaagtgctattgagaaaagtattaaagtaacacggttgaacgagaaagaaatcgaagaagaaatggacaagctagcagaaggttctacccgatttaacaagaagaataaaaagagctacttactctatcaagagaaatatacaaatgatagactaattcaaaagctctttgctcatggaggtagtgttacatattacactaactctatcataccttactatgtaataaaacatatggcacagtccactacaagtaaagccgtatactctgtaaagaaagagttcacagaagaacaggtaaaaaatatagagctaaccaaaatggctactacagtgataattgacgttcctgttgttgttcctgatgttagcccctatgatgttctgttctcactacaccctttaaaaacagatgtagacggtgtacagttttcctttccaagattaggtaaaagtgaaatacaaaaaaggcacctaccgttctatgagttgaaagggaaagtatacgaattacgacccgaaattaaatatgaatatgtaaaatacactcaagtatctttatctatttgggggatgtatctctggttagtctgtaatagtgaagaagacttccaagaaatagacaagctactacaaaaagacctaaaaagaagagacagtggcagacgacctagtgcaagtcagcaactaggagggaaaacccatgcctaagaagatgataaacagaacagatatagcccgtgaagtagcacaaagaacaggtttttatatcaaagatgtagaacagatactagcagaaagagacaccgttattatagatgcccttcaaggcggagtcactaagattaaagagcacaaactgtatcagattgaagtagaaacaaaaccagcaaagaaaaatgcttggaatgggttagacaaagtatactttagtataccagagaagaaaataattaagtttaaacccttagtagcagtagaagaggtaatagatagcttaaataaaaaagagacgtaatagtctttttttttattgtacttgacaggataatcaactcatgctatactaaggtataagaaaataagaggagaggtgccatgtgaagatactttttttacaagagtacattagggtaaaccacgtccttgtacaagatacaggtacagtcaatatttttcttaaaactgacttagggaagaagctagtacaaaagcttaaatcagtaggtataaccaaacaagacttttatattgactatgcttatgctaagatacctgaaccaaaaaaacaagtaaacggtaaaatactaacgtacaaggagccaacagcacaagagaaaaaggaaccacaaacattactaacagagagaattatacaagaaaaacctgatttaatcgtacctatgggtaacattgggtgtaaataccttcttaatgttgcatccattaggtctttaagaggtataccagaaaaggtgaccttacaatcagagcaaacaggggaaaagtttactccatgggtaatgcctatgtattctatggaatatctaatatcaagtcctaacgtagagggtatgtttgatgcggatttaacaacactcagtcggtacaaaacccaaggagacgaggcgtttactcctgcggaagtatcttatgagctagttacttccttagaaagagtacaggatatttttgaaaatgaagttaaaaactatcctgttattgcatgggatttagagactaacaccttactacctgagatgttaggagccaagcctctagtcatgtcagttagttggaaagaagggcaaggtgttactattccgctagaacaccgagagagtcattgggaagcctatgaacttgaagagatttataactatattaaagaatttgtagcagataaaaatattgttaaagtaggtcacaacatttaacacaatgggtgttgtaaaactcctctccatcggtgaacgctaagtgcaaagcatatgccaataccgagcctacattactggtgacagtagatggggtgtagagactgaaccgagccattgtagggcaaggtgagaagtggtacatatcacttaaacgaggagcattgttccatataggtaactacaaataaaacctattggtaaggggcataaaggtatagtccacgcttatatgaaaatataagactacgtgcagtatgatatacgttttcttatgtcgtctaaagggtttactaagtttaataataatcgtgatacgaagatagggtactacttgtctgtaacacaacagatagaggactccttcaaattatcagatttagcctatgagatgacagatatgggtggttacgacaaaccacttgccgatttcaagaaaaaatatattaaagactacaacgataaaaaaacagcagaacaggaagaaagaaaaaagaaagtagtagaggaaataaacaaacagtttgtagaagaaacactaaaatttaaagaagatactaagaaatacaacgaagaaaaggcaaataataagtattctgtactagttaagcctgtaaaacctaaaaaaggtaaggcacctacaaaggcagaactagcttacgagcctttagctaacgaagtagatggaggaaactttaactatgactggattcctctagagatactacatccctatgctagtggagacactgactgttgcttacgtattcataatgtacttacggagataattgcaaaaaaccaaaaaatgtggttccagtttgctaatttttaccctagtgctattgtggcattagctagaattgagtctaacggtatctatgctcagacggactatatggaagatattaatgagaagtacactgaggaggaagaacgcctcgtagaagaaatgcgtaagatagcagaggtatccgagttagaagagcaacacatggagttgtacgaacgcggggtagaggagatggcaatacctcctgcaaaaagagataaggatatagctaagttaagagacaaatacaagaagaagctaaaatttaatcccaactctgcaacagacaaagggaaagtcctttatcaaattatgggtattacacttccctacgataaagagcatattaaagagtctgcttttgatgtagggaaacctgagaataagctaacatgggaggactacaaaacagataagaaagcactaaactatattgtagaccattttcccgaccaaaaggcgtttgcttctctcctactagagcactccttagtaaaaacaagaaaaaataacttcacagccaagcttctaaacttcattagtaataaggacggaagggtacatgggtcgtttaacgctacaggaacagagacaacccgtttaagctctaataatcctaatatgcaacagctacctgcaaaaacttccgaccctaaaaggttcgactatcagtacccgataaaacgtatgttccagactgcatttgagaatggagcactactacaactggattactcttcactagaaatgcgtatcttagctttagcagcaggtgacgaagctatgacacaagcattctttgatggggcagacttgcacaaagaaactgcttctattgtatggggaatgcctgtagacaaagttccagcagaatttcgtaaaaaggctaaagcagtaaacttcggtattgcctacggagaaacacctttcagttttgcaccaaaacatgatatgagtgtagaagaagcagaagacatcttccaacgttattttgcaaataaacctaatatcaagacatttattgatgaaacacacagagaagctctacgtgacggttttgttgatacattacaaggtcacagaagacttatccgtgatgccttttctaaagataagcagatacaaaatggtgctttacgtaaatcagttaataccaagattcaaggaacaggtgcttactgtacaaatatgtctctaatttatatcgaccagtatatacaggataacaatatgaaatctaaattagtattaactgtacatgatagtattgtagtagactgcccaccagaagaggttgagattatggctcgggtgtgtaaaacaattatggaaaatctaccaattgacttcttgagtattgaatggaaaggtgaaaaaactaagtatcctattgaagcagacatcgagattggtactaactataacgatatggtagaatacaatcctagtgagttaaaagagttcaactctttaacgggttatgtgaagtactacaaagatttagggaaaatttccgattacatggaggctggtataattacagaggagagtatggatttaggacaaactagaattaaagaatcaaaagaaatttacaaaaaaattgcctaaaaggttgcttttgttttaaaaatagtgtataatgatacagaaagctatttctaaaaacagaggagggtaaaatatgacttaaggagggatttgtttggacatacatgtagattcattagaatttagccaattacgaattatagatggtaacggacaagttacagagcttgatttagagagagagttgagagtaaatgaatactcgatgcaggaggagtttctcaaccaagcaccaaaatatatctattgggcttctattctagagaaagtacgtatgtatcaggagtctgcatcactagctttagaggtagtgggagcacagatgaatgagaaggcaagagagtattacaagactgtagacactaagccaacaaaagaccaagtagaagcctatatacttcaacaagaggaatatcaagtggcttacaaacagttgttaacctataatcacttggttaaacaactacagtacgtagtaaaagcgtttgaacaaagaaaagacatgctaattcaatacggagcagatttaagaaaaaataaagataacggtataagcccaaacttcgggtaaaaaaaaataattataaacgataggagacaataacagtatggtaaactttaaagaatttttaaaacaggagaaccaagaaaacgaaagtaataataatgaagaggtagcatttccaaaaggaaaacatcaacgcttattcttgtctaaaaaatcaaataccgtggtagttcgtattttaccaccagctaatttaacagataactatgcacaaaagtatcgtaaaattatgctgaacaacgttatgacagatacaggaaaagcattaaatccgtcatttacgttaccatgggagcctaacgaggattccccactagaacaggctttagctgattggtataaccgtggagtagttcctaactactatggaggaaaatcacgaccaagcaaagctactgtagtaaacgctgtggcattagtaacagatgcaaacgggaactgggtacatgaggttgatgaatacggtcaactagtagttcgagtgttggacttaccttattcagcatatacagctatcaatcgtaaattagcagacccaatgctaaacactacaggctctgactatagctttatctctgatgtaggagctaacccaatccgtattactaaacctacatcaaatacagagaaagcatatcaagttgaagtatatacaggtattacattaccacctcttcctgagaattggagagaccaattggaggacttagcttatcaagttagcccgtcagaagactataacagcaaatttgttaactttgttattgaacgagttaatgctgtaactagtggaggagctcaacagcaacaagctaacccttacggaggacagcaagcaccacagcaacaaggtggttatcaacaacaagttcctcaacaacaagcttaccaacaacctgcccaacaagcacagacgtatcaacaaccagtacagcaacagcaagcacaaacataccaacaaccaacacaggcgtaccaagccccaacacaacaagtacctcaacaacaagcgtaccaagcacctgtgcaacaaccaattcaacaagctcctgtagcccagccacaacaggctcctgtacagcaacaaacgtatcaggctcctgtacaaccacaggcacaacaacctattacgcaaggaatggtggactcagcaatgagcgggttagcatctcctaaggaacctgtacaacaacagaaaccagtaaccccaccaccatctccacaagcacagcaaccagtaagcaatgattcaagcttacctgatgtagatgagttactacgcagtatgcaagaaggttgataagaactaacatgaggtctaggatttattttctagaccttaataagaattttagagagggttgttttcatggcaagagctaaacagagtgaaaaagataacaagacagaaattgatttaggtaaaaaaattgggttaactactttgcaggatagtaaatgggcggagatttcagataagttacctacatttatccctcagttagactatattctagggggagggattccttttggtcgtttgacggagattatgggtaaaaacgcaagtggtaagtctactcttgcagtccacttaactaaggtagccttacagctagattgtaaagttatttggattgacactgagggaacggcagaccctagtaggcttagtcagctaggcgtagatgtaaacaaaatattttttgttcaaccaaaaaaatctaagaatgctgaggtaaacaaagaggattctattacagtagaatttgtagcagaacgagtagaagctattttaaatgccttctctagctctgaacaacgaattgtgataatttgggattctattggacatacaccttctatcaaagagattgaacaaggggtagcaggtaaacaaccgggtattaaagctaaggcaatggctcagtttgctaccattattgctcctatggtaactaaatctcattgtgcctttattggtatcaaccaagctagagacgaaatgggaagcatgtttggtgcagtagacagccccggaggaaatgcactacaccattgggcttctctaagactagaggttaaacaggcatccaaaatagaagaaccgatgcaaaatgcttttggtactatggataatgtatatgtaggacataaattacgtgttattactaaaaagtctaaggtatctacacctagacaaagtgcagaaatgttccttttatccgcatcaggtttgcaatatgaagagaatatttatcgttcttgtgtggacagtaaaatgtacggtcttatcaaaggactacaatacaagtcttatattgcagatgatggaaaagaatacaaaatgtatggtaaggactgggttcctttcttacagtcaaaagacggggaaaagatacgaaaagagttattccagaaaatgatgttaattagtttccctactggatatagtccacttgataatgaggtagtggatgttaacttatgggaagatatgaaggggttaacagaactgtacaaaaagaataaagtaaagcaacctaaggtgaacgagggagcaaaagaagagtctattgaggaagtactagcaggaattgactcggataaataactttataggggagggaaacttccccttttttcttttagtactattgaggaggaagtaaaatatgagagctaatagaaatattgatttaaccgagcatgtggaagtttggtctgaaaaaacaagaagtattccaccctctaagttaaatccatttctagatattaaaaaaagatttctatatgcaaaagagcataatatcccaattattctagaaaacagcgagctaaataactctcaatctgtggttctagacctaagctttgtgggagataaatgggcaatgggatacgtaacacattattcagctattcgtacagagtctactccatacactatccattacagtgatttgtattgtgcaggtaaacaaagtaaaaagaataaaagagtaaaagtaatatttaaaggggataacccttatactatggaggaagcctgatgcgtaatattgataaagaaaaggaatatattagaaacggcaatcgttatgtacaagagaacagtgggagaggggtatttgctagagatagtgaccgactattccatcagtacagtaatctacggtggagtgtctacaataagcataaagactccttcaatgacgaaactactagagcagaattacaaagctatattgacgagcagtttattaaactagtgaaggagtacgaaattaacggtgcagtagacttcccggggtacattaaaacaaagttaaaccagagagtaaagcatacttttgttaagaatgtattccgagacaaagctagagaacgtctaggtgctgatgagtttgaagtagctaatctagtagaccgacagcatactgatgaagcactactagaagaccagatgttacaagagtatgtatttggtaagacagagttcacacctattcaaaaagagttgcttgtctatttactagaaaaagggatggtttctgatgaacgagtaattatcagaaatgtggcatctaaattcaaagtgcctagagaaaaaataaatgaggaattatcggaactacgcttttttgtttcttctaagatagctaagtatgagtcagaaaaaggctattaatattgaaataacgtctgctatattagagttagaaattcagtataggaggctttaaacaatgacagaaattcatacaccgcaagacgaagaagtaaaagatgaagtagttcaagaagttattgtagaagaacaacccgagaacgtagaagaagtagtcgttcctagtgacgatgaagtaaaagcaaaatttatgtcttatgttccgttattgttactttagttcttttcttgaacaccttattagcatcgttgggttgggaaccattagcagtttctcaagaccaattgtatctagtaggttcttctgttgcaacagttattgcaactgtatggtcttggtaccgtaataacaacgttactaaaaaaggcttgaagagagaaaacatcgcaaaccaagtattacctaaaaaagaggacaaataagaaaggatggaatattaatggctattaagaacattaaagatttgcttaaagagtctacagtcgttttagatgggaagtatctacaagctagaacggtaaccgatggttttacaggaacctatcaaggtaaccactcgtttaaagtagaaaaaacagggagtaactactctgtataccctatgtattttgacaaagatacaaaacaaatggagaagagaaaagacccgattatctatacagatgacggagaaattttctttgtagttcgtactacagtagacccttacaattacccagttattgagcaagactccttaacaggaaaaaatacagagaaacagattctacaagcatttattgctttctctgctgacgagtatagttttgctaactataacttgtttatcaatgacgatttaaagttctatgacggttctgctataaaagaagttcaaagtattaagttccctaatgcgacagaaacaataaattcttttggagaagataaaacattaactgttatctttacacctgcggatgctagcgataaaacagtaacattcacaagttctgacgagagtgttattaaagtgagcaacacaggtaagttaactggctctgtcaactccactgaaaagtctgctgtggttactgctgaaacttctaatggaaaaaaagctactgttactgtaataatgaaagcatcagagctaccagaaccgtgataaactcatagaaaagaaagggactttcgagtctcttttttttattgactttacaggtaaactgtgttatagttacatagaagatacactaagaaaggagtgacaagatggaagacaattttgtaacagcctataataaaggagaacagacatgggatgacttagcattttgggaggagtcttggaacaacgaccctaatcatattgccagtctagcggagtatctagggatgacagatagagactactggaattatgtagtaatggacaaagaagtacctaaagagctcctacctcgaagtgagcaaccccaactagaaactgaatacatctaccacatttgccataaaaagaatgaagtacaaatttttaaagaaggttctttagtctctcataatggaaaagtggtacaagtagataaaataatctctgtaatcatcacagagaataactcagtagccattcatcttaagattaaggagggaactaaatgattcagctaattacaaaaaataacatgcagggatactatgcagactatctgttacgaaaagtatctacacatgaagtgagaacgacttacttagagaagttcagactgcaagacatcactagttatagtgcagatgctactattattatcgtactaggatttagctacagtgacagagaggcgggagctactgttagtaacttcgaagagtatagtgcaacatttaataacccattcactttcttttaccactttgctagttttggtgataagtttaaatacccaaatattgactctctagttgacgaaaaagtaagtcctgttatggtaatgtacaacaccttgaaggaaaaaaatcctgatttcttaatagcagagaaagacaaagaagctatgttggttgctactgctgtagacgactaccactgctaccgttttgcagaaacagggaatgggctgtctctacctttaaaagagctaggagacctctatggacgttatttagctgaattgtacggaaacatgtctttggtggagctaattcagtcaaacaagaccattatcaaagtgagacaagagtatcggaattattacatcaagaaaaaaatagagcagtctaaacgacaacttgttaaagatgttatgctttattttgtctatgcagaggaatacgagaatgagattgcacaccatattattgataaggctaaagaagttaacgatagggtgcttgtcttagtaggaaagcatacaacaaataatgatatgttccatgtacgctcccataatattcacgcaggagaggtagctaagctaataaataaaggttcaggtaaagaaagtacagcggtggtattcttaggggattccaaaacacctacatttaatgctgttgtgaaagttttgctagatgctagtgatatttaaccactaaatgtggtataatagaagagaagtacacactaattagttaggagaaaataatttgaaaaatttaacgaaggcagaaagaaaagctattgagaaaggacacttaggaaaaacagccgttattctaggagtgctaaatgcttctcacggcaaaattgttatgtctcagtataataaaatagcggaaagtttaggaatggaaaaggtagctaaaccagacttcttagcactaaatgaaaccattcaagaagataaggttttacttggactgtaccaaaaatcgtctgtaggaaagattacactagatgatttaacaggaggaagtaacgaagacaagtatagccagtttaatgctataacagcttatacagtagataatgacaaagtattagcagacttacgagaatttagaaaaattcaacgagatggagcttatacacaacatctttttaacggagtgaaagcagatttaaagaaagagctctcttctattgctaaacctaaatttattattaaagagaacaagaaaaaaacaggtaaagaaatgattgtagccttatctgattggcacatcggagctttagtaatcaactctgatacaggaggatacgactacaaaaagctagtaagctatctgacatatttactaggtgaggttgttcagacagtagaagagcatgatattgacgtatgtcatgtatatcatattggagattttattgagcatattaatatgagaagtgtcaaccaagcatatgaagccgagtttaatgcaacagagcagatttctaaatctattcgtacaatgcttgacttcttaaacgtattagccgaccatgtacatgtaaaatttggtatggtaggcggtaaccacgataggttccaaggaaataaaaatgaaaaaatttataatgataatgtagcttacattattctagatacattgtttttactacaagattccaaagcgctacacagtaacattacgctaattgataatcggaaagatgtttacagctttaaagataatgttgcaggtaagaatattaaagtagtgcacggggacacagaaggtaagaaagtagatgtaaaaataccaaaacacattaagagtgaagtagtagactatctattcatggggcatatccatacaactagaattgtacaagaagattatagcagattccacgtatatgtaggtagcccgatgggagctaattcctattctgcggaaaataacctaccgacaacacaaggttcccaattattagttattctatctaaaggtagtaacaatccaatgtttttacctatctttactaaataaaggaggaggaaaacaaaatatggcatttttaatcacagtagtgctagtaacgtaccaagttattgtacttttgcaggcggttagtggaactatccgtaaagaacgaattatggtagaagaaggcggagaccccctaccaacatcctcctatgttttcttaatcattaatcaaggagctattgcaggattcctattcttgtggggggcagtactaagctttattctagctatggttactaatgttatcctagctgtaatactatttacgctagcagtaaccatcttatcaattgtaagtgcaacgattctaagtacaatcgttcagacaattgcagttattattgtaagacgaaagcagatagcacatacgaaaaaagttattgctgaaaaagaaagagagtagataaaatatgtctgagtatgaagcaagcagagcagacttaagtagagagttcctacaaggtgatttacagcctagtattatgagacgagggattctctacatcagaaagaaacctgaatatattgttgagtatgtagaagaggctagtagcgatacattaatggtaagtgtgtttgcagttcaaccaaagtctaaaccgcctttaaaggcttggacattagttgataaaacattccagtataacacaggaaccactctaggtttactagctaaggagctgaaacatgtacctaaaaaaacagagaaaagagaaaatccccctttgtttatcgcacctgcactagatggtgtggacacctttacaggaagagcaggtaagggtttctatgaccgagaacctgatagagccacaagcgacggtaaaatcgttcaagggaagcacacaggtgtatttgttgggtttgactctattacttgggagaaagctattgtgctaactgaggagatagctagtaggtatttaactgcccgagcccaatggtataacatttagtaatgctatattaaatagcatgttaacaattgttaacaggagaggatggaagatatgacctttacagttgttttattcctagctgtaatactcttgtttttatccgcattatgttttaccagtcataaacagagttggaacgtagtaggcggtgtactattagcctttggtctaatcgcattgttctatgtgcttataatggcaagtgctgtttcaggtataaatggagaatggtttgggatgaggttataatacacacattgggggctctgtaaggggctcccttttttatggaggttggaatgaaaaaattttatgaagatgatattaagcaggtactactaaaaaaaggaaagatgtttggtgatataggaaagagcactattgtatttgagaaaggtattactatctcctctacaatcgcagactgtcttatatttacagagaaaaaaggagtcattggtgtggagataaagacagagagagactccactgcaagattaaataagcaactaaaaaactactccttagtgtgtgattatgtatatgtaatgtgtcatgataaccatattaaaaaggtagagcagataatgaaaaggtaccatcatgaacatgtaggcattatatcttatattgagtttatgggagagcctgctattggtgtctataaacctgcaagaaagtcaccgttgaaagatgtataccacacgattaatattctatggaaggaagaaatactaaaaatattaggtgcctttagacattatggtgcaagagctcctaaaagctcctacacagtagcagatagaagcggaggactaaatgggttatccataaaatccagtttctctgctcgtatgagtaagccagaactaataagaaatttgattgctagggttggacgaccagaggctaatcggattgtttgtgatattttcattgaacatagaaacaatcctgccaaggctataaagtaccaccacttctttcctagcaaactagaaaggtaagtgaagaatagtggcaaacaatactagaggtaatctacctagggggtatactgcactaggagacgggagtgagtctcacaaaacagtagaaagagcgctaagaggttactatacagtagactttagtaatatatacgaacacatgacagaaagagacttagaagtacgacaggcatacgctaaagacctcatatctcaaagaacaggcatccctttagacatgattttatttagaaagaagacagagaagcagacattaacagctatagatgaagtcttttatgtaaaacaaggggtaaaaacaataggaaaagtatctatcaggtcacaaagactactaagcagtaatatgctgattattatttacatgtctaagagccatatgcctcgtgtagttagaaacacaggaatgaaaacttatacaaaacgtagtaagtcagacatacgtgtagcaaaaagacaaaggaaaacaacaaaaagacagagctattgacaacagagtctacttatgctatgctaatattagaaaaataaacgaaatgaggtacaagtatgcaaatagtagaaaaggagagttccttaaacagctttttaagcagtatgagaaatgaaaaaggtacagtggtaggggtctattataaaaatgcaatgatagacgaaagtatcttagcagaagccagtcttcaattagaggtgcctaataagacggagaaggtcttacaagccacggtagcactaaaacctatgccaagtcaggtaaaccagttaacgaccttagagagcctcctagaggactctggaatgggatggttttttgatagagaacgacctatcacaccaacaatgagtacccccttgtttaacgacttagctattagtcaaagtgcactatcaaatatacaaaattatagactagtagttaacatggctgaccctaaagtaacaaaacaaacctttactttttataaaaatacctatacattgtttaaagtagaggacagacagttggatagtaaggttgcaggtaacattattcacagtatccatgtcttatataaggaagagcagtatgacggtgaaaccttattcggtagattgtttgtagaaagtggtagtaaagaagctaaacttatgaagagagatactaaaatcttgttggacttgttatgtgatgaaaacatcactagtctttttattggaggaatagagttatctttagaagaccgtttatccaatgctaagcttttccctctgtacacagcagttacagctacaagaacaaactatgatgcttatagattcctaacagattcaggagagactactattgtttcggatatgattctagagagtaagttagttaaagtagctgactataagtttacctttgtagtaaaattaaaaggaaatgcacaattaactatatatttaggatagaagtaaaagaagtcagcatttaattgttgacttttttctttatatatgttatgatgtagttaataaaaaagaaaaggggttgacagaagtgacgaattttttaacattagtatttattctagctattattgtagctgttcctacgttattagctaaagcgatgagaaaccactataccaacaaggagacaggtgtttggtggatgccatatacacactggtttgtgtacaactttttatatacacagaagctaggatggctaattttaggctccggtattattatctccatttatattccatttattggtggatttttaggaacagtcattggatttgcagggggactacttatgttatttggtgggctaccttatgtatttggtttactagggggagcagtctttggtgtttttgtagacaacattgtgtatcatctaccagaatggtatcgagcttggtttagaaaccgatttgggattgagtttccttacctagaagacaaggaagatgaaaacgaggagtgaaagtaatgaaaaatgaagatacagcttggagtttactaggtttaatttcagctattgccttggtagttatttcttttctattcttagcagggtgttcacaagagacatcacaggttaaagaaacagagcaagagaggtatgagaagttcactcctatgattgaatatatttactctcagaagttaattaaggtgtataggaatgaatcagacgagcttgtagagtacacctactatgcccgcacagaagaggaagaagaagaagggctcatccacgaagaggagtataattattacaaaagtcaaggagttactgatagaggtgttattaaaacaacaataaataggtatggtaagggtaagtacccattaaaactatgggatgtgcaagagtacgaaggaaatgtcagcttagatagaatagataatgaagtctactttgagggtgattaatatagaaactacaagtacagtaggatactggaacatagataccttttcgtatgaatatttgtactgtgctacagagggagagccttggagcatagggactatgctaacggagtactacactgattataaacaagtaaggcaactaatgcaggacaaagagtttggtaataagaagtttagcttcacgtcttcaaaggtaagcaaggcaactaataaaaagaagctaaaggacaaagagacctattttcagtatatatttgaaaaagggcagtggttctgtgtgtatactaaagggttcatggctaacgtagagatacctgctatagaaaacccataccgatttaataagtggttagacaagaatgctaacaagttaagtaacaaaagagtaaaactgttacgagaactagttgtaagtagcagtgaggttaaagaggaaacagagagagtagttcctgctatggttaaattagaacctccgttacgattattgctagacgacactcatagtgtcagttctcataatatactggaatttttaaagtcagggaatttgtactctttagctaagagagagttctatgttgatgactatggaaccgaagtagactgctttactataaatgagttaacaaaaccaagttcccaagtacctagtcaaataacggttagtaaagagcatacatctaattggtatcctctattagacctacttattgactgtggttatatatcagactatgtagttgaggatacagggtctaatagtaaagaagtaaaaatagagataaaagaaggaggataacgggatgtctatatatgagtctatcgaggggctagaacaaagtattatagagggattacgtacaactttagaaacagaaggtaagaatatagccctagacgttatcagaaaagaattagaagacagcccgttcttacggttttttgagttgtctcaaggtagccctgcgggagatgtataccaagtagatactactaagatagctagtaaccctacacagcattacgaaactgcccgtgtagctagtatcgtgtctgctaacgttcctgtgtggttgtgggggcaggctggaacagggaaaacttctattgctcaaagtattgccaaaatgctagatttagaattttaccctatgcagaaaataatggacgagttcgaacttaaaggttttgtggatgccaactctagattcgtaccaactccactgtatgaggcaatgaaaaatggtgggttgttatttattgacgaaatagatgcttccgtaccagaagcactaatacttattaacactgctttagctaataattttatgcgatttgctgacgagactgtatatgcacataaagactttagggttattgttgcagggaacactctaggtaaaggaggtaacaactcctacgcacgtaataaaatagactcagctactttagacagatttgtacctattgagataggatacgatgaaaaaattgagttattaattacaaatggagaccgagaattagtatattttgttgcactagttcgaatagagatggctagaacaaagttagaccatgttatctcttacagagcgttaagtcattataaaaaatttgagtcttttggtgcggatttcttgattgaaaatgttttgtttaaaacactatctttagaggatattactagtgtagctcgttctcttagcagaataatcaaaaaaacagataaggattctaagtggaagaacaacaaagtgtggaagcagttacagacatacctttcaaacaaaggagtagttgtagatgacacgaactaatcatacgtattcatctaaatttatagcagaaaaccaaggaaatgtctataggttgcactatgcaggagaggactatacaaagataatagatgatatgtcagctatcccccactatgatatagataatatcaaaaaccctcaaatgaaactctcatggtctacaggaggggaacctattccagaatttatacacaagttccaaacaaattatatggaacctgagaaggcacaagagcttaatgtaatgctctctaaagtatctgaattagtaagtagccaaccacctatttatcatttttacggtaatactatatcagttcctagagccattcaagggcaccctaaaagttttaaacatcggaaaaaagaagtaactccttatattagtatatttgtagctatcaatgttctttatgacgttagtgtagagaaccataataagtttataaaaaatatgttgaaagttattttgatactgtcaaaagaccatactgtaaaagtaacactaggtaaagttagtgcctctaggggaagaatagctgtggagagctttgtactaaaagaggctaatcagagttttaacttatctagactttggtaccctttatctagtatagccacatatagaattgttctttttgagatgctagaaaggcatatgacactagagcctaaactatcctcttacaaaggtaattatgggtttacaatacacagtacggaaggaatagcagaagcagtaaacttgtttgagaaatacgactatgtagtcggttattacgaaacaataggcatgacagaaaaagaaattatagaaagatattttgtgtagttgactttatctttcatatatggtatagtagctatgtaaagtacaaagaggaggtaatactatggaagtagcagagttagtaggtaaactggtaggtaaagaagttatgattagtacagaaaggcatgagcgccaacaggtttatagttttagaacacacttaatagaacgtgttgagttgaagcctgcccctaggacgattgacaataaatcagttattgggaatgccttttttgtagtctatttaaaaggactacctaatgaatatcgactagaacagttaagaattagaatggatatactagacagaggatgcttattacctgttgagattgacacagaaacaggagaggtaacacttgttgtaagcgactctctagctaaatatttctttgctagtcttttagaagttgaagcacgtactgacgaggacaagaaaaaggagctagtacaggagtttctagcagtgtctaaattatttcacggtggcgggtggactaacggtaactattatgcagaggaggattaaaactccgtatataagaaatgaggtgaatatgtatgattaaagtagataaagtagaatgcaaggatggaagagtttactatgtctataatagtaataatggatataataaaaagaacatatgtattaatggaaaggtgcaaacagtacctaaagataaatatgagaagtatttacctaaactttcagagataaaaaaactagatacattgtatatgagtaacaaaaaccaactgtacaaagaagttgtatgtgccagcagtggaaatacaagtaataaagacaaaaaggtacgaatcaatataaacaatgaagtagtcagctacctgccgaagaaggcaaataacttggcgatacttctaacgcctgacaattgccatctggttagtagggtagagccatctatactagtagaacttctttctaaaagaacagtcattatctctagagatggcggtggggggaatgtaagaagaataggttctgcttgttgtggggacgtactaacgttagcatataaatatataaatggtaaacagcaggtaagtattaatgtactatctgttaaggatttttaccaatatgaactaggagggttatttttatgattaaagtagagcatattagatataagcctgtagattttggcttgtccagtacaactcgtacatcagaacactacgtatttaaaagtaagagaggtcaaaaggtggttacaatgaaaattgtgggtaatggtattcgggcaatgccttttgctaagcttaaaaagcaccatttagacaaggttactgtattgaaaacattttatgtacctaatcgaaaacagctagctaagcaactaggtttaagttactacaatagtgggaaagcagtacaaaaaacagtaaaacgtttagatgatattcaggactcgattgcatttagtaatgctattcaggcactacctgaccactcagcattgagatttaagttagtaggggaggagaaaacccctaaaactaaacttaacacagatggcagtacaaactaccttattagaacttccgctaagctattgggagaggatgtaaaccaaattaacttacttaacgtatctggtgtgctaaataaagcggaggcagaagcagagaagttcttacgagtactaaatctgtataaggaggctaccaaagatgattaaagtagaaaaatatacagctagtaccacaaaaaaagcaggttctattagagggacttatgtagtaggtggtactagaaaccagaacgaatcagatttacacttaacagggactacacactacaagtataaaaacaaaaaaggtaatacgatgattacatttgtcaattattatacaaaagaattagtctctattcctgaaaaggacttaagccctcaagacctagataccttttatagtacagtaaataacagactaactactgtgttctatgtccctaactggaagcaaatgaataaaaatgtactaaaaaagggtacaaaatagcaaaattataggtagttcataaagattttatgatactacctagtactgttttctaatctgtttcacttatcttgtttcatttacttcttttatactctagattgtttcaaatgcttataagtagttatgcctagtaaaccctactctctagtacactttatgggtgtttcacttacaaatgagataatacaaagtgtatgctatattatatattaagaacactgggaggcaacagataaccagtaggtcataaaatagtataaatgtagaggagttacatgtgaaatggcagataacaaagataaaaacaaggatttagaaaataaaaaagaggcacctacaacatcggttttacttagactgcatgagatgcctatcttagcttctaaggtaaatgatgctttagatgaggacaagtcgtatgactatattgtaaacttatgtagtcagtataactttgtaatctctaaatcagcaatcagtagatacaaagtaaaaagagaagaatctatagaaacaggaattgatttaggtgctttattagataaaaggcgtaaaaacgaaacaacagctactgtgtctatgattgcccctaaagagaaaaagtcatctcccgaagataagagatacgaggaaacttttagtactgtagacaaggtatacaacgatgtaatggttttggaagagattatccaaaaaggaatggaaggcttacaatacacagagactatagaggcacctattgcgatgaaagctattgaaataaaagctaaaatcacaggaaaccaacttcaaggactaagtattataggactacgtgaattgaaacttagaagtgtagctaaagagacagcaatcacagaaattttgatgcagtatatccccgaagataaacaaggagaagtgcttactaagatgagagaagtagaaaaggaattctataagaacttagacttatcagatgaggatagaagaataactgaggcgcttaaagtcgcaggtataaacagctagaaggaggtttttttatggagatagtagtgccagaaggttatacaaaaataagcatgactaagtttgataacctagaagataagttactagcactattaacaggagagacactgttagtgcgtggtttagaaaaaagtacgaacgcagacgtactagttaagctagatgacagaaagtaccctgtgacacagataagctatgaaattagtattacagacaatgtgtttcgagaacgttattggactatatatgacatatctattaatgcactaagtttgtatgatgttttcctttttgaggaagacaaccctgcatatgagctaaagatacaagtatcagacaatgtgcagtatactagtatcagcggtgttaaagacacagctaaagtaatagctatttatgaagatgaagaaaaaaacttgttttgcgagttatcaagggaagacacattgtacccattagtagaacttacaaaaatttagcaaaaggtaaaatcccttgaaagctctaagtagccgactagggatttttttgttgacattatatagaaagtatgctatactacatttataacatagaaggaggagatttaaattatgaacaacaaaaaagaatttgataaaaaggtgactgaattaaagaacgcgacagagattattaaagagcgggtagcaaaagtaacacacggtctaaaccaagcaacaaacctgttgaacttaaatgtaaaagaagtaaaacgagttgcttcaacactgcaacctctagaggaagttgtttatgagttgcagaaagaggttgacggattagcacagtatagcaaagcggaggttgtttactacgatggaagagaggttaaccttgaggagattgacttagaagaggacgactatgaagaggatactacagaagaggaaatccgagataccgtctctactgatggagggggagcacggattagagtatatcctcctgaggaaactttagtatatatggcagagcatattggacaaggagtagagactgatgaccttgctgtttatgaagctgttcgagacacaaacggtcacacaggtttccgacaagaaggctattatgtagaccctacggaaacatatgctaaccgagataaacgattaaaagaaacctcaaaagtagaagaggttgaggaagtagaactagaagttactattattagaccgaaaagtaaatctaaatctaaatctaaacctaaagctaagaaaaagcaaaatagtaaaacaaagtctaccacaactgagtttaaaaatgatacagaaaaaggtattgtaactgactatattaatggagcaggaacaagtgctattgcacgaacttacaatgttagtatgggtactatctacacagcgcttaatcgtcataagattcctaagcgtaacatcgctaataacagcgtagaggttcaaatggttcctgtaatcaataataagaaactactaaatgcaattattgacagctacaacaaaggagtaccagttaaagacatctacgaaaaatacagtattcataaaaacgggctatactacttgctagattatgtaggtgtagaccgtaaagtacaccataaagggaaaaaaggagacgtagaacttaagatggactaacctccatcttagttttataattgtagttaccgaaggaggttagttagttgaaagtccatttcaagaggtctgtgataatagttgctttagtagtaactagttttattctaggagtagctttaggttatactatccatgtagataatgctgttcaagaacaaatagcattacaaaataaaaaagattcttctactagtaaaaaggaacctagcaccaataaagacattgcagacacaaaagctgtagactatgagactgacgtagctgtaaggttgaacctcctacgagtaacagtaaaagaattttctgagaagtttaacgaggtagaggataatgtacaagtacttaatgatatagaatactataatagttatattgcactaattggagacgtgaaacgccaagttagtgatatacaaagaatgaagtatcgtagtagtgagtctgctttagctagttctgtaaaccaagcagtagacaacttctatcaagcctcctccctagaagaaaataacgttcagtcaagacagacggacatttctactatggttgttaacaagctaaatacaggtaatgatgaattagaaaaagtctttagaataataggaggtaactaaatttgggaattgaagcaatagtatgcgccttatatggtgtttgggtagcacggaacctgatgaccgtgacaagtgttgatgaaacactgacggaaaagattattctagaaagcagtaacacacaagtagcccacttagcaataagtgaagccaaaaaacttctagagaaagaaacaaaagagaaaaatagaaaaattattattgattacattgaaaaatggcatcgggcactaagtgacgatactttaatagataccctagtagagaatgaaagaaaaggggctaggaatgctagaattattgctaatatatttgtaaaaattgggacattacttggaatcctagtattaaatattccgttctttcttgcattagctaacttcacagtagcaggttgtgtggcttttgttatattaggcgtaatatatggttgtcagacggaagcaaagcaagaaggatacaaagcaagtaaggactatattgtagtctcttatgtgctaaagcaggctttattcttgctggtagtgttaatctagtaggaggttttattatgagagaatatgatgaggaaaaagaacgagagctagctaagaagaaccataggataaaaagagaacgagctgaggcacttgaaagaaaacgagagataatagagtatgtaagagaactagataataaggagaaaaagttagtagaggaacaggcaaaagaaaaaataaacggtaagaaaagtagtactgtagaaaagtatggtaacgcactggatatggtaaagaacacagactttgaagctttgtgtcgaatacagacagcttaccacctacagagagaatatcctatggttttaagctctctaaataacaaaggttcttatattcatctaggagtagctaagcaagttgcagataagaaaaccttgtcaggagatgctacttacgagtgtgagctattattttcagttcaagtacctaaagcaggtcaaaagaaacaaaaagatttactactagagctagaaaagagaaaaacacttgagggtaactatactaacgtagaaggtagttttactagtcaccaatacattccttttctaactttcttaaacagaagaaagattgatgcaaagattgattctatgctagatgactactctctagctaaaacgaaggcacagaagaagaaaatccaaggagaaataaatacttgggtaggataccaaaaagtagtaaaaaataaaggatacgttctttatagctatgcaagtgccaaagtggtagcagtttatcacagagaaataagtgtatacagttaaggaggggttaaattgagcggactaaccgtatctgataagcaaagaaaagaaatgctttctatgatagagactctatggttaaagagaaatcccagccaaaggtttaatcaatttatgtttaacttagaaagagagtttgcttcctataaaaagaatgaaatttcaaaacgaaatagagttttctaccaaatgagccacttagggtacaacaaaggatacgtctatgtagcccaccaagtccctgatttatttgatacatctgaacaagacttctattcttatttgaaggataggataggataggataggataggataggataggataggataggataggataggataggataggataggataggataggatagaccttcctatcttttcttttgttctaataaagtagtgggttacagctacactttcagttgtttacaagttgtaggactctctaagtagccagctatagttttatctgtaaaaagagataacccagaccccattatagagcatatataccctaagtagcaggctacacaaaataaccaatctatagataactaatgtcattcagagactatagatgacacagcatccttttgtaaatttatatgttcgggctagattaagaggcgataccccacccctcctcttagagtacttgtagagggggtatacagagtttcgttttcctacctagtatttatatagaatatataagtatcttccttaaaaagcctgtggtggcttgtgagagcatacaggaaggtattgtaccttgtatttatacggtagcaggtaccttaacccctcatacaagaggaagctttctaccccatataaacgataaaacactaagtagcaggctatactttctgatataggacagtatgacttatcaatatgtaaaagggacaaagctctaagtagcaggttacagaaaaaaggaaaaagctctaagtagccggctacagaatttcctaagtagcaggttatagatattaaaaccatttatggggtaggtgaatgcaggtagatggcggggtggtgaggactttgaaataccttgtgaacgttggtgttatgccgtttactgctagatgataaatagtttcttctatataatacaacgaaagtaagctaaaaagaatttaaaaatgatgtataaaaactgttgacactttttatattgtatgctattataaaagtagttaaagaaagcaaccaaataaataaaaaaaaactttataataactgttgacttttaaaagtaagcatgttattataagagtatagaaaagagcaacaaactaaaaaagttaaggtggtaatctcaatgaaaaaattaaaagcaaaaagtaaactaagctttatcctcccaaaattaacaaacgacaacaaaaaaattgactactctaaaattcttacatcagcttgcaacgttttcggaggctacacattaagagaggttcagcggttgggttgacaataatacattatatactgacgaaagcaacgaactagaaattttacacgaaggcttaaacgaagacatgaaaaacacagtagaggaaattcttgattttcttttcagtgaagaagaagcgggacagcttgcagtatacgcaaacaacaataacgaagtttacattttagaaaacgaagatgtaaaagaatttaaaaatattttataataactgttgacttttagaaaagaacatgatataataaatgtacagtaaaaaacaaataaaaaaaaattaaaaggtggtaatattaatgaatattgaaatttgggcgtacgcaacgaaagaagacgaaatttgtagtgcagagcaattcatttgttcagtaagtaatgaagaagaggcagaacaagaattaagctacgcagagatggaaaactaccacgcttggaaaacttactaataaatttatatagcaaaattataataagtgttgactttaataaataattttgctataataagtttatcagttagataggtaaacaaactaaacaaaggtggtagttaaaatgaatgtagtaactaaaatcagtacaacgatgaagctagacgactatagcaacttgacaggacaagaatttacaacatctttctatcagttagcaaaattgcataaaaacgaaattaaagacggagtaatgaaagaactatctaattatcctgaggagttacagaaaaaaatacaggattgccagaaagaagcagaaggacttaaaaaagtagtacaatttgttagtgaagacgtgaagaacaataaagtaaaattacaaaaaaattcttatggtgaccttaacactgtttctttgaaagcaagtgtgcggagagctttagcgcaatacggtgacaatattaataattgggaaaaaaattacttactagaggattgtgctatacgccttcaatacaatgtttctaatgtgtatctttcggaactagctaacttgcggtcagatgcaagtgaaaatatgattgcatggagctataaaaaagattattcattagaagcagagtttgaaaaaattgatgactacatggcggtaaaacttatagacttagttacaaaatcacagcatgagcatagaaacacgcatgacgatataagaatcaaactgttagcaattctagaatttcaaaaaaaatacgaggttattagctgggacttatacaacggtaaaaagctttctgatgaagaaatcatagcaattcatagtaaactaccagaatttatttcaaagtttcatgctgtttacactgatattgaggagttaagaaattatttagaaattgagtaaaaaacgcttgacttagtgatgatacaatgctataatataagtatagtaaaggttatcactaagtcaagtacctacataaaattaaaaaaaagttataaaaggtgttgactttrgakactaaacatgwtataatraakgkatrgtaaragttaaggacgtgttaattatgaaaaaacctattcaggaactaagacaattaattgaggaggaagaaagagtattggtagactacatcagtctagaagacgacaacggcagaaacatcatttttattgaaaacctaaacgcagaaaactcagatgacagcactatgctagaaacttactccccttatgaagcattagagtttttagaaaatgaggggttgcttgcttaataaatttatataacacagttgtaaaaggtgttgactttaaatagtaactgtgttataataagtttatcaagtaaaggtaaacaaattaaaaaattaaaaggtggtaatattaatgaaaaatcaaattgttgggtataaaaaattaacggttcaaaaaatttggagagcaacaaaagcgcaattagtatttgaaggtgctagaatggactacgctagatacctaaaaatgaaaaaacaagggaaagaaatgggtattacagtaccaaaattcacgttgaaagagatgtaccaaaatcgtgctagcatgaaacgtgatgaactaagaaaagacatgttcctgtgccttgatattttaaatgaccgagtaggaaaataaaatgtggtttgtaattattggtatcagctttggactaattttagggctacgaaaatacaataaggggaaaggagcggaaaaaggtgagcaaaaaaaaccttagtattagtaagatgaaagaactctgtcaaaaagaagggagtaccttctttgaacctagcgccatgaaattcttcgggacaaaaattgtaaccaaaccaaaaaataacttatttattacggaagatttagcaaaagaccacagaactaaaaagtttacggtaagaaaatttgagaatggtgaagtgattagtgttaacggttttttgaagtacagttccttggcagaagctaaagaagttctaaaaactattttataaatttatatagcatagctgttgacttttaataataactatgctataataagtatataagggaagagggtaatttaaatggcrgacggtatttgggatttaggagtataccctattgacaggaacccatcaaggctatatagcacaaatcaattttgacggyacaggaaacgaatatcctttaccagctacatgggatgagttcattgaaacatttaaagatggttggaatggtacttatgaagacgaacaagcgttcttcaacgatatgcaggaagttgcattaaaagaaatcttagacgaactaatagacgtattatataatctagacattactacatataattttactatagatgatgttaaaaagaaagttattacattaaacaaacctacagacgaagaagatgcagaagacttagtaatagaatttgattctacttgctttygggatgctacagtagaaaatgataaaataaaggttactgtaagaaacaaaagtaaataaataaataaataaataaataaataaataaataagggttatcactaatatatttatatagcgtatctctacctttcacttgcaatactagcggaggtatgctacaataaacatattagacctaaataaaaaggagaaacttatatgatgacaattgaagagttagcaggtctacaaaaagtaaaaaataagctagaatgtgcatataaacaactaaacgatgctacagaatcactagaaaccactctaattaatgaacctctcgctataataaataccrattactcttttctagctaataacatagaggatgccagagaggtattaaacaaaacaatcactaagttagacaggtataacgaagcactagcaactaataaagaagagatagacaagtaccttcatcttactaagtctaaaactaaataataaatagtataatagacaaaaaaaaataataaaaccaaaacctgtaaatcacctcagcatttttttcacacggtgtcaactcaaaaggcttatatatgcgcagtctttctacctattttttagaggggataatagtactatataaataacctctataggtataacttcttatatttaggattatttcaaactaattaggtctaaactcacattattataatgtcaatagggtataactcctaaaatatataaatctatgtacctattgactaataatggtgtgtaaggtataatagaggggggaggtgtaggcatcttatgaaaaagaagaaattcagtttwgttatttctytattactacytcttcgattataytagtgagggtmcgcttgtagatctaaaagaaacaaaagtaaaataaaactgttgacttattaggttaactatgctatacttaaggtataaacagttacaagtgggataagagggtataggagctcgtatataggcttttactttattagataggtaaatagcagtaaataaactaatcccgctcagaaaggcttgtaggaggttagagaagatgtttgtattaaatggtttactatgtactttactagtattgatgattctagtgggggctattgctttaaacgcagataagcagactaagcttattgtaggaattatacaggtggtatttatgactatgtgcgtagtcctctatatcactattatagggattagcattactattattccttttggtgtattatttgcgatttgtggggctgttgctttgttcaatctacttacttttaataaggaggagatatgatgacaaaatacaagttaaatacggttagtaataataatttaaaagcaggggacgtggtacttgtcgagaacttagaatgctacgaattaaaatcaggtgtttacaagattattagtgagtgcgacggtctatataattttgtgggtattgatgtagacgtgtacgaacctgtttctgtagaaaacattaaccgactgcttgggagctgtgtgttacgtgtatccgaattaactttggatgtagtcgttcaaaactaagattagaaaaggaggaagggactatgttgttatttatcatgaatgttatcagtgtagctatattaatgagtactttttactatatacgcaatatatcccctttctattacgagcggacgactagactagttaatcacttagtagatatagagctgattttaggattatttatctggttagtgtttattatctctattacttttgtatagggaaaggagtagaactatatgcttattataaagtctttaattgtagctattatatttactatcttactagtttatactatagacgggttttctcgtattcgtaaaggggctaaaagtaatggtattttagctgttgtttgcgggattatagggataattaccttttctattctagtagttattatcttttagaggtataccaattttttaagggttcatttagctaaaaatagctttggggtgtgccaacgagtttcgcgttttggagtgaaaatatccctaggggggaattcaaaaagttcacctataccaatttgaaatgagggggttttattgtttattgtactgatgggcgttttgggaatagtgttagtcggcttgggtaactattatacaatgaaaggtctagcgatgtcacataagcttagtaaagacgtagctagtgaagaatgggtaaatgaacagtttgaagattctatagaaggtcgtataaagctaaataactatgctagagctactatgaatcgttcagactatgtatttaacaaagggatgttattactatttataggtgccttattacttatcccgctagtaactttaatttaaaaggaggtagaaaatgggtttaacattagtaaatgacgatgataagtatctacgtgcgtttaaactagattcttttcaatcagtaaaaggaattatctatgtatatattgttattactcccgaagtattaacagagtacctctcacagagctcagatggacagtattttaagagtagtaaagattatcaagtaaatgtacaggaattagctagatggcttaaaacgtctaatagaggctttatagagacactatctcataatgagaacattatggcaagtgtaggagaaaataatatataaaagagttgacttctttcttttgatgtgctatagttagtgtatcaagtaaaggagttctttttatatacttaaaaaaaagttataaaaggtgttgactttataaaagaactatgctatactgaatatataataaaaacaaaggtggtaatattaatgaacaacaatcttaattactggacggatacactagcaatcaacttggcgggattaacattagtagaacgggaagaggtaactacacaggaagcactcatatcagagatgtatacaagagacttacaagctaacaaacctatgtttaataacttattagataacctagaagagttaaacgacaaagcacgagaagctcactcagaggctttctatcatgcagtagatgctattatatcgctgtgggacggaataagcaaaaacactttataaaggaggaatagttataatgacaaataaaaagaaaccaactgtacagatgacaggggaggacggaaatgcctttcttatcttaggaagagttactagtgcgctgaaacgggcgtgatacaccgaagaagaaagggaacagtactataccgaagccacatcaaaggactacgaccatctactggcggttacggctgaatgggtgaatgtagagtaagcatataaaccaatactattagaaaattattagataaaagggttgacaagttcaaccccttatggtatacttagtatatagtaaagagaggggacgagttacatgctaacactaaagaaactattgaaagctaacaaaggacaaaagaaagagattgagaaagtattatccgaaagagaacaagctatcgcaaacttagaaaagctagttaaactacaacaatctgcttaagcattcagttacgtcattagtggtaggcatatatacccaccccgtggttctgtgaccgtactttggggctatgctacacagggggctcctctcggggcacgtttttcgggaactttttttaaatttttttattcttaaagtcaacaaggaatggctctatagagggctttacaagaatctttataaaaagggcaccccgttgttttttcttaggtaattttaaggagaggcttctataaataaagtgaaaaaattccggtaaaaatttttaaaatagggaataaggaggtgttcctatagatatgaataaagataaagacaacgcattagagtttcgagcttggtgtaagataaaacagatgatggcagtggttatctctattaatttttataaaggagatatacaaggggaggatgtatttctctcttaccctgcacttgggtcatttaacttaagaggcttttccgaggttatccttatgaggtacacagggttacgagataagctagggacaaagatattcgaaggggatattgtagaggttcctttagaggcagaccatggatatgcaggctctgtgggggttgtcacgtatcgtaactatggtttaggggttatgacacctcaagggttcttttatttagaggaacctgagttgcttcttgttacagaagataaatatacgaataaagtagaggagggttaagaatgaaagcacctatttcagaatgtccttattgtggttctacagaagggtacacacttaggtcacaagcaagtggtactatctactggcggatgaaatttaatgaaaaagaggcagataattcagagtcacatgataatttacagtatagtccccataagtacgcttattgtaccgtatgtgagagtaaacttttcaaagcagaggaggtgaatgggtaagatggagcttcctaagcacattatagtaaaaattaaacgagcagggcaccataataaaaaagcaaatgacttacaaacggaagtacgcaagtggttagagagtagattaacagatggggaatctctccctgactatataggagaccaactgatagatgcgttggagcttggtgtagatggtagtgaggcgttacttaaattcttagctgaatatgaggagtgaatcttaatggaaaaattgaatgagaaccagcagattgtactaaagtggttaaagaattacttattagatgattctgatttttataacacggtatatgcttctaaacaattatacgtgaacggctttcctagtgataaaagtaatattgcttttatgaaactaacagacaaggagttcgctagcgtactactagcgttctctacatggtacttactagaattagaaaaggaggatttaacatgaaagagaaatgtagtatagagatttcttactataacggtgaagacaagtttacgggaaaagtagtgtgggttgtactagacaaagaagaaggtatccaatttgtacttactgagaaaccagaggacgaatactttgtttttgacagtgaggagtcggcaggtattgtaggtggtcttattagactaagactagcatgtgacaacatagaagatgactcccctgtaacaattaattaccttaaggaggaggagaataatgaatagctgggagtctattttagggctaggtagcacgcacaaaatggtaatagcacaagagttggtaagttttctttataaggaaaattgtgatactactacgctaggggagaatgagcttttcatactaaagtttctcctcgataacaaagtggagtttaacagaaaagaaacttattatggaacaacgttctacagaggttatactgtaaccgtaaatgttagggactctgtgttagcaactataggtaaggatatagcttccgtagataaagacgaagataaggctactccagaagcagtaagttggttagaagtggaatgttgtgatatactgtatgagttcctgtctactaaactaactgctgagaagggtagtggtagacttagtgacacgcttaaaataagtcgttcttatgaagaaaaggtaaaaggactagaaataactataacaaaaacaaaagtttttaacaaattaggaggagagtaataatgaaaaagagtttatggagtacaatactagaagacacagtactagaggataataacaactcaatagcatacgcaagtgttgttaacacacatcgcaagtttagacgattactagagaatgaggactatgaggctactattttacgtatgcttcttgaaaggaaacaagaaattgacggggtgtttcaaagcaacgttgagtttagtcccctaagtgtacgcccatcagcgccacctgtaaaagaacgagggtacgaaatagttattgatgtgaagagctttgtagccgaagcattagatatgcctaacgacctagatattaacctaggtattaatggctttgagcctgtctttgctaatatgctacaaaattatttaaatgaaaagatttttgaagaaaacgaggtaatatatgagtttgagggtagtacatctgtggagcaatctataaaagtagagggtgacggttgcatcgtagttgtgaaaaacataactcgtacttggtaaggaggaacttatgaatagaaaaacgtttagacctagttggcttggagaagcgtataaggggtatataccaccaaaaaatgattttcctgtgatacgtataaaaggagacatggtttcttggtttactatccttttagaacaaaatgaagaatttgtacgggagtggatgttaaagtataaaagaacaacacgtacaaaagaaacaggtgctcattatataggggatagtgactactccacttattgcgtgacaacctaccttgttaccacacgtacagcatcattcaatcccaaaaggcttgttatagctctatttactaaagaactagctcttttttctagggaatcgacatcagtaacactaacagaagaaaatctaaatttaaatgagcgtattactgaatatgttgaggcattgagtgagtatctagagtctgtaggtatttttccgttctctcttcctagttggtgtcatcccgactatcattacacatacgatggctgtcgttttactatctccaagagagaaagggaagtaatatctagcactaaaatgaacttagaagaatataaccaacatttactaaaaagcaaaggagagaaactaatgaaagtaactcaattagaaacatttaacgattttattgactacatggttaaaaagaatgaagaaggctggaagtgggaaaacaaagaccctctagtacctgtaagtttcgaaaggtggcaaatagatagagaccaaacacttatagctaaaaactttagaaataaggttatttatacagttagggtggaagaccttgaccaaacacctacaattgtatcatataagacagatgcggactgggtcggtagcttagacccaaaagaatatgaatgggagacgatgaaacctaaaaaagtacctattgtccacccgtgtacattaactgttgaagaactcgaaagggattggaagactaatacaaaagagtatgacgaagttaataatccttctcattatacgtctgggggcattgagcctattaaatatatccagagtcacaaaatgaactacgaaaaaggtaatgttattaagtatattactcgggcaggtaaaaagaaaggtcagagcgagattaaagaccttaagaaagcaatgaaatatattgggttactgattgaagatatagagaaatatgagaatagctaatttaataacaaaagtaatagggttgtggattctgtgtagactagtaagtgtctaagggttcacaaccctaatttaaaatgtgaaaacaaacgaagtataactgttgactttttacttatttcatggtatagtaagtatatattaaagaacaggagtgattacatgagtagagcaggctacaaaattcgttatgtaaaccagagaggtaagatggtttgggttaaaagtattgggtttggaacaaaggcattagaagtcgatactactaccaacaaagagaaggttaaagtatttttggataagaaatgggcacttcaagtatgtaaactcattgatggcgacctagatgttaattgtatgatagacgacattacttataagtattttatcttctatgaagcagaagataagaagtactttctaaagtcgctaactgtagttcaagataagattgagcctgaatggacagaaaacgagccagaacgagcggttattcataaatacaatgatttagatagaattttagggctagttaacgatagaattgataaggaagcaagagcacgtcacgatatgtttttactttaaacgaggaggtaataaaaatgattttatatggagtgtctctttatggggaggtaactgagtggactacagataaagctactgcaaataaatgggtagataatgcagtacaatctatagaggataatgatatagatgaggactgttatgttattttgttaagactagacttagaaaaacttctatatggcacattcgaccgtgaagtagatttaaacgaccaactgcatgatgaagctacagtactaaaagaattacgttggcaggtggacgaggaagggctatataatattgtggaggtggcggaatgaataataaaaagacagattataaagtatacaaaataacctacaagcaacgttttatgggggaggttcttattgactcgtatgaaagagtagtgagggatgataacgaactacagtctgcaattaacgctttatatgatgacccacatgtgttctcggttagtagtgaggaggtgacagaataatggagtttaaatcagtaaaagaaataaatacagagattagtttgttagctgacaaaggaggtatctcagacggctcacacacatttgaggagctttactttcatagaatggttttatttgcaacaatatgtaatgctaatcactctaaaagttggaagtctaagaaacatgaggacggttctatgtttgataactattttatcgttggtatttctacatcgaaaggtatgttttcttaccattatcacttagacaattgggactattttagtgttccagaactagaatttgcccctacatgggatggacatacagctaatgatgttactcgacttttagatatataagaaaatgtaagtaaacaaaggaggtactactatggatttaaataaatttttaactgaggtagataaagttacttctgcttacggggaggatgcaaactacgagatttcttttattcgtgagaaatatgcggacttagacgaaggtgccaaacaggactgtgaggaaacggacttctttgaggaggtagaatacatcagtcagatgcaaacaggagaagactgttttgagggctatatttttagaaaaataaaaaacacagattggtgtctttgtgtcacgtatgctatctgaggtcttggaattactactaactttttaggttggcttaaaatgtagtcaattagtaaacatattaaggaggtaaacataaatgatacaagcttatagagttcgttcacaagaagattttgttaacctaatggttaacttagaaaaaaaggattttaaatggtatagtgggttacctcctctagacggtgtatactacagacaaaacccagataaagacttagctagtgattctcttcttgttgttctaaactatgaacatcgtactattagatactatacagttgctaatttaagggaagcttacccactagcaaccatctatacgtacacccctagtagaacatctaaaacaaactcagtagacggttttactcctactcagttagtctatgacgaagaactttacctataaatgtactctacacaactaaaggcattatacatgtctttttttgttgactttatttatcttatatggtatactagtattataaataaaggaggtgtctttatgaggctatccctaagaaatatattggaaggttttcttctattctgcctagctttaccacctatcctagcagttatgggatttttattaggtattatatggattacattttactttcctatgtttggtctagctttttggagtattttaagtgcatgtttagtagagaacttgctagaaaaatgggataacagaaagactcttagaataaggaactttagtatgtggtatgttatagccttacttattgctcttactattactggtatagctatactagtgctgtttttattctagaacataaagagttatgtcttaagctagtttggtaataaaataaacataagaaggagaggttattttgaaagctttgttttttgtaataggcattctagtatgccttttaggtgttgctatcttagctacagcaatactgtggattactactagtcttcctgcaatcggtgtcccttttctaactattactttacttgtaggaatcctactagttctgcttatagaaggaaataagaaatactaaagaagtaatggaggttgttaaaatgagaaagatagacataaaccaagcaagtgtggatttacaactactaatcgaatttgagaatgaggactatacagtagcctatagaggagaagagagtaggcatacagaagaaatgaaggaagcaatcctttcagctcacaaagatacaatagaccgtttagaggtagctaacgataaactactagagacttatgaagacctgatagaattgaaaaaagaatatcacaccacgtataagaaaaatgagaagttagctgtaaaaaataaagaactaaaaaaagagttacgagatattacaaatgaaaaagaaaagttagaaaaagagctactccgcataaaagtgtctaagggggaactttaatgctagtatcccttacttttttagaggtacttttatgggtactgactttgttatactttggtatggtaatctacgtactcttatcccacctagttactagcgtagcttaccccgagtctgatggagttgctagttcttttatagactatagttatttatggggggaatctgatgagacgagcaaggctagtaactataaatgctacgaccatataaactacagaaaaacgagctattttaaccccacttataaaagcggtgaatttctaactagcacagaaaaggtgtctaagcataaaagaaaagtctttaaaaataaacaaaaagttaacttagatgaagaagaagagttataaaagacacataagaaaaggtacctgctaagggtaccttttacttttatactattttttgttttactggctctactatgttaaattcccctttatttgttatagtgatagcattaagttgcccttcaaaacagcaaccgccatctattcctataagtttttttctgtcgtgaacccacacgtcaaaattagaataattacggtctctaaacggtaagtaacaggtaggtgtgtgaccgaatacaattacttttcctgtgaggttctcttgcttataccagtccctaatccacataaactcgtggtctgttgtcttcttccagtcttccccgtataaagattgaatacctgcatgagtaaacagaacgtcaccaaactcgtaatacggcttagaagctctcaggagctctatatgatgtctatatttcttttgtatgcaaatacttgtaaagtgataaaaagacgatagcctatgcctagaacgctcctcctgtggcatattattgaagtagctagacaaggtgtttcctcctccactagcaagccatctgttaatagtgtactcattctgctcttcttctataaaatcaagcaacatttggtcgtggtttcctcttataacaaccacttggtcaccgtactccatctttaaatcacaaataatatctaaaacctctctagactcgtcccctctatctatatagtccccacacaagacaagcaattgtttatccctatcccaattgtttaacatttctaaaaactctaagctacaaccatggatgtcactaattgcaaaagcttctttgaatttagtcaccgaggcacctccttaatcaaaattaatttcttctttccaaccttttatataagatgttaggtctccttcgtaccccataagacgtatttccattaccaactgacctatcgtagcatttgaagtgctcataccctctaaaacctctataaacggtcttagtcttctgtccataatctcgtttgcagtttctatagcactctctttgtttctaccgttctctatgagtaaatttacgtaatcttcataagtcatgttaacatgggttacaccgtccaccacactagaaaacctacagtatctaccatttggttgttgtgagataaagctactcatccttgatattcatctcctctttaacagtactttttagccttttgttttccgcttttaccttttcgtataaccagagagctgtagacaagtcctttcgtgttacctcgctgttgtttcttacttttgctagtagctcctcatacgttttatctttaaagtccatagtagctccttcttctgcattgtcccaagcgacctcgtaagcaaccattcctgtcattccttgcatcacacccacaatagtacctatagatgtgttataacgatgttgtaccagttcaaactttttaaacttattcattcttatctatcccctagcaattatttgtaaaagtaattccaaaagattccatcctaccatatctaacagaaacacaagctgttaggtagtcacttactcgtatctttactttaacaatgtcaccatctataggagacttaaattttatttctatgtcaactgcaaaatgattgttgtgattaatccaagattttaaatggaatattccttcacgcatctttgttacagttgtgtatggatactctagtgaatgtttgtgtatatttcttggaaattctccaaacattgcaattacttcttttgggtttgttggtgctaacatgtgatgtctattttgatttgataataactctctgccttttttgtcaagctctaaaaagtcttctcctctatttttcagtaaatcaaataataatttttctgcaattacctttccttctttttgtctttcttttctctgctgtttatcatatttcttcttttctttaattatcctattccttgcatgactaaatttttccataattactgtcacctcacatctttatttacttataaaatctactaatattgccaaaataataagaaataatatcactatccaaacaaacaccactattactacccaaataattattggaataaacactgtccaccaaacaatattaataaaaccagttaattgaagtattaataaaactaggaaagctaccccgcatactgtacttaatctcatacttattattcaccttccacactggcttcttttaataattttattgtaacgttttcatcatctgttgctgttcccgcaagaacctgcttaaccatctcttcaaaattctctctttttggctcttcaaaatcttcttccgtatacacctcaacttctacttcctttttaggaaccaaaagtagtccaccaaattcatcattttcgactccataagtcatcttacctgcttctacatcttcgtttgttttaattgccattattgcaattacatctttaatagtatccaccatattcactctcctattagtttagtaagcttagccgggtaccacagtttttcattattattaatatcccattctattaaataggcaaaatcttcaccttgtactctagtgtatcattataaacttcctctactgtacctaactgttttttactctgtaaaatgaactctacttggtctcctactttaaacttcatgcttcttcctccatttctaatatttcgtgagggtctacctttacaaaatcagataaagagtacctaaatacaaccctgtctttttgagatacaagggcttcttgtttttcttttttatgacccctacaatgtttgcaaacaagaagtacatcacgagtgtaaatattcgtgttctctatagatttccacttagaagccgtaccctcccatatgtagacgtgactgcaagtaggcttacttctaactaaactagctagtttactagtgtatattttcatagtgtaaacacctccaactacattatatcataaaaaaagaagagtgtcaataaacactcttactttagatgttcccacccgtaggcactaaatatagcaattaccgctaatggtaaaaacgtaataaaggacacacgtaagataaatcttgagataggatttctgtagtaattccatagatatgttactttaacatcatggctatcatctttagactctaaataaataagtaacaggaacagtgggtatattgctacataaatttgaaggattagtaacaccatctatttagtatcctccttaagagacaaattatcgaggaaggctactaagtcttttttagctaagtttcctttgtgattgcctacaaagtcctttatagcagccaccagttcttcgtgaagtcttctgttctcttcgtaaacctcattctttaccgtccgctctaggttacttcttgcactctcttctcttttaagtaaaacgcaagcttccccctttagtctagtgttgtcttcctttagagtctctatctctttttccttttcttccatctgcccgacaaccctttgtaactctcttagttgccgttctttctcacccaaaataacttcaagagcgctcttttctcctacagatttccctagtttgctggttacctcttctagttcagtagtcatatttttaatagcttccttatctttcttttgttcttcataagcatctacaatatattcttttaaattcttatatgggatttctttctgttctttttcctttagtttgctattttttcttttagaactaattacctctaaaaacttcattcggattcctccttcattgcttcaatctctctatgcacttcttcatagtaactagctgtaaaagttactcttgaaaactcctcgtaagcctttagctcccgctcgtccctctcagttatctgctcccatgtaaaagttttgtcacactccttgcagtataaatcgtaatctaagtcataattacattgtaaacactctactgattcgtctccttcgtagtgcatatctatttaccacctttagagatactatcgaacttttccgctaaatctgcatacatatctgatagggagtcataccagactgttctagagaaagcaacagagtatacaatggctttttggtagtaaagtctgtcaactcttaccgcaagacgtctaaaactatctctactctcttctaggaaatatgtgttactagggtcaactgcttcttttatcacagggtaaatctctttaacaaaataagcttcttctttggagtcaattcccttttgagtcagtatacctaaaagaatacctgcacgttgttttacctcctctggtgtatctcctgatgctactcgtttaactaattctttaatatcttcttctgtacctacgcctataaggtgacaataaacttctaagtagaatttatttactcctaggtgatattcttcgtcttcaacatttttccagataaactctttaaggctttcagcaggtaagtctagcatttttaaaacacagctaacgttaagggctctgtacttgtctaataactcctcttctttttttctatcataagcaagggaagctgtatgtctgctaaatgtatctacgaaacattggtctttgaaaagatagttcagcttttcttcaccagattgttttacgcataccttaaaaagtttatacagacggtcataatcagcagaggataagtaagtaaaccaatcagctcctactttaggtgtgaaccctataaccttataactgttctcattacaaatacgaaactcttgtcttttaattatactagcgtaaacgaaaaatttacctttcttttccacaccaaaaatatttttgtctttaacaggttctgtagtgaccttaatatcccaacaggacttcaactccacccctaaaccacctaagatactgcccgctacattttctatcttaatactatcatttaatttgaactgactgtacttaagccccaattcctcagtaactacttctatcaccaaaggcattatatcctgtatacgttcctctgggttctcacttgacttgcattcaactataataagaggtagccatatagtcccattacccccttcacttgtactacccgcatctcttttttcataattatccttaatacgttttttatttagttcttctactatacctctgtagtaaatgttcatcattcgagtgttatctttgatgtctactaacggaatgttcttatactccttatgcttagccatactattcctcctcactacctattatttctttgtagaagtcctttacaaagtctgcttgctcctctttaggaacaatactattaatttcctcctcaaagcaaaatagaataaagcccactgttttgatagagccctcattatatagcttactgcttagaaccatgtagtccaacaattcttctttcttcgtattgtctctatgaagcttgtcaaaggtattaacagaaaaaccaaaaggactcagctcctttataactaacggctttaactcaccaaactctactttagggttagttttacttccgaaagaaagctccaactctttagtgtactttttaacaagttcttctacttctgtgtacgttcctacgctacagttgtttttctcctcctgtgagctatattttacgtaatactctttactcatactatcaactccttattctaattgtatcacaaaaactctattaagtcaacaaaaaaagaagatgttactcttcttttaatggcagtctagtttttagttttcgtgttcttcctttgtatctgagattgctagtatcagtgtgtactgaccttacttttacaatactctctttgacactatcatatagaaaatcccctacagctaccttacctaacaagctgtcatctaaactaaataaatatgttttagcataggtacagcttaggtcatcctgaacatagcttatgtaaacgtacttgttctcataatctgtcgctccccaagactcgtctacttcccatccaccaagtatagtttcaatattggcatttagtatctctgcaatgcttctagcttgcgttaagcttgtcagcggtcttacctgattaggttctcgaactttttcccagtttaatacaaaagtacctcttttgttcttaataaatgatgttgtgtagtatgcgtatggaaaccccttctggcgtagaacatacatatttcttttttttaccatataaatcaacctcctagtatttttggtaagtcctctgataagtttacagatacgtcttttcctagtaaattaagtaactctttgatagcttcttctagttctacaaagtactgtggtgacctatttcgactatcttgttgcaacttaatcacgcaatccatattcaaatcagggcatgaaccctctcttattcttataatattcagtttattatttaacgcatgttcgcacttctgtctatctttttcagcattttggtgccaacgacctccatcatattctaccactagtttcacagaaggtacgtagatgtcaaatctttttcctgctatatttttttctaatacaattcgagggaacgctttttttaaatagtaggcaattatcttatttggtttagaaacagtagaggaacacttagggcaagatttaaaccctacaaacttagacttcacactctgctgataagagccatgctccttacatttccaccaaacctttatgtcgcttcctcgacttacgttcttaggttttatacctatagcattgtttttttcatagtcccatagttttcttagaacagagctatgggactcactaataggtatacaccctctcttaagagctttagtagccatagttatctcctccgctctctactatatcttaggttgcaaaggtctacttctgcaaagaacatgtagtctacctccgcatgagacatccctaagtctcttaactcatatactttatcttttacagtttcccttacacgttccagttctttaataccttcggaagttaggttaccttccgcatctcttaatttaatagccatagaattttctcttattggagttagttgctctcctaaaattgtctgaatctgataatatagcggttgcctttaataactctccttttgtaagccagttgaataggtcatcaacagataccttcataagaacagctagttgctccataatgtcatcttggtctgtatgacatgacctgcttgcttggtctagtagtccgtaacaagcttttttaagtatcttcatgggtttatctgagatagtgctagaaggctcacgagaggataaagaaacaacaccagagcttgctctacaactagtgcacatgtagcacattccattaccgtactccttgccatataaatcagcattagatgtgtactttatattaccttgacataacgggcactttgtaggaatttccttcttcatacgtgccaacccccaatcgtcagtccatcgaataacataccagccgttattgctacagagacaatcaaaatcattaaaattattgacccccataaggtaagccctctggtgaagaactccttaaccgtaactaacagccaaataaatgaaataataaaaattatccaagtagttaatcccataactttgtctcctctcggtatttcttaatcttcaatcttgcatctgatataactgttgacaaaggtactagtagccttcctgccatgtaatcactatctacaccaatgttaatttggtctttacttggactaggtttactatgaatgtgtccatgaatgttgaatctacttttattacatccaagttccataggataatgggaaaaatgaaactctactgagtatgcgtgaataatcataccaatatcgtgtacttctactacactagggcataggtcactattgtataagtttactttttttcgtgtatcatggttccctcttataataatctttttacctcgtagagcattcataattttaaccgctttttcttcgtcacctaatgcaaagtctcccatgaagataacgacatcattatcattaacagtatcgttccactccattataaactgcgagttcatgttgtcaataaaaacgtatttatctcctctataatgttcaaacttaagtatgttatcgtgattaaaatgtgtatctgatgctaagaaaacattgttagaacttgaaatgttatatttaaccaaaaaatcagctagactaatcaattaaaaacacctgcctctagaggtacttctttataacctcctgtataaaaacttaagttgtactccgtcatagacttatctttactatatacttttactacacgattactggttactagaatactagctacctcttttttacctactacatgtttatgctttcttccgccaccatctgtgatttctacttctagtaattcacgaataccgaacataccaaacaactcctatctattctataataatattttttctgatttctaaccagtctacctctttgccaactgcttttaaagccttatcatcaatgtataagtcagcgccaatttttctagtatcatttttccatattgctatcttatctgggttgttttcatttacaaagtggtacggtatactgttattggcaagaaaatccaaacacaaatccataccaagctcttctctacatgtccaaatagtgataactgccccaagttcaacaagttcttctattgtctctttagcataaggtaggagctcccctactaaggggtagctctcctcctgctcactagcaatagttccatcaaagtctatagctatatacttatagcgtaaacttctcatctctacgcctttggtttgctatctttaaattttaactgctcttttgttgtagatggtaaacgataagactcatacaagtgcgggtggtctttctctaacttctkaccgttaaatcgtaaaccttccacctccgcttgataagttagttcaaaatctcctaaaatctctaatgattttacgtttgccttcaccatcataggcattaattcatctttaagtgcatctaactgtttttttaatatctcgtagttacgttttgttgttgcataatttttagctttgtcctgcaaggttttagtaatagacacccctgcaccctctttaagttctaccgcttgtaatgcaccttgttttgccatttataatctcctcctgtcttatttataaattaactataacacatattttattataagtcaacaaaaatgtatattttttatttactatcctctacttttaggattatttctctgagatgtactggaacactacgtccaagagactttttagctgtatcctcatcgtaaacaaaagctaagtcatctggtgattcctcactcataatatcatcatacacaattgagggtactaccttcattagttgggaaaagttagaaaattctaagttaggcaacttattttcctcttctagtctctgtagagctttcctaaccctagtctcagtgacgaattgtcgaatccactcggtactttcaggtaaatctttaacttccctttttctagattggactctttctgcgtgttttggggaaacacttttaaatagtttctggctaccatcgatgtctacttcgtctaaaaatttaacaataataccttctccttcattagtttccttagtaaatctggactgacctacaaattcgttaactgtatcaaagtcagtaaatacccctaagtaaaacatatctggcataggtagatttagtgtaaaagcaatactacgtacctcatctaaaggtaagtatcgcccttctacgttactgtacacgtcaaataagtagaagttatttctaaattgctccttatacggtgtatttaacttcttagaacctagccactcaccaaagaaagttaggtttactagacctgaatggacaacatcaggaatacttaccttgtctttaatgtaagtagtgaaacctgatagtgtgtttacctctcctacttcattaggaatgtctaactctatatttcttgagaatttacgcatagcatagccaccatcaggaagtttctctacaaaaaaactagcgttgctaccatctaacttctctagaatgataatttcacctgtatatgtttttgccatttgttgagctcttccaattctttgaatttttctatatctattcatattaactctcctcctaaatttttaagtctttgaacgaaatattatctagattgctgggttcaagctctcctgtacgaatataagtagtcatgagcttgctgtctaactcagtatcctcttttaataattcttcattaactttatttataagaatgccaatatcttttcctttagcgggaataggtatatttaatgatttagctatcttgtctacttctaacttaattgcctcagggagagtttcctgataaggcaagaactcatatagtaagccaatctccatacccaagtcatcagcttcactctctgttctagcaaaatcaatttgttgacctatttgttttacctcctgctttactgcttggcgtacctgctcatcagtaagttctgatacacttttaactttaacttctactcgcatgttatcaagtctagcaattaaattttggtatacattgatgaaatcacttttactttttctggcatgtataagactttctttcactctttctaccaaactagttcctttgtctttttcttctaacttcataaattttccctactttctctatattctctttttgtatcatcttttataagttttctctcatatctgtttcttagttttttgtctcttccttggcagtgtggaaacatcccatccttagtgaagagtagctcctcgtacactctattagccttattcttaaaatatttatttgtagattttttactcatttagttatctctcacacgttgtaaaattttacctaacaagtttgaacctctccataaatctttgtttaaaatacgagggtcactttctcgtagtcctacgccccatatagtgtcataagggcttgcctctactaaagtagttcccttagtagcttctagtttatcttttaattccttattttgagagaacttagccattaagataataaaaacaatagcctctttgttatcatcccacgtagcctcgttaaaacctctaacttttctacccagagatttaactgttttagggtctccactaggaacggatagaatttgtgaagccactaactcatcattgaatagacgtgccttctcatacatgaatgcctgttctgcattatgaaacataatagacttgttagcttctttcccttcttgcttgtactctaaagaaaaatttgatgggtgccaattactaaaggggctttcctttccccaaaaaaatgtgtatctatctctcattttataactcccctccttgacttagtaaataagtaggcgtttctgtgttagccattctgcttactcgggtagctaatgtctcaataaattttttattatcaccataaatattaatagtctgcgcttcttttttagttagtgtagtgaactttgtagttttgtcaaactgtacaaaacggtaataaaatttttgtgcttcttccacagaactagctaatacccaagttttttctttatttcgttctgaaatttctactactaattgtttcacgtgaaacaaccttcttatttattttagtttgtgttctcttatttaattaatgcgccttaaaattatagattggtttaacaatgaagtcaatgtcaaccgtatctgtaatattctctatgatactctctttctttttataggcaaatggtgcctcatctagtgtagactccaacacagaggaagaccaaacatctttcattgtattcttaaaatcttccatttctagtgtttctttagctttagaacgggacagtaccctacctgccccatgaggtgctgaattgttccagtcttcgtttccctttcctgtagcaataatgctaccatcacgcatatttagtgggataactagttgttttcctttagaggcatctgtcgcacctttacgaataaccttattagggatgtcaatataattatgaatagagtcaaactcagaaactaccaagttttcccaacccattttagacataattactttatgaatagcttttcggttatcctccgcaaatttttgcgcaatagccatatcatttagatagtcctctactaatttaccgtctagataagcaaggtctttattaaaaggtgctgtacttagttttaagcgcaataactcttcctgtatgtctttttccctaccttgctcttttagagctctaatgagctcctgtggcttctcagtgttagaacaatacttaacagctagttcttggtgtgttttagctacttgaacgcctaagttacgggaacctgtgtgtgtcataaggaagattcgaccatcgtaggcacctagttcaatatagtggttccctcctcctagtgtaccttctgataaagcgattctattttttacctcttcttttaagtttccatagatagtcagattcttaagtgacttacttaccttctcctgatttagtgctactcctcttacggagtttcccgaaggaacaaactctttaataacactatctaatttttctaatgtagtattgtttacctctaaatctaattcggttactcgtattccgcaaccaatatctactccgataaggttaggtactactttgttagtataagtcatagtggttcctacaacacaccctttgcctgcatgaatatcaggcataaatctaacacgctctcctttaacaaattcctgacttaacaattctattacttgacccacagcggtttcttctacctcactagtaaaaaccttagctgtattataccttccaagtagttctatcattctgctcacgctccttttattatacttcttactaaaaaccattataccatatgtcagtttataagtcaacactaagttaaaaaaagaggaacacgtatgcccctctattttagttctttaaagttcttagagaagaactcctcataatattggtcttctgtagtaactttcctacctgagtctgatactactgttatatctcctactagaacaccgtcttctgtgatactatttacagtaaacgttctgttcttgttacctttttcatgaaatgtcttatttaaatatttttttccttttttagtcatacataacatcctcttttgttaattgtgctagtgctttacaatatttttctttttgtgaggcaaaagcaatagtgttatttgaaaaatagtctagaatctctgctagttttacttttttagctagattatcctgcttcaatctctggatatagcaagtatacgcctcattttgaatgcgtgttaggtgaactaaacttccaagtaaataatccttgtccactagagataaaaaagtttggacatcactaaatctgacggcggtatcctcaaatatatcatgcagtaaacttaaataataagcatcttcatctttgtctttcctaaagttttctgctactcggtacacgtgcgctagctgattttcctcataattagctaaaacagtctttaaatgattacttacgttgtctttaattgtaaacaatacgtagcctcctttaaaaaataattggtagaaccaatagtacacatattcctagagaaacaagtaataacataatagtatctcttactgaaagtaggaacttaagtttaggtacatatggcatagatacccctgctctgcctaaaagattagaagcaataggcacaaggttatactcaataagaaaagctaaatataaataagagaatattaaaataaaagtgattataatccccatagatgtctcctctctactcccctaaggagctattttcatgcttatctattttaaaaagctcactagaaggaacaccaaaatatttttctagtttcactcgatttctctctctaggaaacttcttacctgtctcccaataagaaattagggagtagttaacatctaaatcctgcgctaattgtcgaatagacaactgctgattctctctaattcttcttaaatttcttactttcccaatatcttctttatttgtcattttttttttcaccccttactttttatatatttattataacataaaagtcaacgtctgtcaagcgtacaaaaaaaaagagcacgtattaggtgctctctaggttactatagaatgctgtcaataaagctgtaggcttcacttcttaaaacagataccataaaatcttttggtgtatttttttctaaagacattacatgtttaaagacagtaggtgtaattccagatactaaagcaactccgctttcagaaaaccgaactggtgagttatcattgcgggcatttacattccaaaaagttactgttggaagtaagtaacctgcttttgaaaacttacgtctcatactttcgtagaaagagctgtcgttctctttttgtgaactacactggtcgaactccatgtcagagataatgacaatatcagtaggtagctcactttgtggcagtttgctctttgttgcggtattcagaattaagttaaaaacagcctctaggttagtggacatgccccagtcgctactaatagcagtagaaacccgctcccgtaagcttaactcactaggaacatgaactaaagctggtttagaactgaatgtaataaagtggtctttgaacactccatttagtctctctgcgcagtatagccctaaagaaatagaaacgtccataggtgacacttcttttgccccaccatacatagaacctgatacgtcagaaactacaatagcatttctagtcatattttctacgtagttaggtagtgatttccaagttgcctctactgtgttgtctacagagtcgtcccagtcaccacttgcggtgtacttcttaactaactcataaggcataatgttgctggcatttagttttttatctgtggagtttactctgttaataaacatttcataggatgtgggcaggtgttttctaaaagcccctctatatttaaacagggcacgagaaggtaacttgtcaaaattgatagtatcgtaatccttgtcggctaagctagtctccactaagtttaactttgctcgtaaagcggaaagtaatttacgatactctcgactggtgtagcctagggaagcagacaagtctctagctgtatgtttcttctccttgtttggtgaagactccgaaggtaaccacttagctaataaagatatagattcatctgattttaagttctttttatctagttctagttgttcagaaattagttccactagcaccattgttaagactttgtttttagtttcacaactatagcgatatagagatacaacatcgtcccaacgaccaatttcactcataaactctgtaaacttaattagagtgtctaagtcaagctcagctagccccttaatggactcgttatacactgctcgttcacctaagccactaagtcggatattacgaagatgtactaagttcttgagtgctagctctcggtcttctatatatgccttcttaaataacagcttagcatcatttgttctatcacgcattgctcctgctcgtgagaaaaaatctaagttatagtctaaagtagtgctaaatgtgttagcccccttttctgttttacttaaagtagtctcttcttttaatttatttaaaaattctgacataataatctaccccttttcttttttataatcttatagctgacctaagctatattcaggtcagctatcgtatgtacaggaagcgtttttttctcgtacattttttttttacatggttagacaccatgtcgtactaactgctaatatgcgctgttttagtttttagtatttgctgtgtgcttcctaattaatttttacagagagcgtttttactttttcaaatagtataggttagtaattgctgttagctctctattttatattctgtagattatttttttatcacaggaatcatttttatattttatccaataatatagtagtaattgctgttagattcctaagtttattttatcttaaatttatgataacatattttgtctagtatgtcaacatctttttacattttgtttttgttgtcattctgccagttttcccaaaattgctctttttcactttttcggctctcgacaaaaccttgtgtcttcttacaatatgggcaatacatatgtttaatatgaccgtccttcttttgcttagctgaccttctgaatatagagactttttccccacactcagggcatgttaacgtggtttgtactgtctttaccatagttataaccagattgccccgttgggaatcgaacccaaaccgaggagttatgagctccttgctcgaccttctagctacgggacaatgagggaaacttctcttagttagagaaggctccaaaatgttttagttttccaccgagtgcctttgtcttgtctgatagttctatttttgatgttagacgagacttaccttctgccacgtacagaaaatctttctcaacagtaactaacttcatttcttttttgccagtctctttgttgtatcgttcctgtaattctttaacaggttcggaaattacgccgtaaaattttttcattgtaaaacaactcctaattttttattttttaactaactttgtaatttaataatacaataaaagaaatttggagagattccactctcaactacttcgtgcatcaatatattcagtcacggctaaactagattcttggtaaggagctaccttgtcctccatccagtctgtcaattaagactaagtttaaacacatcacgttggaagtgattaaaaatttaccgacaggactattttacctgtacatttcaccaagcatggaagagtatatttttattaataccaattttaaagatgtagattttggctactttgactcccatcatcccttatactcggctttcaccgaatcaactacataaatgacttttagtaagtagttttaaaagagtacttacattcccttctaaaacgcttcatcacgggcaggtaggatacttttatcttataacacacgtagcagtacctttgtctcacactaaccataactatggctcagtccccaacgcatactgatatttaccacatatactaatcgaggcggaaaatttaagtacattagaagagctaccttcttgttatgttacaaatttcctttattctattatcaatgttcttagtaacactataccatagatttcatatagtgtcaagtaataaatcaaggtggtaggattcgaacctacgacttcccgtctccaaagcgggtactctaaccaaactgagctacaccctgtcggttccgtacacaaaccttttaattgcacggcatatttttttgactagctacggacgtctagaacgccagccgacatctcctgtatcagagaagatgaagcagatatatagtattatctgtagtttaaggtatgaggatactagaaccagaaagaagcacctctattagttttcctgttcctatagtatcggaagcaaaaccgaagttataggtggatttcctatcttttttactcgttgtaactcactttagttaccgtttgcttttagcggtgtgagtggtaatctctttactgtacacctagtgtaccacactttgcgtttattgtcaacacttattttctaaaatttttcctgtaccaattttctatcatactatttatatagttattcatgtagtagtcagtaactataatacccttgtctgctgctaatgtatactcatgaacccgtaatatatagtaagtacttataggaagtacaacgttacctgaataaggtttagtagttctctctacatgttttataaatgactctatttctttcatttcttgttggaaagataggtattttccatgtctagtcctaaaaactagtttgcggtaaaacccaccatcagggatatgctcttctctagatattctagacctttgaagtttattcaaccttattttgtctagtctcctctcctttgaatgccagtccgctttactcttgggcgtcttcttataacttctgctcataggacacctcctaatgattatctacctagtatttctctctgatagtcattttaccattcttttcttctgctttacgaaaattctctctaactttagctactctagcttgctcttcttttttctcttcgggtgtcagctccattaaaccgagaactttcatagcatggtcatactcttcttgtgagttacacactactgtatgtttaaaattaatctctgtcaatattgaagcctcccttgttgataattatgtacctatggctgttaactcctattcatttaacttccagtctataagaagcaccacagtttgaaacagatttataaggttcctcaacctacatataagttaactttattatgtatttacaaagtcctcagcaggattcgaacccgcgctaagggagtagaaatcccttgtgcatccgctacaccataaggacacgattctcctatacacacagccccgctcttaaccagatgagacgtaacggtcaccgaagcaaccgtaagggagtcgaaccctttccgagctgtgcaaaggaaagtaacataggtctcttattatgttacttagtctagtgtccgtccacataggttgagactacctcgggacacgtccgcattctgttaattcagatttatttagtattgtgttgcaacacaacgctgggatgactaatccctaaagcgggtagggggattcgaacccccactacatggttggaaaccttgggtactacccttatactatacccgcagaggcaacctacttagggcaagtaagttaccaatacaaaaggagtaaaaattaattaaaacaaaaaagcgtatcctgctggatttgaaccagcgacttccgacttaacaggacggcactctgacctctgagttaaggacacaaaatactcggcaaggattttcaccttgcaataaccttcatcattcgtttcagctatcaattcaagcatcggatatgctctcagcttacttctattataggttataccagtcctactgtgcgatattttttccgccacgagtataaaaaatagtaatttaagtacaataggtagcaagtttttgtaagtgctagcaagcgcaatactgtaccactcagtattaaaaaaggtgggatatacaattgtaatttcattttactgtgtttgtaattgttatcgtacatggtttcatacacattgctattgagttaaataacttgctaccgaggtaactgtactatttttgcctgctattcctaaagctcttacaattgacaggtaaggagtcgaaccttaataactaccactagcttgtctttaatacttatcggtgcaccttatagtagacttagggttactagacaaactaaagtatactacatacttgacttactacccgacatttgaagggaggttagaaattgattttagcctctttttatttgctgataatagttttttctactatagcagtaaggtgcaccgtttgtttgtctcaccacatagtaatcctaattttaaggaaggtaagggatttgaacccttgtaccctgtattccaagggcaaactagttttcaagactagcgtattaccaagctctaccaaccttccattttgttgctatggagtccgcaggtatcgaacctgcctctcctgcttttcaagcaagcgctaatccatctcagctaggactacgttaatggagccactaggattcgaacctagaataacggggtaaaaacccgcagtgatagccatttcaccataactcctttgtatagaggttagagggatttgaaccctccgtaactagctatatagtctagtatgtcgtcatttcatcttaacctctataataacattataacatactttaagatgtttgtcaactgttttacagggatagtaggacttgaacccacctcattggagttggaaaccaatattttaccagataaactatacccctaagctggtctagttggattcgaaccaacgtatcacggaatcaaaatccgttgccttaccgcttggctacagaccaatagtaccaagtaagctatgcttgtagtaggacttgaacccacgtctgaggcttacaaaaccactgttctaccaattgaactatacaagcttatttttttattaagtcagctctctaagtctccgctctttacgttagtaattattagctgactcgaagagctaacctattatgcgccatcaaggactcgaaccttggaaccccgaagggaggggagttacagtcccctgcaattgcctctatgcgaatggcacgttgtaaagctagagtacatacttcttgcttattaagataccctagctaatttttactacttgttatatacttcccagtcattagcaaatacatctaagttgctaatacaccacattacatgacttccagtagctgttctcatttgcaagtaaggttctactttaaatagtgcaccttcctctaactcataagcatctgctgtttgtttattagcagggataccctctggatagcctttctgagctactacaaacatgtctttaccattccaacctgttctgctagctttctttccttttttaattaaaggtaaaatctcttcgaatgtcataattttctcctccttctatgtcttattaataccgagagtgggattcgaacccacatatccttgggacagcagattctaagtctaccgtgtataccagttccaccatctcggcaagtgtttctactagtctttgattacattataatagttagcataatgagtagagataacaaacccacctcgaatgtttaagtatggagggattctttctaagtcctgtaaatcaataccatctactaagtaaccgtcattaggggcatctgttaatggggatacagcaagagagttgtctaccgaaatagttttcacgttaggatattttttatcaaaaatagttttctgaaactttgtgcttacaataacagagtatccaaattcctcagctatctccattatagcctgtgtttttccttttctcctatacttaacagtactgataaagaatagaggctccattcgatacatagccttcttaaatcgccttacttctttactctctttaaacttagacttttttactttcttatgagtgttcttttggttcagcatattcatcagtccctttcatttttattcaagcgcacgtaaggggaatcgaacccctatttctcgatagacagtcgagtataatagccattatatcatacgcacagtactgaggtacttctagctaaaaagttacctcagagtttaacttggcaatagactggtcggctgtgtttctttagctaaggaagccttctacgcactattagtaccaacacttggaattgaaccaaggactaccgattatcggtcggttgctttgcctcctaagctatgctggtatagtatactctctcgttgactactatacgggggaatgtattaataggaacgagagaatacctatttttaattataccacactctaacagctttctgagattttaatctcttgggttttcctttttcaaggtcaaggtttctggtttagtgtgtctactacaaaccttttttcgaaaggagagtaagggattcgaacccttggtactattactagtacaacgagttagcaacccgtatcattaagcctctctgacaactctccaagaaacaaaatttaaacttatatttatattataacataaattagtacgtttgtctactaataacttattttttttcataaagcgggtaatgggaattgaacccatatacgtagatttgcagtctactacctttccagttggttatacccgcaagttctagaagactaataagactcctagagtttttaaatatttagtgaaggtaaagatataaacctgatgataagaaaaacgtgttactaacaatcgtctaacttacctgtatatacaatatagcatacttactgataaaaagcaacagttttctataatatttttcatactgagctaagaaaatgtatagcaaccctaaaactatataattatctgacaactgtcctatttaattttattcgtcccctatattatattctaatatattatataattatttaaagatatgtaattagacttagaatataaaaggaatataatataggagaaccttaaaattaaataggacaattatctgaatattttataacgactactattacttgagtaacaatagaaacttgatactagtatcaaataaaaagtaatttaatattgatagtaacccacctcaggtacctaaatatattcagaaaatttatactactgtcctatttaattttatctacctcctatattatattctaatatattatataaagactataataaattattattattattattatttataatatagaagagtcctaaaattaaataggacaattatctgaatataaagtttaattaacataaaaagacacctgtaaaggcatctttattaaacatctaaaaatttcttttcgtaggatactacaagtgaggcatttacataaacttcttctccatctaatgctttagtagttctaaccttaataaatggtaactcagaacctcttacagcagagtttaacttagtactagggtcagtaacagtatcaaatacatcttcactgaaaagataaaatacatctcctgcaatagtctttagttctaaaacagtttgtcttctagccatttttattgttcctcctctgttatgtggtataattaaagtatatcacacattacttcttaactaaactgaaaacataggtaactaccgcacctagtataatcataaatactttttctactaagtcttttttaccttcatcttctttttggttcgtttgtcttagcgcttccacacctttctctaattgacctatctgatagaagatgtgtgactgcttttcagattgtagggcagaatctttatctagagccactacaatatctcgtaattccttgatagcttttgttagctcctcattgtcagtcttacgtgttagttcacccgtttgtactttatcttcaaggttttttattttatctagcagactttgaatatttgtatctttttccactcacatcctcctcctctcagttctttatttttggtagggtttacgaatgtgttctgatacatccgagctttcagggtagttatctaagaagttcctaattatatcttcataatcaggtgttacaattctcccgtaatgtgtaacaaagtttaggtgaaagtcaattctaccgtaggaacctaggtcaaggtacttaccaaccatgtcgtcttcatcaactttaaaataaccaaagtcaaaacctataagggttcccattgatagagccctactaataaagtagatatgatattcttcacgattttccgagaggaagttacgtaatatatctctaaattctacttttagcatatccctgtattctttcattgcagactcctttctttacatactccatgcaaccactactataaccataaaagatacaactattacaaaatatataataaaggtgtaaactaacgacataagtagtacaatgttagccacacctgctataaacgttgctataattagcgtaaagttttgcgtaataccttgtatatctcctagagaaacatggcgttgcaagtcacttataacgtggtatgaagtccaaaaaatacccacttgaaacaagacagaggctagtacagctataaacattatactcatagtaagacaacctccttaaagctagttctaatatagtatacgacatagaataatttaaagttag

SEQ ID NO: 2 - DNA, 135028 bp, Unknown
  Bacteriophage P100+
aaggttgtgctaaattaatacagtagaactatgtggaatggaggaaatattatgaacaagccagacaaaaatttctttatgaaaatgattatcgttattctatcaggattacttgtgttatttgttataaacctagcatctatctactttgggtactctcctcacgttgtagctacgtactttatgacagggggctttattgcgtatatcgtgataaacttatacgaccgaattgttttccaaaaaggttctcctagggaagaacgagagaaacaagagagacaaaagatagcatctagacaaaaagctttagaaagcgtagtaactcaacgactaggtatccctatagaggagctagatttagaagtactcattcagatggaaaaggatttagcagacaaaactgctaaaggtaataaaaatgaaaaaactgagtagagacgagataaagaaaagacgaaaatatttcattaacgaagcaaaaagagttacacatatacgagcaggagaccgtagaatagcaacaggttcaagaatatgtaccttatgtggtagaagtctatcatctcagaaacggtctacaggagagacctttgcctcagtaaaccacatccattgttactttaatgaacttcttatcgttaacttatgtgaagatgcaacttcttgttatagaagacatgaaaggagttagatagtatgaatttagacaacatagaaagaaacttaagcaagaaaaaaactacaggagatgccgtaaaggatgagttcaatgacatggctatgaagctactagcaggctttaaagcagatgtttcggcaggagataaaaaattagaagatgtagcagacatggctaggctgttacaaacctttatacaagttaacaaagaagatagtggtagcggaggaacatccacttctttaccagagcttagtagcttgcaacaagaagcaataaccaatcagataaagacagagagcgtagtttcaggtgataaagagcaagaactagtagacctacaggacttagctaacatgacagaagaggacgtacaacggatgataatggataaagaaaaagcaatgaacatggaaaatgaggagacgttttaatggatggcaacactcttttaaagatggcgcaagaaacttttcaaacagaaaatgtaactacagagcagttaaactatgttctggatatgctaactccatcaacatatttactaagaaaccacaaagtaagaaatcataacattacgttctctattgcaaacaagtctactgaaaaggcgcaggcacatcgaccttggcaacctaagattataaatgactcccacccgaatgtagctgttattaagtcaagacaattagggttaaaattagcccctctataaagtaatttataaagaaaactctgttaaacgggcataggaaaataaccaatatcctgataagagaccctaaatcctaagtaggacagtgggaatcccgtgctaaatcaggcgcaagcttgtaaatgcctaacgactaaatttctaggtagctaaccaaatggggttagtgagaactagataagaaaatatattctcttacgataccaactgaaaaactctatcagatgttaaaaaattcttagaagaaaatataaagtaaagcagagcaacctaaaccaaaaaattaggttgatgatatagtctagtcccctaataaatatcgggaaaccgagggtataacgtaagtgagatgggtgtagggagtatgattcattttgcagatacacacagctatgcagctgttaaatgtttatacacgtttcctacaggtgaacagatgcgtaaatttgtacagtcacggttagaccctgtacttgagtctagttactatagttctattctagacaaagataacaacagtctgaactacaaaagaatacgaaatagtcacctttactttagaacatctagtaagcccggagctgtagagggtgtagatattgactacttatccatggatgaatatgacagagttcctgttttctcagaggcttccgctttagagtctatggctagttcaccctttaaaattgttagaagatggtctacaccatcagctcccggtatgggaatacaccgattattcgaaaactcagaccaacattggtacttgcataaatgtgaaaaatgtaaccactggaaccaactagactacgaggactacgacagtagctctgtagaagcaggaggaaatatactatgtgtaaatccacaaggtgttgataccttagctaaaacagttgtagaaggttcgttccaattcgtatgtaaaaaatgtggagccccattagaccgttggtataatggtgaatgggtagcaaagcaccctgatagaacaaaaaatggagacggtattagaggatatatgatttctcagatgaatgctgtttggataagtgctgatgacttaaaacgtaaagaactaaattcactttcaaaacaagcattttttaactatacactagggtacccattcgaagatgctaaactagcagtttatagtgaagatattatagaaaatccaagcccaagagtaaaggttccaatgcacagcagaggagattacaagtacatttctgttggtatagactggggaaatacacactgggtttctgttcatggtatgacggaaagaggagaaatagacttgataagactgtttagtgttgagaagagtcgaggagtaggtaatattgagtctgatttagataagattatcctagaagtatctatgtacaaccctgatatgattgtagctgacgtaggggatagcgggaactacgtggacaaattagtaaaacatttcggagaagaccgagtatttggttgtatttataagtccagccctaaatctacaggtcagttgaaaccgcagtggaatgaagcagggaatgtagttactgtagacaagttaatgcagaacaaaagatatattatagaaatgaagacaaagaaagttaacttctatagctttatagaccctatgctaaaactattggtagaccattggagaaacgttgtaatacaagacgaagaggacgaaaaagatggaactttctaccaagttattggcagaagaggagacgaccatttagcgcaggcatcagtgtacagctttattggtttagacagactagctgatatgtatttgagaggctctaagtatgagttcaactctacttttgtaacaagcaactaccaagatgtttcgtcaacaccaccagatatattcagtaaatattaaattaatttagttagggggctatgctccctttcttatttgtgttataatatagacaaggaggttttactgtgacaaatgaagagaagtttgacaaagtaatgtctatcgttaaagaagacaactctaccgtcctacctgactccgcttctattagtaaaggaattttaagagagttgacacaggaaagtttacaagactacggagaggtcattgaaaagaatgattttttaagcagggagtttaaacatattgtttccctctacggttttgacgacttttactcactatatgcctatgctgatttttgcgatactatgagtaatctagctattaaaggcgggaacaaagatttatctaagttaaaaaaggtaaaaagaaccgttatgagaaacggaaaacctacagagatgactttttatgagtcaactaccgaagaagagaataaagacaagaaagaagaaagtaataccaagcgccaaggagtaagcacagtatctgcaaaagacttacttgcagagctactaaagtctaaaggggaaccagaaaaagtaactcagttaataaaaataattcttaaattccctgatacacctgctaaccccaaagtatctgattatttttaccagttaaaagacgactcgggagaagtagtcgctgtggcaggttacagtataggagcccaatacgtctctcttgatttttataccagtaatggctctattagcggagtagcatcaagagcgttctttgaattactacggatagcaatagaaaaagacttaggagctgaattaaatgacgaggataacgagctagctcaagtactattttcaaaatttaattttcagctagatgagggaaaacagagagtgtctgctagaaacttaaaaaaacactttaaggggtaactatatgttgccattctattcaatatatgctataataatagcatgtgttatatacacagttatctttattttagtagtacgcagaatgtatcgtaaagcagacaaacgagagaaggaaaaagaagacattcttaacactatgacagaaataacagaggaaataaaaacatttattggaggagaaaataacaatgacaattgagaacacgaagactttagaacaaacactaaatactgtagccgaggaaaaagattatgacttattcctatcaacgttggcgggggtagttaaatcactatacaaagagtaccaatttcttagacaagacccagcacccgaggataaaacatcaggaaacattttgattttccaaattttccgaggagaccctgacggttatgctaaagattttgatacactgtttaaaattgcagtagacagaaatttagagcacagaaaaatatcccaaaaattctttgaatgggtagagaaaggtaagttcttagagttgggtgataacatcgttattagcagggatggtacaggagtaagctctattgttgagtctgttgaacttaaagtttttattacttttatgactaaaagttatgccgaagaacgagaagattttttagctaaagagaaggaaaagcaagaagaggctaaaaagaaagaagaagagcgactagctctaaaagaggaatcgtataaatctttaggagaggctacagaaatgctaaatactctcatggaagagcaaggtgtatctcctgaggagcttgctttagctttagaacaggggaaggagagttatcgtaatgcacgataatgaatttgaagtacctgagagtgttattaatgcttctgctacaccttttgaggcgcctgatagaacaaaagatacctaccaaagttcattaaagaactggttggacagagtaaaatcagtcggagatgcaggaatgtcacgagagtttgttattcgtattccagcaaaggcactagaggagaacaacccagaagctaccttatttttaaaacagtgtaacgaaataaaaagaaaccctaagtgcaaccttcctattaaaattgaaaaaacagtagagaagtacaaagacaagaaacacttaatgttagacttggtgttcacttatagtaataaaacagatattgatgcagtaaatagtgtaataaaataacaaacaggttcctctttacgtctgctatattagatagtagaaaacgtaaggaggaatttttatatggtaaaatataccgtagagaacaaaattattgcaggattacctaaaggtaaactaaaaggggctaactttgttattgctcatgaaactgcaaatagcaagtctactattgacaatgaagtaagctacatgactaggaactggaagaacgcatttgtaactcactttgtaggtggcggaggtagagtcgttcaggttgctaatgtaaactatgtttcttggggagcaggtcagtatgctaactcttattcctatgcgcaggtagagttgtgccgtacaagtaatgcaactacatttaagaaagactatgaagtgtactgtcaattactagtagacctagctaaaaaagcaggtatccctattacacttgactctggtagtaaaactagtgataaaggtattaaatcccataaatgggttgctgataagctaggaggaacaacacaccaagacccatatgcttacttaagctcatggggtattagtaaagcacaatttgctagtgacttggctaaagtatctggcggaggaaacacaggaacagcgccagctaaaccaagcacaccagcacctaaaccaagcacaccatctactaacctagacaaacttggcttagtagactacatgaacgctaagaaaatggactctagctacagtaacagagctaagttagctaaacagtatggtattgctaactattcaggaacagctagccagaacactacactccttagtaaaattaaaggaggagcacctaaaccaagcacaccagcacctaaacctagtacatctacagctaagaaaatttatttcccaccaaataaaggaaactggtctgtgtatccaacaaataaagcacccgttaaggctaatgctattggtgctattaaccctactaaattcggaggattgacttacactatccaaaaagatagaggaaacggtgtatacgaaatccaaacagaccaattcggcagagttcaagtctatggtgcacctagtacaggagcagttatcaaaaaataaataatggtaaactagaggagacttttatagtctcttcttttcttttgtaacagtagtgaaataaaacttaacccctttgtaatattatggaattacttctgtaacacatctatgttatatttagttggtaatttaaaaacgcatatgcggaatatagaaaggaaaccagctaatgaaaaaaggaagtaagattgcaattgctactgtagtagctgtacttattgtcgttacagttagcctagggtaccatacagttaagttgaatgagacaaatgaggcaaagacagaagaggtagttaagctaaaggagaatatctctagtgtatctaaagatttagataatttaggtaaagaacatgaaaagaaagtctcagaatccgaagctcttaagaaagaccttgacaataaaaagaaagagttagaggataagaacaagaaagaagagcaacttaaaaaagaaaaagaagaactacaaaaacaagtaagctataaaaagagcttaaagaaaaaggcagtagcagaaaacattccaaatgcaactagtagtaataaaagtgctaaaacggcagaaagcaaaggaaatggaagaagttttaacgtagaagttacttactatgctattggtgacggttttactcccggtgtacataccgcaaatggtactaacatcaaaaacacaataacaacatcagaaggacatcgaataattgctgtagacccatcagtaatccctatgaactctattgttaaagtaactacaggtaacggagagagttttgtagctaaagcttgtgatacaggtggagctatcaaaggtaacattattgatattttagtagaacctagcgcagatatttatcaactagggagaacaacaggtactgtagaaattattggataatttaaagaaataagagtctgcaaggtataacaaccttgtgggcttttttattatgctatattatatatgacggattatgaagtttgaggaggtatattaacgtggttataacacagatagtgatgattatagcgattattattactacatttcttattatttatggtatatactcttctaggagaaaaacgtctaagtcgtttagactagtactcagatatgctataatggtagtaggtcttcttattgtctactatctatttcaagtttcctctattgacattcaacaggattttttacagcacttaaaaacaggaggtaacgggatagaagaagtttttttcacaaatttctttcttatcatggtaggagcagtaggtacacttgtagtacaactattccaaaatagaaagaaggattaatgtgtcatatacaaaagactccttgtggttggagacaaaaaaggttgctagagataatgggtttttaggtaactggatagagatggttgactactatgagagtataggaggaagccatgtattcgtttatgcggttatggaaggtcagaagtaccgaattttaggcgttcttagcacggatgaggtattactactaaaaagagatttaacgctctcaaaagccctatatgaagatgttatgaatagcaaaaagatgtttttctataaagaggacagtgaaaagaaaaaaataaaattgacagatgaaataagtaagttagaagataaagatagtatcatagtatatatgtaggtaggtgaaaaataaattgggactatttgaaagtataagattagcgggggtaaataagagcgatgcagtaaaacacattgaggttgatgataactactccattgcaattcaacaaagggaacaggaacaaatatctaaagcaatgaataacaaagaagtagcttattctcaaccagtaattggctctatgtctgctaacccggggtttaaaacaaaacctagtattcgtaataaccaagatttacacggtgttcttaaaaagtttggtggaaacatcatcctgaacgcaataatcaatacaaggtcaaaccaagtttctatgtattgtaaaccagctagacactctgagaagggtgttggttttgaggttagattaaaagatttagataagaagcctacttctcatgatgaagcaacaattaaaagaatagaaagctttatcgaaaaaacaggagtagataacgatattaaccgagatagcttttcttcctttgttaaaaaaatagtacgagacacttatatgtacgaccaagtaaactttgaaaaagtatttaacaggaaccaaagcatggttcggtttgtagctaaagacccaacaactatattttttgcgacaacagcagatgggaaaattcctgataacggtaatcggtttgtacaggttatagaccagaaaatagtagctacgtttaacgcaagagagatggcatttgccgtaaggaacccacggtctgatatttatgctacaggatacgggtaccctgagttagagatagcacttaagcagtttatagcacatgagaatacagaagcgttcaacgatagattttttagtcacggtgggacaacacgaggtattctacaaataaaagcagcacaacaacagtcgcaacatgctttagagatatttaaaagagaatggaagaactcgttaagtggtatcaacggttcatggcaaatccctgtagtatctgccgaagacgttaagtttgtaaatatgacaccttctgcaagagatatggagtttgaaaagtggttaaattatctcattaatgttatctccgcactgtatggcatcgacccagcagaaataaacatacctaacaacggaggagccacaggttcaaaaggtggtagcctaaacgaaggaaactctgccgaaaaaaatcaggcatccaagaacaagggcttacaaccattattaggatttattgaagactttataaataaacatattgtagctgaatttggagacaagtacacattccaatttgtaggcggagacataaaatcagaactagagtcggttaagatactagcagaaaaagcaaaagttgctatgacagtaaatgaagttcgtaaagaactaaacttaccgggagatgtaattggaggggacattccattaaacggagtaatcgttcagcgtataggacaacttatgcaacaagagcagtttgagcatgagaaacaacagtctaatttacaaatgttacaagaacagacaggtaacagagtaagtactgatgtagaagacattcctgatgggaaagatacaacaggagacataggaaaagacggacaaagaaaagataaagataacgcaaacgctggtaaacaaggcatgaaaggtgataaacctaacgattggcagacctaatattttgtagttctgccttttaggtaactaactagtagaggtatatgctatattaataacagaatgtgttataatagatatgagaggtgatttcttaattgaataacgtcgtagagaaagagatgaaaatctttgtacctattgatattgaaggttctttagaaaaatcccataagggagatgacaaggactggtatgttaaaggttacgcatctacacctgctctagacttacagggagaaatggttaaccctgctggaatagacattggttattttgtagataatggttacattaactttgagcatagacaagaaccagactatgtagttggagttcctacaagtaactgttatgtagacttccaaaaaggtctgttcgttgaagcaagactgtttaaagacaacgaacacgcccaatctatgtggaagttagctaacagcattagtaagacaggtattgatagacaacttggtttttctatcgaaggtgcaataagaaagagaaacacagatgacagccgaattattgaagaggttgtaatcaaaaatgtagcagtcacaatgaacccagcaaacccagaagctacgtgggaaacatttatgaaatcatggactactggacatggaacaacacctgatacgcaagtaaatgcaggagctttaagaagagagagcttagctaacagcatatcagtactagcatcagcatacaaaatttctgaccctattgaatttgacaacttatggaaagatgtaagtaagtttttagatgaaaaaggagagtataacgagaatgaggcaatagttatgttacaattagctaaaggcgtttctcgtgtaagtgcatgtgagtctttaatgaatagacgaaaggggaaacaaaatgaataacagtgaagaaaagttcgaagatgttttaaacgaatttggtaatacagtagaaaaagataaagaagaaatttctaagtctaccgaagaaaaagaggaggttacagatattaataaagataaaaaatcagaagaagaaactgctgaggaagaagtaactgaggctcccgaagtagacgttgcagaagacaaagaagtgtccactgagactcccgaagaagagtctactagtaagtcagctaaggaagaaaagaaagaaaaagatagtgaagaagacaaggacgaagacgagaacaaaaaaggtaagaaagacaaaaaagacaaaaaagaagaagacagcaaagaagaaaaaacggagaagtctgatgaagaagattccgaagaggctgtaaaaaatactaactctgatgcagatattgcaggagcattgaatgctgttgctaagtcttatgagaatgttaaagaaagccaaagagagcaaaatacaactgtagaacgcttagagaagaatatcggtagcttagtcagtaaattagacagcttggtagatttaatgagtgaacgtgtatctaagagtgtagtagaagatattgaaactcttgaaggagctagtgaaaaagctgttggctatgtagcaaaaaacgtagatagcagtgtagtagaagttattgaaggagaagaaacttctaagagtgtagatgcagaagtgagtcctagagatgagtttctagctaataagagtgctttcctagagtcttttgaatatgcaagtcgtaacggtgctgagcgtagagatttaactgatgttcgtcacgctcatgggaacattacaaatgatattgctagtaaagcagattataaccgagttaacgactttatgaaaaaatggagtaagtaataaaggtaactgtatagagtattttttgtaaacagtatgctatattataggaacatgggaaacatgttacgttatgagagagcttgcccgttttaggactcctcccctagggcaggttatctcactaacaaaaaaataaaactatgaaactgaaaggtgataaatataaatgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacagcttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagacttagcagcacacgaatacaaagttgttgtaagttctgacgatgcagagtctattgcaagtgaagtggctacagctacagttactgcaaaagatgacggcgttaaactagaaatcgagttagctccaatgtacagctcccgtccacaattcgtttcaatctatagaaaaggtgcagaaacaggtttattctacctaatcgctcgtgtacctgctagcaaagcagagaacaacgtaatcactttctatgacttaaacgactctattcctgaaacagtagacgtattcgttggtgaaatgtctgctaacgtagtacacttgtttgaattactaccaatgatgagattacctctagctcaaattaacgcatctgttacatttgcagttttatggtatggagcattagctctaagagcacctaagaaatgggtacgtattagaaacgttaaatatattcctgtaaaaaacgttcatagcaactaataattataggataattgaataaaaacagtatagagagcagataaatactgctctctattttactaataaggaggatttaaattgctaaaaaatacaaacttagctaattataaaaaagtgaatacacgatttggaaatcttagttttgatgataaaggtatttctaatgacctaacggaagagcagcaaaaagaattaggtaagcttagaggattcgaatatattaagacagaacagaaaacgaaagaagaacctaagaaagaagaacctaagaaagaaagtacagaaaatgaattagacagcttcttagctaaagaaccttcaatcaaagaattaaaagaatttgcgagtaaaaaaggcattaaaattgaaaaaactaagaaaaatgatataattgaagaactaaagagagggtaatgtacaatgtatggaggttatgaaggacaagattcttacgaatacccttactcacacgggaaccctaagcatgtagagccagaaaaagttgacgaatatgttctttctgattatggctggactgcggaaacaattaaagcatacatgtatggtgttcgtgtagtagaccctgaaacaggagaggaaatgggagacaccttctacaatcatattatagaggttgccgttgataaggcagagaaagaactagatatagctattctcccaagacgggagtcagaacaccacgattataaccaaacagaatttagtagttatatgtatacccatgttcataaaaaacctatcttacaagtggatgacttaaaactagaaatgaatggtagggcaatatatagataccctgctaactggtggaaagtttattgcttagctggacatatagaactgtacccgacagccttgatgcagacttctcaaagtatgacatatgactctgtatttggaggatacccgcagttggcaggaacagccccttctataggtagaacatatgcccctcaaatgattcatgtagaatatatagcaggtatgctaccaaggaaaagagcaggtgttagtcgtgactgggaagtacctgctgcattagaacagttagtaactaaatatgctttagtagaaatattccaagtttggggtagacttatcatcggagcaggtattgctagtagaacactagtttacgacggagtttcagagtctattgatacaactcagtctgccatgtatacaggttcagcagcagatattgagcttataaataaagacatagacagtttagtagctcaactgcgttctcactttggaataaacatgattggactataagagaggagaatgtttaaatggcagagcctactagattttcaagtgatactcctattaaaaacctaacaacacaaaaagtagagtacaacatatcacagcatagaaagtttattaaagaccgtagtatccatacgttatgggaaagagcttatttatgcccttgtatgaacaagtcctcgggttctgctgactcttcttgtcaaagatgccatggtaggggatgggcgtatttaccagctaagcccgcaaatatgattattcaaggacaggaaaaaggggttactaacacagacctaggactatatgactcaggaacggctatcggtacaaatgattttgatgctagaatggcttttagggacagaataacagtacctgatgttaaaataggtcaatcccttatctactctataaccgaaagcagacaaaaacgaggtatatttttaacatatgacgttgatactatagactttataacaagtatggaaagagaactaatagaaggtaaagactacacctttgataaacacagtaataaactattccctgatattagtttgataggtgaagttatatctttaaacgtatctgttactctaaggtatattgttattgacttactaaaagaaagtcgctatcagtacaccacattaaatagaacagaccctaagtttgagcagttgcctcagaagttattactaaaaagagaagacactattgtattacctgaaccatttgtacttgatgctgatacagagactagagtagagttagttaatgaccctaaaaggaaaccaagacaacaaggcgggttctacgaagggatgtttgactagtgaaacagagtacacagagaccatctctattcaataatccaaagcaggtaatgagaaatatggcaagagctatagaatcagatgttattgcaacaggagctcaagaggcatctcgtatacccaatgtagagattaaaattatgcctaagtatttagaagtaactgaaaagagaatggaaaaaaatggtgtaattgaccttaaaccttattttgctaaaagtaataaaaagaaatggaatcaagaaggtaactggtacttagtaatccctataagattaaagacaagagatatgtctcgaaaattatatgatgacttaaggaaacagtcttccccaccagttggaagttcaacaacaattgtatcagatttcctatatgaccgcagaagaacgagccctagtgtcccttctataaactataatccaaagtcaaataatgttacaaaaaaacgtattagtaacagtagaacaacatatacagtataccgtacagtaactgctaagtcacctgctaatagttggattgttaacagggacagggttaatgaagaggatatgagtaaaacgatgttagacagaatagacaggctaatgaaatggaagcttaaaaacttgtagaggaggagtacaatgcctattccaagccttgattcttatgtagcaagaagagtaagagagagccttaaaattatgctttcagagtgctatattatagaggaagctttgagtgacatggatgtcgaagcaaaaagtaactttatgaagacatattgtggagataatcctaaaaaagaaatagaagtgatatacgagttccctcaggcaaaaacatcagtagatgctacttatgtaattcagctaggaggaggttccgaagatactaaaagcatcggcggtattgaaggtacttacgagtataaagaggattctttaagaaaagagacagtagttgctactagagaaggaaccgagttatttctagaaacaaaagagctaattggaagtatagagggaatacctaatttatccttttcagaaagtgataataaacgaatagaaggtaaacgattatacttctcttatgaaggtaatgcagatattgagggagtttccttcgatattatctatacaagtaaagtagacctagagagcgaccccgttggactaaaaaaagggtttacttctagagacaatatcactgttgtaggtttaagcactaacatggatactgcaaggtgcctagatgctatcttaaaagttatccttattatcatgagagaaaacttagaagagaagagttactactccctacaaacaactcagttttccgatatgtctcctgtaattatgggagaggaaaaacctgtatttggtagacctgtaacaatgagttacactatatcatatactgtagactttaatctagtcaaccaaataaataaaattatagtacaggagcgtaataagtgatggcagaaaaaaaagaggtaactaaagaacctattgtagacgaagtaaaagtaacaaagaagaaccttaaggaacctacaacagaagaaaataaagtagtgaagaaaaaacttaaagaagaacaaacagatactgtagagaaagttactacaggatatgttcatatagatgtattccttggaacagccaagccgtggtacggtttatcagaaatgcaagtacaagggtttaaatcttttatgcaagggaaacaataccagttcggtgaacaggcttttgttccttatttagaaaaatatctaggaagggaagtaaaataaaatatggcaatcgaaatttacccaagaaaaccagtttctagacctcatacagaaatttctgtagatacaagcggtattggtggttctagctctggaagtgaaaaaatactttgtttagtaggttcagctacaggaggaaaacctaatgctgtttacaaagtaagaaactattctcaagcaaaatctgttttccgttcaggagaacttttagatgcaattgaaagagcatggaaccccggtgaaggaacaggtgcaggagacattttagctatgcgtgttgaggaagcaaaagaagctacctttgaagctgagggggttaaggttagctctactatttatggagctgatgccaacgacatccaagtggcactcgaagataacaccatcacaggtacaaaacgcttaagcattgtatttgcgaaagaaagagtcaaccaagtttatgacaacttaggtagcatcttctctatcaagtataaaggtaccgaagcttctgcaacatttacagtagctgtagacccagtaacatttaaagctacaaagctaactcttaaagcaggggataaaacagttaaggaatacgatttaggttcaggagcgtatgcagaaactaatgtacttattagtgatattaacaacctacctgactttgaagctaagtttttcccaatcggagataaaaacttaacaacagacaacttcgatgcacaaatcgatgtagatattaaaacaaaagaagcttatgtaaaagctgttggcggagacattgagaaacaaacagcatataatggttacgtagactttgaatttgaccgctcaaaagaaattgctaatttcccactaactaagctaactggtggagacaacggtacaattcctgagtcttgggctgataaattcagttactttgcaaacgagggtggctactacctagttccattaacatctaaacaagctgttcatgcagaggcacttcaatttgttagagattgctcttataacggtaatccaatgcgggtattcgtaggtggaggaatcggagagtcaatggagcagttgttcacaagagctatcggcttacagaatgaacgagcaggtctaatcgggttctcaggaacagtgaagatggacgatggacgttctctgaaaatgccgggatatatgtttgcagcacaagttgcaggacttacatgtggactagagataggagaagcaatcactttcaaaaacattgcgattgagacattagatacaatttacgaaggttctcaactagaccaattaaacgaaagtggtattattaccgcagaatttgtacgtaatagagcagttaccaatttccgtattgtagacgatgtaactacatttaatgataaaacagaccctgttaagtcagaaattggtgtaggagaagctaatgacttcttagtttctgagttgaagataagcctagacaacgagtatattggaactaaaattattgacacctctgcttctttagttaagaacttcgttcaatctttcttagatagaaagaaattagctaaagaaattcaagactacagcccagaggaagttcaagttgtaattgagggcgacgttgctcgtatctcactaacagtattcccaatccgcagtatgaagaaaattgaggtatctctagtataccgtcaacaaatccttactgcctaatctaatacaggggagggcttagctctccctatgtaaataaaaaataaaacaggagttgaaacataaaatggcaagtgttactaatcagacggttcatacaggtaatacggtctaccttatgataaaaggtaaaatcgtaggaagatgtcagtctgcatcaggtgaacgttcttatggaacaactggtgtatacgaaatcggttctatcatgccacaagaacacgtttacttaaaatacgaaggtagtttaacagtagacagactgcgcatgagaaaagaagatttagctaagctaggtattacagcattaggagaagacattcttaaaagagatgttatcgacattgttatgatggataatactactaaagaagtaatcattgcttatcgcggatgttctgctgagacatacaatgaagaaatcaaagctaatgagattgtttctgaaagtgcaagattcttgtacctaagtgcagcaaacgtaaaataatactaataaaaggctaccttaatctggtagtcctttttttatagtttactaaccttagcaaatatggtataataaagtaagagaacgaaaaagtattactagtttgttatattagataatgagagagtaaacaaggaggagacaaattaacatgtcagaagaacaaaaagtaaatgaagaacaaatagaagaaagtaaagagaagatgttgacgaaagaagacatcgaccgtatcaactatgaacaacaaacgaagcaaaagaaaacacttgaccgtattattaaaggggtaaatgatgtttggacacatgagtatgactttaaggagttagatttaaagttcaccattagattacgagcacctaatgctttagaacaagcaacaattcaagctaacagagaagcttatttaaaaggaacaggtgcattagttagtgactacatttatacaatctatcatacattagccactataagaacagttggtgtagatattccaaaggaactagcagaagacgataacatttacaatactgctattttataccaaattggacgtgattttgccgaatggctagacacgtttcgctactgagataaaagatttaggtggaataaaagctttagtaaaaactccctatgcaagaaacatgtggggaataatgaaagagttcaatgttttacctacagacgagaggttcagagcgcttactgaggagcaaataaacttcatctactataacaaagagagagactacaaagaggcagagagagcttctagaggcgtgtctatggagtctgaatttgaagactataactcggatgactggtgggaagcttctcatgaggacttccaagcaattcaggaaggtcatgatgaagatgacatcgctcaacaagtagagaacatgactagtatcgaagaccaaaaacgcctaagagacagattccagtctcatgaggagtacgaagagtttataagaaatggtggtaaagatactagacaggaaatgattgatgaacacatcaataacaaactgaaagaactgtttgcagatgccgaagagttaaataaacaaggcttgagtaagtggggagaagtatcagaaatgacaactatccaagaagctgtagcagacgagttagaagagctaacatcagatagtgttaaggaagcaatcagtatgttcgaagatgacgaagttcaggtaataccgggtcaagatgaagacgatgattttgaaatttaatactaaaaagaaagggtagagctgtgtactctactcttttttactaaggaagtgaaagtaattggaaaacactaactatcgcttttcggtagaagccttaacggcagatgcagagaaaaagttagagcgtatcgctagaataatggacgacattgagggaacaaataggaaaggtgcaagtaatttcttcaccagctctcaaaaagatatagacaacaacataagagctatgagagaatatgtttcattagttaatgaagttgagaaggaaatgaaaaacgcccagtcttctatgtctcgttctggaaataacgaaggtgttcaaaaattaataagagaccagcaaagactaagggaagaagcggaaaaaacacggaaaacattccaagatactacacaaaataaaacaggtagtaacgcttatcttaaagacctgttaagagaccaaaaagcatacaacgatgaaatagacgaaggtactcgtagattaaaagagttgaaagacttacaaagagatgtagccagaacagatagcagactggctgccagaacaaggcaagctacttcatcaggtaatatgaactcacaacaagcaggaacagctaggtctgacttagcaaataaaaccgcatatatgagtcaattacaagccaaccaacaacgaagagatgagttaagaaaagggctagagtccgaccaagcaaaaaaaagaaacttaaatgctaactttagaggtaacgaccaagaaagacagaatgagttgtcagcgttaaaccataatattgagactcgtagaaaagaaatagaagctttaaaagagctcgatagtcaaataaataagacagtaagtaatatggaaagctacagtaaccaattaaataatgtaaatgtaaaaccagaccgtaactcgtggcaaggggtcgcttatgaaagagcaccatctatcgctatggctgcattaggtggagcaggtgcaatatttggcggtctatatgctaaaggtagctctataaaagaccagattagacctgacactatctctatagggcaacgaacaggtaactatgatttccgttctatcagaaaacagaacgaagaagtaggtatggataggtctcttggttacaacgcagtagacatgattgatttccaaaacacctatttgtcttctgctgggtttaaaaaccaaaaagacttagatgcaggaactagacaaatggcagaagggtctagagctgtacccgtagataaagattctctaaatcagtttatgggtgggctaatgacgaatggagccgtatcaggagcagaccaagtaaaagcggtacaacaaggttttattggagcagtaaaagcttctggaatggaaggtagggaaaaagaccaactaaaagctttagaagctatgagtacccaaacctttgcaggaagaaatggttctaatgctgagctaaataacctgttagctatccagtcactgttttcttcatcaggtgaccgttctatgcaaggaacagcaggagcagaagccttatctagtctagatgcaggaatgaaacaatctgcatataacccacaagcaagattactatttggtatggggagtgagtaccaaggagtaggtggtatgtatcaagttcaaggacaaatggaaaaaggtctcggagatacagacaatttaaatagactacttaaattctccgcaggtgcaggaagaggtaaagaagaacaaaaaggttacttcaagaacatggctgagaatgtatttggtactacattaacaaatgaccaagtcgatgcaatgtataaacaagtaggctcagatggaacaatagataaagacaaaatgaaaaacatccaaagtattattgatggaggaaaagacggatacgataagaacaaaaaagggtatgaagactctaaggaagctacagaaaacgcctctaaagcagtaactgataagcaggctacagcgatgaacgacatgggggacatagttagaaaactaaacagtgttctcggtggtatgcccgcaccattgtatggtttggcagccgcagcagttgcagcagcaggagccttagctatgtctggcggaatgagtattctatctggtggtattaaaggaaaactaggtaagaaatttggaggcggaataggtaaagcaggtagcagtacagtagcttccgcaggagcaggcgcaggagcctcttctacagctagtaaagtatttaccaataaaacatggggagcaggagcagaaggagctacaaaggcttcctcggggctctcatggggtagtaaagcaatggaaggcggaatttctgcaactaaaggattaggtggagcaggaaaactagcccagtatggaaaaggagccttaaaaggtctcgggccgcttggtcttctaataggcggaggagcctcaatcgccaatattgcaggtgcagaaaacaaaaccgaagccatcggagaggaagcgggtggctggggaggcgccatgggtggtgcagcaggcggtgcagccatcggagccatgtttggtggagtaggtgctataccgggagcccttattggaggggtactgggaggtattggaggaagcaaagtaggtaagtgggctggcggaaaaataaagagtggctgggattctctatggggcggagacaaagtagatgctgctgagataaacggcacaggaggagctaaaaaagctcaaacagagaaaaaaaccaacatgaacggtgcttctatggctaactctggtgtaggaactatgctaggaaatacagtaatgcccggtgtaggaggaattatcggaggagctataggaggttcaggtatcctagaacaactttctaagctaactggtattggaagaaacgcacaagaagaaacaagttcaggaaaacaagatgaaaaagacacagtaggtaagaaagctacagcagagaaagagagacaaagtaatacaagagaagaagcagcaaacctttctttttacagtaaacttcttgacagagccgaagcattattgatgcaagctagaaatcaaaatggattattcggaaaagaaggcttaggaactactgctacaggtagcggtggaggagatgctctcaattcaggtcttaacttcttaacagatggtaaaaagtggactaacaaagatttaacaaagcatgacttaggttcaactattcaaggtattactgctgatgagttagacgaatggattaactcgaaagcacctgagggttctgcaatgagagggatgggttcaactttcctaaaagcagcacaagaatcaggattagacccaagatatttagtatctcatgcagccacagaaactggttggggaacatcgaacatcctaaaagaaaaaggtaactggtttggtattggggcatatgataactcacctatggcaagtgcctacaaatacggggacaaagaaactggtgtagtagaaggagctaaatggattgctgaaaactactacaataaaggtcaaacaacactagagagtatgcgacataacggaggacaacacgagtatgctaccgaccctaactgggataaaacgatagctagcataatggggggctcagagaaatacgtgcaaggtaaaggtgcttctttaacaacttctgctaatataactgttaacatgaatggagactcatcgaaagataagaagacagcaggaaaagcagtaggagatgccgttgctaaatcgtataaagaacaatcagagttcttctctaaacaatacaagagaacataatgttctcttgttattacatataagataaggaggataaaattatatatgactacagttaaaagaatgcctgaatttgatttaaagtttgtaacagaaaagaatgactatcttataagatatgatgcaaggaacccttcctcagatacattagctgagaaagtaataagtgtaactactaaaaatgcaatgtctgacgacagcgccgttttttctattgtagtagctggtgatatggaatgggataaaatattagactctaatgatgtagtaatactaaaaatatacccaaacttaagagtaatgatgcctgacaacgttgttgttttagtgggtctcatatccgaagttcgaagagaaggcgactactctaataactctattatttatcgtataacagggcaatcttttgctaaatccttcatgcagttccaattaggggtaattcaagaggtatctgtagtaattaccgatattgggtggttgcctgactctaaggcagatggtgttgagtttacaggaaaaacagcagcagagataggtaagtctatcacagataggtttaaaaaatatatgaaatacaattttaacagagaatatacaatggaaaacttcttagactactctttcagcagttggaaagattacgaaaaattagcagaccctaccccatttattaactatgagggtagcctaaaacaacttctagatgatgttacagcaaagccatttaatgagttattctttgagtccacaagtgatgagaagtgtaaaatgattatgagaagaacaccttttaataaagaagactgggacaagttaccatcttataaaatatctacagaagcagtaatatcagattctttagctaaaggggacacagaagcctactctatttttaacgtcactagtggaaatatggcaggagcaacatcagtagacttaaattcattccctcaatatcaccaagctttagtagataaatatggatacaaaaagttagaggtagacaacaggtatttatttgagtcctcaacagatggctctagtaccacagaaaaggcagatgtaggaagtaaggaaaaaacaaaaacagtaattacttacagcaagtttaactcttttatgcgtagttatacttccgaccaagttcgtatgaatcaaagtagtattgcaaaaagtttagtggacagctacgacaaattgacaacttctcaagccaaccaactgcttgctaaatattctgctgtaggtgcgataagtgaggcagattttaaaaaaattgtaggagacatagcggagggagacaacacaggaacagctactttagattttgatagtgtaaactcttggttctctctaaattatagttctttgtctgaggtttctacaaatagagatgctacaataaaagcttttgtaaaaaactttgctaacacagatgaagaccaagcaactaagatagtagcactatatatatcaagtcaaggagtcatgacaaaagaaaaatttgatgcaatcataaaggagtctactagttctagcactaaagacccagataacacaacaggcaactctagttcagcactacagtacttctctaaaacgatatataactggtacagtgaaaacgctaatttttatgcaggggatataaaggttattggtagtcctgtatataggttaggaagtaaactccttgtagaagataaacaacaaggtgatgagtgggagttttatattgaaagtgtctctcatgaatatagctatacagcaggttatactacaacactcggagtcactaggggattgaacaataaagggaaagatagatttactcatttatggggaaaaagttcggactttaaaggcgggttacttggagaaaaaacaagtgctgaacttatacaagaagcaggttctacctcgtcaggttccgatggaagtggcgatgtaagcgcaccagatgtacaaggtagtgatgttgccgtagccgctttaagatacgggctagctcataaaaagccagagaaaaaatcagtatattcttttggtggaggtagaggtagtagtaacccaatggagggtaaggaaccttacgcaatggactgttcttcttttgtatggtggtgttataaagcctgtggtgttactttagcaggagcacaaacacaagcgatattaggggacgataggtttaacacagtaagctcaagaggttctaagagtaaagaaatatttaaaaaaatgcaagtaggcgaccttgtttacttttacgataacaacactcatattggtatgtatgcaggagaaggaaagttcttaggatgtaatggagatggaagttgggacaccaacggaggagtacagttgaaaccaatggactctggttattggtggacacagtttcaaggacatgtaatacgttttgtttaaaaaggaggaatgactagatgagtgatgtagaatcaggagcaccacgaacacaggctagtttaggtagtgagtgggaacgagtatacaaagcaggagaaaaagtagacacaatcgtattagcccagataataaaagtaaactataaatataacaccgcagaggttatgatactaaaaaataaagaagtgatgtctaagtcgtatatttctaaaggtaagttctcagctccactacctcaagattttgctggaagaactctagaaggacttccatatggacaaattaaaccaattcaagaaggagctattgttcttatagggttcctaaatagcaagaaggacaacccaattattttagcagtctacggggactcagatgtaaataataagctagctagaactccttatgaaagtgcagacccattagaccctaagctagctagaataaataaccagttttttaacttgtacccttccttgacgtatgataatatagacggggacggaaatagaatatccacttaccccggcaagacattttctattatgacaagtttaataaacgagtcttccaacatttcagacagcaataatgggactgaatatgcagacttacccgaatcatactacacagactacaaactgatagagccgagaagccctttagcaccatcaatgctatttaaacaccaaggaataagaacactagacgggaaggaagacaatcatttagtaactgcctttataaatacagatggaacaacaaggctgtctttcctaaataaggttatgaactggcgttcctattatgaggctgatactgagggaggtcaaaagttcagacttcaaagagacagtaaggtaccaaaccaaggaagcatattttcagagtttgggttaacaaaaaacggagacttctttattgtaaatggaaaccataagtttgaagtaagagagaacggtatttttattaatgactctgctttagcagacgacttaggggaagttttagaagaaattcaaaatagaatactagggataaacgaggagattgctaatatcatccgtatgttggatgagattgatgttgagaacctaggaaagttatttgaccgtatgacaaaagtagaaacagacattattagcttaaggaatgattataaaggaatagacagtagagtgaacgatatgtcagaagaccttaacagtctgattaaatcctttgataactttaaacagacaatgtctgcctacaccgaggctaatactataagggttactgctattgagaccgctactgcaaataatacagtatctataggaaacattaataatagtattactgcgattgagcagaagaacacagcccaagatacgaaaataagtagcctagagagtaaagttacgtcactagagacaagtaatgctagcttagtaacaaaagtaggggagttagaatctgatgttactttcctaaaagcagaaaatatagatttaactaatgcactagcccaattagcagaaagagttactgctttagaaaatagctgatagtagtactatattataaaggagacgaggtggaaaaatgccacaagcagacggaaaaggtggagtattaagacgagttgctttccaaataggagagaataaatggtatcgttttgcgataaaccccggtagttactcttttacaggaggacaaagggttgctgtgtttaaaacgaagtccttaaacgtagtagaagactttggtagagatattcctactataactttctcgggaacgacaggtcttagaaaagactctaacggtaaaacaggagcagataggttaaaagagcttaaaaaatatattgaagattatgcagactcatcaggaaacggaaatagacctaatacagaccttacttttcataattttacagacgatgaaagctatattgtacatttatcacctgaggcatttaagatagaaagaagcgcagaaaaacctctgttatatgactacacaataactttagtagttttaagggatgcttctcaaccagaccaaggagacattgtagacccagacttaggtaataaacaacctagtgtaggggacaagacaaaggataatgacgatgtttatggggatgtaggaggcgcagacaagacaccagaagctaatattactggaagagtccctctagaagtaggaggtaacgggggaggggcagtaaacccccgaacaacaggtaaagcatactcctacggtgtagatgaattaaaaaaacaaattggttatggggagggttaacaatatggaggtatatcagtcatcagatttaatgaggtttattacagggttaaaagttaacccagataacactattgtagtaaacatcttagaccagcaatcaccatttatttctaggttttacactcctaagcttattctaagttctttatcagtaaagcttattcctgaattgcaaggagtaaacattatacaaacaaacaaagcattaagtaaaaatacagttacagctaaaatatcagcaagtaggttaaaaatagaggctccttacatgtatctaatcgcacagtctatagtcatggaatgctttgctctaatctacagtatagaagaaaagcaagatatgctaaggtacattgaagtaggagaagttaaacaagttagagataacataaattatttagctgactacttagctgacaaaccagaatacagcagtattgttacggacttacatgatatggaaattgctgtagggtatattgagaaccaaataccaattatacagagggagataagattaaatggctcgttataaaaaacacgtagtacaatatggcgatactatgcagtctatctctcagctagagactggtagtgttggaaactgggtagatatagtagaatataataagttaaagtatccttatattgtacaaacaaatgaggaaagaaaagaaaatcttgagagtacagttactattggggatgttattatcatacctgtagcatcagacctaacagaaatagacccttatgctctaacaaaccaagataagaacttaataatgagtttagctctaggtagggatttaagtatgacctccaatcaagattactataataaaagaggaactagtgacgagatactagaagttgtagctaacggcaagggagacttagcgactgttgacggagtagaaaacctaaagcaggcactaatagctagactattaactcctaaaggagccttgatgctacatccagaatatggctctaacttacacaagatgtttaaaaaagctacacatgaacaagcatcactaatagatattgaaataacaagaacattgttactagacggtagagtagaaacagtagaaagtctcggatacaaagtagaagaggacgtatatgtaggaaaattcctagttactatagagtctttacagacagcgtttgagttcgttgttcaaggtgatactacaggttctattattttgctagataattgaggaggaaaacaggttgagattaaaaagaatttcagaaattttatctcgtttaatagataagattatgattaacacatcagagctaaatgatttttctgtagggtctactatcttgtctatactagaggcagtttctatggagttggagcagtactacatcctatctagagagaacattgtgtggggtattcagcaaggggtatttcaggctttcgacttttttaagagagagccaaaaagagcttatggagatgtcctaattgagtttcatagcgcagttcaaagtgatgtatatattccaaaaggaaccacttttagttcaacaattaacggatacgaccagcaattcgagacagtcatagactactacgttcctaaagattccatattagctcgtgtagaagtgtattgtaaagtagcgggggaaataggtaatgtcccagcaggaactattaatttgattatgaatagtgtaagtaacgttaagagtgtcataaatgagagtaacttccaaacaggaactgatgaagagtccttagaatcagtaaaaaataggtttcatgctttcgtagagagcagagggagagctacagtccctgctataaactatggtacccgtttagtagaggaagtatcaggcgtatatgtaaaggaatatgtagggtatgtaattgtctatgcacacgatagaaacggaaacctttcagctaacatccaagataacattaaaatagcattagaagactatagaccagcaggtataaaattagatgtaaaacccgtaaacaaaaggttgctacctgtcgaggttactgtaactattactaacaaaaacagaataggggatacactacaggcacacattcaaggagtgataacagactacttaaatagtatgcaagtatctaacaacttaattttatcagagcttattcaaataattatgagtatagacgactcccttatttatgactgtaagattgaaaactttacagaaaatgttataataaatagtgaggatattattagagcaggagaagtaaaggttaacttaaaataacatagttaacaaatgagaggagctgacaactacccttgccaaactttttaaaaaatatacaccctttattaaaaagaaaaaaagatgacaaccaagtaagtagcgtaaactacgctgttttacaagcgctcgaaacagagctgagtgaagtagaacaggaaaccttagagagtaaagtacagtcatcccttggaacagctacaggagactacctagatatttggggagactggtttaatgtataccgtagagaagatgaaggagacgaacgttacagagataggattattcggcatttgttattaaagagaggtacaataccagctataatcgaggcaattcgtgattttctagaagactatagcgcagatattactatctatgagccgtttaaaaatatcttttacacaaataaatcagacttaaacggtgaagactacttgatggggtactactatcgttttgctatcattgatgttacaattggtagacggtttcctttagaagtaattgacattataaacgcatttaagccagcaggtgtaaaattctatctgacttatgacggtagtggagaaattgatgaagaaaacagtatagttgaactaccaccttatagtatagaaattgaaacatatcaagaggtagatattctaaacgggttaaatacaacacttcacgggcacttcactttaggagagtctaatattgtagagggtgttatccttgagttgtttcgaacaaatagaagtccactaaactctaatgatgtactatctggctcttttaatgcaggtagacgagtatacaatctagcaacaattggagacaactcctataaacctacgatgagttctcttattggggaaataaaaaattcgttcgaggaattacccgaagacttctacgtagaaactaaccgcttagatgataggtttgctagcatagacatggcaaataatacacaagatactgcattctatttcaacatggatttaggaacttatatcaatactaactacgcaggatatttagctgagttagcaggaagccagattataaatagagcacactatgtatccttagctaaagagccaaagttaaccgtggcgtttaaagctaaaatatccccagcaaacccagtaaattacaatatagaaggttatgacttagtttctaaaaaatgggttgttcttgagacaggagctatagcggttgaaaagaaaatatcacagatacctttaggttcattagaagactatcttaatagtaactatatatgttttttccgaattgtagtgaaacctaaacgggaaacctactcctttgaattagactatttcgaattaggatttttaaacaactttaaagaaggggagtatacaattaagccttttacaggtattgtagaaacttatgcagagttgttcggtagagttatagaagttcctatgtatcggataataaattcaggaacagaaaatattatttctaaacaaggtcatatacctgttagatatattagagatactttagagggtggtagtactacaaacactgctaacctatggatagaaattcaggctaaaaataaagacaatatcaatgttgcgcttaataagcctgtatatagaccacaagctgaacctacaaatggtaatattcttctatatccacagttccttaatattggagctacttacatgaatggtaagtggagaggaaaactaactagtgaggtaaacatggcatatactgtagacggtgtaagaacttacggaggaaacatgaacgcagatggaacctatacagtctatatcgggaacaaagtaacctctactagccaaaaagtagttgtggaggtatatgcagacactgcaagaacgaagttaatagacaaagctaatgttcctattacaaatgcaggggacggaagccctatagaggctatcacagacggagtaattagtggctctaatatattctatattgcaccaacaagcgccaaatactcagtaactgtagacttagaagctacctttgtagacttatcagaggtaaggatatggcactacccaacacgaacgtttaagtctgttttgacagaagtatcagcagatggtgtaaactggactactatatataactccgctgttgatgggacttatctagaaactagtgaaggaaaagcgtttcctgtaccagagatggacttatatggattgaaagctactcctctaacaagtgtactagcaggttctgaaagacaaattattacattcctagactacaaaatagaggactttgagaacccagctaatactattgaaaacctaaacaatgattactttaacgcagtgtggcaagagattgaccgacttgaagagtttgttctaggcaagttcactattttagctgatgttggctctgacggagtactagtaaatgactggggtcttattgaggctaaaatacctacaggtgctacagcataccaagaagcggacagtagaagtgaattgtcttccgtgtacactgtggagctcacagcgggctctcacgaggttattatggatgttgctaataaagttgttactacagaggcagtaaccctaaaaacggattacagggtgtctaacgagcttaccttggaagggctacctacaaaaatacctgtgagatacattcgagactccttgtggggaggaaacagtataaatacagcaaaccactgggtagagctacaggctagagacttgaaaggggacaatgttgcccttaataaaaaagtggtagagaacaacggagttagcttactatctcctgaaaatctaatgttagactcgaagacagagattactagtacaacaaacaatggttatacttttgcttctgatatagcttatatatttgataattatggagtagataaagagtatactattagttttgacatcaagtccactacggtaggcacagttactgtttatagtaggtacaaagatgaagctaaatattctataggaaacaaaactgttcctgtaacgactgcttacacaagacagcaagttgtagtaacacctactagaaacacagcagaacaagctaaatatgcagtcagctctattctaggttttcttaatagtacaggagcaatatctacagttaaggatataaaaatatcagtaggaaacatctcgaatatgatttggacacctgcacctaaagactacagtattcttactgatggagtaacaacaagtgctaacggattcttgagtggagttaactccttaactattgacttaggtagccctagatatgatattgacaacatcactatgtggcactattggcaaggggctgaacctaggtactacaaatatgtaaaactagaagtatcagcagatggtgtgaactggactacactgtttgatagtgatgtaagtggaacttattacgagactagtggaggtaagcaggttagcgtacctgctataaaagaataccttaagagtactaatgctatatttgcacctagcgtagcaggaacaggtacttttgagagagtaaccgaagcaaacggagtaagttctataaaagtgacacctgatactgataagacagctagaatgtacaagtttttcggaacttctacagaccctaacgctaattcgagagaccttctagaatcagggctaactacaacgaagaaatacacagtagcagtatccctaaaacctcaaaataatagttcggttatcttagcggtaaaagatggaagtaacactaacatagagtcgggctccttaaatattgcaaaagatgtgtggacaagagtttcttacaatgtaacgttacctgcaacagctattgataggctagctttatgtttgtacaatgctaacacaggagaatatattaattataaagattggagcattaaagaaacgatgtttccataaagtaaaaattatggtataatagagatagggcacaaagtagtgccttatctttgctatattagtaatgagagtacgaatacaataccgattttaaaataaggaaatgaggtaaataaatggcaatagcaacatataatgcgcatgtatatgcagcactaaacctaaaaagtaaggtcgatacaacatttatggcaattggtaaaacaacagcgtggactgatgagactaaccctccagaaccagacccaaatgctacagggttaacagaagttatcggttacaagaagctaaaaactatgtctctatgtagaccacaaagaacaggtgaaacaccaacactacctacagtttcttacggaaataaaacatgggttcttgtacctgatgcacaagcttatacagaaggagctaaatggctttattgtgaggcagagttcgtaggggatgaactaccagtaggaacatacagacaagtaggtgtatttacagacttagcgccaaaatctggtgtaacaaaaccgaatttattaccaagtgaagtagctaatgtaggtgtactacagttttttgagaacaagcagttccaaaacagaacaccacaagttacagctagagaaagattcgtagcggaattataagaaagggtgaataagtttggcagatttcaattttagttcagcaccatatcataataggttcaatgccttgaatgattggacattagcacttttccaaccagacagaccccttcaacagtccgagttaaacgaaatgcaatctattttgcattattacctaaaatctgttggggatgctgttttttctgatgggaacatgcaaacaggtatggagtttgtcttggataactcaggaaacttaactgttggtgatggtcaagtttatgttgagggcaaagtaagaaacttcaaaaaacaaactataaaattaaataagataggtagagagtttgtaggagtaaaagtagaacagtctattattaccgcagaccaagacagtagtcttctagaccaaacagcaggagttcctagtgctttttctaaaggagctgacagactaaaagaaactgttgttttaacgttagacgaccctcagtcagcaactatctaccagtttgataacggaaaactttttatctcctctgataacccagagatgacaaaaataaacgaaattctagctgaaagaacatacgatgaatcaggctcttatcgtgttaacggttttaacatgtggacagaacctcatccgacagacactagtaaaattcagttagttattgacacaggtagagcttatgtgttaggatacaaggtagataaaccaacatctacacgtatacctttagataaagctagagatactcgtagtgtagccaatgaagggcaatactacgatacagcagttaaccgtattaagctaggaaacagtcctgtagaaacagtaacacgagttacaggtcaagttcaagtacctaaggagcaagtgagcagaggttctcaatcaggaggcaccgacttcttgagtagaacttccgtattccagatagaccgtgtgtggacagaaaacccaacatcaggagaagttattaaggaatacactcaagggacagacttccagttaataaatggacaagcggtttcatgggcacctaacggagcagaacctacggcaggagcaacttactacgtgtcttaccgttataacaagtctatggtactgaatacagactacaagataactattgaaggtacaggggatacaaaaacttggtatattgattttagtgatatgacaggaagtaaacctatccaacaaagtttagtaaacgttgattacaactactacttggctcgtaaagacctaatagtacttgatagatacggagaagttacagtacacaaaggacaaccagataacttaagattagcggaagcacctgcacaggtagacccctcaacacttcaaattggggtagttactatttaccctgaaagtgagaccgctagtgctatgtcattctctgttactcgtctttctatggctgatttacaaaaaatgagaacacgtattgataatattgagtataacgaagcagtaaatgctttagataatcctgcaatggcaggagaaaacccagtgacattacgttctgtattctctgacggttttatttcactagacaaatacgatagcacgcatccagatgctattatagcttttagttttgaggatgcagaaattaccctaccttaccaagaagttaacaaggtagttccaaaaatgttagagaacgcatcagaagcttatgtttggggacgtttagtatcagcaccatttacagaagagaaaacaatttctcaaccttttgcatcagaagctatgaacgtaaacccatataatgtgtttaataaacaaggggttattactttaacaccttctgcggataactggattgaggaagaacgagtaactgtaactcaggacgaagttaagtcttatacggtaaaccgttggtggctacatggaggtaactactattctgatgaagctagatacttgcataacaacctagcactagatgcagggcagtcatggagtgggactccttctcagaacaaacaagtaacaggaacaacattaacagcaggtggacaacaaacacttgagtctatgatagagtttatgcgacaaattgatatagtggtagaaggtaaaaactacttgcctaacgctaacaacttatacgcactattcgatggtgttcgggtacctattacaccagcttctggttaccgaaaaggtacagacacaggaacattaatggctaatgcagatgggacagtaaaaggaactatcactattccagcaggtatccgttgtggtgtgagagaagtaactttacggaatgcagataacttaggtatttctacattcactgcacaaggtagaaagaaaaccgtgcaagacatcattatcagaactcacgtaacagctaacttgtatgacccgttagctcagtcattccaattcacagatgaccgagttataactagcttaggattacattttgctagtaaagatgcaggagaaaacaacgttatcgttcagattagaggtatgagtgacggaggacaaccaaataaaactatttatgcagaaagggtattaaccccagctcaagtaaatgtgtctgctgacagtagtaaggaaactaaagtcacttttgatgacccaattatgtgtaaagcaggtcaagagtactgtatcgttattgtaacagactctgatagttacactatgtggttaggtactagaggtaaacctagaatagactcacctagcgacttaattacgtcaaacccttatgtacaaggtgtactatttagttcttctaatgcttctacatggacagcacaccaagattctgatttgaagtttaatgtatatactgctaagtttaacgaacaagctgtactagaatttgacacaatgaaaaatgttaaagttgatagagtagtactaatgagtacgtacctaactcctaataacacaggatgtatctgggatatgaagattgttctagacaatgaacctacaggagttacagtagcagacaaagaatggcaaccaattggtaactaccaagatttggatgtagagcaattagctagagaagtcaagttaagagctacttttgttgctaacaactacatttcaccattgttatcattggacgacatcatgtttgcaggtttcttaactgctttaaaaggtagttatgttagtagaaatattaatatgacagactctccttataatacagttaagattagttacgaagctttcctaccagctaacactacagtaaaagcacagtttagtactgatgcaggtattacatggaaagacttgacgtctattcctgtagtaacacaacaaacagccgatttctacaagtatgaatacgaagagaaagtcatccaatcaggcacatataaaggatttaaggttcggctgaacttagcttcacaaaacagtttcttaagacctcgtgctcggagactaatgtgtttaatgaaagatgaataggggaacttttgttcccttatttttaactaaaaaggggcggtaaacatgccagagtcatatagagacccccattcaggggctttaaagtttattcctacgaaacaggagaaaataattaaagatgtgactgaaaaactaagagaagacgaagaaaaaattcgaaagaaaatagaagagttagatattaaactagcacaggcagacagcaggttgcaagaaataactaacttgtatgaagaattaaaagaactaaaggagggtaactaaatttgtctagatacgaccatagtactgtagagtacacagacaaaattaaggacttaacagattcggtaaaccgagtaggaaactacttatcaggagacaatagcccttatgacgatgttcagaaattaaaagcaattacacaaaatattaagctaactaaagatacaggtttagctaaaagcattacagcaggaacaactgcgttgaggtcagtagtcgaagtaggggtttactatattaatagcacagaagctctagccctaactgataaacccccagagctaacaggagcctttatactagtaaactacccgacaacagcctctacttctgttaagcaagaagtacacatgtttgctacaggaacaacaggtagctacgtagggtaccgatggatttctgcttctagtgtcagctcatggtggacttatgaaaacacactaggttcacaagctaaagcagataaagcattagcggacgggaaaacatatactgatagctcagtaaactccgctttgcaagcaattcaaaactcagcacaaatgtacaagttaacagcggatgatggtaagcctattgatgcttctgctatggctactccaccaactagtgtagcttctctaacgaagacaggaatatattactttactgccgcttttggaaatacaatgccagataccccttgcacagggcaaccattctggttggttgttttacagcatgtaacagataacagtatatcacaatctgtgacagccaacactgtagcagtagagagggtagtcgcagataggattattactactctaggggtaccgagtaagtgggaatacagagctaaagcctctaatttcttctttagtgcttccaattcgtcacgtataacactagtatcgacccaacagaatattataacacctaataaatttattaataaccccggtagtttaccactatctgcgataaaccccgcaataactattccagaagatgggatgtaccaagtgatggcggccctaaacattaacggtattttaaaaaaagtgtacctagtaagtgatctaactctactaataaatgacgtagtacacccagctacatttggaatggtaaaaacagcagataatgctaacgggcagcactctttagcagggaatggcatatatcaactaaaaaaaggggacaagttaaaactaagaagttactgtaacaatacagggaataacccttacttggatgtagacaaactttatatatctgttggtaagattgcagacatttcattatttaaataaacttagtatgctgagaaaggaagtcacaataatggcagaagaaaaattttcattagacggaaggcagtatgtaacagcagtcctatatatgtaccctaacctagaccctcagtacgactttggagttgtatcatccgaagggaaggtagaggcttttgaatggaacgtaaaaaatataacaaagccttctattgcacagttagaccaagcatataaagactatgtaaaatctatcggaagtccaagtaacccaccagccctagaaccagaggtctcaaaagaagcctcaataggtaagattctaactgaaaaatttagtgcgttagaggaagttaatgctaatatgttattggaaaatttagagcttcaagctagcctagataaaaaacaacaacaaaattcagaactagtatttgagctagtagacaggggagtcttatgagtgtaaattggtatgagttagttttagattactacacaaaaggttattacacaccaagtcaggtaaaaagatttgctgaggtaggaaaactaactttcgagcaagccgaagaaattatagctacaaaatcatagagtatgtttaaactatataggtaattaagactaggactaatatcctagtctttttatgttataataaagatatagtaaatatatttggaggagtacattaatgagaattgttgtagatataatgaatacacgagttttattcgataaagaagaagaggcaattaaaaaaagatttcttagggatatgcaccaaaaattaggggtaaaagtagatgggttcttctttagcccagcatataaatcaggtcattgggacggtatccaagacttctatgactttaaaacaaatacgttccctacagggctgttgccacaagtgcaagagatactaggagctatgcaaaatgatgccgtatttcaatttgagattattgatgacagaccagacccgtttttaacagtagaggacttagatagtgagataactcttataggagatgacggaggagatatacagcttagagattatcagtatgactctgtaaagtctatcattgaaaactatagtgggattatcaatattgcaactaatggaggaaaaacggagattgcttctggtatcattcagcagattttaccgcatttagagtctggggagagaatagccttttttactcactcctcatctatatttaaccaatcaattgaccgtatagaaaaaagactaggtattaaagtaggtaggctaggaaatggtaaaaaggacatcagacaagttacttttgtaatgatacctacgttaacagcctccctaaaagaccccgaagttggtttgaaattaacgcataaggaacgagttattaagaagttagctaaagaggtagttcctcgtttcactatgcaaaaaaatcatagaaagcttcttaatatttatattgagaacaacagaccgaaaaataaagtagaagaagatataaaagctcgactagaagacttgttatattcctgtggaacagactccgaagttctaatgggactaaaaggttttaacgcagaatatgaaaaaattattgagaaaaaaaataaaaaactatatgacaagaacaaacaagctagagaatttctatcatctgtatctgttatgattgcggatgaggcgcaccataccagttcagaaacttggtacaactctttgatgtcttgtgagaatgctatgtacagggtggctttgacaggttctatagacaaagaaaataaaatgttatggcagagaatgcaaggagtattcggtcaaattacggctagaacaagtaatgagaccttaattacacttgggcactctgctaaaccaacgattaaaatattccctatcaaggctcctacagatattgaaaatgtagataactatgctactgcttatgaaaaaggaatagtagaaaatgattatcgtaatttacttattgcaaaattagcaaagatgtggtatgataaagacaaaggtgttcttattatagtaaatagaatagagcacggtaacagattatctgaacttcttgagtctctcgaagtgtcgcactacttcattcatggagacttagacatagatttaagagaacaacaactccaaaatatgagagatgggaagcttaaagtgatgatttctacatctattgtagatgagggagtagacatctcaggtattgatgcgcttattttaggtgcaggagggaagtctttaagacagacacttcaacgtgttggcagggctttacgtaagaagaagcaaggagaaaacacttgccaagtatttgactttaaagatatgactcatagatttttactaagtcactcagaccaacgaagacagatttacgtagatgaaaactttgaaattcaagacatttagggggaagaacaagaatggatttagtatatgtaggatgtcagatggaaataggaatgaggaaaggtttacgagactttctagtggatgttgctaaaacagctcagcaatggggagaaagcgtggctatggttactcaaaaacatagcctagctgagcaaacaggagtaaacattagaacagtctcacgttatatagctgaacttgaaaacaaaggtattattaaaacagaagcaaaaagaggaagaaacggtggactagttatcatgtttaataaaaatatggttacttttaaattaacagacaaccctataacctctgattcacctgatgcggtagaacttagagaccgtatgttccctaaaaagaatgaagacccgaataaagagaaaagacctcgtagaacaaaagaacaaatggcagaggaaaaagtactaaaaaatcgtaagagaagtcttaataatgagttaaacgatatgttagacgatacattattccctacagaagagatatttagtaaaacagaggagcctgagaggtactttaaagcttatttactgtctcgaatggttaatgcttatgcgtatatttatcctacagagagagagctagacatgctaagcagaggggtgcaggtagaagcagagaaggcacgaaaaaatagagtacgagcttactcttttaactcactacctgctagattttttgggacatctcagtttacttgctacttaaaactacaagacttattagaaaaagagaacattaacccattatcttatttaacagttcaatttgactacttagactacagattagataacagtcttggaggaactccactagcatatagaggtatccctgaggcaaactgcttaataggtgagactgcctttagtcgttacacatctgctaaggacttctacactgttatgcgaggtaaaaacatgttacgcaccagtacagcagatgttgcctttaaaggtggaaaataccctattgtggaggcgttattacacgcttacaatgtaggtaagcaagatattactgcactaaaccaacaaatagaggatatgaagacacaagctagtgtgttccataaatccgaagccctagcagacttttatgaaacaacaaaactattagttgaagaaagtgctttagaaacaaatgaaaaagacgagctatacaaatttgttactgaacaagttgttttacactctcgtaagaatgcaatgagtcttagccaacagttgctaagcttcccacttcaaattcaacgtgtaagacagacagccgaattaaaaggattagacaaaaaggactactatacttttgtaggcaacttagagaaacaagtgaaagtaagtagaaacacgtttaactcgtttactgaaaaaggtaagcttttacacgagtctctagaggcaaacagtactttttatagtaccattcgtatgctaggagactaccgaggactaaccgtaaacatcccgttactcggagaagctatcaagaaattaggagaggaacatattcctctagattcttatggaatgttagatataacacaggtttacgacaagtatttaacagacctgcaaatgcaagaagatgaagagaactatgaaacacctacaagttttattgttaagtaaggaggaggagatatacgttgagttccattattcaaaagcaagttatataccgagctattagagaccctttatttggtaaggatgtgttagcacagttacctagtaatgtatttgacgagaatgagcactacaaaaatctagtaattattattaaaaagcattaccagacaagtaacaaggcgcttgatgaaagtacgttgctaactattacggaggaacatttagttagacagaaaaagagcttagaagtacaggaaagctttaatgagacaattcatgatttgtataaaattgatgaactaggtatagacgatgaggctgtcggagaaagcattaaacgattcgtcaaaaaaaatctggcagctcacttgataaaggagactttgtctaagggtgaagggctagacaacgaagaaacattagaaaaactatacacaggtatcaaagaaatctctattttagatattaaccgtagtgaaaacactttatttagtttctttgatgatgtagaagccaagaaaaagatgctacaaagtatccaccaagataagttctctacaggattcacagacttagatgttattgctgagggtggtattggacgtggagagctcggtctagttattgcccctacaggtggtggtaaaactatgtgggcagtaaaccaagcaaaaaactatgttgctcaaggtttaaacgttttgtatgtagtgttagaggaacgattagaacgtatgattttacgttttgaacaaaatatgatgggtatccctaaatctcgacttatgccagataatgaacttgacgaagaagctttcgacaaattacaaacgatttatgatattaaacgtaaacaaggattaggtaatctttacattgaaaagagaatgcctcaggaagttagtcctacaatgttagagcaaattatcgttgatgctagtgttcgtaaagggaaacatttagatgtggttattattgactatcctgacttaatgaaaaaccctcatgaaagagataatagtgagtcccgtgcaggaggaaaactatacgaagaaatacgtaagttagctcaaacctataagtttgctggttgggtactctctcaggttaaccgcggaggatattcagaggatgtacttactgctaaaagtatcgagggttctaaacagaaactaaatgctgtggaaattgctatgaccttaaaccaaaaacctattgagtttgaaaaaggttttattagagcatatgccgacaagattcgtaataataacggtaaatcttataaccgtatgcttagctttaaggttgttccagaaacaatgactatcagaaatataacacctgatgaaagagtagaacacgatatgattttaaataacctcgaagaaaaagaaggtcgtgtatttaaaaatacgaaaaaggctacacctgacccccaagcagttgtagacgaagtaaaagcactaaatagtagattataaggaggagatagtttttggataaaattatattttttactgattttcatgcccatatttttaccgaattttctaaaccagatgtggagtatgaaacagacagatttagagaacaaatggaagtcttacaacaggtatttgacttagctagggagaataaggcagatattgtttttggaggagacttattccataaacgaggagctgtagatactagagtactaaaccatgtttttcgtgtatttgaaagaaacacggatattgtagttcacctactgagaggaaatcatgatagctttacaaactcgatgaacacagagagtagtttggagacacttagaggtttacctaatgtggaagtttacagcaacccttctattgtagagactgaaaaatataagttatatatgctaccttatggggatgaagtgcaagagatgaaagactggattaacagtttagacattgaagcaggtaaaagtaatatcttagttgctcatataggggtagagggagcgttaacaggtaaaggttcccacagactagaaggtgcatttacaacaggagatttaagaacaagcgactttgatgttattgctcttgggcattatcacaagagacagctactactagatgttcctaatatattttatggggggaacaccattcagaacactttttctgatgaagggcaagagaagggtgtatggctaattgactgttcagatgttaaaaatacaacaagtacttttattcctattagtagccgtaagtttattactattgacggagataacattcctgatgatatagaagaacttattgaacaaggcaactttattaggtttattggaagtaaagaacaggctaaaaaaattgacttatatggagaagaaagtacaggtattagagtagagctacgaaaagagtatacaactgatgttcgactagggattgatgaaaagtcctctcccgaagctatttctaaagcctatgcagagaaatttagtccctctgcaacaagtgaaatagtagagtgtataaaagaggcacttgtgagagaaaaataagcctaaagctgaggatattcctcagcttttcttattgacttttttagtaaagtatagtatactaaagttacaaataaaaagaaggaggaaaagaatgttcacttttaaaaaagtaaagataaaaaacttcttatctatcaaagacatggagcttaatttagacaaacaaggtctagttctgatagaaggaaagaataagacaaatgaagcctttaagtctaatggtgcaggtaagacaagtatgattgatgctattacttacgctatctttggtaagacagtaggtgggcttaaatcggattcagttgttaataacaaagagaagaagaacactgctgtaattttagacttcgaagttgacaagaataaataccgaattgaacgctatagaaaagacaaaaaagagggtaatatagtaaagctctaccaaggtaaaaacaatattacaaagagtacagtggacaacacagataaagagattttatctctattcggtattgattttaatacgtatgtaaatgctattatgtacggtcaaggagacatgcctattttctcccaagcaacggataaaggtaaaaaggagatactagagagaattactagtattgacatctatcagaaggcacaagaaatcgctaaagagaaagttaaagagaagcaagctgagcaacaagagatagaaaatgaacttgtaaaatatgactatgagcttgtagctattgacaacttagaagagcaagaaaaagaaaactataaagcagtccagcaaacaatagaaggtatccaagcaaccctatcagtagctagtacagagctagaaaactttatgacagaggcttaccctaagaaggcagaattagagaatagtattgcacagtttatagtccctcaagtagataatacaaaagttaacgagctaagagctgacttaacggaggcagaacgtaaattattacaaattaacagtattaaagaaggttttgacagagacttacgaaagctagctaaagactatgaaaacctttctagtagcaagatttgtcctacatgtggaacagacctcggggtggatagtgaagcacacattgcagaaacaaaagcagatattgaaaaacaaatgagtgttcttggtgcaaaaacccataaaattttagtagcgttaccttctgtaaatacaactattgagagcaaaaaggagctcgtacaacaagagatagctaaaaatgaggagatacaaagaaattacaatcagctagtgtcaacagtcaggggatatgaacaagaggtacaacaactagaaaacacaaaaaatagtttagaagagaaagtggcttcacaacaaacatatttatcacaggtacaaagtatgcctaagccaaaaagtagagacaaggatagagaagaagtacacaaaaaaatagaagcaactaagttgcaagtgctagacaaacaaaaagagactgagaagtatgaggaagcagtaaaagccttctcaaatgctggtattcgttctatggttcttgacttagtaactccttttttaaatgagaaagctaataaatatttaacagtgttgtcaggttcagatattgaaattaattttactacacaagttaaaaactctaaaggggaactaaaagataagtttgatgtagaaataatcaatggtagtggaggctctaactataaagctaactctgctggtgagaagaaaagaatcgaccttgcaatttcttttgcaatccaagatttagttatgagtaaggcagaactatctacaaatattgcattgtatgacgaatgttttgacggtctggacagcataggttgtgagaacgttattactttgttaaaagaaagattagaaacagtaagcagtatatttgttataacgcacaatgagaacttaaaacctttatttgagaaggtaatcactgtgcagaaagaggatggggttactaccctaataaactaaggatttggagagctataaaatgaaagtaaaaattatgacagaagaaaaaggggtactcacctcagaagtaagcaagaggtcaactaagaaagaggctatctactccgcaaatgtttttcctattagtagcttgtacaagtggtacccctttaatactaactacgcctatgtagtagacaagcatggggtatacagactcactgaactagatgactgtattcatacatcgtataacttggtaagtgaagaaaaagctaagttagacccgacaaagtggcacaataaaaccgtaacacaaagcaacttattactttgtagactagcaaataagctagggataaaaagcattaaagatgattttaaccaacaagatgtagaaaacggatttgaggttatcagtacagagttagcctatatcaaagaacaagcgacagcacgttgctacggggataaagatgtctttgaaagaagtgaactactcaagaaaagtaaagaagaaagagaacaagcatactttgagtacttaactgtagaggaggtaaacttccaatataagttaggagagctatacgctatgcttgtacttatgcggaaggtggtaaactactaatgtttttagactacattacaagtgagctaggaacacataaagatatagggcatgaattacggttctgttgccctttttgtggagaagacgactacaaattttatgtgaaggtatcaggggacaaaacagaaggtctttggcattgtaaaaaatgtaatgaaaaaggtaaccctgttacttttgttatgaagtatcttaacattaattttaaagatgccttagatatactagaggtgtacgactatgagatagacgaaaacagcctatctagcagagacgtaggggtaacagaggaagagtttctgcttatactattaaagaaatctagtgagcctgaggaagtagaaattagtgaggaagtgactctcacgccacctaggctacctaccaattttaaaagactagcagacaacatgaataaccccgaggcattaccttttctaaactacgcacactcccgaggtataactttagaccaactattcctacacaacataggatatgttatagaggggcaagtagaaaaagtagatggtaatatgatgacacttagaaatcaccttgtttttctaacacatgggaatgatggacaataccaatattggaacacccgagccataacacctatggggataaaagcttttaatgctcctagcaaacaggacgagtactctaaaagaacaactgtttttaacctaaatattgctaagaatacgccttatgttattgttcacgaaggggtaattgatgccttaacaatgggagagtcgggagtagctacttttggtaaacagataacaagagaacagatagagttactattaaataatgttagagaagaccagcctatttatttattcctagatacagaggcttatgaagttactactaagatagctagcagtctttatgaaaagcataaagaaacctacatcgttatccatggagatgaggatgccaatgatatgggatatgaaaaatcttgggactgtttaagaaataattcagtaagagctaatgaggaaggtataagtatttttttagtatcttgcattcagtaataaagtatgatataatagagcagacaagtaagaaggaggaattttattgagtaataaagtaaatgtaaaagttaagttagagggaggagctactatgccctcacaagggtatgataatgatgtagccttcgatttatacacatcagaggatgctattatttgccctgatgcagtgggagctacactaattcctacaggattaagaacagagtttgaccccactaagtttgggctattcatcagtcctcgttcaagtattatgaacttacctttaaccctagctaacaacacaggaattgtagaaggaacatatcgtggaggtattggtattcctttaaaaaatacgttgtctatgacaggtttttctgattttgcactaacatggaacccagacaagaaggtagtagaacgtatccctgtagagaagttacctacaggtaaacttagaagtgcggaaaaaatgttttacacagaccaagcattattgtatgataacacactaccagaagaactagcagaccaattgtttgttactttggtgccgactgggacattctttttaccaaaaggcttccgtattatgcaagcttaccttctacctaaatatcatctagacctagaaaaaacagacaaactatcagacagccctagaggggataaagggtttggcagttcaggacatggaggaaagtgattcctttagaccctattatccttattgccgactctataggcataggttattcaaaaggaaatataacactgtctttaaaactacgggataagtatacgggtaccgaggtagaaaaaatagctttattagccgttggtagcttctcagaaaaagtaacgcttgttagtagtgagctagtagataatgtgtataaaataaaagttaagatggaggaattatgaaagattttactcagtattttaaagaagtacaagatgcacaaaaaagagaggactctgaaactgtagaaagacttgcagtagaatatggagataaaactattacgtataaagaacttagacatacgttaggttccgtcttgttgccactaattattggaacaatggaaatcaataagcagttggcagacacctctttagacatcgttatcaatatgttagaaaaagagaatgtattatcagaggataccttaattgatttaaggcaactaattactagcatgaaacaatcagtagaaggaggagaaggagaaaatgagtaaagaacaaaagcagttaagtaaagaggatttaactttatgggttaactttgtagtaactggtaaggttgaaaaagggacggagaaacagctaaatcgtatttctaaacgagaggtatctgtaggagatgtaaccaccctaatccgtgctcttacaagtcatcatgatgcgtacattagttctcttattgaagctaattcagttcttagagtggtaatgcgagatgagttaaatatctcagaagctcaaatggacaaggcgttaaatacctataagcaacaagtaggtacagcactccaaaagacacatgatgctatcatggaaaaagcacaagaaaatgtggagttcacagaagaagaactagaggtacagaaaaaactagaagaatatgatactaaaaataaaaaagaggaataagaatggctagtacaggaagaggaagtcaagtaaaaggttctaaatttgaaagagagaccgctaaagctttatctgtttggtggggagctacctttaatagaacaccgggttctggcgggctaaaatgggcggaagaaaacaatgtaggtggcgatatagtagcccctccctccgcaggtttccctttcattgttgaatgtaaaaaacatgaagggtggacactagacaatgtattccttaatacaggagagcctaagtcttggtggaaacaagtagtatcagatgccacaagaacgaataagatacccttgttaatgttctctagaaacagggctaaaagctttgttatgctaccttatgatgacttgctatataacgtattacgtgataaaagaagtacagtagctagaactacagtgtcatttaagaatatcagggaagaaagtgtgttctatgatgtaatggtgttcactatagaagagttaactagcttctctcctgattttttaagagaacactatagtaagtatagttgggatttcttttacaaagaagtggacgagaactctgtagaagatttagaaacaggtatagataaaatgttagacaacctagaaggttaaaataaagaaaaacactaagtaggtcttttgtggtgacatatacataagcctacttttagtgtattgtgctatactagagtagaacggaaaaacaaaatatgtttggaggaatatcgttgacaataatagtaaaagacaacggaagtagaaaactaccttttgaggaagacagactaaacaggtacctagataacataaaaaaagagtttcctaagttaaatatagagagttatcgaaaaaaggcaatacactatattttaagcaaggacgagtattctgtagacgaaatggtagactacctaataagagaagcagaatctcgggtagacgttaccacacctgaatgggagttttttgctactagactatatttacaaagattgtataagaaagctagtaaaaacaggttttacgatgtaattatgaagtatggttcttacgtgggtttgcatgagagtctagcacaaagaggaatatattccgtagatattttagagaagtattcaagagaagagcttatggaagctgaaacgtttattgacagtagcaaagaccttctctttacttacaatggtttgtacctactagctactcgctatctagcaacagatgaggataagaaagtatttgagttaccacaagaacgatggttaactattgctttatttttgatgcaagatgaaaaagaaaatagaatgaagttagtagaagaagcttattgggcgttaagtaacttgtatatgacagtagcaacccccacattagcaaatgcaggtaaagtaggcgggcaactatccagttgttttatagatacggtagatgatagtttgcaaggtatttacgatagcaatacagatgttgctagagtttctaagcaaggcggaggcgtaggagtctatataggcaatgtaaggtcgtcaggagcacctattagaggagttctcggtgctagtgggggaacggtaccttggattaaacaattaaacaatacggctgttagtgtagaccagctaggagcaagaaaaggcgctataactgtaacgttagatgttttccacaaagatatagaagcctttctggatttaagactaaacaatggagaccaacgtttacgggctcacgacatttttacttctgtttctattcctgatttatttatggaagctgtagagaatagaggggaatggtatttatttgacccttacgaggttaagcagaaaaaaggttggtacctacaagactactatgataaagaaaaaggtagtggtttattccgagaaaaatatgaagaactcattcaagatggcacgattagtaagaagagagtgaaagctcttgatatgatgaaacgtatcatgattagtcagctagagacaggaacaccattcatgttttatcgagatactgtaaatagagctaaccccaactcccatgctggaatgatttatgctagtaatttgtgttaattctatggcacctttgggtagcaatactcatcgaaaacttttcttaaacgggcatagctgaatacgatgaataagctggtaagagagcctaagtccttgagtgaggatagaggtaatcccgtgctaaattataataaaatctttattgtataaatgcctaacgactatcgaaaagctactttttagtagaacttagtagagtacactcaagtgagtggaaacagaaaagactcccatgtggagtatgatatagtcttttctttacagaaatgtaaagcagttagtacactaacgggtagggaattaacgacccttactgaaagcaagcaagaaatcgcacaaaatatgtcagctacggtagttacagaggaaattatagatggagataaaattgttgtaacaaaagaagcaggagacttcgtagtttgtaacttgtcttctattaacttaggttctgtattttccgaagaagacgttatcgaccgactagtaaccattcaagttaggatgctagacaatgtaattgagttaaacaaactacctgtaccacaagctctaattactaaccagaagtatagggcagtgggattaggaacttttgggtggcaccacctactagctaaaaaaggtattcattgggacagtgaagaggctactaattttgcagatagcctatatgaaagaattgcttttcacactatcgaagcaagtatgaaactagccgaggaaaaaggagcttacccatactttaaagacagttcgtgggatacaggggagtacttctctagtagaaactatactagtgatacttgggttgacctagctaaaaaagtgaaagagaaaggtattagaaatggttacttaatggcagtagcacctaatatgtccacagcgcagatagcaggctctacagcctctattgaccctatctatgactctttctattatgaagagaaaaaagactacagacgacctgtgatagcccctgagttgaacttgaacacttacgctttctataaaagtgcttacaaagtaaaccagattcaaagtgttaagcagaatggtaaaagacaaagacacatagaccaagctatcagttttaacttctatgtacctagtggcattaaagcaagtaaactacttgagttgcacactacagcgtggagtgaaggtctaaaaacaacttactatgttcgttctaatgacattgatgtggaagaatgtgagtggtgctctagctaacagctattgacaacatgacccttttatgatatgcttatagcataaagtaagagggtttttccttgttgcagagaggtgtgagaaataattcaaggtatttgacaaacgatttaatgtgtggtatacttgccctaaatgtacaggaaaactactaaaaatagaggaggagaactaattgttaaaaaaaataaaaatattagaaccactaaaccctaacaaatcaacagcgattattaatggagaggctagtggtatcgtaaactggaacgatattcggtatccgtctttctatcgagcttacaaagagttaactacaaattactggttgcctgatgaggtagatatgaagagcgatgccaaacagtacaaccacttatccgaagaagagaagtatgcgtttgactccattatcggacttttagctacacttgactcaccccaaacacgttttatttataacattgcagagtatgtcagtgatgcctccgttcatgctattacagctatcattgcacaacaagaagctattcataatgagagttattcttatgtacttgcttctattactaacttacaagagcagaatcgagtatttgaggaagctagaacacaccctacgattattaaacgtaatgcacctattatgaagtcttatgacgatttcatgaataacaaaacacctgagaatatggtaaaagctttagtacagtcatctatcttagagggtattaatttctacagcggttttgcttacttctacaacctcgttcgtcaaaacaagatgacggggacaggtaaaatcattagcttcattaatcgtgatgaattagcccattcgaaatttatttctgagctaattagagctattgtaggggaaaaccctgaactacaaacagatgaactaaatgactacgtgtacgcttcttttagacatgccgtagagctagaacaagcgtggtctagtgaggtactacaaggcattgaaggaattgatgtggaagaaatgttagactatgtaaaatatcgtgctaataaaatgttagggatgttaggtcttcaaccaatgtatcaaggtcacacagaaaattctatgacatggattaaagcttacgccgataactttacagaaacaaaaaccgactttttcgaaatgcgtaattctagttacaagaaaacaaatttagacaatgggttcgatgacttatgagggtagggattttatattcttcattgagtggtaacactagtgaactagctcaaggaattaaagataacttggaggaactagcagtagaggtgaaatcttttcatattgaaagagaagaccaaatgactaatgaagtaatcccttatctatctaattgtgacctgattatggtaggtacatggacaacagattacggagtcatccctgaggagatggaagaggctttagaaaagtatgactatacttgggtagataagcctgttgctgtatttggaacaggagaaacccagtggggtatggaatacttctgtggagctaatgacaaaatagccaaaaagtaccactctccttttgatacacttaaaatagagcagtttattatcacagaagagcaagaaaataagctattaagttgggttactgatatagttaagcactggagggagtagctatgtacgagctagaaaaacttctaggtaaagataaacatggaaatcagataagaattggagatagactaagtttacaactagatgatggggagtaccgagagtgcgtagtagcggttagaaaagttaatactatcgtgcttaaccctgtagctttaactagtaaaagtgaaactgtaagtgtaacaggtgtattctttgtagttactgacccacatacaggagaagaactacctttactacctaaagtagatagtaacggtgtggtagacactcaaaaagcagaaataatcttataattttatagaaagaaggaaaacaagtgcattcagtagaagtagaaaagaaagaacagttagcggacattgtatcacaagacaaggttgtattaaagttttataaggataactgtagcccttgtaagatagtaggtatgtctattgaggaagctagtagtaatataaaagaggatattactatcgctaatgttaagttagaaactttaggagaggaagtcttctttgatttcggcatcaaaggcacacctacactgttgttagtagaaaaaggaatagagaaaaagagacatagtgggtttatttcccctgatgacttccaagagtttgttaaataagagaggcttaggcttctctttttttgttgacatttacaagtttatatggtatagtaacagtagtaacagaggggaggtaaaaaaaatgacaaaaaagaatattaaaataacgagtaagacagaggtaaaaaatagctctgttgaggcaggagacatcttctacattgaaggggtgtctaaggggcttactaatttgtctggtttgtatatagtgacatttgattttcatgcaacagaagagtcgctgtctaggtacgctctagtttctttggagacagggatgttaaacaccatttccatcaaccagttttctctagaagaaaaaatagggtgtatagtgaaggtgaataatatagaaatagtagcaaatactaaggaggtataactatgaaagtaggagaaaaagtagtaattaaaggtgaagtagttcacgtaaatggttctttggtaaaagtagagacaaaaaatagttctttttgggtaaacaaagaagacctaaagaaagataagagtagtggtaagtaggtatggcacacttaattgaaaagctaagactatataaaaaaccagaagaggtcgtaagaggaacatcagtaaaagcacagcatatttatgttgcaaatgatgaagcaaagtcattttcaggagccgaaaaatgggcgggaacgtctgcctataccttgacaagtaagaacgaatttacaaatgtaactattcactctctagacattagaggagagggaggcagagcttaccaagttacactaaatcacgataataatacttatttatgtgacttaagggaaactcaattaatggaaactattcgtaacgtaggaattgaggctggcggaaagttaattggaacttacagctttattgctgacggctcacagaaagttctagtaaggataggttcagacgcgtatgcaaaagcaagtgaacaagcggagactaaaaataaggcaaagagaattcccaagaaggagctagaagtaggaggagtatacgcacctaaggctacttatttaggaacaaatgactccctagacttatatataggggacttgtatagaaatgaaattgtagacgataacccttggtaccgagggtactacgaacgaagaatagaattagggaagcgtaagaagttctctgtaagcttaaatgtagcattacatgagggacaggtatgtgttccaacatacagagaagctgaaatcatacctgtatttctaacagacgatggggttacttcactaaatattagcaaccagctagggcgtttatccccaagaatggacttaaaatcaagccctaatttctttggaaaggtaggggaggctcctgaaattaaaacaacagcagagttcttagaatgtttaagacgtggcttagaagctcgcattgaagctttacctagtaaaatagagggcaatattttaaagggaaacttaaggttacaaaacgttatccctgaaatggagactatactcaagttaataggactcaatactacgattgaaaagcctaaatactccgctagttttgttaacaaaataaaaaatataaaatatatagttatagcagaacgtaaccaaaaagggacgttaaccgcatcggaaagtacagacttccatcagtttagtcgtgcttttgatgacttaatgtccaatattttagaggttattatagtagaggaggaaaataattgacgttatatatgaatggtaaaaaagtacatgagagtactttccccaatggggagattaatatcatgcctgatgaaaatttatgggtacttgaggaggagaaagcagagattacttggcagtactcaggaaacggtgcagaagacttgctagctgtcagactactagtgaacagtataagagagcacaacaagaaacataatagcattaatttaacagttctttatatgccatacagcagaatggatagacttcaaggaaatcagccctttagcttactagagtattttaaaatgttccctcaagttgacaaactagaaatagtggaacctcattcagacgttacagggagtattgctagaaaacttattgacagtggaacttactactcagagcggtttgctacagtagagatgctaccagaagtgttagcagaagagttgtctaatacttggtgggaacctgtattagtctatcctgataaaggagcaaaagacagatatgacaaattagcaactgattctgattattcattcagaagagctactgaaaacagacgagtactatttggaaataaagtacgggactttgatacaggtagaattaaaagtttagagctagtaggggaaccacctactagaaaaggatggacagctattattgtcgatgatttaacttcttatggtggaacttttgtaaaaacagctacagaggctataaagttaggagcaagccgagtagtactactagtagctcatatggaaggtagcgccactaagggagaattatttgaatctattgataaggtgtatactacgaacagtatggaagctagctacacaccagaagagctaaaagaagtagaaagatataaaaaactagggttactagacgtaactaatattataaaataaggaggaaaatatgcgtaaattaaatgaaatttctatgttaatgagtgattgctataaatttggtcacatgtctttatacccgaaaggtatcacacaggtgtatagtacacttgttcctcgggataatacttacttttttggaggtcgtacagataaaatggttgtctttggataccagatgtttgtacaacgtttcctgttagaacattttaataaaaactttttcgagttacctattgagactgtaaaagaggactaccactatgtagttagtaacgctttaggggagaagaatgctcggacagaacatatcgaagcactgcacaagctaggatacttacctataaaagtaagagcccttccagagggtaccttagttcctatgcgtgtacctgtgttaaccattgaaaatacacaccctgactttgcttggttaactaatttcttagaaacagtattactatctcaaaccttcttacctgcaacagtagccactactgctttagaagctaagaaaattatgactaagtatggagaagaaacttgtgataacttagaccatattatgttccaaggtcatgatttttcggagagagggcaacactctaatgaggcttccttgttagcaggtatgggacatttagtaaactttgcaggttcagatactattcaagctagtgtgatggctcacaactactacagtgctgatttagagaaggaactcgttcttaagtcggtgacagcatcagaacactccgttatgcaaagctacggtacagatgaagtagagacattctcaacactaatagacaataaccctgatggtatcttgtctctagttagtgatacgtatgactattacggtgtacttacagaagttcttccaaaacttaaagataaaattatgaatcgaaatggtaaactagtaattagacctgatagtggaaaccctgtagacattattgctggacgtgactatatagagtttggaactaattggtttaactatcatataactaaagagaacttagtaaaagcagtaaccactcgatacgagcagtcatgctcaaacaaaccctcaataggtcaaagagtcgatattgtagactacctaaatctatttgtagatagcgactatatttataataatgatattgttgataaagagaaaaaattagtatcagggtattataaagtaacattaagtgccgttttccttggtgaagacctgtgtaacccttataaagtagacaaacttgaactagaagattaccaaccaacccctgaggataaaggttctctacaactactatgggaaacctttggtggaagtgtaaatgagaaagggtacaaagttttagacgagcatattggtttactgtacggggagggtattgacttagaaatgttagagaagatttttactcgtatgggtgaaaaagggtttgcttctagtaacgtagtatttggtcttggtgcttatatgtactccttctctctttccagagacagttttgcccaagcaataaagagccaaatggtagttattaatggggaagaaaaattaatctttaagaatcctaagacagatactaatcatattaagaaatctcttaaaggaagagtgtgtgttacaaaaaaagaaggaacatatgaagttacagatgctttaacagctaaagaagaagccaaccttgaaaacttaaacgagctgaatgtaatttttgaagatggtaagttagcctatcaagactccttccatactattagagagcgtttagacagagaagcaaaaaaatattagaaaaacgaaagagcctttagtgggctcttttttgttgacaattaagtaaaagtgtggtaagatactagtataaatcaataaaggagatgttttatttgctagttaaagaaagaaaattacgtacctcaaaaaatagtatgctagattttttaaaggagggggagtttgtaagagccttcggtgacttatggtatagaaaagataagaaactatataatgttacacttaaagaaatatttgacatttatctggagcttgacgtttctatgctagaggctactatgctagcttttagcactaaagaaggattctcacctaaaaagtatgcgttagaagatttaaaggaagaatatggtactaatatagtaatcgactttactcttcctgagaataaagaggactttgtagtagataatacaaaaagtctgcctctattatgggacacttacagaagttatatgcctaaaacccaagcagagtttcactacaaaaaacctatttctaagaaagtgttgaaagaaacttgggggcacgtcctagacacaggctcactaagtgaagttagaaagaaaatggttttaggtacaactgctagcattcgaggtctacgtaattcggtaagttcgtcaagaataaaaactagcggagaactaaccgttacctctgctaaaatggattttaaaacaggagtacttacttccgttgttaagcgtgcaatggtagacgaatacgaactatctgcgaacgaatatacagtaaactttaatctttataatgctatggagcttattaatgacatatcggactataaagtattagacaaaggacacagagaccattacgattctaacatgatgtgggagattgttacaaagagtataacccatatatacaaaaatattgaggataagtacaccctcttaaggctagtatcctctcttacctcaggtgtgcaaggaagatactgtagaagtgttcgttatgagatagaaggctcagagttggaacactcagaagtacctatgcctttagtacgtgaagtagtagctaatctaaatttaggtgaaaatatacttgacagagcaggaaactggttttaccaagacgtagtaggcagagaaagtgttgcatctacattaaaaacaaacaattatcaagagttagtagagcgtgcaggttctttagtagcagtttttcctcttttcgacaggattgcttcttatatgaacaaccaaactaaaaaagagcagaagatttttgagagagctattgaacgacattggctaggtggatacaactaccttgagcgcagtaatttaaaaattattggtaatgcactagtacccaatgaagtattaagagacctactcacttataatgcagtacaactcgtcattgagaagtttacacgctaccaacacaatgtggttgcttacgattacttaccagataaagggtatgaacaggagattgaaaaaatacttccaagccttcaaaaagaaagtaagcggattggagaaaaaattcctaagtacattgaggatacattccagaacataaaggaaaatatggtagaggatgtaaaaaatgtacaaaataagattacacctctaaagaaaccatgggaatatattatccatgttaaccaaggtatctggaaaaaagtaatggaattagggaattttgagaaagagtcctatattattatctggaaagctttgtacgaaaatacggaagagaatgtaccatttcttaaatataaatccaagatagaaaacaagttactattcgaaagctctgttttagcaaaccctgctatacaatcatcatttactgaggcatttcgagctattggcgggtatcttgagagactaggaagaacacctgatgagtttatcaaagtcattaactacttgtctacagactgtacattaagacagagaatgagtcttagaaacgcaacagtccaatatagagactatattaattcctatgtagcattaaactcattagacttgaacgaggctaaaagattcgatttgtgtcctttctccttgaagttggctcatgatattgcaacatctgattaccaaactatgcaggattctagagcggatgagtttatggtagcacgatacaaagttatgcctgacgagttcagcttaaacaacagaaaattctctattgataaagaggaatatgttttagtacaacctaaaggagtagacgacctaaaagcggaaggaagttctttaaaccactgtgtcggaacatatgcagctagaattattaaaggggataacttgatttatatgttacgtaagaaagacaacatagaagagagctatcttacaattgaaatccttgtccacgatgactcccgagtatcattaggtcatctacagggagagtcttatcgcagtaagattccaacaaaaataacagataaattaaaggaatttgtggttaaagtaaatgaggctatgttagctcaaagagaaggcagaaattttactaaagacactaaatccgaaactttagttagagccagttaacttgtatttaaactcttaaattatggtataataaaggtagaggctagctaataatatagctagccttataattatactgaaagaggaaattaaatgaaaaaagataatactcggaaaaaaaggttaaacatttataacaccgaccgatattttaatatgaaccttatgaaaagaagtgctattgagaaaagtattaaagtaacacggttgaacgagaaagaaatcgaagaagaaatggacaagctagcagaaggttctacccgatttaacaagaagaataaaaagagctacttactctatcaagagaaatatacaaatgatagactaattcaaaagctctttgctcatggaggtagtgttacatattacactaactctatcataccttactatgtaataaaacatatggcacagtccactacaagtaaagccgtatactctgtaaagaaagagttcacagaagaacaggtaaaaaatatagagctaaccaaaatggctactacagtgataattgacgttcctgttgttgttcctgatgttagcccctatgatgttctgttctcactacaccctttaaaaacagatgtagacggtgtacagttttcctttccaagattaggtaaaagtgaaatacaaaaaaggcacctaccgttctatgagttgaaagggaaagtatacgaattacgacccgaaattaaatatgaatatgtaaaatacactcaagtatctttatctatttgggggatgtatctctggttagtctgtaatagtgaagaagacttccaagaaatagacaagctactacaaaaagacctaaaaagaagagacagtggcagacgacctagtgcaagtcagcaactaggagggaaaacccatgcctaagaagatgataaacagaacagatatagcccgtgaagtagcacaaagaacaggtttttatatcaaagatgtagaacagatactagcagaaagagacaccgttattatagatgcccttcaaggcggagtcactaagattaaagagcacaaactgtatcagattgaagtagaaacaaaaccagcaaagaaaaatgcttggaatgggttagacaaagtatactttagtataccagagaagaaaataattaagtttaaacccttagtagcagtagaagaggtaatagatagcttaaataaaaaagagacgtaatagtctttttttttattgtacttgacaggataatcaactcatgctatactaaggtataagaaaataagaggagaggtgccatgtgaagatactttttttacaagagtacattagggtaaaccacgtccttgtacaagatacaggtacagtcaatatttttcttaaaactgacttagggaagaagctagtacaaaagcttaaatcagtaggtataaccaaacaagacttttatattgactatgcttatgctaagatacctgaaccaaaaaaacaagtaaacggtaaaatactaacgtacaaggagccaacagcacaagagaaaaaggaaccacaaacattactaacagagagaattatacaagaaaaacctgatttaatcgtacctatgggtaacattgggtgtaaataccttcttaatgttgcatccattaggtctttaagaggtataccagaaaaggtgaccttacaatcagagcaaacaggggaaaagtttactccatgggtaatgcctatgtattctatggaatatctaatatcaagtcctaacgtagagggtatgtttgatgcggatttaacaacactcagtcggtacaaaacccaaggagacgaggcgtttactcctgcggaagtatcttatgagctagttacttccttagaaagagtacaggatatttttgaaaatgaagttaaaaactatcctgttattgcatgggatttagagactaacaccttactacctgagatgttaggagccaagcctctagtcatgtcagttagttggaaagaagggcaaggtgttactattccgctagaacaccgagagagtcattgggaagcctatgaacttgaagagatttataactatattaaagaatttgtagcagataaaaatattgttaaagtaggtcacaacatttaacacaatgggtgttgtaaaactcctctccatcggtgaacgctaagtgcaaagcatatgccaataccgagcctacattactggtgacagtagatggggtgtagagactgaaccgagccattgtagggcaaggtgagaagtggtacatatcacttaaacgaggagcattgttccatataggtaactacaaataaaacctattggtaaggggcataaaggtatagtccacgcttatatgaaaatataagactacgtgcagtatgatatacgttttcttatgtcgtctaaagggtttactaagtttaataataatcgtgatacgaagatagggtactacttgtctgtaacacaacagatagaggactccttcaaattatcagatttagcctatgagatgacagatatgggtggttacgacaaaccacttgccgatttcaagaaaaaatatattaaagactacaacgataaaaaaacagcagaacaggaagaaagaaaaaagaaagtagtagaggaaataaacaaacagtttgtagaagaaacactaaaatttaaagaagatactaagaaatacaacgaagaaaaggcaaataataagtattctgtactagttaagcctgtaaaacctaaaaaaggtaaggcacctacaaaggcagaactagcttacgagcctttagctaacgaagtagatggaggaaactttaactatgactggattcctctagagatactacatccctatgctagtggagacactgactgttgcttacgtattcataatgtacttacggagataattgcaaaaaaccaaaaaatgtggttccagtttgctaatttttaccctagtgctattgtggcattagctagaattgagtctaacggtatctatgctcagacggactatatggaagatattaatgagaagtacactgaggaggaagaacgcctcgtagaagaaatgcgtaagatagcagaggtatccgagttagaagagcaacacatggagttgtacgaacgcggggtagaggagatggcaatacctcctgcaaaaagagataaggatatagctaagttaagagacaaatacaagaagaagctaaaatttaatcccaactctgcaacagacaaagggaaagtcctttatcaaattatgggtattacacttccctacgataaagagcatattaaagagtctgcttttgatgtagggaaacctgagaataagctaacatgggaggactacaaaacagataagaaagcactaaactatattgtagaccattttcccgaccaaaaggcgtttgcttctctcctactagagcactccttagtaaaaacaagaaaaaataacttcacagccaagcttctaaacttcattagtaataaggacggaagggtacatgggtcgtttaacgctacaggaacagagacaacccgtttaagctctaataatcctaatatgcaacagctacctgcaaaaacttccgaccctaaaaggttcgactatcagtacccgataaaacgtatgttccagactgcatttgagaatggagcactactacaactggattactcttcactagaaatgcgtatcttagctttagcagcaggtgacgaagctatgacacaagcattctttgatggggcagacttgcacaaagaaactgcttctattgtatggggaatgcctgtagacaaagttccagcagaatttcgtaaaaaggctaaagcagtaaacttcggtattgcctacggagaaacacctttcagttttgcaccaaaacatgatatgagtgtagaagaagcagaagacatcttccaacgttattttgcaaataaacctaatatcaagacatttattgatgaaacacacagagaagctctacgtgacggttttgttgatacattacaaggtcacagaagacttatccgtgatgccttttctaaagataagcagatacaaaatggtgctttacgtaaatcagttaataccaagattcaaggaacaggtgcttactgtacaaatatgtctctaatttatatcgaccagtatatacaggataacaatatgaaatctaaattagtattaactgtacatgatagtattgtagtagactgcccaccagaagaggttgagattatggctcgggtgtgtaaaacaattatggaaaatctaccaattgacttcttgagtattgaatggaaaggtgaaaaaactaagtatcctattgaagcagacatcgagattggtactaactataacgatatggtagaatacaatcctagtgagttaaaagagttcaactctttaacgggttatgtgaagtactacaaagatttagggaaaatttccgattacatggaggctggtataattacagaggagagtatggatttaggacaaactagaattaaagaatcaaaagaaatttacaaaaaaattgcctaaaaggttgcttttgttttaaaaatagtgtataatgatacagaaagctatttctaaaaacagaggagggtaaaatatgacttaaggagggatttgtttggacatacatgtagattcattagaatttagccaattacgaattatagatggtaacggacaagttacagagcttgatttagagagagagttgagagtaaatgaatactcgatgcaggaggagtttctcaaccaagcaccaaaatatatctattgggcttctattctagagaaagtacgtatgtatcaggagtctgcatcactagctttagaggtagtgggagcacagatgaatgagaaggcaagagagtattacaagactgtagacactaagccaacaaaagaccaagtagaagcctatatacttcaacaagaggaatatcaagtggcttacaaacagttgttaacctataatcacttggttaaacaactacagtacgtagtaaaagcgtttgaacaaagaaaagacatgctaattcaatacggagcagatttaagaaaaaataaagataacggtataagcccaaacttcgggtaaaaaaaaataattataaacgataggagacaataacagtatggtaaactttaaagaatttttaaaacaggagaaccaagaaaacgaaagtaataataatgaagaggtagcatttccaaaaggaaaacatcaacgcttattcttgtctaaaaaatcaaataccgtggtagttcgtattttaccaccagctaatttaacagataactatgcacaaaagtatcgtaaaattatgctgaacaacgttatgacagatacaggaaaagcattaaatccgtcatttacgttaccatgggagcctaacgaggattccccactagaacaggctttagctgattggtataaccgtggagtagttcctaactactatggaggaaaatcacgaccaagcaaagctactgtagtaaacgctgtggcattagtaacagatgcaaacgggaactgggtacatgaggttgatgaatacggtcaactagtagttcgagtgttggacttaccttattcagcatatacagctatcaatcgtaaattagcagacccaatgctaaacactacaggctctgactatagctttatctctgatgtaggagctaacccaatccgtattactaaacctacatcaaatacagagaaagcatatcaagttgaagtatatacaggtattacattaccacctcttcctgagaattggagagaccaattggaggacttagcttatcaagttagcccgtcagaagactataacagcaaatttgttaactttgttattgaacgagttaatgctgtaactagtggaggagctcaacagcaacaagctaacccttacggaggacagcaagcaccacagcaacaaggtggttatcaacaacaagttcctcaacaacaagcttaccaacaacctgcccaacaagcacagacgtatcaacaaccagtacagcaacagcaagcacaaacataccaacaaccaacacaggcgtaccaagccccaacacaacaagtacctcaacaacaagcgtaccaagcacctgtgcaacaaccaattcaacaagctcctgtagcccagccacaacaggctcctgtacagcaacaaacgtatcaggctcctgtacaaccacaggcacaacaacctattacgcaaggaatggtggactcagcaatgagcgggttagcatctcctaaggaacctgtacaacaacagaaaccagtaaccccaccaccatctccacaagcacagcaaccagtaagcaatgattcaagcttacctgatgtagatgagttactacgcagtatgcaagaaggttgataagaactaacatgaggtctaggatttattttctagaccttaataagaattttagagagggttgttttcatggcaagagctaaacagagtgaaaaagataacaagacagaaattgatttaggtaaaaaaattgggttaactactttgcaggatagtaaatgggcggagatttcagataagttacctacatttatccctcagttagactatattctagggggagggattccttttggtcgtttgacggagattatgggtaaaaacgcaagtggtaagtctactcttgcagtccacttaactaaggtagccttacagctagattgtaaagttatttggattgacactgagggaacggcagaccctagtaggcttagtcagctaggcgtagatgtaaacaaaatattttttgttcaaccaaaaaaatctaagaatgctgaggtaaacaaagaggattctattacagtagaatttgtagcagaacgagtagaagctattttaaatgccttctctagctctgaacaacgaattgtgataatttgggattctattggacatacaccttctatcaaagagattgaacaaggggtagcaggtaaacaaccgggtattaaagctaaggcaatggctcagtttgctaccattattgctcctatggtaactaaatctcattgtgcctttattggtatcaaccaagctagagacgaaatgggaagcatgtttggtgcagtagacagccccggaggaaatgcactacaccattgggcttctctaagactagaggttaaacaggcatccaaaatagaagaaccgatgcaaaatgcttttggtactatggataatgtatatgtaggacataaattacgtgttattactaaaaagtctaaggtatctacacctagacaaagtgcagaaatgttccttttatccgcatcaggtttgcaatatgaagagaatatttatcgttcttgtgtggacagtaaaatgtacggtcttatcaaaggactacaatacaagtcttatattgcagatgatggaaaagaatacaaaatgtatggtaaggactgggttcctttcttacagtcaaaagacggggaaaagatacgaaaagagttattccagaaaatgatgttaattagtttccctactggatatagtccacttgataatgaggtagtggatgttaacttatgggaagatatgaaggggttaacagaactgtacaaaaagaataaagtaaagcaacctaaggtgaacgagggagcaaaagaagagtctattgaggaagtactagcaggaattgactcggataaataactttataggggagggaaacttccccttttttcttttagtactattgaggaggaagtaaaatatgagagctaatagaaatattgatttaaccgagcatgtggaagtttggtctgaaaaaacaagaagtattccaccctctaagttaaatccatttctagatattaaaaaaagatttctatatgcaaaagagcataatatcccaattattctagaaaacagcgagctaaataactctcaatctgtggttctagacctaagctttgtgggagataaatgggcaatgggatacgtaacacattattcagctattcgtacagagtctactccatacactatccattacagtgatttgtattgtgcaggtaaacaaagtaaaaagaataaaagagtaaaagtaatatttaaaggggataacccttatactatggaggaagcctgatgcgtaatattgataaagaaaaggaatatattagaaacggcaatcgttatgtacaagagaacagtgggagaggggtatttgctagagatagtgaccgactattccatcagtacagtaatctacggtggagtgtctacaataagcataaagactccttcaatgacgaaactactagagcagaattacaaagctatattgacgagcagtttattaaactagtgaaggagtacgaaattaacggtgcagtagacttcccggggtacattaaaacaaagttaaaccagagagtaaagcatacttttgttaagaatgtattccgagacaaagctagagaacgtctaggtgctgatgagtttgaagtagctaatctagtagaccgacagcatactgatgaagcactactagaagaccagatgttacaagagtatgtatttggtaagacagagttcacacctattcaaaaagagttgcttgtctatttactagaaaaagggatggtttctgatgaacgagtaattatcagaaatgtggcatctaaattcaaagtgcctagagaaaaaataaatgaggaattatcggaactacgcttttttgtttcttctaagatagctaagtatgagtcagaaaaaggctattaatattgaaataacgtctgctatattagagttagaaattcagtataggaggctttaaacaatgacagaaattcatacaccgcaagacgaagaagtaaaagatgaagtagttcaagaagttattgtagaagaacaacccgagaacgtagaagaagtagtcgttcctagtgacgatgaagtaaaagcaaaatttatgtcttatgttccgttatttgttactttagttcttttcttgaacaccttattagcatcgttgggttgggaaccattagcagtttctcaagaccaattgtatctagtaggttcttctgttgcaacagttattgcaactgtatggtcttggtaccgtaataacaacgttactaaaaaaggcttgaagagagaaaacatcgcaaaccaagtattacctaaaaaagaggacaaataagaaaggatggaatattaatggctattaagaacattaaagatttgcttaaagagtctacagtcgttttagatgggaagtatctacaagctagaacggtaaccgatggttttacaggaacctatcaaggtaaccactcgtttaaagtagaaaaaacagggagtaactactctgtataccctatgtattttgacaaagatacaaaacaaatggagaagagaaaagacccgattatctatacagatgacggagaaattttctttgtagttcgtactacagtagacccttacaattacccagttattgagcaagactccttaacaggaaaaaatacagagaaacagattctacaagcatttattgctttctctgctgacgagtatagttttgctaactataacttgtttatcaatgacgatttaaagttctatgacggttctgctataaaagaagttcaaagtattaagttccctaatgcgacagaaacaataaattcttttggagaagataaaacattaactgttatctttacacctgcggatgctagcgataaaacagtaacattcacaagttctgacgagagtgttattaaagtgagcaacacaggtaagttaactggctctgtcaactccactgaaaagtctgctgtggttactgctgaaacttctaatggaaaaaaagctactgttactgtaataatgaaagcatcagagctaccagaaccgtgataaactcatagaaaagaaagggactttcgagtctcttttttttattgactttacaggtaaactgtgttatagttacatagaagatacactaagaaaggagtgacaagatggaagacaattttgtaacagcctataataaaggagaacagacatgggatgacttagcattttgggaggagtcttggaacaacgaccctaatcatattgccagtctagcggagtatctagggatgacagatagagactactggaattatgtagtaatggacaaagaagtacctaaagagctcctacctcgaagtgagcaaccccaactagaaactgaatacatctaccacatttgccataaaaagaatgaagtacaaatttttaaagaaggttctttagtctctcataatggaaaagtggtacaagtagataaaataatctctgtaatcatcacagagaataactcagtagccattcatcttaagattaaggagggaactaaatgattcagctaattacaaaaaataacatgcagggatactatgcagactatctgttacgaaaagtatctacacatgaagtgagaacgacttacttagagaagttcagactgcaagacatcactagttatagtgcagatgctactattattatcgtactaggatttagctacagtgacagagaggcgggagctactgttagtaacttcgaagagtatagtgcaacatttaataacccattcactttcttttaccactttgctagttttggtgataagtttaaatacccaaatattgactctctagttgacgaaaaagtaagtcctgttatggtaatgtacaacaccttgaaggaaaaaaatcctgatttcttaatagcagagaaagacaaagaagctatgttggttgctactgctgtagacgactaccactgctaccgttttgcagaaacagggaatgggctgtctctacctttaaaagagctaggagacctctatggacgttatttagctgaattgtacggaaacatgtctttggtggagctaattcagtcaaacaagaccattatcaaagtgagacaagagtatcggaattattacatcaagaaaaaaatagagcagtctaaacgacaacttgttaaagatgttatgctttattttgtctatgcagaggaatacgagaatgagattgcacaccatattattgataaggctaaagaagttaacgatagggtgcttgtcttagtaggaaagcatacaacaaataatgatatgttccatgtacgctcccataatattcacgcaggagaggtagctaagctaataaataaaggttcaggtaaagaaagtacagcggtggtattcttaggggattccaaaacacctacatttaatgctgttgtgaaagttttgctagatgctagtgatatttaaccactaaatgtggtataatagaagagaagtacacactaattagttaggagaaaataatttgaaaaatttaacgaaggcagaaagaaaagctattgagaaaggacacttaggaaaaacagccgttattctaggagtgctaaatgcttctcacggcaaaattgttatgtctcagtataataaaatagcggaaagtttaggaatggaaaaggtagctaaaccagacttcttagcactaaatgaaaccattcaagaagataaggttttacttggactgtaccaaaaatcgtctgtaggaaagattacactagatgatttaacaggaggaagtaacgaagacaagtatagccagtttaatgctataacagcttatacagtagataatgacaaagtattagcagacttacgagaatttagaaaaattcaacgagatggagcttatacacaacatctttttaacggagtgaaagcagatttaaagaaagagctctcttctattgctaaacctaaatttattattaaagagaacaagaaaaaaacaggtaaagaaatgattgtagccttatctgattggcacatcggagctttagtaatcaactctgatacaggaggatacgactacaaaaagctagtaagctatctgacatatttactaggtgaggttgttcagacagtagaagagcatgatattgacgtatgtcatgtatatcatattggagattttattgagcatattaatatgagaagtgtcaaccaagcatatgaagccgagtttaatgcaacagagcagatttctaaatctattcgtacaatgcttgacttcttaaacgtattagccgaccatgtacatgtaaaatttggtatggtaggcggtaaccacgataggttccaaggaaataaaaatgaaaaaatttataatgataatgtagcttacattattctagatacattgtttttactacaagattccaaagcgctacacagtaacattacgctaattgataatcggaaagatgtttacagctttaaagataatgttgcaggtaagaatattaaagtagtgcacggggacacagaaggtaagaaagtagatgtaaaaataccaaaacacattaagagtgaagtagtagactatctattcatggggcatatccatacaactagaattgtacaagaagattatagcagattccacgtatatgtaggtagcccgatgggagctaattcctattctgcggaaaataacctaccgacaacacaaggttcccaattattagttattctatctaaaggtagtaacaatccaatgtttttacctatctttactaaataaaggaggaggaaaacaaaatatggcatttttaatcacagtagtgctagtaacgtaccaagttattgtacttttgcaggcggttagtggaactatccgtaaagaacgaattatggtagaagaaggcggagaccccctaccaacatcctcctatgttttcttaatcattaatcaaggagctattgcaggattcctattcttgtggggggcagtactaagctttattctagctatggttactaatgttatcctagctgtaatactatttacgctagcagtaaccatcttatcaattgtaagtgcaacgattctaagtacaatcgttcagacaattgcagttattattgtaagacgaaagcagatagcacatacgaaaaaagttattgctgaaaaagaaagagagtagataaaatatgtctgagtatgaagcaagcagagcagacttaagtagagagttcctacaaggtgatttacagcctagtattatgagacgagggattctctacatcagaaagaaacctgaatatattgttgagtatgtagaagaggctagtagcgatacattaatggtaagtgtgtttgcagttcaaccaaagtctaaaccgcctttaaaggcttggacattagttgataaaacattccagtataacacaggaaccactctaggtttactagctaaggagctgaaacatgtacctaaaaaaacagagaaaagagaaaatccccctttgtttatcgcacctgcactagatggtgtggacacctttacaggaagagcaggtaagggtttctatgaccgagaacctgatagagccacaagcgacggtaaaatcgttcaagggaagcacacaggtgtatttgttgggtttgactctattacttgggagaaagctattgtgctaactgaggagatagctagtaggtatttaactgcccgagcccaatggtataacatttagtaatgctatattaaatagcatgttaacaattgttaacaggagaggatggaagatatgacctttacagttgttttattcctagctgtaatactcttgtttttatccgcattatgttttaccagtcataaacagagttggaacgtagtaggcggtgtactattagcctttggtctaatcgcattgttctatgtgcttataatggcaagtgctgtttcaggtataaatggagaatggtttgggatgaggttataatacacacattgggggctctgtaaggggctcccttttttatggaggttggaatgaaaaaattttatgaagatgatattaagcaggtactactaaaaaaaggaaagatgtttggtgatataggaaagagcactattgtatttgagaaaggtattactatctcctctacaatcgcagactgtcttatatttacagagaaaaaaggagtcattggtgtggagataaagacagagagagactccactgcaagattaaataagcaactaaaaaactactccttagtgtgtgattatgtatatgtaatgtgtcatgataaccatattaaaaaggtagagcagataatgaaaaggtaccatcatgaacatgtaggcattatatcttatattgagtttatgggagagcctgctattggtgtctataaacctgcaagaaagtcaccgttgaaagatgtataccacacgattaatattctatggaaggaagaaatactaaaaatattaggtgcctttagacattatggtgcaagagctcctaaaagctcctacacagtagcagatagaagcggaggactaaatgggttatccataaaatccagtttctctgctcgtatgagtaagccagaactaataagaaatttgattgctagggttggacgaccagaggctaatcggattgtttgtgatattttcattgaacatagaaacaatcctgccaaggctataaagtaccaccacttctttcctagcaaactagaaaggtaagtgaagaatagtggcaaacaatactagaggtaatctacctagggggtatactgcactaggagacgggagtgagtctcacaaaacagtagaaagagcgctaagaggttactatacagtagactttagtaatatatacgaacacatgacagaaagagacttagaagtacgacaggcatacgctaaagacctcatatctcaaagaacaggcatccctttagacatgattttatttagaaagaagacagagaagcagacattaacagctatagatgaagtcttttatgtaaaacaaggggtaaaaacaataggaaaagtatctatcaggtcacaaagactactaagcagtaatatgctgattattatttacatgtctaagagccatatgcctcgtgtagttagaaacacaggaatgaaaacttatacaaaacgtagtaagtcagacatacgtgtagcaaaaagacaaaggaaaacaacaaaaagacagagctattgacaacagagtctacttatgctatgctaatattagaaaaataaacgaaatgaggtacaagtatgcaaatagtagaaaaggagagttccttaaacagctttttaagcagtatgagaaatgaaaaaggtacagtggtaggggtctattataaaaatgcaatgatagacgaaagtatcttagcagaagccagtcttcaattagaggtgcctaataagacggagaaggtcttacaagccacggtagcactaaaacctatgccaagtcaggtaaaccagttaacgaccttagagagcctcctagaggactctggaatgggatggttttttgatagagaacgacctatcacaccaacaatgagtacccccttgtttaacgacttagctattagtcaaagtgcactatcaaatatacaaaattatagactagtagttaacatggctgaccctaaagtaacaaaacaaacctttactttttataaaaatacctatacattgtttaaagtagaggacagacagttggatagtaaggttgcaggtaacattattcacagtatccatgtcttatataaggaagagcagtatgacggtgaaaccttattcggtagattgtttgtagaaagtggtagtaaagaagctaaacttatgaagagagatactaaaatcttgttggacttgttatgtgatgaaaacatcactagtctttttattggaggaatagagttatctttagaagaccgtttatccaatgctaagcttttccctctgtacacagcagttacagctacaagaacaaactatgatgcttatagattcctaacagattcaggagagactactattgtttcggatatgattctagagagtaagttagttaaagtagctgactataagtttacctttgtagtaaaattaaaaggaaatgcacaattaactatatatttaggatagaagtaaaagaagtcagcatttaattgttgacttttttctttatatatgttatgatgtagttaataaaaaagaaaaggggttgacagaagtgacgaattttttaacattagtatttattctagctattattgtagctgttcctacgttattagctaaagcgatgagaaaccactataccaacaaggagacaggtgtttggtggatgccatatacacactggtttgtgtacaactttttatatacacagaagctaggatggctaattttaggctccggtattattatctccatttatattccatttattggtggatttttaggaacagtcattggatttgcagggggactacttatgttatttggtgggctaccttatgtatttggtttactagggggagcagtctttggtgtttttgtagacaacattgtgtatcatctaccagaatggtatcgagcttggtttagaaaccgatttgggattgagtttccttacctagaagacaaggaagatgaaaacgaggagtgaaagtaatgaaaaatgaagatacagcttggagtttactaggtttaatttcagctattgccttggtagttatttcttttctattcttagcagggtgttcacaagagacatcacaggttaaagaaacagagcaagagaggtatgagaagttcactcctatgattgaatatatttactctcagaagttaattaaggtgtataggaatgaatcagacgagcttgtagagtacacctactatgcccgcacagaagaggaagaagaagaagggctcatccacgaagaggagtataattattacaaaagtcaaggagttactgatagaggtgttattaaaacaacaataaataggtatggtaagggtaagtacccattaaaactatgggatgtgcaagagtacgaaggaaatgtcagcttagatagaatagataatgaagtctactttgagggtgattaatatagaaactacaagtacagtaggatactggaacatagataccttttcgtatgaatatttgtactgtgctacagagggagagccttggagcatagggactatgctaacggagtactacactgattataaacaagtaaggcaactaatgcaggacaaagagtttggtaataagaagtttagcttcacgtcttcaaaggtaagcaaggcaactaataaaaagaagctaaaggacaaagagacctattttcagtatatatttgaaaaagggcagtggttctgtgtgtatactaaagggttcatggctaacgtagagatacctgctatagaaaacccataccgatttaataagtggttagacaagaatgctaacaagttaagtaacaaaagagtaaaactgttacgagaactagttgtaagtagcagtgaggttaaagaggaaacagagagagtagttcctgctatggttaaattagaacctccgttacgattattgctagacgacactcatagtgtcagttctcataatatactggaatttttaaagtcagggaatttgtactctttagctaagagagagttctatgttgatgactatggaaccgaagtagactgctttactataaatgagttaacaaaaccaagttcccaagtacctagtcaaataacggttagtaaagagcatacatctaattggtatcctctattagacctacttattgactgtggttatatatcagactatgtagttgaggatacagggtctaatagtaaagaagtaaaaatagagataaaagaaggaggataacgggatgtctatatatgagtctatcgaggggctagaacaaagtattatagagggattacgtacaactttagaaacagaaggtaagaatatagccctagacgttatcagaaaagaattagaagacagcccgttcttacggttttttgagttgtctcaaggtagccctgcgggagatgtataccaagtagatactactaagatagctagtaaccctacacagcattacgaaactgcccgtgtagctagtatcgtgtctgctaacgttcctgtgtggttgtgggggcaggctggaacagggaaaacttctattgctcaaagtattgccaaaatgctagatttagaattttaccctatgcagaaaataatggacgagttcgaacttaaaggttttgtggatgccaactctagattcgtaccaactccactgtatgaggcaatgaaaaatggtgggttgttatttattgacgaaatagatgcttccgtaccagaagcactaatacttattaacactgctttagctaataattttatgcgatttgctgacgagactgtatatgcacataaagactttagggttattgttgcagggaacactctaggtaaaggaggtaacaactcctacgcacgtaataaaatagactcagctactttagacagatttgtacctattgagataggatacgatgaaaaaattgagttattaattacaaatggagaccgagaattagtatattttgttgcactagttcgaatagagatggctagaacaaagttagaccatgttatctcttacagagcgttaagtcattataaaaaatttgagtcttttggtgcggatttcttgattgaaaatgttttgtttaaaacactatctttagaggatattactagtgtagctcgttctcttagcagaataatcaaaaaaacagataaggattctaagtggaagaacaacaaagtgtggaagcagttacagacatacctttcaaacaaaggagtagttgtagatgacacgaactaatcatacgtattcatctaaatttatagcagaaaaccaaggaaatgtctataggttgcactatgcaggagaggactatacaaagataatagatgatatgtcagctatcccccactatgatatagataatatcaaaaaccctcaaatgaaactctcatggtctacaggaggggaacctattccagaatttatacacaagttccaaacaaattatatggaacctgagaaggcacaagagcttaatgtaatgctctctaaagtatctgaattagtaagtagccaaccacctatttatcatttttacggtaatactatatcagttcctagagccattcaagggcaccctaaaagttttaaacatcggaaaaaagaagtaactccttatattagtatatttgtagctatcaatgttctttatgacgttagtgtagagaaccataataagtttataaaaaatatgttgaaagttattttgatactgtcaaaagaccatactgtaaaagtaacactaggtaaagttagtgcctctaggggaagaatagctgtggagagctttgtactaaaagaggctaatcagagttttaacttatctagactttggtaccctttatctagtatagccacatatagaattgttctttttgagatgctagaaaggcatatgacactagagcctaaactatcctcttacaaaggtaattatgggtttacaatacacagtacggaaggaatagcagaagcagtaaacttgtttgagaaatacgactatgtagtcggttattacgaaacaataggcatgacagaaaaagaaattatagaaagatattttgtgtagttgactttatctttcatatatggtatagtagctatgtaaagtacaaagaggaggtaatactatggaagtagcagagttagtaggtaaactggtaggtaaagaagttatgattagtacagaaaggcatgagcgccaacaggtttatagttttagaacacacttaatagaacgtgttgagttgaagcctgcccctaggacgattgacaataaatcagttattgggaatgccttttttgtagtctatttaaaaggactacctaatgaatatcgactagaacagttaagaattagaatggatatactagacagaggatgcttattacctgttgagattgacacagaaacaggagaggtaacacttgttgtaagcgactctctagctaaatatttctttgctagtcttttagaagttgaagcacgtactgacgaggacaagaaaaaggagctagtacaggagtttctagcagtgtctaaattatttcacggtggcgggtggactaacggtaactattatgcagaggaggattaaaactccgtatataagaaatgaggtgaatatgtatgattaaagtagataaagtagaatgcaaggatggaagagtttactatgtctataatagtaataatggatataataaaaagaacatatgtattaatggaaaggtgcaaacagtacctaaagataaatatgagaagtatttacctaaactttcagagataaaaaaactagatacattgtatatgagtaacaaaaaccaactgtacaaagaagttgtatgtgccagcagtggaaatacaagtaataaagacaaaaaggtacgaatcaatataaacaatgaagtagtcagctacctgccgaagaaggcaaataacttggcgatacttctaacgcctgacaattgccatctggttagtagggtagagccatctatactagtagaacttctttctaaaagaacagtcattatctctagagatggcggtggggggaatgtaagaagaataggttctgcttgttgtggggacgtactaacgttagcatataaatatataaatggtaaacagcaggtaagtattaatgtactatctgttaaggatttttaccaatatgaactaggagggttatttttatgattaaagtagagcatattagatataagcctgtagattttggcttgtccagtacaactcgtacatcagaacactacgtatttaaaagtaagagaggtcaaaaggtggttacaatgaaaattgtgggtaatggtattcgggcaatgccttttgctaagcttaaaaagcaccatttagacaaggttactgtattgaaaacattttatgtacctaatcgaaaacagctagctaagcaactaggtttaagttactacaatagtgggaaagcagtacaaaaaacagtaaaacgtttagatgatattcaggactcgattgcatttagtaatgctattcaggcactacctgaccactcagcattgagatttaagttagtaggggaggagaaaacccctaaaactaaacttaacacagatggcagtacaaactaccttattagaacttccgctaagctattgggagaggatgtaaaccaaattaacttacttaacgtatctggtgtgctaaataaagcggaggcagaagcagagaagttcttacgagtactaaatctgtataaggaggctaccaaagatgattaaagtagaaaaatatacagctagtaccacaaaaaaagcaggttctattagagggacttatgtagtaggtggtactagaaaccagaacgaatcagatttacacttaacagggactacacactacaagtataaaaacaaaaaaggtaatacgatgattacatttgtcaattattatacaaaagaattagtctctattcctgaaaaggacttaagccctcaagacctagataccttttatagtacagtaaataacagactaactactgtgttctatgtccctaactggaagcaaatgaataaaaatgtactaaaaaagggtacaaaatagcaaaattataggtagttcataaagattttatgatactacctagtactgttttctaatctgtttcacttatcttgtttcatttacttcttttatactctagattgtttcaaatgcttataagtagttatgcctagtaaaccctactctctagtacactttatgggtgtttcacttacaaatgagataatacaaagtgtatgctatattatatattaagaacactgggaggcaacagataaccagtaggtcataaaatagtataaatgtagaggagttacatgtgaaatggcagataacaaagataaaaacaaggatttagaaaataaaaaagaggcacctacaacatcggttttacttagactgcatgagatgcctatcttagcttctaaggtaaatgatgctttagatgaggacaagtcgtatgactatattgtaaacttatgtagtcagtataactttgtaatctctaaatcagcaatcagtagatacaaagtaaaaagagaagaatctatagaaacaggaattgatttaggtgctttattagataaaaggcgtaaaaacgaaacaacagctactgtgtctatgattgcccctaaagagaaaaagtcatctcccgaagataagagatacgaggaaacttttagtactgtagacaaggtatacaacgatgtaatggttttggaagagattatccaaaaaggaatggaaggcttacaatacacagagactatagaggcacctattgcgatgaaagctattgaaataaaagctaaaatcacaggaaaccaacttcaaggactaagtattataggactacgtgaattgaaacttagaagtgtagctaaagagacagcaatcacagaaattttgatgcagtatatccccgaagataaacaaggagaagtgcttactaagatgagagaagtagaaaaggaattctataagaacttagacttatcagatgaggatagaagaataactgaggcgcttaaagtcgcaggtataaacagctagaaggaggtttttttatggagatagtagtgccagaaggttatacaaaaataagcatgactaagtttgataacctagaagataagttactagcactattaacaggagagacactgttagtgcgtggtttagaaaaaagtacgaacgcagacgtactagttaagctagatgacagaaagtaccctgtgacacagataagctatgaaattagtattacagacaatgtgtttcgagaacgttattggactatatatgacatatctattaatgcactaagtttgtatgatgttttcctttttgaggaagacaaccctgcatatgagctaaagatacaagtatcagacaatgtgcagtatactagtatcagcggtgttaaagacacagctaaagtaatagctatttatgaagatgaagaaaaaaacttgttttgcgagttatcaagggaagacacattgtacccattagtagaacttacaaaaatttagcaaaaggtaaaatcccttgaaagctctaagtagccgactagggatttttttgttgacattatatagaaagtatgctatactacatttataacatagaaggaggagatttaaattatgaacaacaaaaaagaatttgataaaaaggtgactgaattaaagaacgcgacagagattattaaagagcgggtagcaaaagtaacacacggtctaaaccaagcaacaaacctgttgaacttaaatgtaaaagaagtaaaacgagttgcttcaacactgcaacctctagaggaagttgtttatgagttgcagaaagaggttgacggattagcacagtatagcaaagcggaggttgtttactacgatggaagagaggttaaccttgaggagattgacttagaagaggacgactatgaagaggatactacagaagaggaaatccgagataccgtctctactgatggagggggagcacggattagagtatatcctcctgaggaaactttagtatatatggcagagcatattggacaaggagtagagactgatgaccttgctgtttatgaagctgttcgagacacaaacggtcacacaggtttccgacaagaaggctattatgtagaccctacggaaacatatgctaaccgagataaacgattaaaagaaacctcaaaagtagaagaggttgaggaagtagaactagaagttactattattagaccgaaaagtaaatctaaatctaaatctaaacctaaagctaagaaaaagcaaaatagtaaaacaaagtctaccacaactgagtttaaaaatgatacagaaaaaggtattgtaactgactatattaatggagcaggaacaagtgctattgcacgaacttacaatgttagtatgggtactatctacacagcgcttaatcgtcataagattcctaagcgtaacatcgctaataacagcgtagaggttcaaatggttcctgtaatcaataataagaaactactaaatgcaattattgacagctacaacaaaggagtaccagttaaagacatctacgaaaaatacagtattcataaaaacgggctatactacttgctagattatgtaggtgtagaccgtaaagtacaccataaagggaaaaaaggagacgtagaacttaagatggactaacctccatcttagttttataattgtagttaccgaaggaggttagttagttgaaagtccatttcaagaggtctgtgataatagttgctttagtagtaactagttttattctaggagtagctttaggttatactatccatgtagataatgctgttcaagaacaaatagcattacaaaataaaaaagattcttctactagtaaaaaggaacctagcaccaataaagacattgcagacacaaaagctgtagactatgagactgacgtagctgtaaggttgaacctcctacgagtaacagtaaaagaattttctgagaagtttaacgaggtagaggataatgtacaagtacttaatgatatagaatactataatagttatattgcactaattggagacgtgaaacgccaagttagtgatatacaaagaatgaagtatcgtagtagtgagtctgctttagctagttctgtaaaccaagcagtagacaacttctatcaagcctcctccctagaagaaaataacgttcagtcaagacagacggacatttctactatggttgttaacaagctaaatacaggtaatgatgaattagaaaaagtctttagaataataggaggtaactaaatttgggaattgaagcaatagtatgcgccttatatggtgtttgggtagcacggaacctgatgaccgtgacaagtgttgatgaaacactgacggaaaagattattctagaaagcagtaacacacaagtagcccacttagcaataagtgaagccaaaaaacttctagagaaagaaacaaaagagaaaaatagaaaaattattattgattacattgaaaaatggcatcgggcactaagtgacgatactttaatagataccctagtagagaatgaaagaaaaggggctaggaatgctagaattattgctaatatatttgtaaaaattgggacattacttggaatcctagtattaaatattccgttctttcttgcattagctaacttcacagtagcaggttgtgtggcttttgttatattaggcgtaatatatggttgtcagacggaagcaaagcaagaaggatacaaagcaagtaaggactatattgtagtctcttatgtgctaaagcaggctttattcttgctggtagtgttaatctagtaggaggttttattatgagagaatatgatgaggaaaaagaacgagagctagctaagaagaaccataggataaaaagagaacgagctgaggcacttgaaagaaaacgagagataatagagtatgtaagagaactagataataaggagaaaaagttagtagaggaacaggcaaaagaaaaaataaacggtaagaaaagtagtactgtagaaaagtatggtaacgcactggatatggtaaagaacacagactttgaagctttgtgtcgaatacagacagcttaccacctacagagagaatatcctatggttttaagctctctaaataacaaaggttcttatattcatctaggagtagctaagcaagttgcagataagaaaaccttgtcaggagatgctacttacgagtgtgagctattattttcagttcaagtacctaaagcaggtcaaaagaaacaaaaagatttactactagagctagaaaagagaaaaacacttgagggtaactatactaacgtagaaggtagttttactagtcaccaatacattccttttctaactttcttaaacagaagaaagattgatgcaaagattgattctatgctagatgactactctctagctaaaacgaaggcacagaagaagaaaatccaaggagaaataaatacttgggtaggataccaaaaagtagtaaaaaataaaggatacgttctttatagctatgcaagtgccaaagtggtagcagtttatcacagagaaataagtgtatacagttaaggaggggttaaattgagcggactaaccgtatctgataagcaaagaaaagaaatgctttctatgatagagactctatggttaaagagaaatcccagccaaaggtttaatcaatttatgtttaacttagaaagagagtttgcttcctataaaaagaatgaaatttcaaaacgaaatagagttttctaccaaatgagccacttagggtacaacaaaggatacgtctatgtagcccaccaagtccctgatttatttgatacatctgaacaagacttctattcttatttgaaggataggataggataggataggataggataggataggataggataggataggataggataggataggataggataggataggatagaccttcctatcttttcttttgttctaataaagtagtgggttacagctacactttcagttgtttacaagttgtaggactctctaagtagccagctatagttttatctgtaaaaagagataacccagaccccattatagagcatatataccctaagtagcaggctacacaaaataaccaatctatagataactaatgtcattcagagactatagatgacacagcatccttttgtaaatttatatgttcgggctagattaagaggcgataccccacccctcctcttagagtacttgtagagggggtatacagagtttcgttttcctacctagtatttatatagaatatataagtatcttccttaaaaagcctgtggtggcttgtgagagcatacaggaaggtattgtaccttgtatttatacggtagcaggtaccttaacccctcatacaagaggaagctttctaccccatataaacgataaaacactaagtagcaggctatactttctgatataggacagtatgacttatcaatatgtaaaagggacaaagctctaagtagcaggttacagaaaaaaggaaaaagctctaagtagccggctacagaatttcctaagtagcaggttatagatattaaaaccatttatggggtaggtgaatgcaggtagatggcggggtggtgaggactttgaaataccttgtgaacgttggtgttatgccgtttactgctagatgataaatagtttcttctatataatacaacgaaagtaagctaaaaagaatttaaaaatgatgtataaaaactgttgacactttttatattgtatgctattataaaagtagttaaagaaagcaaccaaataaataaaaaaaaactttataataactgttgacttttaaaagtaagcatgttattataagagtatagaaaagagcaacaaactaaaaaagttaaggtggtaatctcaatgaaaaaattaaaagcaaaaagtaaactaagctttatcctcccaaaattaacaaacgacaacaaaaaaattgactactctaaaattcttacatcagcttgcaacgttttcggaggctacacattaagagagtgttcaggcggttgggttgacaataatacattatatactgacgaaagcaacgaactagaaattttacacgaaggcttaaacgaagacatgaaaaacacagtagaggaaattcttgattttcttttcagtgaagaagaagcgggacagcttgcagtatacgcaaacaacaataacgaagtttacattttagaaaacgaagatgtaaaagaatttaaaaatattttataataactgttgacttttagaaaagaacatgatataataaatgtacagtaaaaaacaaataaaaaaaaattaaaaggtggtaatattaatgaatattgaaatttgggcgtacgcaacgaaagaagacgaaatttgtagtgcagagcaattcatttgttcagtaagtaatgaagaagaggcagaacaagaattaagctacgcagagatggaaaactaccacgcttggaaaacttactaataaatttatatagcaaaattataataagtgttgactttaataaataattttgctataataagtttatcagttagataggtaaacaaactaaacaaaggtggtagttaaaatgaatgtagtaactaaaatcagtacaacgatgaagctagacgactatagcaacttgacaggacaagaatttacaacatctttctatcagttagcaaaattgcataaaaacgaaattaaagacggagtaatgaaagaactatctaattatcctgaggagttacagaaaaaaatacaggattgccagaaagaagcagaaggacttaaaaaagtagtacaatttgttagtgaagacgtgaagaacaataaagtaaaattacaaaaaaattcttatggtgaccttaacactgtttctttgaaagcaagtgtgcggagagctttagcgcaatacggtgacaatattaataattgggaaaaaaattacttactagaggattgtgctatacgccttcaatacaatgtttctaatgtgtatctttcggaactagctaacttgcggtcagatgcaagtgaaaatatgattgcatggagctataaaaaagattattcattagaagcagagtttgaaaaaattgatgactacatggcggtaaaacttatagacttagttacaaaatcacagcatgagcatagaaacacgcatgacgatataagaatcaaactgttagcaattctagaatttcaaaaaaaatacgaggttattagctgggacttatacaacggtaaaaagctttctgatgaagaaatcatagcaattcatagtaaactaccagaatttatttcaaagtttcatgctgtttacactgatattgaggagttaagaaattatttagaaattgagtaaaaaacgcttgacttagtgatgatacaatgctataatataagtatagtaaaggttatcactaagtcaagtacctacataaaattaaaaaaaagttataaaaggtgttgactttagatactaaacatgatataataaatgtatagtaaaagttaaggacgtgttaattatgaaaaaacctattcaggaactaagacaattaattgaggaggaagaaagagtattggtagactacatcagtctagaagacgacaacggcagaaacatcatttttattgaaaacctaaacgcagaaaactcagatgacagcactatgctagaaacttactccccttatgaagcattagagtttttagaaaatgaggggttgcttgcttaataaatttatataacacagttgtaaaaggtgttgactttaaatagtaactgtgttataataagtttatcaagtaaaggtaaacaaattaaaaaattaaaaggtggtaatattaatgaaaaatcaaattgttgggtataaaaaattaacggttcaaaaaatttggagagcaacaaaagcgcaattagtatttgaaggtgctagaatggactacgctagatacctaaaaatgaaaaaacaagggaaagaaatgggtattacagtaccaaaattcacgttgaaagagatgtaccaaaatcgtgctagcatgaaacgtgatgaactaagaaaagacatgttcctgtgccttgatattttaaatgaccgagtaggaaaataaaatgtggtttgtaattattggtatcagctttggactaattttagggctacgaaaatacaataaggggaaaggagcggaaaaaggtgagcaaaaaaaaccttagtattagtaagatgaaagaactctgtcaaaaagaagggagtaccttctttgaacctagcgccatgaaattcttcgggacaaaaattgtaaccaaaccaaaaaataacttatttattacggaagatttagcaaaagaccacagaactaaaaagtttacggtaagaaaatttgagaatggtgaagtgattagtgttaacggttttttgaagtacagttccttggcagaagctaaagaagttctaaaaactattttataaatttatatagcatagctgttgactttaaataataactatgctataataagtatataaaggaagagggtaatttaaatggcagacgtatttggatataatgactttttaacagaaatgggaataaagtcagtaagaggaaatgcaattacagagcaattaaaacaagattcaagctatattgtagaggatattaaaagagcatttaactttaaactttacaataatgagctaacaggggaaaacaacatggtgaacgtaactaacaaacctctgtctttagtagtattcttagcagacaataaaagtaaagtattattttacgatagtgtgaactttactagtaaaaaagctcgggactggaaaccatttcacgctatctttaaaaatggtaaaacgtatattgtacaagataattcaaattttgccttgacattcgttattaaaaatggtactattattaaagaaaggaaagcacctaaaaactcaggtttgattttcttagaaagactagcggaaggggatttataagatgaaaactgttaagaattgggttgaaagcaacatctttgttattctaacttttgtaatgggtttactcttactactagctttattacactattaagtgttgactttacagaacaagcatgatataataaaagtatagaaagagaggcacacaatgaaagattacgaagtaaaacgaattgagacaaaggaagctaaaccttttattttaggcttacactatgcaaagcgtatgcctagcgtgtcgtatgcgttcggtatattccttgatgatgagttacttgggatttgtacaatcgggaagcctgcaagtcacagcttatgtaagggtgtatgcggagaagagtacgcttataaagtatttgaactaaataggctgtgtatgaaagaaaagttagaagctaatgttttaagttattttgttagtaaagtgcttaaagaattaaagaaggaaaatctaatcttagtatcttatgcagattcaggtatgaaccatgcaggttatatttaccaagctacgaactggatgtacacaggtatcacaaacgaaagaacagacaaatatacacctgaaaacaagcattcaagacattataataacgagtatagtcacttacgaaaagtaagaacagctaagcacaggtatatttacttagcaggggataaacgttttgtaaaagaggtggcaaacaaaattaactacaaaaaacaagtatttccaaaatctgaaaacaaaaactacgagttaggaacaagacagaaaacaaaagtaatcaatacaaaggataacactattttttatcaatagaggagtgtcacaaatgaaaaataatacaataaatataaaagatgtaaagctaacaatcagaaaagatactataaaaatgaaaaagctagctaaaaagatagcagagtattacacagtaaaaaaaccttctttagcggatagcttgcgtgtgcaacttattgaaagtgaagctaaaaaactagaaagcactattgcaaaaaatgaggagcttttatatttgatgaataactttactaaataaattagtttttactgttgactttagaggttaaacatgctatactaaatatataataaaaaaaggtggtaatattaatggaaatgacagaactcattaaaactaaattcgaaaaacacggctttgaaaatctaaaagaggaaatggaagacttagcagagggttaccaaatttttatagatactacaccagaagcatttgaactagcttatgaactagaccgtgagggcttcacaacatgggaagaagcggaaagtctttatattgagttggagagtgcgtaacacatgtatactactattccacattacattattaaaaaagtatctgataaggaaaaggtaaactaccaagcaatttggaaggtactacctgtagaggtgcaaaccatttttgtatcaaagaaaacagctagcttctatatagttgatagatacgctaatagccaatttttcagaaacttgctaacaatggcagggaaggcaaattttggggtatcaatcagtagttccagcaaatcccatttagagataaggctcacctttctaaatacacatttttagttttatatagtgtagctagtcctttattgctacactaaaataagattaaaaaaattaatataaagtgttgactttaaatagtaactgtgttatactaagtatataataaaagataaggatgtgtgccaagtggaaaaatttaagtcactaggagaggctacaggtgaagaactagcctttgttttagaaagtaactcccaaatatttgatggtgtgctagacattgcagaaaaaaatcttggaaaagaagactttgacaaacatgttagacataacggtcactatgatgttgcgctatgttaccttgaaatatatgcagaggataatcctaacatcttattcgagaaaaatacctttcaactaaaaaattaaaaaaaaagttatataaggtgttgactttaaaagttaaacgtggtatactaagtatatagtaaagaacaacaaaaaaattaaaaggtggtaatacaaatggcaattaataaaaataagcacagtgtagcagttgttaaaactcaaacggggaaaattgttggttatctttcaactagagacgaagaaaatggcgtggatattcaaatggacatgcaaggttatactgtagaagcgctataaaaataatttataaatttatatagcatagctgttgactttaaataataactatgctataataagtttataagttaaacacaaaggcggagatagtaataaaaaaactagtataaagtgttgacttttagaaataagaatggtataataaatgtatagtaagagttaagaaaaaaactaaaaaaaaactaaaaggtggtaattaagatgacagttaaaacagtttatagcccaaaattagggtacacttatggaaatttagaagtagaatacgcagatgttaaaagtttttatggtaaagcttattttagaaatatgcacacagacgaccacacgcatgtataccacttatactcttacgatactttaattcttgaggtggtagacggagttgtaacagcaatgacagatgacacagaccactatagcatgacaactaatagacacattaatgaatttttagcacagtatgaagatgactacacaggtaaactaagcaaaaaagaactactagaaaaagctaaaaacctaggaactgctttagtatagacataaaaggggcgggttaacgtgactacattcaagaagaaccgcacagttaactgatactagaccaagtaaaaaagagtttataattataaaaagtgttgacttttagaaaagaacatggtatactaaatgtatagtaagagttaagaaaaaaaaactaggagcgtgttaaaaatgagcgtaaaaattatcagtgaaggtttagaaagctatatcccgtggagtggagcagttgtgaactacgaaattattcaagaaaagggactattagatgtttttgagtcattgttagcagaatggtatccggaaggattaacagaaacacaagtcaacgatattttgtggtttgatgaagacttattcagctttttaggaattgaagaggaggaggaataataaaaatgagtataatagcaattaaaaaagaaatccatgcaaaaggttacaaagtgacaggaacccatcaaggctatatagcacaaatcaattttgacggtacaggaaacgaatatcctttaccagctacatgggatgagttcattgaaacatttaaagatggttggaatggtacttatgaagacgaacaagcgttcttcaacgatatgcaggaagttgcattaaaagaaatcttagacgaactaatagacgtattatataatctagacattactacatataattttactatagatgatgttaaaaagaaagttattacattaaacaaacctacagacgaagaagatgcagaagacttagtaatagaatttgattctacttgcttttgggatgctacagtagaaaatgataaaataaaggttactgtaagaaacaaaagtaaataaataaataaataaataaataaataaataaataaataagggttatcactaatatatttatatagcgtatctctacctttcacttgcaatactagcggaggtatgctacaataaacatattagacctaaataaaaaggagaaacttatatgatgacaattgaagagttagcaggtctacaaaaagtaaaaaataagctagaatgtgcatataaacaactaaacgatgctacagaatcactagaaaccactctaattaatgaacctctcgctataataaataccgattactcttttctagctaataacatagaggatgccagagaggtattaaacaaaacaatcactaagttagacaggtataacgaagcactagcaactaataaagaagagatagacaagtaccttcatcttactaagtctaaaactaaataataaatagtataatagacaaaaaaaaaataataaaaccaaaacctgtaaatcacctcagcatttttttcacacggtgtcaactcaaaaggcttatatatgcgcagtctttctacctattttttagaggggataatagtactatataaataacctctataggtataacttcttatatttaggattatttcaaactaattaggtctaaactcacattattataatgtcaatagggtataactcctaataatatataaaactatgtacctattgactaataatggtgtgtaaggtataatagaggggggaggtgtaggcatcttatgaaaaagaagaaattcagtttagttatttctttattactacttcttcgattatattagtgagggtacgcttgtagatttaaaagaaacaaaagtaaaataaaactgttgacttattaggttaactatgctatacttaaggtataaacagttacaagtgggataagagggtataggagctcgtatataggcttttactttattagataggtaaatagcagtaaataaactaatcccgctcagaaaggcttgtaggaggttagagaagatgtttgtattaaatggtttactatgtactttactagtattgatgattctagtgggggctattgctttaaacgcagataagcagactaagcttattgtaggaattatacaggtggtatttatgactatgtgcgtagtcctctatatcactattatagggattagcattactattattccttttggtgtattatttgcgatttgtggggctgttgctttgttcaatctacttacttttaataaggaggagatatgatgacaaaatacaagttaaatacggttagtaataataatttaaaagcaggggacgtggtacttgtcgagaacttagaatgctacgaattaaaatcaggtgtttacaagattattagtgagtgcgacggtctatataattttgtgggtattgatgtagacgtgtacgaacctgtttctgtagaaaacattaaccgactgcttgggagctgtgtgttacgtgtatccgaattaactttggatgtagtcgttcaaaactaagattagaaaaggaggaagggactatgttgttatttatcatgaatgttatcagtgtagctatattaatgagtactttttactatatacgcaatatatcccctttctattacgagcggacgactagactagttaatcacttagtagatatagagctgattttaggattatttatctggttagtgtttattatctctattacttttgtatagggaaaggagtagaactatatgcttattataaagtctttaattgtagctattatatttactatcttactagtttatactatagacgggttttctcgtattcgtaaaggggctaaaagtaatggtattttagctgttgtttgcgggattatagggataattaccttttctattctagtagttattatcttttagaggtataccaattttttaagggttcatttagctaaaaatagctttggggtgtgccaacgagtttcgcgttttggagtgaaaatatccctaggggggaattcaaaaagttcacctataccaatttgaaatgagggggttttattgtttattgtactgatgggcgttttgggaatagtgttagtcggcttgggtaactattatacaatgaaaggtctagcgatgtcacataagcttagtaaagacgtagctagtgaagaatgggtaaatgaacagtttgaagattctatagaaggtcgtataaagctaaataactatgctagagctactatgaatcgttcagactatgtatttaacaaagggatgttattactatttataggtgccttattacttatcccgctagtaactttaatttaaaaggaggtagaaaatgggtttaacattagtaaatgacgatgataagtatctacgtgcgtttaaactagattcttttcaatcagtaaaaggaattatctatgtatatattgttattactcccgaagtattaacagagtacctctcacagagctcagatggacagtattttaagagtagtaaagattatcaagtaaatgtacaggaattagctagatggcttaaaacgtctaatagaggctttatagagacactatctcataatgagaacattatggcaagtgtaggagaaaataatatataaaagagttgacttctttcttttgatgtgctatagttagtgtatcaagtaaaggagttctttttatatacttaaaaaaaagttataaaaggtgttgactttataaaagaactatgctatactgaatatataataaaaacaaaggtggtaatattaatgaacaacaatcttaattactggacggatacactagcaatcaacttggcgggattaacattagtagaacgggaagaggtaactacacaggaagcactcatatcagagatgtatacaagagacttacaagctaacaaacctatgtttaataacttattagataacctagaagagttaaacgacaaagcacgagaagctcactcagaggctttctatcatgcagtagatgctattatatcgctgtgggacggaataagcaaaaacactttataaaggaggaatagttataatgacaaataaaaagaaaccaactgtacagatgacaggggaggacggaaatgcctttcttatcttaggaagagttactagtgcgctgaaacgggcgtgatacaccgaagaagaaagggaacagtactataccgaagccacatcaaaggactacgaccatctactggcggttacggctgaatgggtgaatgtagagtaagcatataaaccaatactattagaaaattattagataaaagggttgacaagttcaaccccttatggtatacttagtatatagtaaagagaggggacgagttacatgctaacactaaagaaactattgaaagctaacaaaggacaaaagaaagagattgagaaagtattatccgaaagagaacaagctatcgcaaacttagaaaagctagttaaactacaacaatctgcttaagcattcagttacgtcattagtggtaggcatatatacccaccccgtggttctgtgaccgtactttggggctatgctacacagggggctcctctcggggcacgtttttcgggaactttttttaaatttttttattcttaaagtcaacaaggaatggctctatagagggctttacaagaatctttataaaaagggcaccccgttgttttttcttaggtaattttaaggagaggcttctataaataaagtgaaaaaattccggtaaaaatttttaaaatagggaataaggaggtgttcctatagatatgaataaagataaagacaacgcattagagtttcgagcttggtgtaagataaaacagatgatggcagtggttatctctattaatttttataaaggagatatacaaggggaggatgtatttctctcttaccctgcacttgggtcatttaacttaagaggcttttccgaggttatccttatgaggtacacagggttacgagataagctagggacaaagatattcgaaggggatattgtagaggttcctttagaggcagaccatggatatgcaggctctgtgggggttgtcacgtatcgtaactatggtttaggggttatgacacctcaagggttcttttatttagaggaacctgagttgcttcttgttacagaagataaatatacgaataaagtagaggagggttaagaatgaaagcacctatttcagaatgtccttattgtggttctacagaagggtacacacttaggtcacaagcaagtggtactatctactggcggatgaaatttaatgaaaaagaggcagataattcagagtcacatgataatttacagtatagtccccataagtacgcttattgtaccgtatgtgagagtaaacttttcaaagcagaggaggtgaatgggtaagatggagcttcctaagcacattatagtaaaaattaaacgagcagggcaccataataaaaaagcaaatgacttacaaacggaagtacgcaagtggttagagagtagattaacagatggggaatctctccctgactatataggagaccaactgatagatgcgttggagcttggtgtagatggtagtgaggcgttacttaaattcttagctgaatatgaggagtgaatcttaatggaaaaattgaatgagaaccagcagattgtactaaagtggttaaagaattacttattagatgattctgatttttataacacggtatatgcttctaaacaattatacgtgaacggctttcctagtgataaaagtaatattgcttttatgaaactaacagacaaggagttcgctagcgtactactagcgttctctacatggtacttactagaattagaaaaggaggatttaacatgaaagagaaatgtagtatagagatttcttactataacggtgaagacaagtttacgggaaaagtagtgtgggttgtactagacaaagaagaaggtatccaatttgtacttactgagaaaccagaggacgaatactttgtttttgacagtgaggagtcggcaggtattgtaggtggtcttattagactaagactagcatgtgacaacatagaagatgactcccctgtaacaattaattaccttaaggaggaggagaataatgaatagctgggagtctattttagggctaggtagcacgcacaaaatggtaatagcacaagagttggtaagttttctttataaggaaaattgtgatactactacgctaggggagaatgagcttttcatactaaagtttctcctcgataacaaagtggagtttaacagaaaagaaacttattatggaacaacgttctacagaggttatactgtaaccgtaaatgttagggactctgtgttagcaactataggtaaggatatagcttccgtagataaagacgaagataaggctactccagaagcagtaagttggttagaagtggaatgttgtgatatactgtatgagttcctgtctactaaactaactgctgagaagggtagtggtagacttagtgacacgcttaaaataagtcgttcttatgaagaaaaggtaaaaggactagaaataactataacaaaaacaaaagtttttaacaaattaggaggagagtaataatgaaaaagagtttatggagtacaatactagaagacacagtactagaggataataacaactcaatagcatacgcaagtgttgttaacacacatcgcaagtttagacgattactagagaatgaggactatgaggctactattttacgtatgcttcttgaaaggaaacaagaaattgacggggtgtttcaaagcaacgttgagtttagtcccctaagtgtacgcccatcagcgccacctgtaaaagaacgagggtacgaaatagttattgatgtgaagagctttgtagccgaagcattagatatgcctaacgacctagatattaacctaggtattaatggctttgagcctgtctttgctaatatgctacaaaattatttaaatgaaaagatttttgaagaaaacgaggtaatatatgagtttgagggtagtacatctgtggagcaatctataaaagtagagggtgacggttgcatcgtagttgtgaaaaacataactcgtacttggtaaggaggaacttatgaatagaaaaacgtttagacctagttggcttggagaagcgtataaggggtatataccaccaaaaaatgattttcctgtgatacgtataaaaggagacatggtttcttggtttactatccttttagaacaaaatgaagaatttgtacgggagtggatgttaaagtataaaagaacaacacgtacaaaagaaacaggtgctcattatataggggatagtgactactccacttattgcgtgacaacctaccttgttaccacacgtacagcatcattcaatcccaaaaggcttgttatagctctatttactaaagaactagctcttttttctagggaatcgacatcagtaacactaacagaagaaaatctaaatttaaatgagcgtattactgaatatgttgaggcattgagtgagtatctagagtctgtaggtatttttccgttctctcttcctagttggtgtcatcccgactatcattacacatacgatggctgtcgttttactatctccaagagagaaagggaagtaatatctagcactaaaatgaacttagaagaatataaccaacatttactaaaaagcaaaggagagaaactaatgaaagtaactcaattagaaacatttaacgattttattgactacatggttaaaaagaatgaagaaggctggaagtgggaaaacaaagaccctctagtacctgtaagtttcgaaaggtggcaaatagatagagaccaaacacttatagctaaaaactttagaaataaggttatttatacagttagggtggaagaccttgaccaaacacctacaattgtatcatataagacagatgcggactgggtcggtagcttagacccaaaagaatatgaatgggagacgatgaaacctaaaaaagtacctattgtccacccgtgtacattaactgttgaagaactcgaaagggattggaagactaatacaaaagagtatgacgaagttaataatccttctcattatacgtctgggggcattgagcctattaaatatatccagagtcacaaaatgaactacgaaaaaggtaatgttattaagtatattactcgggcaggtaaaaagaaaggtcagagcgagattaaagaccttaagaaagcaatgaaatatattgggttactgattgaagatatagagaaatatgagaatagctaatttaataacaaaagtaatagggttgtggattctgtgtagactagtaagtgtctaagggttcacaaccctaatttaaaatgtgaaaacaaacgaagtataactgttgactttttacttatttcatggtatagtaagtatatattaaagaacaggagtgattacatgagtagagcaggctacaaaattcgttatgtaaaccagagaggtaagatggtttgggttaaaagtattgggtttggaacaaaggcattagaagtcgatactactaccaacaaagagaaggttaaagtatttttggataagaaatgggcacttcaagtatgtaaactcattgatggcgacctagatgttaattgtatgatagacgacattacttataagtattttatcttctatgaagcagaagataagaagtactttctaaagtcgctaactgtagttcaagataagattgagcctgaatggacagaaaacgagccagaacgagcggttattcataaatacaatgatttagatagaattttagggctagttaacgatagaattgataaggaagcaagagcacgtcacgatatgtttttactttaaacgaggaggtaataaaaatgattttatatggagtgtctctttatggggaggtaactgagtggactacagataaagctactgcaaataaatgggtagataatgcagtacaatctatagaggataatgatatagatgaggactgttatgttattttgttaagactagacttagaaaaacttctatatggcacattcgaccgtgaagtagatttaaacgaccaactgcatgatgaagctacagtactaaaagaattacgttggcaggtggacgaggaagggctatataatattgtggaggtggcggaatgaataataaaaagacagattataaagtatacaaaataacctacaagcaacgttttatgggggaggttcttattgactcgtatgaaagagtagtgagggatgataacgaactacagtctgcaattaacgctttatatgatgacccacatgtgttctcggttagtagtgaggaggtgacagaataatggagtttaaatcagtaaaagaaataaatacagagattagtttgttagctgacaaaggaggtatctcagacggctcacacacatttgaggagctttactttcatagaatggttttatttgcaacaatatgtaatgctaatcactctaaaagttggaagtctaagaaacatgaggacggttctatgtttgataactattttatcgttggtatttctacatcgaaaggtatgttttcttaccattatcacttagacaattgggactattttagtgttccagaactagaatttgcccctacatgggatggacatacagctaatgatgttactcgacttttagatatataagaaaatgtaagtaaacaaaggaggtactactatggatttaaataaatttttaactgaggtagataaagttacttctgcttacggggaggatgcaaactacgagatttcttttattcgtgagaaatatgcggacttagacgaaggtgccaaacaggactgtgaggaaacggacttctttgaggaggtagaatacatcagtcagatgcaaacaggagaagactgttttgagggctatatttttagaaaaataaaaaacacagattggtgtctttgtgtcacgtatgctatctgaggtcttggaattactactaactttttaggttggcttaaaatgtagtcaattagtaaacatattaaggaggtaaacataaatgatacaagcttatagagttcgttcacaagaagattttgttaacctaatggttaacttagaaaaaaaggattttaaatggtatagtgggttacctcctctagacggtgtatactacagacaaaacccagataaagacttagctagtgattctcttcttgttgttctaaactatgaacatcgtactattagatactatacagttgctaatttaagggaagcttacccactagcaaccatctatacgtacacccctagtagaacatctaaaacaaactcagtagacggttttactcctactcagttagtctatgacgaagaactttacctataaatgtactctacacaactaaaggcattatacatgtctttttttgttgactttatttatcttatatggtatactagtattataaataaaggaggtgtctttatgaggctatccctaagaaatatattggaaggttttcttctattctgcctagctttaccacctatcctagcagttatgggatttttattaggtattatatggattacattttactttcctatgtttggtctagctttttggagtattttaagtgcatgtttagtagagaacttgctagaaaaatgggataacagaaagactcttagaataaggaactttagtatgtggtatgttatagccttacttattgctcttactattactggtatagctatactagtgctgtttttattctagaacataaagagttatgtcttaagctagtttggtaataaaataaacataagaaggagaggttattttgaaagctttgttttttgtaataggcattctagtatgccttttaggtgttgctatcttagctacagcaatactgtggattactactagtcttcctgcaatcggtgtcccttttctaactattactttacttgtaggaatcctactagttctgcttatagaaggaaataagaaatactaaagaagtaatggaggttgttaaaatgagaaagatagacataaaccaagcaagtgtggatttacaactactaatcgaatttgagaatgaggactatacagtagcctatagaggagaagagagtaggcatacagaagaaatgaaggaagcaatcctttcagctcacaaagatacaatagaccgtttagaggtagctaacgataaactactagagacttatgaagacctgatagaattgaaaaaagaatatcacaccacgtataagaaaaatgagaagttagctgtaaaaaataaagaactaaaaaaagagttacgagatattacaaatgaaaaagaaaagttagaaaaagagctactccgcataaaagtgtctaagggggaactttaatgctagtatcccttacttttttagaggtacttttatgggtactgactttgttatactttggtatggtaatctacgtactcttatcccacctagttactagcgtagcttaccccgagtctgatggagttgctagttcttttatagactatagttatttatggggggaatctgatgagacgagcaaggctagtaactataaatgctacgaccatataaactacagaaaaacgagctattttaaccccacttataaaagcggtgaatttctaactagcacagaaaaggtgtctaagcataaaagaaaagtctttaaaaataaacaaaaagttaacttagatgaagaagaagagttataaaagacacataagaaaaggtacctgctaagggtaccttttacttttatactattttttgttttactggctctactatgttaaattcccctttatttgttatagtgatagcattaagttgcccttcaaaacagcaaccgccatctattcctataagtttttttctgtcgtgaacccacacgtcaaaattagaataattacggtctctaaacggtaagtaacaggtaggtgtgtgaccgaatacaattacttttcctgtgaggttctcttgcttataccagtccctaatccacataaactcgtggtctgttgtcttcttccagtcttccccgtataaagattgaatacctgcatgagtaaacagaacgtcaccaaactcgtaatacggcttagaagctctcaggagctctatatgatgtctatatttcttttgtatgcaaatacttgtaaagtgataaaaagacgatagcctatgcctagaacgctcctcctgtggcatattattgaagtagctagacaaggtgtttcctcctccactagcaagccatctgttaatagtgtactcattctgctcttcttctataaaatcaagcaacatttggtcgtggtttcctcttataacaaccacttggtcaccgtactccatctttaaatcacaaataatatctaaaacctctctagactcgtcccctctatctatatagtccccacacaagacaagcaattgtttatccctatcccaattgtttaacatttctaaaaactctaagctacaaccatggatgtcactaattgcaaaagcttctttgaatttagtcaccgaggcacctccttaatcaaaattaatttcttctttccaaccttttatataagatgttaggtctccttcgtaccccataagacgtatttccattaccaactgacctatcgtagcatttgaagtgctcataccctctaaaacctctataaacggtcttagtcttctgtccataatctcgtttgcagtttctatagcactctctttgtttctaccgttctctatgagtaaatttacgtaatcttcataagtcatgttaacatgggttacaccgtccaccacactagaaaacctacagtatctaccatttggttgttgtgagataaagctactcatccttgatattcatctcctctttaacagtactttttagccttttgttttccgcttttaccttttcgtataaccagagagctgtagacaagtcctttcgtgttacctcgctgttgtttcttacttttgctagtagctcctcatacgttttatctttaaagtccatagtagctccttcttctgcattgtcccaagcgacctcgtaagcaaccattcctgtcattccttgcatcacacccacaatagtacctatagatgtgttataacgatgttgtaccagttcaaactttttaaacttattcattcttatctatcccctagcaattatttgtaaaagtaattccaaaagattccatcctaccatatctaacagaaacacaagctgttaggtagtcacttactcgtatctttactttaacaatgtcaccatctataggagacttaaattttatttctatgtcaactgcaaaatgattgttgtgattaatccaagattttaaatggaatattccttcacgcatctttgttacagttgtgtatggatactctagtgaatgtttgtgtatatttcttggaaattctccaaacattgcaattacttcttttgggtttgttggtgctaacatgtgatgtctattttgatttgataataactctctgccttttttgtcaagctctaaaaagtcttctcctctatttttcagtaaatcaaataataatttttctgcaattacctttccttctttttgtctttcttttctctgctgtttatcatatttcttcttttctttaattatcctattccttgcatgactaaatttttccataattactgtcacctcacatctttatttacttataaaatctactaatattgccaaaataataagaaataatatcactatccaaacaaacaccactattactacccaaataattattggaataaacactgtccaccaaacaatattaataaaaccagttaattgaagtattaataaaactaggaaagctaccccgcatactgtacttaatctcatacttattattcaccttccacactggcttcttttaataattttattgtaacgttttcatcatctgttgctgttcccgcaagaacctgcttaaccatctcttcaaaattctctctttttggctcttcaaaatcttcttccgtatacacctcaacttctacttcctttttaggaaccaaaagtagtccaccaaattcatcattttcgactccataagtcatcttacctgcttctacatcttcgtttgttttaattgccattattgcaattacatctttaatagtatccaccatattcactctcctattagtttagtaagcttagccgggtaccacagtttttcattattattaatatcccattctattaaataggcaaaatcttcaccttgtactctagtgtatcattataaacttcctctactgtacctaactgttttttactctgtaaaatgaactctacttggtctcctactttaaacttcatgcttcttcctccatttctaatatttcgtgagggtctacctttacaaaatcagataaagagtacctaaatacaaccctgtctttttgagatacaagggcttcttgtttttcttttttatgacccctacaatgtttgcaaacaagaagtacatcacgagtgtaaatattcgtgttctctatagatttccacttagaagccgtaccctcccatatgtagacgtgactgcaagtaggcttacttctaactaaactagctagtttactagtgtatattttcatagtgtaaacacctccaactacattatatcataaaaaaagaagagtgtcaataaacactcttactttagatgttcccacccgtaggcactaaatatagcaattaccgctaatggtaaaaacgtaataaaggacacacgtaagataaatcttgagataggatttctgtagtaattccatagatatgttactttaacatcatggctatcatctttagactctaaataaataagtaacaggaacagtgggtatattgctacataaatttgaaggattagtaacaccatctatttagtatcctccttaagagacaaattatcgaggaaggctactaagtcttttttagctaagtttcctttgtgattgcctacaaagtcctttatagcagccaccagttcttcgtgaagtcttctgttctcttcgtaaacctcattctttaccgtccgctctaggttacttcttgcactctcttctcttttaagtaaaacgcaagcttccccctttagtctagtgttgtcttcctttagagtctctatctctttttccttttcttccatctgcccgacaaccctttgtaactctcttagttgccgttctttctcacccaaaataacttcaagagcgctcttttctcctacagatttccctagtttgctggttacctcttctagttcagtagtcatatttttaatagcttccttatctttcttttgttcttcataagcatctacaatatattcttttaaattcttatatgggatttctttctgttctttttcctttagtttgctattttttcttttagaactaattacctctaaaaacttcattcggattcctccttcattgcttcaatctctctatgcacttcttcatagtaactagctgtaaaagttactcttgaaaactcctcgtaagcctttagctcccgctcgtccctctcagttatctgctcccatgtaaaagttttgtcacactccttgcagtataaatcgtaatctaagtcataattacattgtaaacactctactgattcgtctccttcgtagtgcatatctatttaccacctttagagatactatcgaacttttccgctaaatctgcatacatatctgatagggagtcataccagactgttctagagaaagcaacagagtatacaatggctttttggtagtaaagtctgtcaactcttaccgcaagacgtctaaaactatctctactctcttctaggaaatatgtgttactagggtcaactgcttcttttatcacagggtaaatctctttaacaaaataagcttcttctttggagtcaattcccttttgagtcagtatacctaaaagaatacctgcacgttgttttacctcctctggtgtatctcctgatgctactcgtttaactaattctttaatatcttcttctgtacctacgcctataaggtgacaataaacttctaagtagaatttatttactcctaggtgatattcttcgtcttcaacatttttccagataaactctttaaggctttcagcaggtaagtctagcatttttaaaacacagctaacgttaagggctctgtacttgtctaataactcctcttctttttttctatcataagcaagggaagctgtatgtctgctaaatgtatctacgaaacattggtctttgaaaagatagttcagcttttcttcaccagattgttttacgcataccttaaaaagtttatacagacggtcataatcagcagaggataagtaagtaaaccaatcagctcctactttaggtgtgaaccctataaccttataactgttctcattacaaatacgaaactcttgtcttttaattatactagcgtaaacgaaaaatttacctttcttttccacaccaaaaatatttttgtctttaacaggttctgtagtgaccttaatatcccaacaggacttcaactccacccctaaaccacctaagatactgcccgctacattttctatcttaatactatcatttaatttgaactgactgtacttaagccccaattcctcagtaactacttctatcaccaaaggcattatatcctgtatacgttcctctgggttctcacttgacttgcattcaactataataagaggtagccatatagtcccattacccccttcacttgtactacccgcatctcttttttcataattatccttaatacgttttttatttagttcttctactatacctctgtagtaaatgttcatcattcgagtgttatctttgatgtctactaacggaatgttcttatactccttatgcttagccatactattcctcctcactacctattatttctttgtagaagtcctttacaaagtctgcttgctcctctttaggaacaatactattaatttcctcctcaaagcaaaatagaataaagcccactgttttgatagagccctcattatatagcttactgcttagaaccatgtagtccaacaattcttctttcttcgtattgtctctatgaagcttgtcaaaggtattaacagaaaaaccaaaaggactcagctcctttataactaacggctttaactcaccaaactctactttagggttagttttacttccgaaagaaagctccaactctttagtgtactttttaacaagttcttctacttctgtgtacgttcctacgctacagttgtttttctcctcctgtgagctatattttacgtaatactctttactcatactatcaactccttattctaattgtatcacaaaaactctattaagtcaacaaaaaaagaagatgttactcttcttttaatggcagtctagtttttagttttcgtgttcttcctttgtatctgagattgctagtatcagtgtgtactgaccttacttttacaatactctctttgacactatcatatagaaaatcccctacagctaccttacctaacaagctgtcatctaaactaaataaatatgttttagcataggtacagcttaggtcatcctgaacatagcttatgtaaacgtacttgttctcataatctgtcgctccccaagactcgtctacttcccatccaccaagtatagtttcaatattggcatttagtatctctgcaatgcttctagcttgcgttaagcttgtcagcggtcttacctgattaggttctcgaactttttcccagtttaatacaaaagtacctcttttgttcttaataaatgatgttgtgtagtatgcgtatggaaaccccttctggcgtagaacatacatatttcttttttttaccatataaatcaacctcctagtatttttggtaagtcctctgataagtttacagatacgtcttttcctagtaaattaagtaactctttgatagcttcttctagttctacaaagtactgtggtgacctatttcgactatcttgttgcaacttaatcacgcaatccatattcaaatcagggcatgaaccctctcttattcttataatattcagtttattatttaacgcatgttcgcacttctgtctatctttttcagcattttggtgccaacgacctccatcatattctaccactagtttcacagaaggtacgtagatgtcaaatctttttcctgctatatttttttctaatacaattcgagggaacgctttttttaaatagtaggcaattatcttatttggtttagaaacagtagaggaacacttagggcaagatttaaaccctacaaacttagacttcacactctgctgataagagccatgctccttacatttccaccaaacctttatgtcgcttcctcgacttacgttcttaggttttatacctatagcattgtttttttcatagtcccatagttttcttagaacagagctatgggactcactaataggtatacaccctctcttaagagctttagtagccatagttatctcctccgctctctactatatcttaggttgcaaaggtctacttctgcaaagaacatgtagtctacctccgcatgagacatccctaagtctcttaactcatatactttatcttttacagtttcccttacacgttccagttctttaataccttcggaagttaggttaccttccgcatctcttaatttaatagccatagaattttctcttattggagttagttgctctcctaaaattgtctgaatctgataatatagcggttgcctttaataactctccttttgtaagccagttgaataggtcatcaacagataccttcataagaacagctagttgctccataatgtcatcttggtctgtatgacatgacctgcttgcttggtctagtagtccgtaacaagcttttttaagtatcttcatgggtttatctgagatagtgctagaaggctcacgagaggataaagaaacaacaccagagcttgctctacaactagtgcacatgtagcacattccattaccgtactccttgccatataaatcagcattagatgtgtactttatattaccttgacataacgggcactttgtaggaatttccttcttcatacgtgccaacccccaatcgtcagtccatcgaataacataccagccgttattgctacagagacaatcaaaatcattaaaattattgacccccataaggtaagccctctggtgaagaactccttaaccgtaactaacagccaaataaatgaaataataaaaattatccaagtagttaatcccataactttgtctcctctcggtatttcttaatcttcaatcttgcatctgatataactgttgacaaaggtactagtagccttcctgccatgtaatcactatctacaccaatgttaatttggtctttacttggactaggtttactatgaatgtgtccatgaatgttgaatctacttttattacatccaagttccataggataatgggaaaaatgaaactctactgagtatgcgtgaataatcataccaatatcgtgtacttctactacactagggcataggtcactattgtataagtttactttttttcgtgtatcatggttccctcttataataatctttttacctcgtagagcattcataattttaaccgctttttcttcgtcacctaatgcaaagtctcccatgaagataacgacatcattatcattaacagtatcgttccactccattataaactgcgagttcatgttgtcaataaaaacgtatttatctcctctataatgttcaaacttaagtatgttatcgtgattaaaatgtgtatctgatgctaagaaaacattgttagaacttgaaatgttatatttaaccaaaaaatcagctagactaatcaattaaaaacacctgcctctagaggtacttctttataacctcctgtataaaaacttaagttgtactccgtcatagacttatctttactatatacttttactacacgattactggttactagaatactagctacctcttttttacctactacatgtttatgctttcttccgccaccatctgtgatttctacttctagtaattcacgaataccgaacataccaaacaactcctatctattctataataatattttttctgatttctaaccagtctacctctttgccaactgcttttaaagccttatcatcaatgtataagtcagcgccaatttttctagtatcatttttccatattgctatcttatctgggttgttttcatttacaaagtggtacggtatactgttattggcaagaaaatccaaacacaaatccataccaagctcttctctacatgtccaaatagtgataactgccccaagttcaacaagttcttctattgtctctttagcataaggtaggagctcccctactaaggggtagctctcctcctgctcactagcaatagttccatcaaagtctatagctatatacttatagcgtaaacttctcatctctacgcctttggtttgctatctttaaattttaactgctcttttgttgtagatggtaaacgataagactcatacaagtgcgggtggtctttctctaacttcttaccgttaaatcgtaaaccttccacctccgcttgataagttagttcaaaatctcctaaaatctctaatgattttacgtttgccttcaccatcataggcattaattcatctttaagtgcatctaactgtttttttaatatctcgtagttacgttttgttgttgcataatttttagctttgtcctgcaaggttttagtaatagacacccctgcaccctctttaagttctaccgcttgtaatgcaccttgttttgccatttataatctcctcctgtcttatttataaattaactataacacatattttattataagtcaacaaaaatgtatattttttatttactatcctctacttttaggattatttctctgagatgtactggaacactacgtccaagagactttttagctgtatcctcatcgtaaacaaaagctaagtcatctggtgattcctcactcataatatcatcatacacaattgagggtactaccttcattagttgggaaaagttagaaaattctaagttaggcaacttattttcctcttctagtctctgtagagctttcctaaccctagtctcagtgacgaattgtcgaatccactcggtactttcaggtaaatctttaacttccctttttctagattggactctttctgcgtgttttggggaaacacttttaaatagtttctggctaccatcgatgtctacttcgtctaaaaatttaacaataataccttctccttcattagtttccttagtaaatctggactgacctacaaattcgttaactgtatcaaagtcagtaaatacccctaagtaaaacatatctggcataggtagatttagtgtaaaagcaatactacgtacctcatctaaaggtaagtatcgcccttctacgttactgtacacgtcaaataagtagaagttatttctaaattgctccttatacggtgtatttaacttcttagaacctagccactcaccaaagaaagttaggtttactagacctgaatggacaacatcaggaatacttaccttgtctttaatgtaagtagtgaaacctgatagtgtgtttacctctcctacttcattaggaatgtctaactctatatttcttgagaatttacgcatagcatagccaccatcaggaagtttctctacaaaaaaactagcgttgctaccatctaacttctctagaatgataatttcacctgtatatgtttttgccatttgttgagctcttccaattctttgaatttttctatatctattcatattaactctcctcctaaatttttaagtctttgaacgaaatattatctagattgctgggttcaagctctcctgtacgaatataagtagtcatgagcttgctgtctaactcagtatcctcttttaataattcttcattaactttatttataagaatgccaatatcttttcctttagcgggaataggtatatttaatgatttagctatcttgtctacttctaacttaattgcctcagggagagtttcctgataaggcaagaactcatatagtaagccaatctccatacccaagtcatcagcttcactctctgttctagcaaaatcaatttgttgacctatttgttttacctcctgctttactgcttggcgtacctgctcatcagtaagttctgatacacttttaactttaacttctactcgcatgttatcaagtctagcaattaaattttggtatacattgatgaaatcacttttactttttctggcatgtataagactttctttcactctttctaccaaactagttcctttgtctttttcttctaacttcataaattttccctactttctctatattctctttttgtatcatcttttataagttttctctcatatctgtttcttagttttttgtctcttccttggcagtgtggaaacatcccatccttagtgaagagtagctcctcgtacactctattagccttattcttaaaatatttatttgtagattttttactcatttagttatctctcacacgttgtaaaattttacctaacaagtttgaacctctccataaatctttgtttaaaatacgagggtcactttctcgtagtcctacgccccatatagtgtcataagggcttgcctctactaaagtagttcccttagtagcttctagtttatcttttaattccttattttgagagaacttagccattaagataataaaaacaatagcctctttgttatcatcccacgtagcctcgttaaaacctctaacttttctacccagagatttaactgttttagggtctccactaggaacggatagaatttgtgaagccactaactcatcattgaatagacgtgccttctcatacatgaatgcctgttctgcattatgaaacataatagacttgttagcttctttcccttcttgcttgtactctaaagaaaaatttgatgggtgccaattactaaaggggctttcctttccccaaaaaaatgtgtatctatctctcattttataactcccctccttgacttagtaaataagtaggcgtttctgtgttagccattctgcttactcgggtagctaatgtctcaataaattttttattatcaccataaatattaatagtctgcgcttcttttttagttagtgtagtgaactttgtagttttgtcaaactgtacaaaacggtaataaaatttttgtgcttcttccacagaactagctaatacccaagttttttctttatttcgttctgaaatttctactactaattgtttcacgtgaaacaacctccttatttattttagtttgtgttctcttatttaattaatgcgccttaaaattatagattggtttaacaatgaagtcaatgtcaaccgtatctgtaatattctctatgatactctctttctttttataggcaaatggtgcctcatctagtgtagactccaacacagaggaagaccaaacatctttcattgtattcttaaaatcttccatttctagtgtttctttagctttagaacgggacagtaccctacctgccccatgaggtgctgaattgttccagtcttcgtttccctttcctgtagcaataatgctaccatcacgcatatttagtgggataactagttgttttcctttagaggcatctgtcgcacctttacgaataaccttattagggatgtcaatataattatgaatagagtcaaactcagaaactaccaagttttcccaacccattttagacataattactttatgaatagcttttcggttatcctccgcaaatttttgcgcaatagccatatcatttagatagtcctctactaatttaccgtctagataagcaaggtctttattaaaaggtgctgtacttagttttaagcgcaataactcttcctgtatgtctttttccctaccttgctcttttagagctctaatgagctcctgtggcttctcagtgttagaacaatacttaacagctagttcttggtgtgttttagctacttgaacgcctaagttacgggaacctgtgtgtgtcataaggaagattcgaccatcgtaggcacctagttcaatatagtggttccctcctcctagtgtaccttctgataaagcgattctattttttacctcttcttttaagtttccatagatagtcagattcttaagtgacttacttaccttctcctgatttagtgctactcctcttacggagtttcccgaaggaacaaactctttaataacactatctaatttttctaatgtagtattgtttacctctaaatctaattcggttactcgtattccgcaaccaatatctactccgataaggttaggtactactttgttagtataagtcatagtggttcctacaacacaccctttgcctgcatgaatatcaggcataaatctaacacgctctcctttaacaaattcctgacttaacaattctattacttgacccacagcggtttcttctacctcactagtaaaaaccttagctgtattataccttccaagtagttctatcattctgctcacgctccttttattatacttcttactaaaaaccattataccatatgtcagtttataagtcaacactaagttaaaaaaagaggaacacgtatgcccctctattttagttctttaaagttcttagagaagaactcctcataatattggtcttctgtagtaactttcctacctgagtctgatactactgttatatctcctactagaacaccgtcttctgtgatactatttacagtaaacgttctgttcttgttacctttttcatgaaatgtcttatttaaatatttttttccttttttagtcatacataacatcctcttttgttaattgtgctagtgctttacaatatttttctttttgtgaggcaaaagcaatagtgttatttgaaaaatagtctagaatctctgctagttttacttttttagctagattatcctgcttcaatctctggatatagcaagtatacgcctcattttgaatgcgtgttaggtgaactaaacttccaagtaaataatccttgtccactagagataaaaaagtttggacatcactaaatctgacggcggtatcctcaaatatatcatgcagtaaacttaaataataagcatcttcatctttgtctttcctaaagttttctgctactcggtacacgtgcgctagctgattttcctcataattagctaaaacagtctttaaatgattacttacgttgtctttaattgtaaacaatacgtagcctcctttaaaaaataattggtagaaccaatagtacacatattcctagagaaacaagtaataacataatagtatctcttactgaaagtaggaacttaagtttaggtacatatggcatagatacccctgctctgcctaaaagattagaagcaataggcacaaggttatactcaataagaaaagctaaatataaataagagaatattaaaataaaagtgattataatccccatagatgtctcctctctactcccctaaggagctattttcatgcttatctattttaaaaagctcactagaaggaacaccaaaatatttttctagtttcactcgatttctctctctaggaaacttcttacctgtctcccaataagaaattagggagtagttaacatctaaatcctgcgctaattgtcgaatagacaactgctgattctctctaattcttcttaaatttcttactttcccaatatcttctttatttgtcattttttttttcaccccttactttttatatatttattataacataaaagtcaacgtctgtcaagcgtacaaaaaaaagagcacgtattaggtgctctctaggttactatagaatgctgtcaataaagctgtaggcttcacttcttaaaacagataccataaaatcttttggtgtatttttttctaaagacattacatgtttaaagacagtaggtgtaattccagatactaaagcaactccgctttcagaaaaccgaactggtgagttatcattgcgggcatttacattccaaaaagttactgttggaagtaagtaacctgcttttgaaaacttacgtctcatactttcgtagaaagagctgtcgttctctttttgtgaactacactggtcgaactccatgtcagagataatgacaatatcagtaggtagctcactttgtggcagtttgctctttgttgcggtattcagaattaagttaaaaacagcctctaggttagtggacatgccccagtcgctactaatagcagtagaaacccgctcccgtaagcttaactcactaggaacatgaactaaagctggtttagaactgaatgtaataaagtggtctttgaacactccatttagtctctctgcgcagtatagccctaaagaaatagaaacgtccataggtgacacttcttttgccccaccatacatagaacctgatacgtcagaaactacaatagcatttctagtcatattttctacgtagttaggtagtgatttccaagttgcctctactgtgttgtctacagagtcgtcccagtcaccacttgcggtgtacttcttaactaactcataaggcataatgttgctggcatttagttttttatctgtggagtttactctgttaataaacatttcataggatgtgggcaggtgttttctaaaagcccctctatatttaaacagggcacgagaaggtaacttgtcaaaattgatagtatcgtaatccttgtcggctaagctagtctccactaagtttaactttgctcgtaaagcggaaagtaatttacgatactctcgactggtgtagcctagggaagcagacaagtctctagctgtatgtttcttctccttgtttggtgaagactccgaaggtaaccacttagctaataaagatatagattcatctgattttaagttctttttatctagttctagttgttcagaaattagttccactagcaccattgttaagactttgtttttagtttcacaactatagcgatatagagatacaacatcgtcccaacgaccaatttcactcataaactctgtaaacttaattagagtgtctaagtcaagctcagctagccccttaatggactcgttatacactgctcgttcacctaagccactaagtcggatattacgaagatgtactaagttcttgagtgctagctctcggtcttctatatatgccttcttaaataacagcttagcatcatttgttctatcacgcattgctcctgctcgtgagaaaaaatctaagttatagtctaaagtagtgctaaatgtgttagcccccttttctgttttacttaaagtagtctcttcttttaatttatttaaaaattctgacataataatctaccccttttcttttttataatcttatagctgacctaagctatattcaggtcagctatcgtatgtacaggaagcgtttttttctcgtacattttttttttacatggttagacaccatgtcgtactaactgctaatatgcgctgttttagtttttagtatttgctgtgtgcttcctaattaatttttacagagagcgtttttactttttcaaatagtataggttagtaattgctgttagctctctattttatattctgtagattatttttttatcacaggaatcatttttatattttatccaataatatagtagtaattgctgttagattcctaagtttattttatcttaaatttatgataacatattttgtctagtatgtcaacatctttttacattttgtttttgttgtcattctgccagttttcccaaaattgctctttttcactttttcggctctcgacaaaaccttgtgtcttcttacaatatgggcaatacatatgtttaatatgaccgtccttcttttgcttagctgaccttctgaatatagagactttttccccacactcagggcatgttaacgtggtttgtactgtctttaccatagttataaccagattgccccgttgggaatcgaacccaaaccgaggagttatgagctccttgctcgaccttctagctacgggacaatgagggaaacttctcttagttagagaaggctccaaaatgttttagttttccaccgagtgcctttgtcttgtctgatagttctatttttgatgttagacgagacttaccttctgccacgtacagaaaatctttctcaacagtaactaacttcatttcttttttgccagtctctttgttgtatcgttcctgtaattctttaacaggttcggaaattacgccgtaaaattttttcattgtaaaacaactcctaattttttattttttaactaactttgtaatttaataatacaataaaagaaatttggagagattccactctcaactacttcgtgcatcaatatattcagtcacggctaaactagattcttggtaaggagctaccttgtcctccatccagtctgtcaattaagactaagtttaaacacatcacgttggaagtgattaaaaatttaccgacaggactattttacctgtacatttcaccaagcatggaagagtatatttttattaataccaattttaaagatgtagattttggctactttgactcccatcatcccttatactcggctttcaccgaatcaactacataaatgacttttagtaagtagttttaaaagagtacttacattcccttctaaaacgcttcatcacgggcaggtaggatacttttatcttataacacacgtagcagtacctttgtctcacactaaccataactatggctcagtccccaacgcatactgatatttaccacatatactaatcgaggcggaaaatttaagtacattagaagagctaccttcttgttatgttacaaatttcctttattctattatcaatgttcttagtaacactataccatagatttcatatagtgtcaagtaataaatcaaggtggtaggattcgaacctacgacttcccgtctccaaagcgggtactctaaccaaactgagctacaccctgtcggttccgtacacaaaccttttaattgcacggcatatttttttgactagctacggacgtctagaacgccagccgacatctcctgtatcagagaagatgaagcagatatatagtattatctgtagtttaaggtatgaggatactagaaccagaaagaagcacctctattagttttcctgttcctatagtatcggaagcaaaaccgaagttataggtggatttcctatcttttttactcgttgtaactcactttagttaccgtttgcttttagcggtgtgagtggtaatctctttactgtacacctagtgtaccacactttgcgtttattgtcaacacttattttctaaaatttttcctgtaccaattttctatcatactatttatatagttattcatgtagtagtcagtaactataatacccttgtctgctgctaatgtatactcatgaacccgtaatatatagtaagtacttataggaagtacaacgttacctgaataaggtttagtagttctctctacatgttttataaatgactctatttctttcatttcttgttggaaagataggtattttccatgtctagtcctaaaaactagtttgcggtaaaacccaccatcagggatatgctcttctctagatattctagacctttgaagtttattcaaccttattttgtctagtctcctctcctttgaatgccagtccgctttactcttgggcgtcttcttataacttctgctcataggacacctcctaatgattatctacctagtatttctctctgatagtcattttaccattcttttcttctgctttacgaaaattctctctaactttagctactctagcttgctcttcttttttctcttcgggtgtcagctccattaaaccgagaactttcatagcatggtcatactcttcttgtgagttacacactactgtatgtttaaaattaatctctgtcaatattgaagcctcccttgttgataattatgtacctatggctgttaactcctattcatttaacttccagtctataagaagcaccacagtttgaaacagatttataaggttcctcaacctacatataagttaactttattatgtatttacaaagtcctcagcaggattcgaacccgcgctaagggagtagaaatcccttgtgcatccgctacaccataaggacacgattctcctatacacacagccccgctcttaaccagatgagacgtaacggtcaccgaagcaaccgtaagggagtcgaaccctttccgagctgtgcaaaggaaagtaacataggtctcttattatgttacttagtctagtgtccgtccacataggttgagactacctcgggacacgtccgcattctgttaattcagatttatttagtattgtgttgcaacacaacgctgggatgactaatccctaaagcgggtagggggattcgaacccccactacatggttggaaaccttgggtactacccttatactatacccgcagaggcaacctacttagggcaagtaagttaccaatacaaaaggagtaaaaattaattaaaacaaaaaagcgtatcctgctggatttgaaccagcgacttccgacttaacaggacggcactctgacctctgagttaaggacacaaaatactcggcaaggattttcaccttgcaataaccttcatcattcgtttcagctatcaattcaagcatcggatatgctctcagcttacttctattataggttataccagtcctactgtgcgatattttttccgccacgagtataaaaaatagtaatttaagtacaataggtagcaagtttttgtaagtgctagcaagcgcaatactgtaccactcagtattaaaaaaggtgggatatacaattgtaatttcattttactgtgtttgtaattgttatcgtacatggtttcatacacattgctattgagttaaataacttgctaccgaggtaactgtactatttttgcctgctattcctaaagctcttacaattgacaggtaaggagtcgaaccttaataactaccactagcttgtctttaatacttatcggtgcaccttatagtagacttagggttactagacaaactaaagtatactacatacttgacttactacccgacatttgaagggaggttagaaattgattttagcctctttttatttgctgataatagttttttctactatagcagtaaggtgcaccgtttgtttgtctcaccacatagtaatcctaattttaaggaaggtaagggatttgaacccttgtaccctgtattccaagggcaaactagttttcaagactagcgtattaccaagctctaccaaccttccattttgttgctatggagtccgcaggtatcgaacctgcctctcctgcttttcaagcaagcgctaatccatctcagctaggactacgttaatggagccactaggattcgaacctagaataacggggtaaaaacccgcagtgatagccatttcaccataactcctttgtatagaggttagagggatttgaaccctccgtaactagctatatagtctagtatgtcgtcatttcatcttaacctctataataacattataacatactttaagatgtttgtcaactgttttacagggatagtaggacttgaacccacctcattggagttggaaaccaatattttaccagataaactatacccctaagctggtctagttggattcgaaccaacgtatcacggaatcaaaatccgttgccttaccgcttggctacagaccaatagtaccaagtaagctatgcttgtagtaggacttgaacccacgtctgaggcttacaaaaccactgttctaccaattgaactatacaagcttatttttttattaagtcagctctctaagtctccgctctttacgttagtaattattagctgactcgaagagctaacctattatgcgccatcaaggactcgaaccttggaaccccgaagggaggggagttacagtcccctgcaattgcctctatgcgaatggcacgttgtaaagctagagtacatacttcttgcttattaagataccctagctaatttttactacttgttatatacttcccagtcattagcaaatacatctaagttgctaatacaccacattacatgacttccagtagctgttctcatttgcaagtaaggttctactttaaatagtgcaccttcctctaactcataagcatctgctgtttgtttattagcagggataccctctggatagcctttctgagctactacaaacatgtctttaccattccaacctgttctgctagctttctttccttttttaattaaaggtaaaatctcttcgaatgtcataattttctcctccttctatgtcttattaataccgagagtgggattcgaacccacatatccttgggacagcagattctaagtctaccgtgtataccagttccaccatctcggcaagtgtttctactagtctttgattacattataatagttagcataatgagtagagataacaaacccacctcgaatgtttaagtatggagggattctttctaagtcctgtaaatcaataccatctactaagtaaccgtcattaggggcatctgttaatggggatacagcaagagagttgtctaccgaaatagttttcacgttaggatattttttatcaaaaatagttttctgaaactttgtgcttacaataacagagtatccaaattcctcagctatctccattatagcctgtgtttttccttttctcctatacttaacagtactgataaagaatagaggctccattcgatacatagccttcttaaatcgccttacttctttactctctttaaacttagacttttttactttcttatgagtgttcttttggttcagcatattcatcagtccctttcatttttattcaagcgcacgtaaggggaatcgaacccctatttctcgatagacagtcgagtataatagccattatatcatacgcacagtactgaggtacttctagctaaaaagttacctcagagtttaacttggcaatagactggtcggctgtgtttctttagctaaggaagccttctacgcactattagtaccaacacttggaattgaaccaaggactaccgattatcggtcggttgctttgcctcctaagctatgctggtatagtatactctctcgttgactactatacgggggaatgtattaataggaacgagagaatacctatttttaattataccacactctaacagctttctgagattttaatctcttgggttttcctttttcaaggtcaaggtttctggtttagtgtgtctactacaaaccttttttcgaaaggagagtaagggattcgaacccttggtactattactagtacaacgagttagcaacccgtatcattaagcctctctgacaactctccaagaaacaaaatttaaacttatatttatattataacataaattagtacgtttgtctactaataacttattttttttcataaagcgggtaatgggaattgaacccatatacgtagatttgcagtctactacctttccagttggttatacccgcaagttctagaagactaataagactcctagagtttttaaatatttagtgaaggtaaagatataaacctgatgataagaaaaacgtgttactaacaatcgtctaacttacctgtatatacaatatagcatacttactgataaaaagcaacagttttctataatatttttcatactgagctaagaaaatgtatagcaaccctaaaactatataattatctgacaactgtcctatttaattttattcgtcccctatattatattctaatatattatataattatttaaagatatgtaattagacttagaatataaaaggaatataatataggagaaccttaaaattaaataggacaattatctgaatattttataacgactactattacttgagtaacaatagaaacttgatactagtatcaaataaaaagtaatttaatattgatagtaacccacctcaggtacctaaatatattcagaaaatttatactactgtcctatttaattttatctacctcctatattatattctaatatattatataaagactataataaattattattattattattatttataatatagaagagtcctaaaattaaataggacaattatctgaatataaagtttaattaacataaaaagacacctgtaaaggcatctttattaaacatctaaaaatttcttttcgtaggatactacaagtgaggcatttacataaacttcttctccatctaatgctttagtagttctaaccttaataaatggtaactcagaacctcttacagcagagtttaacttagtactagggtcagtaacagtatcaaatacatcttcactgaaaagataaaatacatctcctgcaatagtctttagttctaaaacagtttgtcttctagccatttttattgttcctcctctgttatgtggtataattaaagtatatcacacattacttcttaactaaactgaaaacataggtaactaccgcacctagtataatcataaatactttttctactaagtcttttttaccttcatcttctttttggttcgtttgtcttagcgcttccacacctttctctaattgacctatctgatagaagatgtgtgactgcttttcagattgtagggcagaatctttatctagagccactacaatatctcgtaattccttgatagcttttgttagctcctcattgtcagtcttacgtgttagttcacccgtttgtactttatcttcaaggttttttattttatctagcagactttgaatatttgtatctttttccactcacatcctcctcctctcagttctttatttttggtagggtttacgaatgtgttctgatacatccgagctttcagggtagttatctaagaagttcctaattatatcttcataatcaggtgttacaattctcccgtaatgtgtaacaaagtttaggtgaaagtcaattctaccgtaggaacctaggtcaaggtacttaccaaccatgtcgtcttcatcaactttaaaataaccaaagtcaaaacctataagggttcccattgatagagccctactaataaagtagatatgatattcttcacgattttccgagaggaagttacgtaatatatctctaaattctacttttagcatatccctgtattctttcattgcagactcctttctttacatactccatgcaaccactactataaccataaaagatacaactattacaaaatatataataaaggtgtaaactaacgacataagtagtacaatgttagccacacctgctataaacgttgctataattagcgtaaagttttgcgtaataccttgtatatctcctagagaaacatggcgttgcaagtcacttataacgtggtatgaagtccaaaaaatacccacttgaaacaagacagaggctagtacagctataaacattatactcatagtaagacaacctccttaaagctagttctaatatagtatacgacatagaataatttaaagttag

SEQ ID NO: 3 - DNA, 135029 bp, Unknown
  Bacteriophage P200
aaggttgtgctaaattaatacagtagaactatgtggaatggaggaaatattatgaacaagccagacaaaaatttctttatgaaaatgattatcgttattctatcaggattacttgtgttatttgttataaacctagcatctatctactttgggtactctcctcacgttgtagctacgtactttatgacagggggctttattgcgtatatcgtgataaacttatacgaccgaattgttttccaaaaaggttctcctagggaagaacgagagaaacaagagagacaaaagatagcatctagacaaaaagctttagaaagcgtagtaactcaacgactaggtatccctatagaggagctagatttagaagtactcattcagatggaaaaggatttagcagacaaaactgctaaaggtaataaaaatgaaaaaactgagtagagacgagataaagaaaagacgaaaatatttcattaacgaagcaaaaagagttacacatatacgagcaggagaccgtagaatagcaacaggttcaagaatatgtaccttatgtggtagaagtctatcatctcagaaacggtctacaggagagacctttgcctcagtaaaccacatccattgttactttaatgaacttcttatcgttaacttatgtgaagatgcaacttcttgttatagaagacatgaaaggagttagatagtatgaatttagacaacatagaaagaaacttaagcaagaaaaaaactacaggagatgccgtaaaggatgagttcaatgacatggctatgaagctactagcaggctttaaagcagatgtttcggcaggagataaaaaattagaagatgtagcagacatggctaggctgttacaaacctttatacaagttaacaaagaagatagtggtagcggaggaacatccacttctttaccagagcttagtagcttgcaacaagaagcaataaccaatcagataaagacagagagcgtagtttcaggtgataaagagcaagaactagtagacctacaggacttagctaacatgacagaagaggacgtacaacggatgataatggataaagaaaaagcaatgaacatggaaaatgaggagacgttttaatggatggcaacactcttttaaagatggcgcaagaaacttttcaaacagaaaatgtaactacagagcagttaaactatgttctggatatgctaactccatcaacatatttactaagaaaccacaaagtaagaaatcataacattacgttctctattgcaaacaagtctactgaaaaggcgcaggcacatcgaccttggcaacctaagattataaatgactcccacccgaatgtagctgttattaagtcaagacaattagggttaaaattagcccctctataaagtaatttataaagaaaactctgttaaacgggcataggaaaataaccaatatcctgataagagaccctaaatcctaagtaggacagtgggaatcccgtgctaaatcaggcgcaagcttgtaaatgcctaacgactaaatttctaggtagctaaccaaatggggttagtgagaactagataagaaaatatattctcttacgataccaactgaaaaactctatcagatgttaaaaaattcttagaagaaaatataaagtaaagcagagcaacctaaaccaaaaaattaggttgatgatatagtctagtcccctaataaatatcgggaaaccgagggtataacgtaagtgagatgggtgtagggagtatgattcattttgcagatacacacagctatgcagctgttaaatgtttatacacgtttcctacaggtgaacagatgcgtaaatttgtacagtcacggttagaccctgtacttgagtctagttactatagttctattctagacaaagataacaacagtctgaactacaaaagaatacgaaatagtcacctttactttagaacatctagtaagcccggagctgtagagggtgtagatattgactacttatccatggatgaatatgacagagttcctgttttctcagaggcttccgctttagagtctatggctagttcaccctttaaaattgttagaagatggtctacaccatcagctcccggtatgggaatacaccgattattcgaaaactcagaccaacattggtacttgcataaatgtgaaaaatgtaaccactggaaccaactagactacgaggactacgacagtagctctgtagaagcaggaggaaatatactatgtgtaaatccacaaggtgttgataccttagctaaaacagttgtagaaggttcgttccaattcgtatgtaaaaaatgtggagccccattagaccgttggtataatggtgaatgggtagcaaagcaccctgatagaacaaaaaatggagacggtattagaggatatatgatttctcagatgaatgctgtttggataagtgctgatgacttaaaacgtaaagaactaaattcactttcaaaacaagcattttttaactatacactagggtacccattcgaagatgctaaactagcagtttatagtgaagatattatagaaaatccaagcccaagagtaaaggttccaatgcacagcagaggagattacaagtacatttctgttggtatagactggggaaatacacactgggtttctgttcatggtatgacggaaagaggagaaatagacttgataagactgtttagtgttgagaagagtcgaggagtaggtaatattgagtctgatttagataagattatcctagaagtatctatgtacaaccctgatatgattgtagctgacgtaggggatagcgggaactacgtggacaaattagtaaaacatttcggagaagaccgagtatttggttgtatttataagtccagccctaaatctacaggtcagttgaaaccgcagtggaatgaagcagggaatgtagttactgtagacaagttaatgcagaacaaaagatatattatagaaatgaagacaaagaaagttaacttctatagctttatagaccctatgctaaaactattggtagaccattggagaaacgttgtaatacaagacgaagaggacgaaaaagatggaactttctaccaagttattggcagaagaggagacgaccatttagcgcaggcatcagtgtacagctttattggtttagacagactagctgatatgtatttgagaggctctaagtatgagttcaactctacttttgtaacaagcaactaccaagatgtttcgtcaacaccaccagatatattcagtaaatattaaattaatttagttagggggctatgctccctttcttatttgtgttataatatagacaaggaggttttactgtgacaaatgaagagaagtttgacaaagtaatgtctatcgttaaagaagacaactctaccgtcctacctgactccgcttctattagtaaaggaattttaagagagttgacacaggaaagtttacaagactacggagaggtcattgaaaagaatgattttttaagcagggagtttaaacatattgtttccctctacggttttgacgacttttactcactatatgcctatgctgatttttgcgatactatgagtaatctagctattaaaggcgggaacaaagatttatctaagttaaaaaaggtaaaaagaaccgttatgagaaacggaaaacctacagagatgactttttatgagtcaactaccgaagaagagaataaagacaagaaagaagaaagtaataccaagcgccaaggagtaagcacagtatctgcaaaagacttacttgcagagctactaaagtctaaaggggaaccagaaaaagtaactcagttaataaaaataattcttaaattccctgatacacctgctaaccccaaagtatctgattatttttaccagttaaaagacgactcgggagaagtagtcgctgtggcaggttacagtataggagcccaatacgtctctcttgatttttataccagtaatggctctattagcggagtagcatcaagagcgttctttgaattactacggatagcaatagaaaaagacttaggagctgaattaaatgacgaggataacgagctagctcaagtactattttcaaaatttaattttcagctagatgagggaaaacagagagtgtctgctagaaacttaaaaaaacactttaaggggtaactatatgttgccattctattcaatatatgctataataatagcatgtgttatatacacagttatctttattttagtagtacgcagaatgtatcgtaaagcagacaaacgagagaaggaaaaagaagacattcttaacactatgacagaaataacagaggaaataaaaacatttattggaggagaaaataacaatgacaattgagaacacgaagactttagaacaaacactaaatactgtagccgaggaaaaagattatgacttattcctatcaacgttggcgggggtagttaaatcactatacaaagagtaccaatttcttagacaagacccagcacccgaggataaaacatcaggaaacattttgattttccaaattttccgaggagaccctgacggttatgctaaagattttgatacactgtttaaaattgcagtagacagaaatttagagcacagaaaaatatcccaaaaattctttgaatgggtagagaaaggtaagttcttagagttgggtgataacatcgttattagcagggatggtacaggagtaagctctattgttgagtctgttgaacttaaagtttttattacttttatgactaaaagttatgccgaagaacgagaagattttttagctaaagagaaggaaaagcaagaagaggctaaaaagaaagaagaagagcgactagctctaaaagaggaatcgtataaatctttaggagaggctacagaaatgctaaatactctcatggaagagcaaggtgtatctcctgaggagcttgctttagctttagaacaggggaaggagagttatcgtaatgcacgataatgaatttgaagtacctgagagtgttattaatgcttctgctacaccttttgaggcgcctgatagaacaaaagatacctaccaaagttcattaaagaactggttggacagagtaaaatcagtcggagatgcaggaatgtcacgagagtttgttattcgtattccagcaaaggcactagaggagaacaacccagaagctaccttatttttaaaacagtgtaacgaaataaaaagaaaccctaagtgcaaccttcctattaaaattgaaaaaacagtagagaagtacaaagacaagaaacacttaatgttagacttggtgttcacttatagtaataaaacagatattgatgcagtaaatagtgtaataaaataacaaacaggttcctctttacgtctgctatattagatagtagaaaacgtaaggaggaatttttatatggtaaaatataccgtagagaacaaaattattgcaggattacctaaaggtaaactaaaaggggctaactttgttattgctcatgaaactgcaaatagcaagtctactattgacaatgaagtaagctacatgactaggaactggaagaacgcatttgtaactcactttgtaggtggcggaggtagagtcgttcaggttgctaatgtaaactatgtttcttggggagcaggtcagtatgctaactcttattcctatgcgcaggtagagttgtgccgtacaagtaatgcaactacatttaagaaagactatgaagtgtactgtcaattactagtagacctagctaaaaaagcaggtatccctattacacttgactctggtagtaaaactagtgataaaggtattaaatcccataaatgggttgctgataagctaggaggaacaacacaccaagacccatatgcttacttaagctcatggggtattagtaaagcacaatttgctagtgacttggctaaagtatctggcggaggaaacacaggaacagcgccagctaaaccaagcacaccagcacctaaaccaagcacaccatctactaacctagacaaacttggcttagtagactacatgaacgctaagaaaatggactctagctacagtaacagagctaagttagctaaacagtatggtattgctaactattcaggaacagctagccagaacactacactccttagtaaaattaaaggaggagcacctaaaccaagcacaccagcacctaaacctagtacatctacagctaagaaaatttatttcccaccaaataaaggaaactggtctgtgtatccaacaaataaagcacccgttaaggctaatgctattggtgctattaaccctactaaattcggaggattgacttacactatccaaaaagatagaggaaacggtgtatacgaaatccaaacagaccaattcggcagagttcaagtctatggtgcacctagtacaggagcagttatcaaaaaataaataatggtaaactagaggagacttttatagtctcttcttttcttttgtaacagtagtgaaataaaacttaacccctttgtaatattatggaattacttctgtaacacatctatgttatatttagttggtaatttaaaaacgcatatgcggaatatagaaaggaaaccagctaatgaaaaaaggaagtaagattgcaattgctactgtagtagctgtacttattgtcgttacagttagcctagggtaccatacagttaagttgaatgagacaaatgaggcaaagacagaagaggtagttaagctaaaggagaatatctctagtgtatctaaagatttagataatttaggtaaagaacatgaaaagaaagtctcagaatccgaagctcttaagaaagaccttgacaataaaaagaaagagttagaggataagaacaagaaagaagagcaacttaaaaaagaaaaagaagaactacaaaaacaagtaagctataaaaagagcttaaagaaaaaggcagtagcagaaaacattccaaatgcaactagtagtaataaaagtgctaaaacggcagaaagcaaaggaaatggaagaagttttaacgtagaagttacttactatgctattggtgacggttttactcccggtgtacataccgcaaatggtactaacatcaaaaacacaataacaacatcagaaggacatcgaataattgctgtagacccatcagtaatccctatgaactctattgttaaagtaactacaggtaacggagagagttttgtagctaaagcttgtgatacaggtggagctatcaaaggtaacattattgatattttagtagaacctagcgcagatatttatcaactagggagaacaacaggtactgtagaaattattggataatttaaagaaataagagtctgcaaggtataacaaccttgtgggcttttttattatgctatattatatatgacggattatgaagtttgaggaggtatattaacgtggttataacacagatagtgatgattatagcgattattattactacatttcttattatttatggtatatactcttctaggagaaaaacgtctaagtcgtttagactagtactcagatatgctataatggtagtaggtcttcttattgtctactatctatttcaagtttcctctattgacattcaacaggattttttacagcacttaaaaacaggaggtaacgggatagaagaagtttttttcacaaatttctttcttatcatggtaggagcagtaggtacacttgtagtacaactattccaaaatagaaagaaggattaatgtgtcatatacaaaagactccttgtggttggagacaaaaaaggttgctagagataatgggtttttaggtaactggatagagatggttgactactatgagagtataggaggaagccatgtattcgtttatgcggttatggaaggtcagaagtaccgaattttaggcgttcttagcacggatgaggtattactactaaaaagagatttaacgctctcaaaagccctatatgaagatgttatgaatagcaaaaagatgtttttctataaagaggacagtgaaaagaaaaaaataaaattgacagatgaaataagtaagttagaagataaagatagtatcatagtatatatgtaggtaggtgaaaaataaattgggactatttgaaagtataagattagcgggggtaaataagagcgatgcagtaaaacacattgaggttgatgataactactccattgcaattcaacaaagggaacaggaacaaatatctaaagcaatgaataacaaagaagtagcttattctcaaccagtaattggctctatgtctgctaacccggggtttaaaacaaaacctagtattcgtaataaccaagatttacacggtgttcttaaaaagtttggtggaaacatcatcctgaacgcaataatcaatacaaggtcaaaccaagtttctatgtattgtaaaccagctagacactctgagaagggtgttggttttgaggttagattaaaagatttagataagaagcctacttctcatgatgaagcaacaattaaaagaatagaaagctttatcgaaaaaacaggagtagataacgatattaaccgagatagcttttcttcctttgttaaaaaaatagtacgagacacttatatgtacgaccaagtaaactttgaaaaagtatttaacaggaaccaaagcatggttcggtttgtagctaaagacccaacaactatattttttgcgacaacagcagatgggaaaattcctgataacggtaatcggtttgtacaggttatagaccagaaaatagtagctacgtttaacgcaagagagatggcatttgccgtaaggaacccacggtctgatatttatgctacaggatacgggtaccctgagttagagatagcacttaagcagtttatagcacatgagaatacagaagcgttcaacgatagattttttagtcacggtgggacaacacgaggtattctacaaataaaagcagcacaacaacagtcgcaacatgctttagagatatttaaaagagaatggaagaactcgttaagtggtatcaacggttcatggcaaatccctgtagtatctgccgaagacgttaagtttgtaaatatgacaccttctgcaagagatatggagtttgaaaagtggttaaattatctcattaatgttatctccgcactgtatggcatcgacccagcagaaataaacatacctaacaacggaggagccacaggttcaaaaggtggtagcctaaacgaaggaaactctgccgaaaaaaatcaggcatccaagaacaagggcttacaaccattattaggatttattgaagactttataaataaacatattgtagctgaatttggagacaagtacacattccaatttgtaggcggagacataaaatcagaactagagtcggttaagatactagcagaaaaagcaaaagttgctatgacagtaaatgaagttcgtaaagaactaaacttaccgggagatgtaattggaggggacattccattaaacggagtaatcgttcagcgtataggacaacttatgcaacaagagcagtttgagcatgagaaacaacagtctaatttacaaatgttacaagaacagacaggtaacagagtaagtactgatgtagaagacattcctgatgggaaagatacaacaggagacataggaaaagacggacaaagaaaagataaagataacgcaaacgctggtaaacaaggcatgaaaggtgataaacctaacgattggcagacctaatattttgtagttctgccttttaggtaactaactagtagaggtatatgctatattaataacagaatgtgttataatagatatgagaggtgatttcttaattgaataacgtcgtagagaaagagatgaaaatctttgtacctattgatattgaaggttctttagaaaaatcccataagggagatgacaaggactggtatgttaaaggttacgcatctacacctgctctagacttacagggagaaatggttaaccctgctggaatagacattggttattttgtagataatggttacattaactttgagcatagacaagaaccagactatgtagttggagttcctacaagtaactgttatgtagacttccaaaaaggtctgttcgttgaagcaagactgtttaaagacaacgaacacgcccaatctatgtggaagttagctaacagcattagtaagacaggtattgatagacaacttggtttttctatcgaaggtgcaataagaaagagaaacacagatgacagccgaattattgaagaggttgtaatcaaaaatgtagcagtcacaatgaacccagcaaacccagaagctacgtgggaaacatttatgaaatcatggactactggacatggaacaacacctgatacgcaagtaaatgcaggagctttaagaagagagagcttagctaacagcatatcagtactagcatcagcatacaaaatttctgaccctattgaatttgacaacttatggaaagatgtaagtaagtttttagatgaaaaaggagagtataacgagaatgaggcaatagttatgttacaattagctaaaggcgtttctcgtgtaagtgcatgtgagtctttaatgaatagacgaaaggggaaacaaaatgaataacagtgaagaaaagttcgaagatgttttaaacgaatttggtaatacagtagaaaaagataaagaagaaatttctaagtctaccgaagaaaaagaggaggttacagatattaataaagataaaaaatcagaagaagaaactgctgaggaagaagtaactgaggctcccgaagtagacgttgcagaagacaaagaagtgtccactgagactcccgaagaagagtctactagtaagtcagctaaggaagaaaagaaagaaaaagatagtgaagaagacaaggacgaagacgagaacaaaaaaggtaagaaagacaaaaaagacaaaaaagaagaagacagcaaagaagaaaaaacggagaagtctgatgaagaagattccgaagaggctgtaaaaaatactaactctgatgcagatattgcaggagcattgaatgctgttgctaagtcttatgagaatgttaaagaaagccaaagagagcaaaatacaactgtagaacgcttagagaagaatatcggtagcttagtcagtaaattagacagcttggtagatttaatgagtgaacgtgtatctaagagtgtagtagaagatattgaaactcttgaaggagctagtgaaaaagctgttggctatgtagcaaaaaacgtagatagcagtgtagtagaagttattgaaggagaagaaacttctaagagtgtagatgcagaagtgagtcctagagatgagtttctagctaataagagtgctttcctagagtcttttgaatatgcaagtcgtaacggtgctgagcgtagagatttaactgatgttcgtcacgctcatgggaacattacaaatgatattgctagtaaagcagattataaccgagttaacgactttatgaaaaaatggagtaagtaataaaggtaactgtatagagtattttttgtaaacagtatgctatattataggaacatgggaaacatgttacgttatgagagagcttgcccgttttaggactcctcccctagggcaggttatctcactaacaaaaaaataaaactatgaaactgaaaggtgataaatataaatgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacagcttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagacttagcagcacacgaatacaaagttgttgtaagttctgacgatgcagagtctattgcaagtgaagtggctacagctacagttactgcaaaagatgacggcgttaaactagaaatcgagttagctccaatgtacagctcccgtccacaattcgtttcaatctatagaaaaggtgcagaaacaggtttattctacctaatcgctcgtgtacctgctagcaaagcagagaacaacgtaatcactttctatgacttaaacgactctattcctgaaacagtagacgtattcgttggtgaaatgtctgctaacgtagtacacttgtttgaattactaccaatgatgagattacctctagctcaaattaacgcatctgttacatttgcagttttatggtatggagcattagctctaagagcacctaagaaatgggtacgtattagaaacgttaaatatattcctgtaaaaaacgttcatagcaactaataattataggataattgaataaaaacagtatagagagcagataaatactgctctctattttactaataaggaggatttaaattgctaaaaaatacaaacttagctaattataaaaaagtgaatacacgatttggaaatcttagttttgatgataaaggtatttctaatgacctaacggaagagcagcaaaaagaattaggtaagcttagaggattcgaatatattaagacagaacagaaaacgaaagaagaacctaagaaagaagaacctaagaaagaaagtacagaaaatgaattagacagcttcttagctaaagaaccttcaatcaaagaattaaaagaatttgcgagtaaaaaaggcattaaaattgaaaaaactaagaaaaatgatataattgaagaactaaagagagggtaatgtacaatgtatggaggttatgaaggacaagattcttacgaatacccttactcacacgggaaccctaagcatgtagagccagaaaaagttgacgaatatgttctttctgattatggctggactgcggaaacaattaaagcatacatgtatggtgttcgtgtagtagaccctgaaacaggagaggaaatgggagacaccttctacaatcatattatagaggttgccgttgataaggcagagaaagaactagatatagctattctcccaagacgggagtcagaacaccacgattataaccaaacagaatttagtagttatatgtatacccatgttcataaaaaacctatcttacaagtggatgacttaaaactagaaatgaatggtagggcaatatatagataccctgctaactggtggaaagtttattgcttagctggacatatagaactgtacccgacagccttgatgcagacttctcaaagtatgacatatgactctgtatttggaggatacccgcagttggcaggaacagccccttctataggtagaacatatgcccctcaaatgattcatgtagaatatatagcaggtatgctaccaaggaaaagagcaggtgttagtcgtgactgggaagtacctgctgcattagaacagttagtaactaaatatgctttagtagaaatattccaagtttggggtagacttatcatcggagcaggtattgctagtagaacactagtttacgacggagtttcagagtctattgatacaactcagtctgccatgtatacaggttcagcagcagatattgagcttataaataaagacatagacagtttagtagctcaactgcgttctcactttggaataaacatgattggactataagagaggagaatgtttaaatggcagagcctactagattttcaagtgatactcctattaaaaacctaacaacacaaaaagtagagtacaacatatcacagcatagaaagtttattaaagaccgtagtatccatacgttatgggaaagagcttatttatgcccttgtatgaacaagtcctcgggttctgctgactcttcttgtcaaagatgccatggtaggggatgggcgtatttaccagctaagcccgcaaatatgattattcaaggacaggaaaaaggggttactaacacagacctaggactatatgactcaggaacggctatcggtacaaatgattttgatgctagaatggcttttagggacagaataacagtacctgatgttaaaataggtcaatcccttatctactctataaccgaaagcagacaaaaacgaggtatatttttaacatatgacgttgatactatagactttataacaagtatggaaagagaactaatagaaggtaaagactacacctttgataaacacagtaataaactattccctgatattagtttgataggtgaagttatatctttaaacgtatctgttactctaaggtatattgttattgacttactaaaagaaagtcgctatcagtacaccacattaaatagaacagaccctaagtttgagcagttgcctcagaagttattactaaaaagagaagacactattgtattacctgaaccatttgtacttgatgctgatacagagactagagtagagttagttaatgaccctaaaaggaaaccaagacaacaaggcgggttctacgaagggatgtttgactagtgaaacagagtacacagagaccatctctattcaataatccaaagcaggtaatgagaaatatggcaagagctatagaatcagatgttattgcaacaggagctcaagaggcatctcgtatacccaatgtagagattaaaattatgcctaagtatttagaagtaactgaaaagagaatggaaaaaaatggtgtaattgaccttaaaccttattttgctaaaagtaataaaaagaaatggaatcaagaaggtaactggtacttagtaatccctataagattaaagacaagagatatgtctcgaaaattatatgatgacttaaggaaacagtcttccccaccagttggaagttcaacaacaattgtatcagatttcctatatgaccgcagaagaacgagccctagtgtcccttctataaactataatccaaagtcaaataatgttacaaaaaaacgtattagtaacagtagaacaacatatacagtataccgtacagtaactgctaagtcacctgctaatagttggattgttaacagggacagggttaatgaagaggatatgagtaaaacgatgttagacagaatagacaggctaatgaaatggaagcttaaaaacttgtagaggaggagtacaatgcctattccaagccttgattcttatgtagcaagaagagtaagagagagccttaaaattatgctttcagagtgctatattatagaggaagctttgagtgacatggatgtcgaagcaaaaagtaactttatgaagacatattgtggagataatcctaaaaaagaaatagaagtgatatacgagttccctcaggcaaaaacatcagtagatgctacttatgtaattcagctaggaggaggttccgaagatactaaaagcatcggcggtattgaaggtacttacgagtataaagaggattctttaagaaaagagacagtagttgctactagagaaggaaccgagttatttctagaaacaaaagagctaattggaagtatagagggaatacctaatttatccttttcagaaagtgataataaacgaatagaaggtaaacgattatacttctcttatgaaggtaatgcagatattgagggagtttccttcgatattatctatacaagtaaagtagacctagagagcgaccccgttggactaaaaaaagggtttacttctagagacaatatcactgttgtaggtttaagcactaacatggatactgcaaggtgcctagatgctatcttaaaagttatccttattatcatgagagaaaacttagaagagaagagttactactccctacaaacaactcagttttccgatatgtctcctgtaattatgggagaggaaaaacctgtatttggtagacctgtaacaatgagttacactatatcatatactgtagactttaatctagtcaaccaaataaataaaattatagtacaggagcgtaataagtgatggcagaaaaaaaagaggtaactaaagaacctattgtagacgaagtaaaagtaacaaagaagaaccttaaggaacctacaacagaagaaaataaagtagtgaagaaaaaacttaaagaagaacaaacagatactgtagagaaagttactacaggatatgttcatatagatgtattccttggaacagccaagccgtggtacggtttatcagaaatgcaagtacaagggtttaaatcttttatgcaagggaaacaataccagttcggtgaacaggcttttgttccttatttagaaaaatatctaggaagggaagtaaaataaaatatggcaatcgaaatttacccaagaaaaccagtttctagacctcatacagaaatttctgtagatacaagcggtattggtggttctagctctggaagtgaaaaaatactttgtttagtaggttcagctacaggaggaaaacctaatgctgtttacaaagtaagaaactattctcaagcaaaatctgttttccgttcaggagaacttttagatgcaattgaaagagcatggaaccccggtgaaggaacaggtgcaggagacattttagctatgcgtgttgaggaagcaaaagaagctacctttgaagctgagggggttaaggttagctctactatttatggagctgatgccaacgacatccaagtggcactcgaagataacaccatcacaggtacaaaacgcttaagcattgtatttgcgaaagaaagagtcaaccaagtttatgacaacttaggtagcatcttctctatcaagtataaaggtaccgaagcttctgcaacatttacagtagctgtagacccagtaacatttaaagctacaaagctaactcttaaagcaggggataaaacagttaaggaatacgatttaggttcaggagcgtatgcagaaactaatgtacttattagtgatattaacaacctacctgactttgaagctaagtttttcccaatcggagataaaaacttaacaacagacaacttcgatgcacaaatcgatgtagatattaaaacaaaagaagcttatgtaaaagctgttggcggagacattgagaaacaaacagcatataatggttacgtagactttgaatttgaccgctcaaaagaaattgctaatttcccactaactaagctaactggtggagacaacggtacaattcctgagtcttgggctgataaattcagttactttgcaaacgagggtggctactacctagttccattaacatctaaacaagctgttcatgcagaggcacttcaatttgttagagattgctcttataacggtaatccaatgcgggtattcgtaggtggaggaatcggagagtcaatggagcagttgttcacaagagctatcggcttacagaatgaacgagcaggtctaatcgggttctcaggaacagtgaagatggacgatggacgttctctgaaaatgccgggatatatgtttgcagcacaagttgcaggacttacatgtggactagagataggagaagcaatcactttcaaaaacattgcgattgagacattagatacaatttacgaaggttctcaactagaccaattaaacgaaagtggtattattaccgcagaatttgtacgtaatagagcagttaccaatttccgtattgtagacgatgtaactacatttaatgataaaacagaccctgttaagtcagaaattggtgtaggagaagctaatgacttcttagtttctgagttgaagataagcctagacaacgagtatattggaactaaaattattgacacctctgcttctttagttaagaacttcgttcaatctttcttagatagaaagaaattagctaaagaaattcaagactacagcccagaggaagttcaagttgtaattgagggcgacgttgctcgtatctcactaacagtattcccaatccgcagtatgaagaaaattgaggtatctctagtataccgtcaacaaatccttactgcctaatctaatacaggggagggcttagctctccctatgtaaataaaaaataaaacaggagttgaaacataaaatggcaagtgttactaatcagacggttcatacaggtaatacggtctaccttatgataaaaggtaaaatcgtaggaagatgtcagtctgcatcaggtgaacgttcttatggaacaactggtgtatacgaaatcggttctatcatgccacaagaacacgtttacttaaaatacgaaggtagtttaacagtagacagactgcgcatgagaaaagaagatttagctaagctaggtattacagcattaggagaagacattcttaaaagagatgttatcgacattgttatgatggataatactactaaagaagtaatcattgcttatcgcggatgttctgctgagacatacaatgaagaaatcaaagctaatgagattgtttctgaaagtgcaagattcttgtacctaagtgcagcaaacgtaaaataatactaataaaaggctaccttaatctggtagtcctttttttatagtttactaaccttagcaaatatggtataataaagtaagagaacgaaaaagtattactagtttgttatattagataatgagagagtaaacaaggaggagacaaattaacatgtcagaagaacaaaaagtaaatgaagaacaaatagaagaaagtaaagagaagatgttgacgaaagaagacatcgaccgtatcaactatgaacaacaaacgaagcaaaagaaaacacttgaccgtattattaaaggggtaaatgatgtttggacacatgagtatgactttaaggagttagatttaaagttcaccattagattacgagcacctaatgctttagaacaagcaacaattcaagctaacagagaagcttatttaaaaggaacaggtgcattagttagtgactacatttatacaatctatcatacattagccactataagaacagttggtgtagatattccaaaggaactagcagaagacgataacatttacaatactgctattttataccaaattggacgtgattttgccgaatggctagacacgtttcgctactgagataaaagatttaggtggaataaaagctttagtaaaaactccctatgcaagaaacatgtggggaataatgaaagagttcaatgttttacctacagacgagaggttcagagcgcttactgaggagcaaataaacttcatctactataacaaagagagagactacaaagaggcagagagagcttctagaggcgtgtctatggagtctgaatttgaagactataactcggatgactggtgggaagcttctcatgaggacttccaagcaattcaggaaggtcatgatgaagatgacatcgctcaacaagtagagaacatgactagtatcgaagaccaaaaacgcctaagagacagattccagtctcatgaggagtacgaagagtttataagaaatggtggtaaagatactagacaggaaatgattgatgaacacatcaataacaaactgaaagaactgtttgcagatgccgaagagttaaataaacaaggcttgagtaagtggggagaagtatcagaaatgacaactatccaagaagctgtagcagacgagttagaagagctaacatcagatagtgttaaggaagcaatcagtatgttcgaagatgacgaagttcaggtaataccgggtcaagatgaagacgatgattttgaaatttaatactaaaaagaaagggtagagctgtgtactctactcttttttactaaggaagtgaaagtaattggaaaacactaactatcgcttttcggtagaagccttaacggcagatgcagagaaaaagttagagcgtatcgctagaataatggacgacattgagggaacaaataggaaaggtgcaagtaatttcttcaccagctctcaaaaagatatagacaacaacataagagctatgagagaatatgtttcattagttaatgaagttgagaaggaaatgaaaaacgcccagtcttctatgtctcgttctggaaataacgaaggtgttcaaaaattaataagagaccagcaaagactaagggaagaagcggaaaaaacacggaaaacattccaagatactacacaaaataaaacaggtagtaacgcttatcttaaagacctgttaagagaccaaaaagcatacaacgatgaaatagacgaaggtactcgtagattaaaagagttgaaagacttacaaagagatgtagccagaacagatagcagactggctgccagaacaaggcaagctacttcatcaggtaatatgaactcacaacaagcaggaacagctaggtctgacttagcaaataaaaccgcatatatgagtcaattacaagccaaccaacaacgaagagatgagttaagaaaagggctagagtccgaccaagcaaaaaaaagaaacttaaatgctaactttagaggtaacgaccaagaaagacagaatgagttgtcagcgttaaaccataatattgagactcgtagaaaagaaatagaagctttaaaagagctcgatagtcaaataaataagacagtaagtaatatggaaagctacagtaaccaattaaataatgtaaatgtaaaaccagaccgtaactcgtggcaaggggtcgcttatgaaagagcaccatctatcgctatggctgcattaggtggagcaggtgcaatatttggcggtctatatgctaaaggtagctctataaaagaccagattagacctgacactatctctatagggcaacgaacaggtaactatgatttccgttctatcagaaaacagaacgaagaagtaggtatggataggtctcttggttacaacgcagtagacatgattgatttccaaaacacctatttgtcttctgctgggtttaaaaaccaaaaagacttagatgcaggaactagacaaatggcagaagggtctagagctgtacccgtagataaagattctctaaatcagtttatgggtgggctaatgacgaatggagccgtatcaggagcagaccaagtaaaagcggtacaacaaggttttattggagcagtaaaagcttctggaatggaaggtagggaaaaagaccaactaaaagctttagaagctatgagtacccaaacctttgcaggaagaaatggttctaatgctgagctaaataacctgttagctatccagtcactgttttcttcatcaggtgaccgttctatgcaaggaacagcaggagcagaagccttatctagtctagatgcaggaatgaaacaatctgcatataacccacaagcaagattactatttggtatggggagtgagtaccaaggagtaggtggtatgtatcaagttcaaggacaaatggaaaaaggtctcggagatacagacaatttaaatagactacttaaattctccgcaggtgcaggaagaggtaaagaagaacaaaaaggttacttcaagaacatggctgagaatgtatttggtactacattaacaaatgaccaagtcgatgcaatgtataaacaagtaggctcagatggaacaatagataaagacaaaatgaaaaacatccaaagtattattgatggaggaaaagacggatacgataagaacaaaaaagggtatgaagactctaaggaagctacagaaaacgcctctaaagcagtaactgataagcaggctacagcgatgaacgacatgggggacatagttagaaaactaaacagtgttctcggtggtatgcccgcaccattgtatggtttggcagccgcagcagttgcagcagcaggagccttagctatgtctggcggaatgagtattctatctggtggtattaaaggaaaactaggtaagaaatttggaggcggaataggtaaagcaggtagcagtacagtagcttccgcaggagcaggcgcaggagcctcttctacagctagtaaagtatttaccaataaaacatggggagcaggagcagaaggagctacaaaggcttcctcggggctctcatggggtagtaaagcaatggaaggcggaatttctgcaactaaaggattaggtggagcaggaaaactagcccagtatggaaaaggagccttaaaaggtctcgggccgcttggtcttctaataggcggaggagcctcaatcgccaatattgcaggtgcagaaaacaaaaccgaagccatcggagaggaagcgggtggctggggaggcgccatgggtggtgcagcaggcggtgcagccatcggagccatgtttggtggagtaggtgctataccgggagcccttattggaggggtactgggaggtattggaggaagcaaagtaggtaagtgggctggcggaaaaataaagagtggctgggattctctatggggcggagacaaagtagatgctgctgagataaacggcacaggaggagctaaaaaagctcaaacagagaaaaaaaccaacatgaacggtgcttctatggctaactctggtgtaggaactatgctaggaaatacagtaatgcccggtgtaggaggaattatcggaggagctataggaggttcaggtatcctagaacaactttctaagctaactggtattggaagaaacgcacaagaagaaacaagttcaggaaaacaagatgaaaaagacacagtaggtaagaaagctacagcagagaaagagagacaaagtaatacaagagaagaagcagcaaacctttctttttacagtaaacttcttgacagagccgaagcattattgatgcaagctagaaatcaaaatggattattcggaaaagaaggcttaggaactactgctacaggtagcggtggaggagatgctctcaattcaggtcttaacttcttaacagatggtaaaaagtggactaacaaagatttaacaaagcatgacttaggttcaactattcaaggtattactgctgatgagttagacgaatggattaactcgaaagcacctgagggttctgcaatgagagggatgggttcaactttcctaaaagcagcacaagaatcaggattagacccaagatatttagtatctcatgcagccacagaaactggttggggaacatcgaacatcctaaaagaaaaaggtaactggtttggtattggggcatatgataactcacctatggcaagtgcctacaaatacggggacaaagaaactggtgtagtagaaggagctaaatggattgctgaaaactactacaataaaggtcaaacaacactagagagtatgcgacataacggaggacaacacgagtatgctaccgaccctaactgggataaaacgatagctagcataatggggggctcagagaaatacgtgcaaggtaaaggtgcttctttaacaacttctgctaatataactgttaacatgaatggagactcatcgaaagataagaagacagcaggaaaagcagtaggagatgccgttgctaaatcgtataaagaacaatcagagttcttctctaaacaatacaagagaacataatgttctcttgttattacatataagataaggaggataaaattatatatgactacagttaaaagaatgcctgaatttgatttaaagtttgtaacagaaaagaatgactatcttataagatatgatgcaaggaacccttcctcagatacattagctgagaaagtaataagtgtaactactaaaaatgcaatgtctgacgacagcgccgttttttctattgtagtagctggtgatatggaatgggataaaatattagactctaatgatgtagtaatactaaaaatatacccaaacttaagagtaatgatgcctgacaacgttgttgttttagtgggtctcatatccgaagttcgaagagaaggcgactactctaataactctattatttatcgtataacagggcaatcttttgctaaatccttcatgcagttccaattaggggtaattcaagaggtatctgtagtaattaccgatattgggtggttgcctgactctaaggcagatggtgttgagtttacaggaaaaacagcagcagagataggtaagtctatcacagataggtttaaaaaatatatgaaatacaattttaacagagaatatacaatggaaaacttcttagactactctttcagcagttggaaagattacgaaaaattagcagaccctaccccatttattaactatgagggtagcctaaaacaacttctagatgatgttacagcaaagccatttaatgagttattctttgagtccacaagtgatgagaagtgtaaaatgattatgagaagaacaccttttaataaagaagactgggacaagttaccatcttataaaatatctacagaagcagtaatatcagattctttagctaaaggggacacagaagcctactctatttttaacgtcactagtggaaatatggcaggagcaacatcagtagacttaaattcattccctcaatatcaccaagctttagtagataaatatggatacaaaaagttagaggtagacaacaggtatttatttgagtcctcaacagatggctctagtaccacagaaaaggcagatgtaggaagtaaggaaaaaacaaaaacagtaattacttacagcaagtttaactcttttatgcgtagttatacttccgaccaagttcgtatgaatcaaagtagtattgcaaaaagtttagtggacagctacgacaaattgacaacttctcaagccaaccaactgcttgctaaatattctgctgtaggtgcgataagtgaggcagattttaaaaaaattgtaggagacatagcggagggagacaacacaggaacagctactttagattttgatagtgtaaactcttggttctctctaaattatagttctttgtctgaggtttctacaaatagagatgctacaataaaagcttttgtaaaaaactttgctaacacagatgaagaccaagcaactaagatagtagcactatatatatcaagtcaaggagtcatgacaaaagaaaaatttgatgcaatcataaaggagtctactagttctagcactaaagacccagataacacaacaggcaactctagttcagcactacagtacttctctaaaacgatatataactggtacagtgaaaacgctaatttttatgcaggggatataaaggttattggtagtcctgtatataggttaggaagtaaactccttgtagaagataaacaacaaggtgatgagtgggagttttatattgaaagtgtctctcatgaatatagctatacagcaggttatactacaacactcggagtcactaggggattgaacaataaagggaaagatagatttactcatttatggggaaaaagttcggactttaaaggcgggttacttggagaaaaaacaagtgctgaacttatacaagaagcaggttctacctcgtcaggttccgatggaagtggcgatgtaagcgcaccagatgtacaaggtagtgatgttgccgtagccgctttaagatacgggctagctcataaaaagccagagaaaaaatcagtatattcttttggtggaggtagaggtagtagtaacccaatggagggtaaggaaccttacgcaatggactgttcttcttttgtatggtggtgttataaagcctgtggtgttactttagcaggagcacaaacacaagcgatattaggggacgataggtttaacacagtaagctcaagaggttctaagagtaaagaaatatttaaaaaaatgcaagtaggcgaccttgtttacttttacgataacaacactcatattggtatgtatgcaggagaaggaaagttcttaggatgtaatggagatggaagttgggacaccaacggaggagtacagttgaaaccaatggactctggttattggtggacacagtttcaaggacatgtaatacgttttgtttaaaaaggaggaatgactagatgagtgatgtagaatcaggagcaccacgaacacaggctagtttaggtagtgagtgggaacgagtatacaaagcaggagaaaaagtagacacaatcgtattagcccagataataaaagtaaactataaatataacaccgcagaggttatgatactaaaaaataaagaagtgatgtctaagtcgtatatttctaaaggtaagttctcagctccactacctcaagattttgctggaagaactctagaaggacttccatatggacaaattaaaccaattcaagaaggagctattgttcttatagggttcctaaatagcaagaaggacaacccaattattttagcagtctacggggactcagatgtaaataataagctagctagaactccttatgaaagtgcagacccattagaccctaagctagctagaataaataaccagttttttaacttgtacccttccttgacgtatgataatatagacggggacggaaatagaatatccacttaccccggcaagacattttctattatgacaagtttaataaacgagtcttccaacatttcagacagcaataatgggactgaatatgcagacttacccgaatcatactacacagactacaaactgatagagccgagaagccctttagcaccatcaatgctatttaaacaccaaggaataagaacactagacgggaaggaagacaatcatttagtaactgcctttataaatacagatggaacaacaaggctgtctttcctaaataaggttatgaactggcgttcctattatgaggctgatactgagggaggtcaaaagttcagacttcaaagagacagtaaggtaccaaaccaaggaagcatattttcagagtttgggttaacaaaaaacggagacttctttattgtaaatggaaaccataagtttgaagtaagagagaacggtatttttattaatgactctgctttagcagacgacttaggggaagttttagaagaaattcaaaatagaatactagggataaacgaggagattgctaatatcatccgtatgttggatgagattgatgttgagaacctaggaaagttatttgaccgtatgacaaaagtagaaacagacattattagcttaaggaatgattataaaggaatagacagtagagtgaacgatatgtcagaagaccttaacagtctgattaaatcctttgataactttaaacagacaatgtctgcctacaccgaggctaatactataagggttactgctattgagaccgctactgcaaataatacagtatctataggaaacattaataatagtattactgcgattgagcagaagaacacagcccaagatacgaaaataagtagcctagagagtaaagttacgtcactagagacaagtaatgctagcttagtaacaaaagtaggggagttagaatctgatgttactttcctaaaagcagaaaatatagatttaactaatgcactagcccaattagcagaaagagttactgctttagaaaatagctgatagtagtactatattataaaggagacgaggtggaaaaatgccacaagcagacggaaaaggtggagtattaagacgagttgctttccaaataggagagaataaatggtatcgttttgcgataaaccccggtagttactcttttacaggaggacaaagggttgctgtgtttaaaacgaagtccttaaacgtagtagaagactttggtagagatattcctactataactttctcgggaacgacaggtcttagaaaagactctaacggtaaaacaggagcagataggttaaaagagcttaaaaaatatattgaagattatgcagactcatcaggaaacggaaatagacctaatacagaccttacttttcataattttacagacgatgaaagctatattgtacatttatcacctgaggcatttaagatagaaagaagcgcagaaaaacctctgttatatgactacacaataactttagtagttttaagggatgcttctcaaccagaccaaggagacattgtagacccagacttaggtaataaacaacctagtgtaggggacaagacaaaggataatgacgatgtttatggggatgtaggaggcgcagacaagacaccagaagctaatattactggaagagtccctctagaagtaggaggtaacgggggaggggcagtaaacccccgaacaacaggtaaagcatactcctacggtgtagatgaattaaaaaaacaaattggttatggggagggttaacaatatggaggtatatcagtcatcagatttaatgaggtttattacagggttaaaagttaacccagataacactattgtagtaaacatcttagaccagcaatcaccatttatttctaggttttacactcctaagcttattctaagttctttatcagtaaagcttattcctgaattgcaaggagtaaacattatacaaacaaacaaagcattaagtaaaaatacagttacagctaaaatatcagcaagtaggttaaaaatagaggctccttacatgtatctaatcgcacagtctatagtcatggaatgctttgctctaatctacagtatagaagaaaagcaagatatgctaaggtacattgaagtaggagaagttaaacaagttagagataacataaattatttagctgactacttagctgacaaaccagaatacagcagtattgttacggacttacatgatatggaaattgctgtagggtatattgagaaccaaataccaattatacagagggagataagattaaatggctcgttataaaaaacacgtagtacaatatggcgatactatgcagtctatctctcagctagagactggtagtgttggaaactgggtagatatagtagaatataataagttaaagtatccttatattgtacaaacaaatgaggaaagaaaagaaaatcttgagagtacagttactattggggatgttattatcatacctgtagcatcagacctaacagaaatagacccttatgctctaacaaaccaagataagaacttaataatgagtttagctctaggtagggatttaagtatgacctccaatcaagattactataataaaagaggaactagtgacgagatactagaagttgtagctaacggcaagggagacttagcgactgttgacggagtagaaaacctaaagcaggcactaatagctagactattaactcctaaaggagccttgatgctacatccagaatatggctctaacttacacaagatgtttaaaaaagctacacatgaacaagcatcactaatagatattgaaataacaagaacattgttactagacggtagagtagaaacagtagaaagtctcggatacaaagtagaagaggacgtatatgtaggaaaattcctagttactatagagtctttacagacagcgtttgagttcgttgttcaaggtgatactacaggttctattattttgctagataattgaggaggaaaacaggttgagattaaaaagaatttcagaaattttatctcgtttaatagataagattatgattaacacatcagagctaaatgatttttctgtagggtctactatcttgtctatactagaggcagtttctatggagttggagcagtactacatcctatctagagagaacattgtgtggggtattcagcaaggggtatttcaggctttcgacttttttaagagagagccaaaaagagcttatggagatgtcctaattgagtttcatagcgcagttcaaagtgatgtatatattccaaaaggaaccacttttagttcaacaattaacggatacgaccagcaattcgagacagtcatagactactacgttcctaaagattccatattagctcgtgtagaagtgtattgtaaagtagcgggggaaataggtaatgtcccagcaggaactattaatttgattatgaatagtgtaagtaacgttaagagtgtcataaatgagagtaacttccaaacaggaactgatgaagagtccttagaatcagtaaaaaataggtttcatgctttcgtagagagcagagggagagctacagtccctgctataaactatggtacccgtttagtagaggaagtatcaggcgtatatgtaaaggaatatgtagggtatgtaattgtctatgcacacgatagaaacggaaacctttcagctaacatccaagataacattaaaatagcattagaagactatagaccagcaggtataaaattagatgtaaaacccgtaaacaaaaggttgctacctgtcgaggttactgtaactattactaacaaaaacagaataggggatacactacaggcacacattcaaggagtgataacagactacttaaatagtatgcaagtatctaacaacttaattttatcagagcttattcaaataattatgagtatagacgactcccttatttatgactgtaagattgaaaactttacagaaaatgttataataaatagtgaggatattattagagcaggagaagtaaaggttaacttaaaataacatagttaacaaatgagaggagctgacaactacccttgccaaactttttaaaaaatatacaccctttattaaaaagaaaaaaagatgacaaccaagtaagtagcgtaaactacgctgttttacaagcgctcgaaacagagctgagtgaagtagaacaggaaaccttagagagtaaagtacagtcatcccttggaacagctacaggagactacctagatatttggggagactggtttaatgtataccgtagagaagatgaaggagacgaacgttacagagataggattattcggcatttgttattaaagagaggtacaataccagctataatcgaggcaattcgtgattttctagaagactatagcgcagatattactatctatgagccgtttaaaaatatcttttacacaaataaatcagacttaaacggtgaagactacttgatggggtactactatcgttttgctatcattgatgttacaattggtagacggtttcctttagaagtaattgacattataaacgcatttaagccagcaggtgtaaaattctatctgacttatgacggtagtggagaaattgatgaagaaaacagtatagttgaactaccaccttatagtatagaaattgaaacatatcaagaggtagatattctaaacgggttaaatacaacacttcacgggcacttcactttaggagagtctaatattgtagagggtgttatccttgagttgtttcgaacaaatagaagtccactaaactctaatgatgtactatctggctcttttaatgcaggtagacgagtatacaatctagcaacaattggagacaactcctataaacctacgatgagttctcttattggggaaataaaaaattcgttcgaggaattacccgaagacttctacgtagaaactaaccgcttagatgataggtttgctagcatagacatggcaaataatacacaagatactgcattctatttcaacatggatttaggaacttatatcaatactaactacgcaggatatttagctgagttagcaggaagccagattataaatagagcacactatgtatccttagctaaagagccaaagttaaccgtggcgtttaaagctaaaatatccccagcaaacccagtaaattacaatatagaaggttatgacttagtttctaaaaaatgggttgttcttgagacaggagctatagcggttgaaaagaaaatatcacagatacctttaggttcattagaagactatcttaatagtaactatatatgttttttccgaattgtagtgaaacctaaacgggaaacctactcctttgaattagactatttcgaattaggatttttaaacaactttaaagaaggggagtatacaattaagccttttacaggtattgtagaaacttatgcagagttgttcggtagagttatagaagttcctatgtatcggataataaattcaggaacagaaaatattatttctaaacaaggtcatatacctgttagatatattagagatactttagagggtggtagtactacaaacactgctaacctatggatagaaattcaggctaaaaataaagacaatatcaatgttgcgcttaataagcctgtatatagaccacaagctgaacctacaaatggtaatattcttctatatccacagttccttaatattggagctacttacatgaatggtaagtggagaggaaaactaactagtgaggtaaacatggcatatactgtagacggtgtaagaacttacggaggaaacatgaacgcagatggaacctatacagtctatatcgggaacaaagtaacctctactagccaaaaagtagttgtggaggtatatgcagacactgcaagaacgaagttaatagacaaagctaatgttcctattacaaatgcaggggacggaagccctatagaggctatcacagacggagtaattagtggctctaatatattctatattgcaccaacaagcgccaaatactcagtaactgtagacttagaagctacctttgtagacttatcagaggtaaggatatggcactacccaacacgaacgtttaagtctgttttgacagaagtatcagcagatggtgtaaactggactactatatataactccgctgttgatgggacttatctagaaactagtgaaggaaaagcgtttcctgtaccagagatggacttatatggattgaaagctactcctctaacaagtgtactagcaggttctgaaagacaaattattacattcctagactacaaaatagaggactttgagaacccagctaatactattgaaaacctaaacaatgattactttaacgcagtgtggcaagagattgaccgacttgaagagtttgttctaggcaagttcactattttagctgatgttggctctgacggagtactagtaaatgactggggtcttattgaggctaaaatacctacaggtgctacagcataccaagaagcggacagtagaagtgaattgtcttccgtgtacactgtggagctcacagcgggctctcacgaggttattatggatgttgctaataaagttgttactacagaggcagtaaccctaaaaacggattacagggtgtctaacgagcttaccttggaagggctacctacaaaaatacctgtgagatacattcgagactccttgtggggaggaaacagtataaatacagcaaaccactgggtagagctacaggctagagacttgaaaggggacaatgttgcccttaataaaaaagtggtagagaacaacggagttagcttactatctcctgaaaatctaatgttagactcgaagacagagattactagtacaacaaacaatggttatacttttgcttctgatatagcttatatatttgataattatggagtagataaagagtatactattagttttgacatcaagtccactacggtaggcacagttactgtttatagtaggtacaaagatgaagctaaatattctataggaaacaaaactgttcctgtaacgactgcttacacaagacagcaagttgtagtaacacctactagaaacacagcagaacaagctaaatatgcagtcagctctattctaggttttcttaatagtacaggagcaatatctacagttaaggatataaaaatatcagtaggaaacatctcgaatatgatttggacacctgcacctaaagactacagtattcttactgatggagtaacaacaagtgctaacggattcttgagtggagttaactccttaactattgacttaggtagccctagatatgatattgacaacatcactatgtggcactattggcaaggggctgaacctaggtactacaaatatgtaaaactagaagtatcagcagatggtgtgaactggactacactgtttgatagtgatgtaagtggaacttattacgagactagtggaggtaagcaggttagcgtacctgctataaaagaataccttaagagtactaatgctatatttgcacctagcgtagcaggaacaggtacttttgagagagtaaccgaagcaaacggagtaagttctataaaagtgacacctgatactgataagacagctagaatgtacaagtttttcggaacttctacagaccctaacgctaattcgagagaccttctagaatcagggctaactacaacgaagaaatacacagtagcagtatccctaaaacctcaaaataatagttcggttatcttagcggtaaaagatggaagtaacactaacatagagtcgggctccttaaatattgcaaaagatgtgtggacaagagtttcttacaatgtaacgttacctgcaacagctattgataggctagctttatgtttgtacaatgctaacacaggagaatatattaattataaagattggagcattaaagaaacgatgtttccataaagtaaaaattatggtataatagagatagggcacaaagtagtgccttatctttgctatattagtaatgagagtacgaatacaataccgattttaaaataaggaaatgaggtaaataaatggcaatagcaacatataatgcgcatgtatatgcagcactaaacctaaaaagtaaggtcgatacaacatttatggcaattggtaaaacaacagcgtggactgatgagactaaccctccagaaccagacccaaatgctacagggttaacagaagttatcggttacaagaagctaaaaactatgtctctatgtagaccacaaagaacaggtgaaacaccaacactacctacagtttcttacggaaataaaacatgggttcttgtacctgatgcacaagcttatacagaaggagctaaatggctttattgtgaggcagagttcgtaggggatgaactaccagtaggaacatacagacaagtaggtgtatttacagacttagcgccaaaatctggtgtaacaaaaccgaatttattaccaagtgaagtagctaatgtaggtgtactacagttttttgagaacaagcagttccaaaacagaacaccacaagttacagctagagaaagattcgtagcggaattataagaaagggtgaataagtttggcagatttcaattttagttcagcaccatatcataataggttcaatgccttgaatgattggacattagcacttttccaaccagacagaccccttcaacagtccgagttaaacgaaatgcaatctattttgcattattacctaaaatctgttggggatgctgttttttctgatgggaacatgcaaacaggtatggagtttgtcttggataactcaggaaacttaactgttggtgatggtcaagtttatgttgagggcaaagtaagaaacttcaaaaaacaaactataaaattaaataagataggtagagagtttgtaggagtaaaagtagaacagtctattattaccgcagaccaagacagtagtcttctagaccaaacagcaggagttcctagtgctttttctaaaggagctgacagactaaaagaaactgttgttttaacgttagacgaccctcagtcagcaactatctaccagtttgataacggaaaactttttatctcctctgataacccagagatgacaaaaataaacgaaattctagctgaaagaacatacgatgaatcaggctcttatcgtgttaacggttttaacatgtggacagaacctcatccgacagacactagtaaaattcagttagttattgacacaggtagagcttatgtgttaggatacaaggtagataaaccaacatctacacgtatacctttagataaagctagagatactcgtagtgtagccaatgaagggcaatactacgatacagcagttaaccgtattaagctaggaaacagtcctgtagaaacagtaacacgagttacaggtcaagttcaagtacctaaggagcaagtgagcagaggttctcaatcaggaggcaccgacttcttgagtagaacttccgtattccagatagaccgtgtgtggacagaaaacccaacatcaggagaagttattaaggaatacactcaagggacagacttccagttaataaatggacaagcggtttcatgggcacctaacggagcagaacctacggcaggagcaacttactacgtgtcttaccgttataacaagtctatggtactgaatacagactacaagataactattgaaggtacaggggatacaaaaacttggtatattgattttagtgatatgacaggaagtaaacctatccaacaaagtttagtaaacgttgattacaactactacttggctcgtaaagacctaatagtacttgatagatacggagaagttacagtacacaaaggacaaccagataacttaagattagcggaagcacctgcacaggtagacccctcaacacttcaaattggggtagttactatttaccctgaaagtgagaccgctagtgctatgtcattctctgttactcgtctttctatggctgatttacaaaaaatgagaacacgtattgataatattgagtataacgaagcagtaaatgctttagataatcctgcaatggcaggagaaaacccagtgacattacgttctgtattctctgacggttttatttcactagacaaatacgatagcacgcatccagatgctattatagcttttagttttgaggatgcagaaattaccctaccttaccaagaagttaacaaggtagttccaaaaatgttagagaacgcatcagaagcttatgtttggggacgtttagtatcagcaccatttacagaagagaaaacaatttctcaaccttttgcatcagaagctatgaacgtaaacccatataatgtgtttaataaacaaggggttattactttaacaccttctgcggataactggattgaggaagaacgagtaactgtaactcaggacgaagttaagtcttatacggtaaaccgttggtggctacatggaggtaactactattctgatgaagctagatacttgcataacaacctagcactagatgcagggcagtcatggagtgggactccttctcagaacaaacaagtaacaggaacaacattaacagcaggtggacaacaaacacttgagtctatgatagagtttatgcgacaaattgatatagtggtagaaggtaaaaactacttgcctaacgctaacaacttatacgcactattcgatggtgttcgggtacctattacaccagcttctggttaccgaaaaggtacagacacaggaacattaatggctaatgcagatgggacagtaaaaggaactatcactattccagcaggtatccgttgtggtgtgagagaagtaactttacggaatgcagataacttaggtatttctacattcactgcacaaggtagaaagaaaaccgtgcaagacatcattatcagaactcacgtaacagctaacttgtatgacccgttagctcagtcattccaattcacagatgaccgagttataactagcttaggattacattttgctagtaaagatgcaggagaaaacaacgttatcgttcagattagaggtatgagtgacggaggacaaccaaataaaactatttatgcagaaagggtattaaccccagctcaagtaaatgtgtctgctgacagtagtaaggaaactaaagtcacttttgatgacccaattatgtgtaaagcaggtcaagagtactgtatcgttattgtaacagactctgatagttacactatgtggttaggtactagaggtaaacctagaatagactcacctagcgacttaattacgtcaaacccttatgtacaaggtgtactatttagttcttctaatgcttctacatggacagcacaccaagattctgatttgaagtttaatgtatatactgctaagtttaacgaacaagctgtactagaatttgacacaatgaaaaatgttaaagttgatagagtagtactaatgagtacgtacctaactcctaataacacaggatgtatctgggatatgaagattgttctagacaatgaacctacaggagttacagtagcagacaaagaatggcaaccaattggtaactaccaagatttggatgtagagcaattagctagagaagtcaagttaagagctacttttgttgctaacaactacatttcaccattgttatcattggacgacatcatgtttgcaggtttcttaactgctttaaaaggtagttatgttagtagaaatattaatatgacagactctccttataatacagttaagattagttacgaagctttcctaccagctaacactacagtaaaagcacagtttagtactgatgcaggtattacatggaaagacttgacgtctattcctgtagtaacacaacaaacagccgatttctacaagtatgaatacgaagagaaagtcatccaatcaggcacatataaaggatttaaggttcggctgaacttagcttcacaaaacagtttcttaagacctcgtgctcggagactaatgtgtttaatgaaagatgaataggggaacttttgttcccttatttttaactaaaaaggggcggtaaacatgccagagtcatatagagacccccattcaggggctttaaagtttattcctacgaaacaggagaaaataattaaagatgtgactgaaaaactaagagaagacgaagaaaaaattcgaaagaaaatagaagagttagatattaaactagcacaggcagacagcaggttgcaagaaataactaacttgtatgaagaattaaaagaactaaaggagggtaactaaatttgtctagatacgaccatagtactgtagagtacacagacaaaattaaggacttaacagattcggtaaaccgagtaggaaactacttatcaggagacaatagcccttatgacgatgttcagaaattaaaagcaattacacaaaatattaagctaactaaagatacaggtttagctaaaagcattacagcaggaacaactgcgttgaggtcagtagtcgaagtaggggtttactatattaatagcacagaagctctagccctaactgataaacccccagagctaacaggagcctttatactagtaaactacccgacaacagcctctacttctgttaagcaagaagtacacatgtttgctacaggaacaacaggtagctacgtagggtaccgatggatttctgcttctagtgtcagctcatggtggacttatgaaaacacactaggttcacaagctaaagcagataaagcattagcggacgggaaaacatatactgatagctcagtaaactccgctttgcaagcaattcaaaactcagcacaaatgtacaagttaacagcggatgatggtaagcctattgatgcttctgctatggctactccaccaactagtgtagcttctctaacgaagacaggaatatattactttactgccgcttttggaaatacaatgccagataccccttgcacagggcaaccattctggttggttgttttacagcatgtaacagataacagtatatcacaatctgtgacagccaacactgtagcagtagagagggtagtcgcagataggattattactactctaggggtaccgagtaagtgggaatacagagctaaagcctctaatttcttctttagtgcttccaattcgtcacgtataacactagtatcgacccaacagaatattataacacctaataaatttattaataaccccggtagtttaccactatctgcgataaaccccgcaataactattccagaagatgggatgtaccaagtgatggcggccctaaacattaacggtattttaaaaaaagtgtacctagtaagtgagctaactctactaataaatgacgtagtacacccagctacatttggaatggtaaaaacagtagataatgctaacgggcagtactctttagcagggaatggcatatatcaactaaaaaaaggggacaagttaaaactaagaagttactgtaacaatacagggaataacccttacttggatgtagacaaactttatatatctgttggtaagattgcagacatttcattatttaaataaacttagtatgctgagaaaggaagtcacaataatggcagaagaaaaattttcattagacggaaggcagtatgtaacagcagtcctatatatgtaccctaacctagaccctcagtacgactttggagttgtatcatccgaagggaaggtagaggcttttgaatggaacgtaaaaaatataacaaagccttctattgcacagttagaccaagcatataaagactatgtaaaatctatcggaagtccaagtaacccaccagccctagaaccagaggtctcaaaagaagcctcaataggtaagattctaactgaaaaatttagtgcgttagaggaagttaatgctaatatgttattggaaaatttagagcttcaagctagcctagataaaacaaaacaaaaaaattcagaactagtatttgagctagtagacaggggagtcttatgagtgtaaattggtatgagttagttttagattactacacaaaaggttattacacaccaagtcaggtaaaaagatttgctgaggtaggaaaactaactttcgagcaagccgaagaaattatagctacaaaatcatagagtatgtttaaactatataggtaattaagactaggactaatatcctagtctttttatgttataataaagatatagtaaatatatttggaggagtacattaatgagaattgttgtagatataatgaatacacgagttttattcgataaagaagaagaggcaattaaaaaaagatttcttagggatatgcaccaaaaattaggggtaaaagtagatgggttcttctttagcccagcatataaatcaggtcattgggacggtatccaagacttctatgactttaaaacaaatacgttccctacagggctgttgccacaagtgcaagagatactaggagctatgcaaaatgatgccgtatttcaatttgagattattgatgacagaccagacccgtttttaacagtagaggacttagatagtgagataactcttataggagatgacggaggagatatacagcttagagattatcagtatgactctgtaaagtctatcattgaaaactatagtgggattatcaatattgcaactaatggaggaaaaacggagattgcttctggtatcattcagcagattttaccgcatttagagtctggggagagaatagccttttttactcactcctcatctatatttaaccaatcaattgaccgtatagaaaaaagactaggtattaaagtaggtaggctaggaaatggtaaaaaggacatcagacaagttacttttgtaatgatacctacgttaacagcctccctaaaagaccccgaagttggtttgaaattaacgcataaggaacgagttattaagaagttagctaaagaggtagttcctcgtttcactatgcaaaaaaatcatagaaagcttcttaatatttatattgagaacaacagaccgaaaaataaagtagaagaagatataaaagctcgactagaagacttgttatattcctgtggaacagactccgaagttctaatgggactaaaaggttttaacgcagaatatgaaaaaattattgagaaaaaaaataaaaaactatatgacaagaacaaacaagctagagaatttctatcatctgtatctgttatgattgcggatgaggcgcaccataccagttcagaaacttggtacaactctttgatgtcttgtgagaatgctatgtacagggtggctttgacaggttctatagacaaagaaaataaaatgttatggcagagaatgcaaggagtattcggtcaaattacggctagaacaagtaatgagaccttaattacacttgggcactctgctaaaccaacgattaaaatattccctatcaaggctcctacagatattgaaaatgtagataactatgctactgcttatgaaaaaggaatagtagaaaatgattatcgtaatttacttattgcaaaattagcaaagatgtggtatgataaagacaaaggtgttcttattatagtaaatagaatagagcacggtaacagattatctgaacttcttgagtctctcgaagtgtcgcactacttcattcatggagacttagacatagatttaagagaacaacaactccaaaatatgagagatgggaagcttaaagtgatgatttctacatctattgtagatgagggagtagacatctcaggtattgatgcgcttattttaggtgcaggagggaagtctttaagacagacacttcaacgtgttggcagggctttacgtaagaagaagcaaggagaaaacacttgccaagtatttgactttaaagatatgactcatagatttttactaagtcactcagaccaacgaagacagatttacgtagatgaaaactttgaaattcaagacatttagggggaagaacaagaatggatttagtatatgtaggatgtcagatggaaataggaatgaggaaaggtttacgagactttctagtggatgttgctaaaacagctcagcaatggggagaaagcgtggctatggttactcaaaaacatagcctagctgagcaaacaggagtaaacattagaacagtctcacgttatatagctgaacttgaaaacaaaggtattattaaaacagaagcaaaaagaggaagaaacggtggactagttatcatgtttaataaaaatatggttacttttaaattaacagacaaccctataacctctgattcacctgatgcggtagaacttagagaccgtatgttccctaaaaagaatgaagacccgaataaagagaaaagacctcgtagaacaaaagaacaaatggcagaggaaaaagtactaaaaaatcgtaagagaagtcttaataatgagttaaacgatatgttagacgatacattattccctacagaagagatatttagtaaaacagaggagcctgagaggtactttaaagcttatttactgtctcgaatggttaatgcttatgcgtatatttatcctacagagagagagctagacatgctaagcagaggggtgcaggtagaagcagagaaggcacgaaaaaatagagtacgagcttactcttttaactcactacctgctagattttttgggacatctcagtttacttgctacttaaaactacaagacttattagaaaaagagaacattaacccattatcttatttaacagttcaatttgactacttagactacagattagataacagtcttggaggaactccactagcatatagaggtatccctgaggcaaactgcttaataggtgagactgcctttagtcgttacacatctgctaaggacttctacactgttatgcgaggtaaaaacatgttacgcaccagtacagcagatgttgcctttaaaggtggaaaataccctattgtggaggcgttattacacgcttacaatgtaggtaagcaagatattactgcactaaaccaacaaatagaggatatgaagacacaagctagtgtgttccataaatccgaagccctagcagacttttatgaaacaacaaaactattagttgaagaaagtgctttagaaacaaatgaaaaagacgagctatacaaatttgttactgaacaagttgttttacactctcgtaagaatgcaatgagtcttagccaacagttgctaagcttcccacttcaaattcaacgtgtaagacagacagccgaattaaaaggattagacaaaaaggactactatacttttgtaggcaacttagagaaacaagtgaaagtaagtagaaacacgtttaactcgtttactgaaaaaggtaagcttttacacgagtctctagaggcaaacagtactttttatagtaccattcgtatgctaggagactaccgaggactaaccgtaaacatcccgttactcggagaagctatcaagaaattaggagaggaacatattcctctagattcttatggaatgttagatataacacaggtttacgacaagtatttaacagacctgcaaatgcaagaagatgaagagaactatgaaacacctacaagttttattgttaagtaaggaggaggagatatacgttgagttccattattcaaaagcaagttatataccgagctattagagaccctttatttggtaaggatgtgttagcacagttacctagtaatgtatttgacgagaatgagcactacaaaaatctagtaattattattaaaaagcattaccagacaagtaacaaggcgcttgatgaaagtacgttgctaactattacggaggaacatttagttagacagaaaaagagcttagaagtacaggaaagctttaatgagacaattcatgatttgtataaaattgatgaactaggtatagacgatgaggctgtcggagaaagcattaaacgattcgtcaaaaaaaatctggcagctcacttgataaaggagactttgtctaagggtgaagggctagacaacgaagaaacattagaaaaactatacacaggtatcaaagaaatctctattttagatattaaccgtagtgaaaacactttatttagtttctttgatgatgtagaagccaagaaaaagatgctacaaagtatccaccaagataagttctctacaggattcacagacttagatgttattgctgagggtggtattggacgtggagagctcggtctagttattgcccctacaggtggtggtaaaactatgtgggcagtaaaccaagcaaaaaactatgttgctcaaggtttaaacgttttgtatgtagtgttagaggaacgattagaacgtatgattttacgttttgaacaaaatatgatgggtatccctaaatctcgacttatgccagataatgaacttgacgaagaagctttcgacaaattacaaacgatttatgatattaaacgtaaacaaggattaggtaatctttacattgaaaagagaatgcctcaggaagttagtcctacaatgttagagcaaattatcgttgatgctagtgttcgtaaagggaaacatttagatgtggttattattgactatcctgacttaatgaaaaaccctcatgaaagagataatagtgagtcccgtgcaggaggaaaactatacgaagaaatacgtaagttagctcaaacctataagtttgctggttgggtactctctcaggttaaccgcggaggatattcagaggatgtacttactgctaaaagtatcgagggttctaaacagaaactaaatgctgtggaaattgctatgaccttaaaccaaaaacctattgagtttgaaaaaggttttattagagcatatgccgacaagattcgtaataataacggtaaatcttataaccgtatgcttagctttaaggttgttccagaaacaatgactatcagaaatataacacctgatgaaagagtagaacacgatatgattttaaataacctcgaagaaaaagaaggtcgtgtatttaaaaatacgaaaaaggctacacctgacccccaagcagttgtagacgaagtaaaagcactaaatagtagattataaggaggagatagtttttggataaaattatattttttactgattttcatgcccatatttttaccgaattttctaaaccagatgtggagtatgaaacagacagatttagagaacaaatggaagtcttacaacaggtatttgacttagctagggagaataaggcagatattgtttttggaggagacttattccataaacgaggagctgtagatactagagtactaaaccatgtttttcgtgtatttgaaagaaacacggatattgtagttcacctactgagaggaaatcatgatagctttacaaactcgatgaacacagagagtagtttggagacacttagaggtttacctaatgtggaagtttacagcaacccttctattgtagagactgaaaaatataagttatatatgctaccttatggggatgaagtgcaagagatgaaagactggattaacagtttagacattgaagcaggtaaaagtaatatcttagttgctcatataggggtagagggagcgttaacaggtaaaggttcccacagactagaaggtgcatttacaacaggagatttaagaacaagcgactttgatgttattgctcttgggcattatcacaagagacagctactactagatgttcctaatatattttatggggggaacaccattcagaacactttttctgatgaagggcaagagaagggtgtatggctaattgactgttcagatgttaaaaatacaacaagtacttttattcctattagtagccgtaagtttattactattgacggagataacattcctgatgatatagaagaacttattgaacaaggcaactttattaggtttattggaagtaaagaacaggctaaaaaaattgacttatatggagaagaaagtacaggtattagagtagagctacgaaaagagtatacaactgatgttcgactagggattgatgaaaagtcctctcccgaagctatttctaaagcctatgcagagaaatttagtccctctgcaacaagtgaaatagtagagtgtataaaagaggcacttgtgagagaaaaataagcctaaagctgaggatattcctcagcttttcttattgacttttttagtaaagtatagtatactaaagttacaaataaaaagaaggaggaaaagaatgttcacttttaaaaaagtaaagataaaaaacttcttatctatcaaagacatggagcttaatttagacaaacaaggtctagttctgatagaaggaaagaataagacaaatgaagcctttaagtctaatggtgcaggtaagacaagtatgattgatgctattacttacgctatctttggtaagacagtaggtgggcttaaatcggattcagttgttaataacaaagagaagaagaacactgctgtaattttagacttcgaagttgacaagaataaataccgaattgaacgctatagaaaagacaaaaaagagggtaatatagtaaagctctaccaaggtaaaaacaatattacaaagagtacagtggacaacacagataaagagattttatctctattcggtattgattttaatacgtatgtaaatgctattatgtacggtcaaggagacatgcctattttctcccaagcaacggataaaggtaaaaaggagatactagagagaattactagtattgacatctatcagaaggcacaagaaatcgctaaagagaaagttaaagagaagcaagctgagcaacaagagatagaaaatgaacttgtaaaatatgactatgagcttgtagctattgacaacttagaagagcaagaaaaagaaaactataaagcagtccagcaaacaatagaaggtatccaagcaaccctatcagtagctagtacagagctagaaaactttatgacagaggcttaccctaagaaggcagaattagagaatagtattgcacagtttatagtccctcaagtagataatacaaaagttaacgagctaagagctgacttaacggaggcagaacgtaaattattacaaattaacagtattaaagaaggttttgacagagacttacgaaagctagctaaagactatgaaaacctttctagtagcaagatttgtcctacatgtggaacagacctcggggtggatagtgaagcacacattgcagaaacaaaagcagatattgaaaaacaaatgagtgttcttggtgcaaaaacccataaaattttagtagcgttaccttctgtaaatacaactattgagagcaaaaaggagctcgtacaacaagagatagctaaaaatgaggagatacaaagaaattacaatcagctagtgtcaacagtcaggggatatgaacaagaggtacaacaactagaaaacacaaaaaatagtttagaagagaaagtggcttcacaacaaacatatttatcacaggtacaaagtatgcctaagccaaaaagtagagacaaggatagagaagaagtacacaaaaaaatagaagcaactaagttgcaagtgctagacaaacaaaaagagactgagaagtatgaggaagcagtaaaagccttctcaaatgctggtattcgttctatggttcttgacttagtaactccttttttaaatgagaaagctaataaatatttaacagtgttgtcaggttcagatattgaaattaattttactacacaagttaaaaactctaaaggggaactaaaagataagtttgatgtagaaataatcaatggtagtggaggctctaactataaagctaactctgctggtgagaagaaaagaatcgaccttgcaatttcttttgcaatccaagatttagttatgagtaaggcagaactatctacaaatattgcattgtatgacgaatgttttgacggtctggacagcataggttgtgagaacgttattactttgttaaaagaaagattagaaacagtaagcagtatatttgttataacgcacaatgagaacttaaaacctttatttgagaaggtaatcactgtgcagaaagaggatggggttactaccctaataaactaaggatttggagagctataaaatgaaagtaaaaattatgacagaagaaaaaggggtactcacctcagaagtaagcaagaggtcaactaagaaagaggctatctactccgcaaatgtttttcctattagtagcttgtacaagtggtacccctttaatactaactacgcctatgtagtagacaagcatggggtatacagactcactgaactagatgactgtattcatacatcgtataacttggtaagtgaagaaaaagctaagttagacccgacaaagtggcacaataaaaccgtaacacaaagcaacttattactttgtagactagcaaataagctagggataaaaagcattaaagatgattttaaccaacaagatgtagaaaacggatttgaggttatcagtacagagttagcctatatcaaagaacaagcgacagcacgttgctacggggataaagatgtctttgaaagaagtgaactactcaagaaaagtaaagaagaaagagaacaagcatactttgagtacttaactgtagaggaggtaaacttccaatataagttaggagagctatacgctatgcttgtacttatgcggaaggtggtaaactactaatgtttttagactacattacaagtgagctaggaacacataaagatatagggcatgaattacggttctgttgccctttttgtggagaagacgactacaaattttatgtgaaggtatcaggggacaaaacagaaggtctttggcattgtaaaaaatgtaatgaaaaaggtaaccctgttacttttgttatgaagtatcttaacattaattttaaagatgccttagatatactagaggtgtacgactatgagatagacgaaaacagcctatctagcagagacgtaggggtaacagaggaagagtttctgcttatactattaaagaaatctagtgagcctgaggaagtagaaattagtgaggaagtgactctcacgccacctaggctacctaccaattttaaaagactagcagacaacatgaataaccccgaggcattaccttttctaaactacgcacactcccgaggtataactttagaccaactattcctacacaacataggatatgttatagaggggcaagtagaaaaagtagatggtaatatgatgacacttagaaatcaccttgtttttctaacacatgggaatgatggacaataccaatattggaacacccgagccataacacctatggggataaaagcttttaatgctcctagcaaacaggacgagtactctaaaagaacaactgtttttaacctaaatattgctaagaatacgccttatgttattgttcacgaaggggtaattgatgccttaacaatgggagagtcgggagtagctacttttggtaaacagataacaagagaacagatagagttactattaaataatgttagagaagaccagcctatttatttattcctagatacagaggcttatgaagttactactaagatagctagcagtctttatgaaaagcataaagaaacctacatcgttatccatggagatgaggatgccaatgatatgggatatgaaaaatcttgggactgtttaagaaataattcagtaagagctaatgaggaaggtataagtatttttttagtatcttgcattcagtaataaagtatgatataatagagcagacaagtaagaaggaggaattttattgagtaataaagtaaatgtaaaagttaagttagagggaggagctactatgccctcacaagggtatgataatgatgtagccttcgatttatacacatcagaggatgctattatttgccctgatgcagtgggagctacactaattcctacaggattaagaacagagtttgaccccactaagtttgggctattcatcagtcctcgttcaagtattatgaacttacctttaaccctagctaacaacacaggaattgtagaaggaacatatcgtggaggtattggtattcctttaaaaaatacgttgtctatgacaggtttttctgattttgcactaacatggaacccagacaagaaggtagtagaacgtatccctgtagagaagttacctacaggtaaacttagaagtgcggaaaaaatgttttacacagaccaagcattattgtatgataacacactaccagaagaactagcagaccaattgtttgttactttggtgccgactgggacattctttttaccaaaaggcttccgtattatgcaagcttaccttctacctaaatatcatctagacctagaaaaaacagacaaactatcagacagccctagaggggataaagggtttggcagttcaggacatggaggaaagtgattcctttagaccctattatccttattgccgactctataggcataggttattcaaaaggaaatataacactgtctttaaaactacgggataagtatacgggtaccgaggtagaaaaaatagctttattagccgttggtagcttctcagaaaaagtaacgcttgttagtagtgagctagtagataatgtgtataaaataaaagttaagatggaggaattatgaaagattttactcagtattttaaagaagtacaagatgcacaaaaaagagaggactctgaaactgtagaaagacttgcagtagaatatggagataaaactattacgtataaagaacttagacatacgttaggttccgtcttgttgccactaattattggaacaatggaaatcaataagcagttggcagacacctctttagacatcgttatcaatatgttagaaaaagagaatgtattatcagaggataccttaattgatttaaggcaactaattactagcatgaaacaatcagtagaaggaggagaaggagaaaatgagtaaagaacaaaagcagttaagtaaagaggatttaactttatgggttaactttgtagtaactggtaaggttgaaaaagggacggagaaacagctaaatcgtatttctaaacgagaggtatctgtaggagatgtaaccaccctaatccgtgctcttacaagtcatcatgatgcgtacattagttctcttattgaagctaattcagttcttagagtggtaatgcgagatgagttaaatatctcagaagctcaaatggacaaggcgttaaatacctataagcaacaagtaggtacagcactccaaaagacacatgatgctatcatggaaaaagcacaagaaaatgtggagttcacagaagaagaactagaggtacagaaaaaactagaagaatatgatactaaaaataaaaaagaggaataagaatggctagtacaggaagaggaagtcaagtaaaaggttctaaatttgaaagagagaccgctaaagctttatctgtttggtggggagctacctttaatagaacaccgggttctggcgggctaaaatgggcggaagaaaacaatgtaggtggcgatatagtagcccctccctccgcaggtttccctttcattgttgaatgtaaaaaacatgaagggtggacactagacaatgtattccttaatacaggagagcctaagtcttggtggaaacaagtagtatcagatgccacaagaacgaataagatacccttgttaatgttctctagaaacagggctaaaagctttgttatgctaccttatgatgacttgctatataacgtattacgtgataaaagaagtacagtagctagaactacagtgtcatttaagaatatcagggaagaaagtgtgttctatgatgtaatggtgttcactatagaagagttaactagcttctctcctgattttttaagagaacactatagtaagtatagttgggatttcttttacaaagaagtggacgagaactctgtagaagatttagaaacaggtatagataaaatgttagacaacctagaaggttaaaataaagaaaaacactaagtaggtcttttgtggtgacatatacataagcctacttttagtgtattgtgctatactagagtagaacggaaaaacaaaatatgtttggaggaatatcgttgacaataatagtaaaagacaacggaagtagaaaactaccttttgaggaagacagactaaacaggtacctagataacataaaaaaagagtttcctaagttaaatatagagagttatcgaaaaaaggcaatacactatattttaagcaaggacgagtattctgtagacgaaatggtagactacctaataagagaagcagaatctcgggtagacgttaccacacctgaatgggagttttttgctactagactatatttacaaagattgtataagaaagctagtaaaaacaggttttacgatgtaattatgaagtatggttcttacgtgggtttgcatgagagtctagcacaaagaggaatatattccgtagatattttagagaagtattcaagagaagagcttatggaagctgaaacgtttattgacagtagcaaagaccttctctttacttacaatggtttgtacctactagctactcgctatctagcaacagatgaggataagaaagtatttgagttaccacaagaacgatggttaactattgctttatttttgatgcaagatgaaaaagaaaatagaatgaagttagtagaagaagcttattgggcgttaagtaacttgtatatgacagtagcaacccccacattagcaaatgcaggtaaagtaggcgggcaactatccagttgttttatagatacggtagatgatagtttgcaaggtatttacgatagcaatacagatgttgctagagtttctaagcaaggcggaggcgtaggagtctatataggcaatgtaaggtcgtcaggagcacctattagaggagttctcggtgctagtgggggaacggtaccttggattaaacaattaaacaatacggctgttagtgtagaccagctaggagcaagaaaaggcgctataactgtaacgttagatgttttccacaaagatatagaagcctttctggatttaagactaaacaatggagaccaacgtttacgggctcacgacatttttacttctgtttctattcctgatttatttatggaagctgtagagaatagaggggaatggtatttatttgacccttacgaggttaagcagaaaaaaggttggtacctacaagactactatgataaagaaaaaggtagtggtttattccgagaaaaatatgaagaactcattcaagatggcacgattagtaagaagagagtgaaagctcttgatatgatgaaacgtatcatgattagtcagctagagacaggaacaccattcatgttttatcgagatactgtaaatagagctaaccccaactcccatgctggaatgatttatgctagtaatttgtgttaattctatggcacctttgggtagcaatactcatcgaaaacttttcttaaacgggcatagctgaatacgatgaataagctggtaagagagcctaagtccttgagtgaggatagaggtaatcccgtgctaaattataataaaatctttattgtataaatgcctaacgactatcgaaaagctactttttagtagaacttagtagagtacactcaagtgagtggaaacagaaaagactcccatgtggagtatgatatagtcttttctttacagaaatgtaaagcagttagtacactaacgggtagggaattaacgacccttactgaaagcaagcaagaaatcgcacaaaatatgtcagctacggtagttacagaggaaattatagatggagataaaattgttgtaacaaaagaagcaggagacttcgtagtttgtaacttgtcttctattaacttaggttctgtattttccgaagaagacgttatcgaccgactagtaaccattcaagttaggatgctagacaatgtaattgagttaaacaaactacctgtaccacaagctctaattactaaccagaagtatagggcagtgggattaggaacttttgggtggcaccacctactagctaaaaaaggtattcattgggacagtgaagaggctactaattttgcagatagcctatatgaaagaattgcttttcacactatcgaagcaagtatgaaactagccgaggaaaaaggagcttacccatactttaaagacagttcgtgggatacaggggagtacttctctagtagaaactatactagtgatacttgggttgacctagctaaaaaagtgaaagagaaaggtattagaaatggttacttaatggcagtagcacctaatatgtccacagcgcagatagcaggctctacagcctctattgaccctatctatgactctttctattatgaagagaaaaaagactacagacgacctgtgatagcccctgagttgaacttgaacacttacgctttctataaaagtgcttacaaagtaaaccagattcaaagtgttaagcagaatggtaaaagacaaagacacatagaccaagctatcagttttaacttctatgtacctagtggcattaaagcaagtaaactacttgagttgcacactacagcgtggagtgaaggtctaaaaacaacttactatgttcgttctaatgacattgatgtggaagaatgtgagtggtgctctagctaacagctattgacaacatgacccttttatgatatgcttatagcataaagtaagagggtttttccttgttgcagagaggtgtgagaaataattcaaggtatttgacaaacgatttaatgtgtggtatacttgccctaaatgtacaggaaaactactaaaaatagaggaggagaactaattgttaaaaaaaataaaaatattagaaccactaaaccctaacaaatcaacagcgattattaatggagaggctagtggtatcgtaaactggaacgatattcggtatccgtctttctatcgagcttacaaagagttaactacaaattactggttgcctgatgaggtagatatgaagagcgatgccaaacagtacaaccacttatccgaagaagagaagtatgcgtttgactccattatcggacttttagctacacttgactcaccccaaacacgttttatttataacattgcagagtatgtcagtgatgcctccgttcatgctattacagctatcattgcacaacaagaagctattcataatgagagttattcttatgtacttgcttctattactaacttacaagagcagaatcgagtatttgaggaagctagaacacaccctacgattattaaacgtaatgcacctattatgaagtcttatgacgatttcatgaataacaaaacacctgagaatatggtaaaagctttagtacagtcatctatcttagagggtattaatttctacagcggttttgcttacttctacaacctcgttcgtcaaaacaagatgacggggacaggtaaaatcattagcttcattaatcgtgatgaattagcccattcgaaatttatttctgagctaattagagctattgtaggggaaaaccctgaactacaaacagatgaactaaatgactacgtgtacgcttcttttagacatgccgtagagctagaacaagcgtggtctagtgaggtactacaaggcattgaaggaattgatgtggaagaaatgttagactatgtaaaatatcgtgctaataaaatgttagggatgttaggtcttcaaccaatgtatcaaggtcacacagaaaattctatgacatggattaaagcttacgccgataactttacagaaacaaaaaccgactttttcgaaatgcgtaattctagttacaagaaaacaaatttagacaatgggttcgatgacttatgagggtagggattttatattcttcattgagtggtaacactagtgaactagctcaaggaattaaagataacttggaggaactagcagtagaggtgaaatcttttcatattgaaagagaagaccaaatgactaatgaagtaatcccttatctatctaattgtgacctgattatggtaggtacatggacaacagattacggagtcatccctgaggagatggaagaggctttagaaaagtatgactatacttgggtagataagcctgttgctgtatttggaacaggagaaacccagtggggtatggaatacttctgtggagctaatgacaaaatagccaaaaagtaccactctccttttgatacacttaaaatagagcagtttattatcacagaagagcaagaaaataagctattaagttgggttactgatatagttaagcactggagggagtagctatgtacgagctagaaaaacttctaggtaaagataaacatggaaatcagataagaattggagatagactaagtttacaactagatgatggggagtaccgagagtgcgtagtagcggttagaaaagttaatactatcgtgcttaaccctgtagctttaactagtaaaagtgaaactgtaagtgtaacaggtgtattctttgtagttactgacccacatacaggagaagaactacctttactacctaaagtagatagtaacggtgtggtagacactcaaaaagcagaaataatcttataattttatagaaagaaggaaaacaagtgcattcagtagaagtagaaaagaaagaacagttagcggacattgtatcacaagacaaggttgtattaaagttttataaggataactgtagcccttgtaagatagtaggtatgtctattgaggaagctagtagtaatataaaagaggatattactatcgctaatgttaagttagaaactttaggagaggaagtcttctttgatttcggcatcaaaggcacacctacactgttgttagtagaaaaaggaatagagaaaaagagacatagtgggtttatttcccctgatgacttccaagagtttgttaaataagagaggcttaggcttctctttttttgttgacatttacaagtttatatggtatagtaacagtagtaacagaggggaggtaaaaaaaatgacaaaaaagaatattaaaataacgagtaagacagaggtaaaaaatagctctgttgaggcaggagacatcttctacattgaaggggtgtctaaggggcttactaatttgtctggtttgtatatagtgacatttgattttcatgcaacagaagagtcgctgtctaggtacgctctagtttctttggagacagggatgttaaacaccatttccatcaaccagttttctctagaagaaaaaatagggtgtatagtgaaggtgaataatatagaaatagtagcaaatactaaggaggtataactatgaaagtaggagaaaaagtagtaattaaaggtgaagtagttcacgtaaatggttctttggtaaaagtagagacaaaaaatagttctttttgggtaaacaaagaagacctaaagaaagataagagtagtggtaagtaggtatggcacacttaattgaaaagctaagactatataaaaaaccagaagaggtcgtaagaggaacatcagtaaaagcacagcatatttatgttgcaaatgatgaagcaaagtcattttcaggagccgaaaaatgggcgggaacgtctgcctataccttgacaagtaagaacgaatttacaaatgtaactattcactctctagacattagaggagagggaggcagagcttaccaagttacactaaatcacgataataatacttatttatgtgacttaagggaaactcaattaatggaaactattcgtaacgtaggaattgaggctggcggaaagttaattggaacttacagctttattgctgacggctcacagaaagttctagtaaggataggttcagacgcgtatgcaaaagcaagtgaacaagcggagactaaaaataaggcaaagagaattcccaagaaggagctagaagtaggaggagtatacgcacctaaggctacttatttaggaacaaatgactccctagacttatatataggggacttgtatagaaatgaaattgtagacgataacccttggtaccgagggtactacgaacgaagaatagaattagggaagcgtaagaagttctctgtaagcttaaatgtagcattacatgagggacaggtatgtgttccaacatacagagaagctgaaatcatacctgtatttctaacagacgatggggttacttcactaaatattagcaaccagctagggcgtttatccccaagaatggacttaaaatcaagccctaatttctttggaaaggtaggggaggctcctgaaattaaaacaacagcagagttcttagaatgtttaagacgtggcttagaagctcgcattgaagctttacctagtaaaatagagggcaatattttaaagggaaacttaaggttacaaaacgttatccctgaaatggagactatactcaagttaataggactcaatactacgattgaaaagcctaaatactccgctagttttgttaacaaaataaaaaatataaaatatatagttatagcagaacgtaaccaaaaagggacgttaaccgcatcggaaagtacagacttccatcagtttagtcgtgcttttgatgacttaatgtccaatattttagaggttattatagtagaggaggaaaataattgacgttatatatgaatggtaaaaaagtacatgagagtactttccccaatggggagattaatatcatgcctgatgaaaatttatgggtacttgaggaggagaaagcagagattacttggcagtactcaggaaacggtgcagaagacttgctagctgtcagactactagtgaacagtataagagagcacaacaagaaacataatagcattaatttaacagttctttatatgccatacagcagaatggatagacttcaaggaaatcagccctttagcttactagagtattttaaaatgttccctcaagttgacaaactagaaatagtggaacctcattcagacgttacagggagtattgctagaaaacttattgacagtggaacttactactcagagcggtttgctacagtagagatgctaccagaagtgttagcagaagagttgtctaatacttggtgggaacctgtattagtctatcctgataaaggagcaaaagacagatatgacaaattagcaactgattctgattattcattcagaagagctactgaaaacagacgagtactatttggaaataaagtacgggactttgatacaggtagaattaaaagtttagagctagtaggggaaccacctactagaaaaggatggacagctattattgtcgatgatttaacttcttatggtggaacttttgtaaaaacagctacagaggctataaagttaggagcaagccgagtagtactactagtagctcatatggaaggtagcgccactaagggagaattatttgaatctattgataaggtgtatactacgaacagtatggaagctagctacacaccagaagagctaaaagaagtagaaagatataaaaaactagggttactagacgtaactaatattataaaataaggaggaaaatatgcgtaaattaaatgaaatttctatgttaatgagtgattgctataaatttggtcacatgtctttatacccgaaaggtatcacacaggtgtatagtacacttgttcctcgggataatacttacttttttggaggtcgtacagataaaatggttgtctttggataccagatgtttgtacaacgtttcctgttagaacattttaataaaaactttttcgagttacctattgagactgtaaaagaggactaccactatgtagttagtaacgctttaggggagaagaatgctcggacagaacatatcgaagcactgcacaagctaggatacttacctataaaagtaagagcccttccagagggtaccttagttcctatgcgtgtacctgtgttaaccattgaaaatacacaccctgactttgcttggttaactaatttcttagaaacagtattactatctcaaaccttcttacctgcaacagtagccactactgctttagaagctaagaaaattatgactaagtatggagaagaaacttgtgataacttagaccatattatgttccaaggtcatgatttttcggagagagggcaacactctaatgaggcttccttgttagcaggtatgggacatttagtaaactttgcaggttcagatactattcaagctagtgtgatggctcacaactactacagtgctgatttagagaaggaactcgttcttaagtcggtgacagcatcagaacactccgttatgcaaagctacggtacagatgaagtagagacattctcaacactaatagacaataaccctgatggtatcttgtctctagttagtgatacgtatgactattacggtgtacttacagaagttcttccaaaacttaaagataaaattatgaatcgaaatggtaaactagtaattagacctgatagtggaaaccctgtagacattattgctggacgtgactatatagagtttggaactaattggtttaactatcatataactaaagagaacttagtaaaagcagtaaccactcgatacgagcagtcatgctcaaacaaaccctcaataggtcaaagagtcgatattgtagactacctaaatctatttgtagatagcgactatatttataataatgatattgttgataaagagaaaaaattagtatcagggtattataaagtaacattaagtgccgttttccttggtgaagacctgtgtaacccttataaagtagacaaacttgaactagaagattaccaaccaacccctgaggataaaggttctctacaactactatgggaaacctttggtggaagtgtaaatgagaaagggtacaaagttttagacgagcatattggtttactgtacggggagggtattgacttagaaatgttagagaagatttttactcgtatgggtgaaaaagggtttgcttctagtaacgtagtatttggtcttggtgcttatatgtactccttctctctttccagagacagttttgcccaagcaataaagagccaaatggtagttattaatggggaagaaaaattaatctttaagaatcctaagacagatactaatcatattaagaaatctcttaaaggaagagtgtgtgttacaaaaaaagaaggaacatatgaagttacagatgctttaacagctaaagaagaagccaaccttgaaaacttaaacgagctgaatgtaatttttgaagatggtaagttagcctatcaagactccttccatactattagagagcgtttagacagagaagcaaaaaaatattagaaaaacgaaagagcctttagtgggctcttttttgttgacaattaagtaaaagtgtggtaagatactagtataaatcaataaaggagatgttttatttgctagttaaagaaagaaaattacgtacctcaaaaaatagtatgctagattttttaaaggagggggagtttgtaagagccttcggtgacttatggtatagaaaagataagaaactatataatgttacacttaaagaaatatttgacatttatctggagcttgacgtttctatgctagaggctactatgctagcttttagcactaaagaaggattctcacctaaaaagtatgcgttagaagatttaaaggaagaatatggtactaatatagtaatcgactttactcttcctgagaataaagaggactttgtagtagataatacaaaaagtctgcctctattatgggacacttacagaagttatatgcctaaaacccaagcagagtttcactacaaaaaacctatttctaagaaagtgttgaaagaaacttgggggcacgtcctagacacaggctcactaagtgaagttagaaagaaaatggttttaggtacaactgctagcattcgaggtctacgtaattcggtaagttcgtcaagaataaaaactagcggagaactaaccgttacctctgctaaaatggattttaaaacaggagtacttacttccgttgttaagcgtgcaatggtagacgaatacgaactatctgcgaacgaatatacagtaaactttaatctttataatgctatggagcttattaatgacatatcggactataaagtattagacaaaggacacagagaccattacgattctaacatgatgtgggagattgttacaaagagtataacccatatatacaaaaatattgaggataagtacaccctcttaaggctagtatcctctcttacctcaggtgtgcaaggaagatactgtagaagtgttcgttatgagatagaaggctcagagttggaacactcagaagtacctatgcctttagtacgtgaagtagtagctaatctaaatttaggtgaaaatatacttgacagagcaggaaactggttttaccaagacgtagtaggcagagaaagtgttgcatctacattaaaaacaaacaattatcaagagttagtagagcgtgcaggttctttagtagcagtttttcctcttttcgacaggattgcttcttatatgaacaaccaaactaaaaaagagcagaagatttttgagagagctattgaacgacattggctaggtggatacaactaccttgagcgcagtaatttaaaaattattggtaatgcactagtacccaatgaagtattaagagacctactcacttataatgcagtacaactcgtcattgagaagtttacacgctaccaacacaatgtggttgcttacgattacttaccagataaagggtatgaacaggagattgaaaaaatacttccaagccttcaaaaagaaagtaagcggattggagaaaaaattcctaagtacattgaggatacattccagaacataaaggaaaatatggtagaggatgtaaaaaatgtacaaaataagattacacctctaaagaaaccatgggaatatattatccatgttaaccaaggtatctggaaaaaagtaatggaattagggaattttgagaaagagtcctatattattatctggaaagctttgtacgaaaatacggaagagaatgtaccatttcttaaatataaatccaagatagaaaacaagttactattcgaaagctctgttttagcaaaccctgctatacaatcatcatttactgaggcatttcgagctattggcgggtatcttgagagactaggaagaacacctgatgagtttatcaaagtcattaactacttgtctacagactgtacattaagacagagaatgagtcttagaaacgcaacagtccaatatagagactatattaattcctatgtagcattaaactcattagacttgaacgaggctaaaagattcgatttgtgtcctttctccttgaagttggctcatgatattgcaacatctgattaccaaactatgcaggattctagagcggatgagtttatggtagcatgatacaaagttatgcctgacgagttcagcttaaacaacagaaaattctctattgataaagaggaatatgttttagtacaacctaaaggagtagacgacctaaaagcggaaggaagttctttaaaccactgtgtcggaacatatgcagctagaattattaaaggggataacttgatttatatgttacgtaagaaagacaacatagaagagagctatcttacaattgaaatccttgtccacgatgactcccgagtatcattaggtcatctacagggagagtcttatcgcagtaagattccaacaaaaataacagataaattaaaggaatttgtggttaaagtaaatgaggctatgttagctcaaagagaaggcagaaattttactaaagacactaaatccgaaactttagttagagccagttaacttgtatttaaactcttaaattatggtataataaaggtagaggctagctaataatatagctagccttataattatactgaaagaggaaattaaatgaaaaaagataatactcggaaaaaaaggttaaacatttataacaccgaccgatattttaatatgaaccttatgaaaagaagtgctattgagaaaagtattaaagtaacacggttgaacgagaaagaaatcgaagaagaaatggacaagctagcagaaggttctacccgatttaacaagaagaataaaaagagctacttactctatcaagagaaatatacaaatgatagactaattcaaaagctctttgctcatggaggtagtgttacatattacactaactctatcataccttactatgtaataaaacatatggcacagtccactacaagtaaagccgtatactctgtaaagaaagagttcacagaagaacaggtaaaaaatatagagctaaccaaaatggctactacagtgataattgacgttcctgttgttgttcctgatgttagcccctatgatgttctgttctcactacaccctttaaaaacagatgtagacggtgtacagttttcctttccaagattaggtaaaagtgaaatacaaaaaaggcacctaccgttctatgagttgaaagggaaagtatacgaattacgacccgaaattaaatatgaatatgtaaaatacactcaagtatctttatctatttgggggatgtatctctggttagtctgtaatagtgaagaagacttccaagaaatagacaagctactacaaaaagacctaaaaagaagagacagtggcagacgacctagtgcaagtcagcaactaggagggaaaacccatgcctaagaagatgataaacagaacagatatagcccgtgaagtagcacaaagaacaggtttttatatcaaagatgtagaacagatactagcagaaagagacaccgttattatagatgcccttcaaggcggagtcactaagattaaagagcacaaactgtatcagattgaagtagaaacaaaaccagcaaagaaaaatgcttggaatgggttagacaaagtatactttagtataccagagaagaaaataattaagtttaaacccttagtagcagtagaagaggtaatagatagcttaaataaaaaagagacgtaatagtctttttttttattgtacttgacaggataatcaactcatgctatactaaggtataagaaaataagaggagaggtgccatgtgaagatactttttttacaagagtacattagggtaaaccacgtccttgtacaagatacaggtacagtcaatatttttcttaaaactgacttagggaagaagctagtacaaaagcttaaatcagtaggtataaccaaacaagacttttatattgactatgcttatgctaagatacctgaaccaaaaaaacaagtaaacggtaaaatactaacgtacaaggagccaacagcacaagagaaaaaggaaccacaaacattactaacagagagaattatacaagaaaaacctgatttaatcgtacctatgggtaacattgggtgtaaataccttcttaatgttgcatccattaggtctttaagaggtataccagaaaaggtgaccttacaatcagagcaaacaggggaaaagtttactccatgggtaatgcctatgtattctatggaatatctaatatcaagtcctaacgtagagggtatgtttgatgcggatttaacaacactcagtcggtacaaaacccaaggagacgaggcgtttactcctgcggaagtatcttatgagctagttacttccttagaaagagtacaggatatttttgaaaatgaagttaaaaactatcctgttattgcatgggatttagagactaacaccttactacctgagatgttaggagccaagcctctagtcatgtcagttagttggaaagaagggcaaggtgttactattccgctagaacaccgagagagtcattgggaagcctatgaacttgaagagatttataactatattaaagaatttgtagcagataaaaatattgttaaagtaggtcacaacatttaacacaatgggtgttgtaaaactcctctccatcggtgaacgctaagtgcaaagcatatgccaataccgagcctacattactggtgacagtagatggggtgtagagactgaaccgagccattgtagggcaaggtgagaagtggtacatatcacttaaacgaggagcattgttccatataggtaactacaaataaaacctattggtaaggggcataaaggtatagtccacgcttatatgaaaatataagactacgtgcagtatgatatacgttttcttatgtcgtctaaagggtttactaagtttaataataatcgtgatacgaagatagggtactacttgtctgtaacacaacagatagaggactccttcaaattatcagatttagcctatgagatgacagatatgggtggttacgacaaaccacttgccgatttcaagaaaaaatatattaaagactacaacgataaaaaaacagcagaacaggaagaaagaaaaaagaaagtagtagaggaaataaacaaacagtttgtagaagaaacactaaaatttaaagaagatactaagaaatacaacgaagaaaaggcaaataataagtattctgtactagttaagcctgtaaaacctaaaaaaggtaaggcacctacaaaggcagaactagcttacgagcctttagctaacgaagtagatggaggaaactttaactatgactggattcctctagagatactacatccctatgctagtggagacactgactgttgcttacgtattcataatgtacttacggagataattgcaaaaaaccaaaaaatgtggttccagtttgctaatttttaccctagtgctattgtggcattagctagaattgagtctaacggtatctatgctcagacggactatatggaagatattaatgagaagtacactgaggaggaagaacgcctcgtagaagaaatgcgtaagatagcagaggtatccgagttagaagagcaacacatggagttgtacgaacgcggggtagaggagatggcaatacctcctgcaaaaagagataaggatatagctaagttaagagacaaatacaagaagaagctaaaatttaatcccaactctgcaacagacaaagggaaagtcctttatcaaattatgggtattacacttccctacgataaagagcatattaaagagtctgcttttgatgtagggaaacctgagaataagctaacatgggaggactacaaaacagataagaaagcactaaactatattgtagaccattttcccgaccaaaaggcgtttgcttctctcctactagagcactccttagtaaaaacaagaaaaaataacttcacagccaagcttctaaacttcattagtaataaggacggaagggtacatgggtcgtttaacgctacaggaacagagacaacccgtttaagctctaataatcctaatatgcaacagctacctgcaaaaacttccgaccctaaaaggttcgactatcagtacccgataaaacgtatgttccagactgcatttgagaatggagcactactacaactggattactcttcactagaaatgcgtatcttagctttagcagcaggtgacgaagctatgacacaagcattctttgatggggcagacttgcacaaagaaactgcttctattgtatggggaatgcctgtagacaaagttccagcagaatttcgtaaaaaggctaaagcagtaaacttcggtattgcctacggagaaacacctttcagttttgcaccaaaacatgatatgagtgtagaagaagcagaagacatcttccaacgttattttgcaaataaacctaatatcaagacatttattgatgaaacacacagagaagctctacgtgacggttttgttgatacattacaaggtcacagaagacttatccgtgatgccttttctaaagataagcagatacaaaatggtgctttacgtaaatcagttaataccaagattcaaggaacaggtgcttactgtacaaatatgtctctaatttatatcgaccagtatatacaggataacaatatgaaatctaaattagtattaactgtacatgatagtattgtagtagactgcccaccagaagaggttgagattatggctcgggtgtgtaaaacaattatggaaaatctaccaattgacttcttgagtattgaatggaaaggtgaaaaaactaagtatcctattgaagcagacatcgagattggtactaactataacgatatggtagaatacaatcctagtgagttaaaagagttcaactctttaacgggttatgtgaagtactacaaagatttagggaaaatttccgattacatggaggctggtataattacagaggagagtatggatttaggacaaactagaattaaagaatcaaaagaaatttacaaaaaaattgcctaaaaggttgcttttgttttaaaaatagtgtataatgatacagaaagctatttctaaaaacagaggagggtaaaatatgacttaaggagggatttgtttggacatacatgtagattcattagaatttagccaattacgaattatagatggtaacggacaagttacagagcttgatttagagagagagttgagagtaaatgaatactcgatgcaggaggagtttctcaaccaagcaccaaaatatatctattgggcttctattctagagaaagtacgtatgtatcaggagtctgcatcactagctttagaggtagtgggagcacagatgaatgagaaggcaagagagtattacaagactgtagacactaagccaacaaaagaccaagtagaagcctatatacttcaacaagaggaatatcaagtggcttacaaacagttgttaacctataatcacttggttaaacaactacagtacgtagtaaaagcgtttgaacaaagaaaagacatgctaattcaatacggagcagatttaagaaaaaataaagataacggtataagcccaaacttcgggtaaaaaaaaataattataaacgataggagacaataacagtatggtaaactttaaagaatttttaaaacaggagaaccaagaaaacgaaagtaataataatgaagaggtagcatttccaaaaggaaaacatcaacgcttattcttgtctaaaaaatcaaataccgtggtagttcgtattttaccaccagctaatttaacagataactatgcacaaaagtatcgtaaaattatgctgaacaacgttatgacagatacaggaaaagcattaaatccgtcatttacgttaccatgggagcctaacgaggattccccactagaacaggctttagctgattggtataaccgtggagtagttcctaactactatggaggaaaatcacgaccaagcaaagctactgtagtaaacgctgtggcattagtaacagatgcaaacgggaactgggtacatgaggttgatgaatacggtcaactagtagttcgagtgttggacttaccttattcagcatatacagctatcaatcgtaaattagcagacccaatgctaaacactacaggctctgactatagctttatctctgatgtaggagctaacccaatccgtattactaaacctacatcaaatacagagaaagcatatcaagttgaagtatatacaggtattacattaccacctcttcctgagaattggagagaccaattggaggacttagcttatcaagttagcccgtcagaagactataacagcaaatttgttaactttgttattgaacgagttaatgctgtaactagtggaggagctcaacagcaacaagctaacccttacggaggacagcaagcaccacagcaacaaggtggttatcaacaacaagttcctcaacaacaagcttaccaacaacctgcccaacaagcacagacgtatcaacaaccagtacagcaacagcaagcacaaacataccaacaaccaacacaggcgtaccaagccccaacacaacaagtacctcaacaacaagcgtaccaagcacctgtgcaacaaccaattcaacaagctcctgtagcccagccacaacaggctcctgtacagcaacaaacgtatcaggctcctgtacaaccacaggcacaacaacctattacgcaaggaatggtggactcagcaatgagcgggttagcatctcctaaggaacctgtacaaacaacagaaaccagtaaccccaccaccatctccacaagcacagcaaccagtaagcaatgattcaagcttacctgatgtagatgagttactacgcagtatgcaagaaggttgataagaactaacatgaggtctaggatttattttctagaccttaataagaattttagagagggttgttttcatggcaagagctaaacagagtgaaaaagataacaagacagaaattgatttaggtaaaaaaattgggttaactactttgcaggatagtaaatgggcggagatttcagataagttacctacatttatccctcagttagactatattctagggggagggattccttttggtcgtttgacggagattatgggtaaaaacgcaagtggtaagtctactcttgcagtccacttaactaaggtagccttacagctagattgtaaagttatttggattgacactgagggaacggcagaccctagtaggcttagtcagctaggcgtagatgtaaacaaaatattttttgttcaaccaaaaaaatctaagaatgctgaggtaaacaaagaggattctattacagtagaatttgtagcagaacgagtagaagctattttaaatgccttctctagctctgaacaacgaattgtgataatttgggattctattggacatacaccttctatcaaagagattgaacaaggggtagcaggtaaacaaccgggtattaaagctaaggcaatggctcagtttgctaccattattgctcctatggtaactaaatctcattgtgcctttattggtatcaaccaagctagagacgaaatgggaagcatgtttggtgcagtagacagccccggaggaaatgcactacaccattgggcttctctaagactagaggttaaacaggcatccaaaatagaagaaccgatgcaaaatgcttttggtactatggataatgtatatgtaggacataaattacgtgttattactaaaaagtctaaggtatctacacctagacaaagtgcagaaatgttccttttatccgcatcaggtttgcaatatgaagagaatatttatcgttcttgtgtggacagtaaaatgtacggtcttatcaaaggactacaatacaagtcttatattgcagatgatggaaaagaatacaaaatgtatggtaaggactgggttcctttcttacagtcaaaagacggggaaaagatacgaaaagagttattccagaaaatgatgttaattagtttccctactggatatagtccacttgataatgaggtagtggatgttaacttatgggaagatatgaaggggttaacagaactgtacaaaaagaataaagtaaagcaacctaaggtgaacgagggagcaaaagaagagtctattgaggaagtactagcaggaattgactcggataaataactttataggggagggaaacttccccttttttcttttagtactattgaggaggaagtaaaatatgagagctaatagaaatattgatttaaccgagcatgtggaagtttggtctgaaaaaacaagaagtattccaccctctaagttaaatccatttctagatattaaaaaaagatttctatatgcaaaagagcataatatcccaattattctagaaaacagcgagctaaataactctcaatctgtggttctagacctaagctttgtgggagataaatgggcaatgggatacgtaacacattattcagctattcgtacagagtctactccatacactatccattacagtgatttgtattgtgcaggtaaacaaagtaaaaagaataaaagagtaaaagtaatatttaaaggggataacccttatactatggaggaagcctgatgcgtaatattgataaagaaaaggaatatattagaaacggcaatcgttatgtacaagagaacagtgggagaggggtatttgctagagatagtgaccgactattccatcagtacagtaatctacggtggagtgtctacaataagcataaagactccttcaatgacgaaactactagagcagaattacaaagctatattgacgagcagtttattaaactagtgaaggagtacgaaattaacggtgcagtagacttcccggggtacattaaaacaaagttaaaccagagagtaaagcatacttttgttaagaatgtattccgagacaaagctagagaacgtctaggtgctgatgagtttgaagtagctaatctagtagaccgacagcatactgatgaagcactactagaagaccagatgttacaagagtatgtatttggtaagacagagttcacacctattcaaaaagagttgcttgtctatttactagaaaaagggatggtttctgatgaacgagtaattatcagaaatgtggcatctaaattcaaagtgcctagagaaaaaataaatgaggaattatcggaactacgcttttttgtttcttctaagatagctaagtatgagtcagaaaaaggctattaatattgaaataacgtctgctatattagagttagaaattcagtataggaggctttaaacaatgacagaaattcatacaccgcaagacgaagaagtaaaagatgaagtagttcaagaagttattgtagaagaacaacccgagaacgtagaagaagtagtcgttcctagtgacgatgaagtaaaagcaaaatttatgtcttatgttccgttatttgttactttagttcttttcttgaacaccttattagcatcgttgggttgggaaccattagcagtttctcaagaccaattgtatctagtaggttcttctgttgcaacagttattgcaactgtatggtcttggtaccgtaataacaacgttactaaaaaaggcttgaagagagaaaacatcgcaaaccaagtattacctaaaaaagaggacaaataagaaaggatggaatattaatggctattaagaacattaaagatttgcttaaagagtctacagtcgttttagatgggaagtatctacaagctagaacggtaaccgatggttttacaggaacctatcaaggtaaccactcgtttaaagtagaaaaaacagggagtaactactctgtataccctatgtattttgacaaagatacaaaacaaatggagaagagaaaagacccgattatctatacagatgacggagaaattttctttgtagttcgtactacagtagacccttacaattacccagttattgagcaagactccttaacaggaaaaaatacagagaaacagattctacaagcatttattgctttctctgctgacgagtatagttttgctaactataacttgtttatcaatgacgatttaaagttctatgacggttctgctataaaagaagttcaaagtattaagttccctaatgcgacagaaacaataaattcttttggagaagataaaacattaactgttatctttacacctgcggatgctagcgataaaacagtaacattcacaagttctgacgagagtgttattaaagtgagcaacacaggtaagttaactggctctgtcaactccactgaaaagtctgctgtggttactgctgaaacttctaatggaaaaaaagctactgttactgtaataatgaaagcatcagagctaccagaaccgtgataaactcatagaaaagaaagggactttcgagtctcttttttttattgactttacaggtaaactgtgttatagttacatagaagatacactaagaaaggagtgacaagatggaagacaattttgtaacagcctataataaaggagaacagacatgggatgacttagcattttgggaggagtcttggaacaacgaccctaatcatattgccagtctagcggagtatctagggatgacagatagagactactggaattatgtagtaatggacaaagaagtacctaaagagctcctacctcgaagtgagcaaccccaactagaaactgaatacatctaccacatttgccataaaaagaatgaagtacaaatttttaaagaaggttctttagtctctcataatggaaaagtggtacaagtagataaaataatctctgtaatcatcacagagaataactcagtagccattcatcttaagattaaggagggaactaaatgattcagctaattacaaaaaataacatgcagggatactatgcagactatctgttacgaaaagtatctacacatgaagtgagaacgacttacttagagaagttcagactgcaagacatcactagttatagtgcagatgctactattattatcgtactaggatttagctacagtgacagagaggcgggagctactgttagtaacttcgaagagtatagtgcaacatttaataacccattcactttcttttaccactttgctagttttggtgataagtttaaatacccaaatattgactctctagttgacgaaaaagtaagtcctgttatggtaatgtacaacaccttgaaggaaaaaaatcctgatttcttaatagcagagaaagacaaagaagctatgttggttgctactgctgtagacgactaccactgctaccgttttgcagaaacagggaatgggctgtctctacctttaaaagagctaggagacctctatggacgttatttagctgaattgtacggaaacatgtctttggtggagctaattcagtcaaacaagaccattatcaaagtgagacaagagtatcggaattattacatcaagaaaaaaatagagcagtctaaacgacaacttgttaaagatgttatgctttattttgtctatgcagaggaatacgagaatgagattgcacaccatattattgataaggctaaagaagttaacgatagggtgcttgtcttagtaggaaagcatacaacaaataatgatatgttccatgtacgctcccataatattcacgcaggagaggtagctaagctaataaataaaggttcaggtaaagaaagtacagcggtggtattcttaggggattccaaaacacctacatttaatgctgttgtgaaagttttgctagatgctagtgatatttaaccactaaatgtggtataatagaagagaagtacacactaattagttaggagaaaataatttgaaaaatttaacgaaggcagaaagaaaagctattgagaaaggacacttaggaaaaacagccgttattctaggagtgctaaatgcttctcacggcaaaattgttatgtctcagtataataaaatagcggaaagtttaggaatggaaaaggtagctaaaccagacttcttagcactaaatgaaaccattcaagaagataaggttttacttggactgtaccaaaaatcgtctgtaggaaagattacactagatgatttaacaggaggaagtaacgaagacaagtatagccagtttaatgctataacagcttatacagtagataatgacaaagtattagcagacttacgagaatttagaaaaattcaacgagatggagcttatacacaacatctttttaacggagtgaaagcagatttaaagaaagagctctcttctattgctaaacctaaatttattattaaagagaacaagaaaaaaacaggtaaagaaatgattgtagccttatctgattggcacatcggagctttagtaatcaactctgatacaggaggatacgactacaaaaagctagtaagctatctgacatatttactaggtgaggttgttcagacagtagaagagcatgatattgacgtatgtcatgtatatcatattggagattttattgagcatattaatatgagaagtgtcaaccaagcatatgaagccgagtttaatgcaacagagcagatttctaaatctattcgtacaatgcttgacttcttaaacgtattagccgaccatgtacatgtaaaatttggtatggtaggcggtaaccacgataggttccaaggaaataaaaatgaaaaaatttataatgataatgtagcttacattattctagatacattgtttttactacaagattccaaagcgctacacagtaacattacgctaattgataatcggaaagatgtttacagctttaaagataatgttgcaggtaagaatattaaagtagtgcacggggacacagaaggtaagaaagtagatgtaaaaataccaaaacacattaagagtgaagtagtagactatctattcatggggcatatccatacaactagaattgtacaagaagattatagcagattccacgtatatgtaggtagcccgatgggagctaattcctattctgcggaaaataacctaccgacaacacaaggttcccaattattagttattctatctaaaggtagtaacaatccaatgtttttacctatctttactaaataaaggaggaggaaaacaaaatatggcatttttaatcacagtagtgctagtaacgtaccaagttattgtacttttgcaggcggttagtggaactatccgtaaagaacgaattatggtagaagaaggcggagaccccctaccaacatcctcctatgttttcttaatcattaatcaaggagctattgcaggattcctattcttgtggggggcagtactaagctttattctagctatggttactaatgttatcctagctgtaatactatttacgctagcagtaaccatcttatcaattgtaagtgcaacgattctaagtacaatcgttcagacaattgcagttattattgtaagacgaaagcagatagcacatacgaaaaaagttattgctgaaaaagaaagagagtagataaaatatgtctgagtatgaagcaagcagagcagacttaagtagagagttcctacaaggtgatttacagcctagtattatgagacgagggattctctacatcagaaagaaacctgaatatattgttgagtatgtagaagaggctagtagcgatacattaatggtaagtgtgtttgcagttcaaccaaagtctaaaccgcctttaaaggcttggacattagttgataaaacattccagtataacacaggaaccactctaggtttactagctaaggagctgaaacatgtacctaaaaaaacagagaaaagagaaaatccccctttgtttatcgcacctgcactagatggtgtggacacctttacaggaagagcaggtaagggtttctatgaccgagaacctgatagagccacaagcgacggtaaaatcgttcaagggaagcacacaggtgtatttgttgggtttgactctattacttgggagaaagctattgtgctaactgaggagatagctagtaggtatttaactgcccgagcccaatggtataacatttagtaatgctatattaaatagcatgttaacaattgttaacaggagaggatggaagatatgacctttacagttgttttattcctagctgtaatactcttgtttttatccgcattatgttttaccagtcataaacagagttggaacgtagtaggcggtgtactattagcctttggtctaatcgcattgttctatgtgcttataatggcaagtgctgtttcaggtataaatggagaatggtttgggatgaggttataatacacacattgggggctctgtaaggggctcccttttttatggaggttggaatgaaaaaattttatgaagatgatattaagcaggtactactaaaaaaaggaaagatgtttggtgatataggaaagagcactattgtatttgagaaaggtattactatctcctctacaatcgcagactgtcttatatttacagagaaaaaaggagtcattggtgtggagataaagacagagagagactccactgcaagattaaataagcaactaaaaaactactccttagtgtgtgattatgtatatgtaatgtgtcatgataaccatattaaaaaggtagagcagataatgaaaaggtaccatcatgaacatgtaggcattatatcttatattgagtttatgggagagcctgctattggtgtctataaacctgcaagaaagtcaccgttgaaagatgtataccacacgattaatattctatggaaggaagaaatactaaaaatattaggtgcctttagacattatggtgcaagagctcctaaaagctcctacacagtagcagatagaagcggaggactaaatgggttatccataaaatccagtttctctgctcgtatgagtaagccagaactaataagaaatttgattgctagggttggacgaccagaggctaatcggattgtttgtgatattttcattgaacatagaaacaatcctgccaaggctataaagtaccaccacttctttcctagcaaactagaaaggtaagtgaagaatagtggcaaacaatactagaggtaatctacctagggggtatactgcactaggagacgggagtgagtctcacaaaacagtagaaagagcgctaagaggttactatacagtagactttagtaatatatacgaacacatgacagaaagagacttagaagtacgacaggcatacgctaaagacctcatatctcaaagaacaggcatccctttagacatgattttatttagaaagaagacagagaagcagacattaacagctatagatgaagtcttttatgtaaaacaaggggtaaaaacaataggaaaagtatctatcaggtcacaaagactactaagcagtaatatgctgattattatttacatgtctaagagccatatgcctcgtgtagttagaaacacaggaatgaaaacttatacaaaacgtagtaagtcagacatacgtgtagcaaaaagacaaaggaaaacaacaaaaagacagagctattgacaacagagtctacttatgctatgctaatattagaaaaataaacgaaatgaggtacaagtatgcaaatagtagaaaaggagagttccttaaacagctttttaagcagtatgagaaatgaaaaaggtacagtggtaggggtctattataaaaatgcaatgatagacgaaagtatcttagcagaagccagtcttcaattagaggtgcctaataagacggagaaggtcttacaagccacggtagcactaaaacctatgccaagtcaggtaaaccagttaacgaccttagagagcctcctagaggactctggaatgggatggttttttgatagagaacgacctatcacaccaacaatgagtacccccttgtttaacgacttagctattagtcaaagtgcactatcaaatatacaaaattatagactagtagttaacatggctgaccctaaagtaacaaaacaaacctttactttttataaaaatacctatacattgtttaaagtagaggacagacagttggatagtaaggttgcaggtaacattattcacagtatccatgtcttatataaggaagagcagtatgacggtgaaaccttattcggtagattgtttgtagaaagtggtagtaaagaagctaaacttatgaagagagatactaaaatcttgttggacttgttatgtgatgaaaacatcactagtctttttattggaggaatagagttatctttagaagaccgtttatccaatgctaagcttttccctctgtacacagcagttacagctacaagaacaaactatgatgcttatagattcctaacagattcaggagagactactattgtttcggatatgattctagagagtaagttagttaaagtagctgactataagtttacctttgtagtaaaattaaaaggaaatgcacaattaactatatatttaggatagaagtaaaagaagtcagcatttaattgttgacttttttctttatatatgttatgatgtagttaataaaaaagaaaaggggttgacagaagtgacgaattttttaacattagtatttattctagctattattgtagctgttcctacgttattagctaaagcgatgagaaaccactataccaacaaggagacaggtgtttggtggatgccatatacacactggtttgtgtacaactttttatatacacagaagctaggatggctaattttaggctccggtattattatctccatttatattccatttattggtggatttttaggaacagtcattggatttgcagggggactacttatgttatttggtgggctaccttatgtatttggtttactagggggagcagtctttggtgtttttgtagacaacattgtgtatcatctaccagaatggtatcgagcttggtttagaaaccgatttgggattgagtttccttacctagaagacaaggaagatgaaaacgaggagtgaaagtaatgaaaaatgaagatacagcttggagtttactaggtttaatttcagctattgccttggtagttatttcttttctattcttagcagggtgttcacaagagacatcacaggttaaagaaacagagcaagagaggtatgagaagttcactcctatgattgaatatatttactctcagaagttaattaaggtgtataggaatgaatcagacgagcttgtagagtacacctactatgcccgcacagaagaggaagaagaagaagggctcatccacgaagaggagtataattattacaaaagtcaaggagttactgatagaggtgttattaaaacaacaataaataggtatggtaagggtaagtacccattaaaactatgggatgtgcaagagtacgaaggaaatgtcagcttagatagaatagataatgaagtctactttgagggtgattaatatagaaactacaagtacagtaggatactggaacatagataccttttcgtatgaatatttgtactgtgctacagagggagagccttggagcatagggactatgctaacggagtactacactgattataaacaagtaaggcaactaatgcaggacaaagagtttggtaataagaagtttagcttcacgtcttcaaaggtaagcaaggcaactaataaaaagaagctaaaggacaaagagacctattttcagtatatatttgaaaaagggcagtggttctgtgtgtatactaaagggttcatggctaacgtagagatacctgctatagaaaacccataccgatttaataagtggttagacaagaatgctaacaagttaagtaacaaaagagtaaaactgttacgagaactagttgtaagtagcagtgaggttaaagaggaaacagagagagtagttcctgctatggttaaattagaacctccgttacgattattgctagacgacactcatagtgtcagttctcataatatactggaatttttaaagtcagggaatttgtactctttagctaagagagagttctatgttgatgactatggaaccgaagtagactgctttactataaatgagttaacaaaaccaagttcccaagtacctagtcaaataacggttagtaaagagcatacatctaattggtatcctctattagacctacttattgactgtggttatatatcagactatgtagttgaggatacagggtctaatagtaaagaagtaaaaatagagataaaagaaggaggataacgggatgtctatatatgagtctatcgaggggctagaacaaagtattatagagggattacgtacaactttagaaacagaaggtaagaatatagccctagacgttatcagaaaagaattagaagacagcccgttcttacggttttttgagttgtctcaaggtagccctgcgggagatgtataccaagtagatactactaagatagctagtaaccctacacagcattacgaaactgcccgtgtagctagtatcgtgtctgctaacgttcctgtgtggttgtgggggcaggctggaacagggaaaacttctattgctcaaagtattgccaaaatgctagatttagaattttaccctatgcagaaaataatggacgagttcgaacttaaaggttttgtggatgccaactctagattcgtaccaactccactgtatgaggcaatgaaaaatggtgggttgttatttattgacgaaatagatgcttccgtaccagaagcactaatacttattaacactgctttagctaataattttatgcgatttgctgacgagactgtatatgcacataaagactttagggttattgttgcagggaacactctaggtaaaggaggtaacaactcctacgcacgtaataaaatagactcagctactttagacagatttgtacctattgagataggatacgatgaaaaaattgagttattaattacaaatggagaccgagaattagtatattttgttgcactagttcgaatagagatggctagaacaaagttagaccatgttatctcttacagagcgttaagtcattataaaaaatttgagtcttttggtgcggatttcttgattgaaaatgttttgtttaaaacactatctttagaggatattactagtgtagctcgttctcttagcagaataatcaaaaaaacagataaggattctaagtggaagaacaacaaagtgtggaagcagttacagacatacctttcaaacaaaggagtagttgtagatgacacgaactaatcatacgtattcatctaaatttatagcagaaaaccaaggaaatgtctataggttgcactatgcaggagaggactatacaaagataatagatgatatgtcagctatcccccactatgatatagataatatcaaaaaccctcaaatgaaactctcatggtctacaggaggggaacctattccagaatttatacacaagttccaaacaaattatatggaacctgagaaggcacaagagcttaatgtaatgctctctaaagtatctgaattagtaagtagccaaccacctatttatcatttttacggtaatactatatcagttcctagagccattcaagggcaccctaaaagttttaaacatcggaaaaaagaagtaactccttatattagtatatttgtagctatcaatgttctttatgacgttagtgtagagaaccataataagtttataaaaaatatgttgaaagttattttgatactgtcaaaagaccatactgtaaaagtaacactaggtaaagttagtgcctctaggggaagaatagctgtggagagctttgtactaaaagaggctaatcagagttttaacttatctagactttggtaccctttatctagtatagccacatatagaattgttctttttgagatgctagaaaggcatatgacactagagcctaaactatcctcttacaaaggtaattatgggtttacaatacacagtacggaaggaatagcagaagcagtaaacttgtttgagaaatacgactatgtagtcggttattacgaaacaataggcatgacagaaaaagaaattatagaaagatattttgtgtagttgactttatctttcatatatggtatagtagctatgtaaagtacaaagaggaggtaatactatggaagtagcagagttagtaggtaaactggtaggtaaagaagttatgattagtacagaaaggcatgagcgccaacaggtttatagttttagaacacacttaatagaacgtgttgagttgaagcctgcccctaggacgattgacaataaatcagttattgggaatgccttttttgtagtctatttaaaaggactacctaatgaatatcgactagaacagttaagaattagaatggatatactagacagaggatgcttattacctgttgagattgacacagaaacaggagaggtaacacttgttgtaagcgactctctagctaaatatttctttgctagtcttttagaagttgaagcacgtactgacgaggacaagaaaaaggagctagtacaggagtttctagcagtgtctaaattatttcacggtggcgggtggactaacggtaactattatgcagaggaggattaaaactccgtatataagaaatgaggtgaatatgtatgattaaagtagataaagtagaatgcaaggatggaagagtttactatgtctataatagtaataatggatataataaaaagaacatatgtattaatggaaaggtgcaaacagtacctaaagataaatatgagaagtatttacctaaactttcagagataaaaaaactagatacattgtatatgagtaacaaaaaccaactgtacaaagaagttgtatgtgccagcagtggaaatacaagtaataaagacaaaaaggtacgaatcaatataaacaatgaagtagtcagctacctgccgaagaaggcaaataacttggcgatacttctaacgcctgacaattgccatctggttagtagggtagagccatctatactagtagaacttctttctaaaagaacagtcattatctctagagatggcggtggggggaatgtaagaagaataggttctgcttgttgtggggacgtactaacgttagcatataaatatataaatggtaaacagcaggtaagtattaatgtactatctgttaaggatttttaccaatatgaactaggagggttatttttatgattaaagtagagcatattagatataagcctgtagattttggcttgtccagtacaactcgtacatcagaacactacgtatttaaaagtaagagaggtcaaaaggtggttacaatgaaaattgtgggtaatggtattcgggcaatgccttttgctaagcttaaaaagcaccatttagacaaggttactgtattgaaaacattttatgtacctaatcgaaaacagctagctaagcaactaggtttaagttactacaatagtgggaaagcagtacaaaaaacagtaaaacgtttagatgatattcaggactcgattgcatttagtaatgctattcaggcactacctgaccactcagcattgagatttaagttagtaggggaggagaaaacccctaaaactaaacttaacacagatggcagtacaaactaccttattagaacttccgctaagctattgggagaggatgtaaaccaaattaacttacttaacgtatctggtgtgctaaataaagcggaggcagaagcagagaagttcttacgagtactaaatctgtataaggaggctaccaaagatgattaaagtagaaaaatatacagctagtaccacaaaaaaagcaggttctattagagggacttatgtagtaggtggtactagaaaccagaacgaatcagatttacacttaacagggactacacactacaagtataaaaacaaaaaaggtaatacgatgattacatttgtcaattattatacaaaagaattagtctctattcctgaaaaggacttaagccctcaagacctagataccttttatagtacagtaaataacagactaactactgtgttctatgtccctaactggaagcaaatgaataaaaatgtactaaaaaagggtacaaaatagcaaaattataggtagttcataaagattttatgatactacctagtactgttttctaatctgtttcacttatcttgtttcatttacttcttttatactctagattgtttcaaatgcttataagtagttatgcctagtaaaccctactctctagtacactttatgggtgtttcacttacaaatgagataatacaaagtgtatgctatattatatattaagaacactgggaggcaacagataaccagtaggtcataaaatagtataaatgtagaggagttacatgtgaaatggcagataacaaagataaaaacaaggatttagaaaataaaaaagaggcacctacaacatcggttttacttagactgcatgagatgcctatcttagcttctaaggtaaatgatgctttagatgaggacaagtcgtatgactatattgtaaacttatgtagtcagtataactttgtaatctctaaatcagcaatcagtagatacaaagtaaaaagagaagaatctatagaaacaggaattgatttaggtgctttattagataaaaggcgtaaaaacgaaacaacagctactgtgtctatgattgcccctaaagagaaaaagtcatctcccgaagataagagatacgaggaaacttttagtactgtagacaaggtatacaacgatgtaatggttttggaagagattatccaaaaaggaatggaaggcttacaatacacagagactatagaggcacctattgcgatgaaagctattgaaataaaagctaaaatcacaggaaaccaacttcaaggactaagtattataggactacgtgaattgaaacttagaagtgtagctaaagagacagcaatcacagaaattttgatgcagtatatccccgaagataaacaaggagaagtgcttactaagatgagagaagtagaaaaggaattctataagaacttagacttatcagatgaggatagaagaataactgaggcgcttaaagtcgcaggtataaacagctagaaggaggtttttttatggagatagtagtgccagaaggttatacaaaaataagcatgactaagtttgataacctagaagataagttactagcactattaacaggagagacactgttagtgcgtggtttagaaaaaagtacgaacgcagacgtactagttaagctagatgacagaaagtaccctgtgacacagataagctatgaaattagtattacagacaatgtgtttcgagaacgttattggactatatatgacatatctattaatgcactaagtttgtatgatgttttcctttttgaggaagacaaccctgcatatgagctaaagatacaagtatcagacaatgtgcagtatactagtatcagcggtgttaaagacacagctaaagtaatagctatttatgaagatgaagaaaaaaacttgttttgcgagttatcaagggaagacacattgtacccattagtagaacttacaaaaatttagcaaaaggtaaaatcccttgaaagctctaagtagccgactagggatttttttgttgacattatatagaaagtatgctatactacatttataacatagaaggaggagatttaaattatgaacaacaaaaaagaatttgataaaaaggtgactgaattaaagaacgcgacagagattattaaagagcgggtagcaaaagtaacacacggtctaaaccaagcaacaaacctgttgaacttaaatgtaaaagaagtaaaacgagttgcttcaacactgcaacctctagaggaagttgtttatgagttgcagaaagaggttgacggattagcacagtatagcaaagcggaggttgtttactacgatggaagagaggttaaccttgaggagattgacttagaagaggacgactatgaagaggatactacagaagaggaaatccgagataccgtctctactgatggagggggagcacggattagagtatatcctcctgaggaaactttagtatatatggcagagcatattggacaaggagtagagactgatgaccttgctgtttatgaagctgttcgagacacaaacggtcacacaggtttccgacaagaaggctattatgtagaccctacggaaacatatgctaaccgagataaacgattaaaagaaacctcaaaagtagaagaggttgaggaagtagaactagaagttactattattagaccgaaaagtaaatctaaatctaaatctaaacctaaagctaagaaaaagcaaaatagtaaaacaaagtctaccacaactgagtttaaaaatgatacagaaaaaggtattgtaactgactatattaatggagcaggaacaagtgctattgcacgaacttacaatgttagtatgggtactatctacacagcgcttaatcgtcataagattcctaagcgtaacatcgctaataacagcgtagaggttcaaatggttcctgtaatcaataataagaaactactaaatgcaattattgacagctacaacaaaggagtaccagttaaagacatctacgaaaaatacagtattcataaaaacgggctatactacttgctagattatgtaggtgtagaccgtaaagtacaccataaagggaaaaaaggagacgtagaacttaagatggactaacctccatcttagttttataattgtagttaccgaaggaggttagttagttgaaagtccatttcaagaggtctgtgataatagttgctttagtagtaactagttttattctaggagtagctttaggttatactatccatgtagataatgctgttcaagaacaaatagcattacaaaataaaaaagattcttctactagtaaaaaggaacctagcaccaataaagacattgcagacacaaaagctgtagactatgagactgacgtagctgtaaggttgaacctcctacgagtaacagtaaaagaattttctgagaagtttaacgaggtagaggataatgtacaagtacttaatgatatagaatactataatagttatattgcactaattggagacgtgaaacgccaagttagtgatatacaaagaatgaagtatcgtagtagtgagtctgctttagctagttctgtaaaccaagcagtagacaacttctatcaagcctcctccctagaagaaaataacgttcagtcaagacagacggacatttctactatggttgttaacaagctaaatacaggtaatgatgaattagaaaaagtctttagaataataggaggtaactaaatttgggaattgaagcaatagtatgcgccttatatggtgtttgggtagcacggaacctgatgaccgtgacaagtgttgatgaaacactgacggaaaagattattctagaaagcagtaacacacaagtagcccacttagcaataagtgaagccaaaaaacttctagagaaagaaacaaaagagaaaaatagaaaaattattattgattacattgaaaaatggcatcgggcactaagtgacgatactttaatagataccctagtagagaatgaaagaaaaggggctaggaatgctagaattattgctaatatatttgtaaaaattgggacattacttggaatcctagtattaaatattccgttctttcttgcattagctaacttcacagtagcaggttgtgtggcttttgttatattaggcgtaatatatggttgtcagacggaagcaaagcaagaaggatacaaagcaagtaaggactatattgtagtctcttatgtgctaaagcaggctttattcttgctggtagtgttaatctagtaggaggttttattatgagagaatatgatgaggaaaaagaacgagagctagctaagaagaaccataggataaaaagagaacgagctgaggcacttgaaagaaaacgagagataatagagtatgtaagagaactagataataaggagaaaaagttagtagaggaacaggcaaaagaaaaaataaacggtaagaaaagtagtactgtagaaaagtatggtaacgcactggatatggtaaagaacacagactttgaagctttgtgtcgaatacagacagcttaccacctacagagagaatatcctatggttttaagctctctaaataacaaaggttcttatattcatctaggagtagctaagcaagttgcagataagaaaaccttgtcaggagatgctacttacgagtgtgagctattattttcagttcaagtacctaaagcaggtcaaaagaaacaaaaagatttactactagagctagaaaagagaaaaacacttgagggtaactatactaacgtagaaggtagttttactagtcaccaatacattccttttctaactttcttaaacagaagaaagattgatgcaaagattgattctatgctagatgactactctctagctaaaacgaaggcacagaagaagaaaatccaaggagaaataaatacttgggtaggataccaaaaagtagtaaaaaataaaggatacgttctttatagctatgcaagtgccaaagtggtagcagtttatcacagagaaataagtgtatacagttaaggaggggttaaattgagcggactaaccgtatctgataagcaaagaaaagaaatgctttctatgatagagactctatggttaaagagaaatcccagccaaaggtttaatcaatttatgtttaacttagaaagagagtttgcttcctataaaaagaatgaaatttcaaaacgaaatagagttttctaccaaatgagccacttagggtacaacaaaggatacgtctatgtagcccaccaagtccctgatttatttgatacatctgaacaagacttctattcttatttgaaggataggataggataggataggataggataggataggataggataggataggataggataggataggataggataggataggatagaccttcctatcttttcttttgttctaataaagtagtgggttacagctacactttcagttgtttacaagttgtaggactctctaagtagccagctatagttttatctgtaaaaagagataacccagaccccattatagagcatatataccctaagtagcaggctacacaaaataaccaatctatagataactaatgtcattcagagactatagatgacacagcatccttttgtaaatttatatgttcgggctagattaagaggcgataccccacccctcctcttagagtacttgtagagggggtatacagagtttcgttttcctacctagtatttatatagaatatataagtatcttccttaaaaagcctgtggtggcttgtgagagcatacaggaaggtattgtaccttgtatttatacggtagcaggtaccttaacccctcatacaagaggaagctttctaccccatataaacgataaaacactaagtagcaggctatactttctgatataggacagtatgacttatcaatatgtaaaagggacaaagctctaagtagcaggttacagaaaaaaggaaaaagctctaagtagccggctacagaatttcctaagtagcaggttatagatattaaaaccatttatggggtaggtgaatgcaggtagatggcggggtggtgaggactttgaaataccttgtgaacgttggtgttatgccgtttactgctagatgataaatagtttcttctatataatacaacgaaagtaagctaaaaagaatttaaaaatgatgtataaaaactgttgacactttttatattgtatgctattataaaagtagttaaagaaagcaaccaaataaataaaaaaaaactttataataactgttgacttttaaaagtaagcatgttattataagagtatagaaaagagcaacaaactaaaaaagttaaggtggtaatctcaatgaaaaaattaaaagcaaaaagtaaactaagctttatcctcccaaaattaacaaacgacaacaaaaaaattgactactctaaaattcttacatcagcttgcaacgttttcggaggctacacattaagagagtgttcaggcggttgggttgacaataatacattatatactgacgaaagcaacgaactagaaattttacacgaaggcttaaacgaagacatgaaaaacacagtagaggaaattcttgattttcttttcagtgaagaagaagcgggacagcttgcagtatacgcaaacaacaataacgaagtttacattttagaaaacgaagatgtaaaagaatttaaaaatattttataataactgttgacttttagaaaagaacatgatataataaatgtacagtaaaaaacaaataaaaaaaaattaaaaggtggtaatattaatgaatattgaaatttgggcgtacgcaacgaaagaagacgaaatttgtagtgcagagcaattcatttgttcagtaagtaatgaagaagaggcagaacaagaattaagctacgcagagatggaaaactaccacgcttggaaaacttactaataaatttatatagcaaaattataataagtgttgactttaataaataattttgctataataagtttatcagttagataggtaaacaaactaaacaaaggtggtagttaaaatgaatgtagtaactaaaatcagtacaacgatgaagctagacgactatagcaacttgacaggacaagaatttacaacatctttctatcagttagcaaaattgcataaaaacgaaattaaagacggagtaatgaaagaactatctaattatcctgaggagttacagaaaaaaatacaggattgccagaaagaagcagaaggacttaaaaaagtagtacaatttgttagtgaagacgtgaagaacaataaagtaaaattacaaaaaaattcttatggtgaccttaacactgtttctttgaaagcaagtgtgcggagagctttagcgcaatacggtgacaatattaataattgggaaaaaaattacttactagaggattgtgctatacgccttcaatacaatgtttctaatgtgtatctttcggaactagctaacttgcggtcagatgcaagtgaaaatatgattgcatggagctataaaaaagattattcattagaagcagagtttgaaaaaattgatgactacatggcggtaaaacttatagacttagttacaaaatcacagcatgagcatagaaacacgcatgacgatataagaatcaaactgttagcaattctagaatttcaaaaaaaatacgaggttattagctgggacttatacaacggtaaaaagctttctgatgaagaaatcatagcaattcatagtaaactaccagaatttatttcaaagtttcatgctgtttacactgatattgaggagttaagaaattatttagaaattgagtaaaaaacgcttgacttagtgatgatacaatgctataatataagtatagtaaaggttatcactaagtcaagtacctacataaaattaaaaaaaagttataaaaggtgttgactttagatactaaacatgatataataaatgtatagtaaaagttaaggacgtgttaattatgaaaaaacctattcaggaactaagacaattaattgaggaggaagaaagagtattggtagactacatcagtctagaagacgacaacggcagaaacatcatttttattgaaaacctaaacgcagaaaactcagatgacagcactatgctagaaacttactccccttatgaagcattagagtttttagaaaatgaggggttgcttgcttaataaatttatataacacagttgtaaaaggtgttgactttaaatagtaactgtgttataataagtttatcaagtaaaggtaaacaaattaaaaaattaaaaggtggtaatattaatgaaaaatcaaattgttgggtataaaaaattaacggttcaaaaaatttggagagcaacaaaagcgcaattagtatttgaaggtgctagaatggactacgctagatacctaaaaatgaaaaaacaagggaaagaaatgggtattacagtaccaaaattcacgttgaaagagatgtaccaaaatcgtgctagcatgaaacgtgatgaactaagaaaagacatgttcctgtgccttgatattttaaatgaccgagtaggaaaataaaatgtggtttgtaattattggtatcagctttggactaattttagggctacgaaaatacaataaggggaaaggagcggaaaaaggtgagcaaaaaaaaccttagtattagtaagatgaaagaactctgtcaaaaagaagggagtaccttctttgaacctagcgccatgaaattcttcgggacaaaaattgtaaccaaaccaaaaaataacttatttattacggaagatttagcaaaagaccacagaactaaaaagtttacggtaagaaaatttgagaatggtgaagtgattagtgttaacggttttttgaagtacagttccttggcagaagctaaagaagttctaaaaactattttataaatttatatagcatagctgttgactttaaataataactatgctataataagtatataaaggaagagggtaatttaaatggcagacgtatttggatataatgactttttaacagaaatgggaataaagtcagtaagaggaaatgcaattacagagcaattaaaacaagattcaagctatattgtagaggatattaaaagagcatttaactttaaactttacaataatgagctaacaggggaaaacaacatggtgaacgtaactaacaaacctctgtctttagtagtattcttagcagacaataaaagtaaagtattattttacgatagtgtgaactttactagtaaaaaagctcgggactggaaaccatttcacgctatctttaaaaatggtaaaacgtatattgtacaagataattcaaattttgccttgacattcgttattaaaaatggtactattattaaagaaaggaaagcacctaaaaactcaggtttgattttcttagaaagactagcggaaggggatttataagatgaaaactgttaagaattgggttgaaagcaacatctttgttattctaacttttgtaatgggtttactcttactactagctttattacactattaagtgttgactttacagaacaagcatgatataataaaagtatagaaagagaggcacacaatgaaagattacgaagtaaaacgaattgagacaaaggaagctaaaccttttattttaggcttacactatgcaaagcgtatgcctagcgtgtcgtatgcgttcggtatattccttgatgatgagttacttgggatttgtacaatcgggaagcctgcaagtcacagcttatgtaagggtgtatgcggagaagagtacgcttataaagtatttgaactaaataggctgtgtatgaaagaaaagttagaagctaatgttttaagttattttgttagtaaagtgcttaaagaattaaagaaggaaaatctaatcttagtatcttatgcagattcaggtatgaaccatgcaggttatatttaccaagctacgaactggatgtacacaggtatcacaaacgaaagaacagacaaatatacacctgaaaacaagcattcaagacattataataacgagtatagtcacttacgaaaagtaagaacagctaagcacaggtatatttacttagcaggggataaacgttttgtaaaagaggtggcaaacaaaattaactacaaaaaacaagtatttccaaaatctgaaaacaaaaactacgagttaggaacaagacagaaaacaaaagtaatcaatacaaaggataacactattttttatcaatagaggagtgtcacaaatgaaaaataatacaataaatataaaagatgtaaagctaacaatcagaaaagatactataaaaatgaaaaagctagctaaaaagatagcagagtattacacagtaaaaaaaccttctttagcggatagcttgcgtgtgcaacttattgaaagtgaagctaaaaaactagaaagcactattgcaaaaaatgaggagcttttatatttgatgaataactttactaaataaattagtttttactgttgactttagaggttaaacatgctatactaaatatataataaaaaaaggtggtaatattaatggaaatgacagaactcattaaaactaaattcgaaaaacacggctttgaaaatctaaaagaggaaatggaagacttagcagagggttaccaaatttttatagatactacaccagaagcatttgaactagcttatgaactagaccgtgagggcttcacaacatgggaagaagcggaaagtctttatattgagttggagagtgcgtaacacatgtatactactattccacattacattattaaaaaagtatctgataaggaaaaggtaaactaccaagcaatttggaaggtactacctgtagaggtgcaaaccatttttgtatcaaagaaaacagctagcttctatatagttgatagatacgctaatagccaatttttcagaaacttgctaacaatggcagggaaggcaaattttggggtatcaatcagtagttccagcaaatcccatttagagataaggctcacctttctaaatacacatttttagttttatatagtgtagctagtcctttattgctacactaaaataagattaaaaaaattaatataaagtgttgactttaaatagtaactgtgttatactaagtatataataaaagataaggatgtgtgccaagtggaaaaatttaagtcactaggagaggctacaggtgaagaactagcctttgttttagaaagtaactcccaaatatttgatggtgtgctagacattgcagaaaaaaatcttggaaaagaagactttgacaaacatgttagacataacggtcactatgatgttgcgctatgttaccttgaaatatatgcagaggataatcctaacatcttattcgagaaaaatacctttcaactaaaaaattaaaaaaaaagttatataaggtgttgactttaaaagttaaacgtggtatactaagtatatagtaaagaacaacaaaaaaattaaaaggtggtaatacaaatggcaattaataaaaataagcacagtgtagcagttgttaaaactcaaacggggaaaattgttggttatctttcaactagagacgaagaaaatggcgtggatattcaaatggacatgcaaggttatactgtagaagcgctataaaaataatttataaatttatatagcatagctgttgactttaaataataactatgctataataagtttataagttaaacacaaaggcggagatagtaataaaaaaactagtataaagtgttgacttttagaaataagaatggtataataaatgtatagtaagagttaagaaaaaaactaaaaaaaaactaaaaggtggtaattaagatgacagttaaaacagtttatagcccaaaattagggtacacttatggaaatttagaagtagaatacgcagatgttaaaagtttttatggtaaagcttattttagaaatatgcacacagacgaccacacgcatgtataccacttatactcttacgatactttaattcttgaggtggtagacggagttgtaacagcaatgacagatgacacagaccactatagcatgacaactaatagacacattaatgaatttttagcacagtatgaagatgactacacaggtaaactaagcaaaaaagaactactagaaaaagctaaaaacctaggaactgctttagtatagacataaaaggggcgggttaacgtgactacattcaagaagaaccgcacagttaactgatactagaccaagtaaaaaagagtttataattataaaaagtgttgacttttagaaaagaacatggtatactaaatgtatagtaagagttaagaaaaaaaaactaggagcgtgttaaaaatgagcgtaaaaattatcagtgaaggtttagaaagctatatcccgtggagtggagcagttgtgaactacgaaattattcaagaaaagggactattagatgtttttgagtcattgttagcagaatggtatccggaaggattaacagaaacacaagtcaacgatattttgtggtttgatgaagacttattcagctttttaggaattgaagaggaggaggaataataaaaatgagtataatagcaattaaaaaagaaatccatgcaaaaggttacaaagtgacaggaacccatcaaggctatatagcacaaatcaattttgacggtacaggaaacgaatatcctttaccagctacatgggatgagttcattgaaacatttaaagatggttggaatggtacttatgaagacgaacaagcgttcttcaacgatatgcaggaagttgcattaaaagaaatcttagacgaactaatagacgtattatataatctagacattactacatataattttactatagatgatgttaaaaagaaagttattacattaaacaaacctacagacgaagaagatgcagaagacttagtaatagaatttgattctacttgcttttgggatgctacagtagaaaatgataaaataaaggttactgtaagaaacaaaagtaaataaataaataaataaataaataaataaataaataaataagggttatcactaatatatttatatagcgtatctctacctttcacttgcaatactagcggaggtatgctacaataaacatattagacctaaataaaaaggagaaacttatatgatgacaattgaagagttagcaggtctacaaaaagtaaaaaataagctagaatgtgcatataaacaactaaacgatgctacagaatcactagaaaccactctaattaatgaacctctcgctataataaataccgattactcttttctagctaataacatagaggatgccagagaggtattaaacaaaacaatcactaagttagacaggtataacgaagcactagcaactaataaagaagagatagacaagtaccttcatcttactaagtctaaaactaaataataaatagtataatagacaaaaaaaaaataataaaaccaaaacctgtaaatcacctcagcatttttttcacacggtgtcaactcaaaaggcttatatatgcgcagtctttctacctattttttagaggggataatagtactatataaataacctctataggtataacttcttatatttaggattatttcaaactaattaggtctaaactcacattattataatgtcaatagggtataactcctaataatatataaaactatgtacctattgactaataatggtgtgtaaggtataatagaggggggaggtgtaggcatcttatgaaaaagaagaaattcagtttagttatttctttattactacttcttcgattatattagtgagggtacgcttgtagatttaaaagaaacaaaagtaaaataaaactgttgacttattaggttaactatgctatacttaaggtataaacagttacaagtgggataagagggtataggagctcgtatataggcttttactttattagataggtaaatagcagtaaataaactaatcccgctcagaaaggcttgtaggaggttagagaagatgtttgtattaaatggtttactatgtactttactagtattgatgattctagtgggggctattgctttaaacgcagataagcagactaagcttattgtaggaattatacaggtggtatttatgactatgtgcgtagtcctctatatcactattatagggattagcattactattattccttttggtgtattatttgcgatttgtggggctgttgctttgttcaatctacttacttttaataaggaggagatatgatgacaaaatacaagttaaatacggttagtaataataatttaaaagcaggggacgtggtacttgtcgagaacttagaatgctacgaattaaaatcaggtgtttacaagattattagtgagtgcgacggtctatataattttgtgggtattgatgtagacgtgtacgaacctgtttctgtagaaaacattaaccgactgcttgggagctgtgtgttacgtgtatccgaattaactttggatgtagtcgttcaaaactaagattagaaaaggaggaagggactatgttgttatttatcatgaatgttatcagtgtagctatattaatgagtactttttactatatacgcaatatatcccctttctattacgagcggacgactagactagttaatcacttagtagatatagagctgattttaggattatttatctggttagtgtttattatctctattacttttgtatagggaaaggagtagaactatatgcttattataaagtctttaattgtagctattatatttactatcttactagtttatactatagacgggttttctcgtattcgtaaaggggctaaaagtaatggtattttagctgttgtttgcgggattatagggataattaccttttctattctagtagttattatcttttagaggtataccaattttttaagggttcatttagctaaaaatagctttggggtgtgccaacgagtttcgcgttttggagtgaaaatatccctaggggggaattcaaaaagttcacctataccaatttgaaatgagggggttttattgtttattgtactgatgggcgttttgggaatagtgttagtcggcttgggtaactattatacaatgaaaggtctagcgatgtcacataagcttagtaaagacgtagctagtgaagaatgggtaaatgaacagtttgaagattctatagaaggtcgtataaagctaaataactatgctagagctactatgaatcgttcagactatgtatttaacaaagggatgttattactatttataggtgccttattacttatcccgctagtaactttaatttaaaaggaggtagaaaatgggtttaacattagtaaatgacgatgataagtatctacgtgcgtttaaactagattcttttcaatcagtaaaaggaattatctatgtatatattgttattactcccgaagtattaacagagtacctctcacagagctcagatggacagtattttaagagtagtaaagattatcaagtaaatgtacaggaattagctagatggcttaaaacgtctaatagaggctttatagagacactatctcataatgagaacattatggcaagtgtaggagaaaataatatataaaagagttgacttctttcttttgatgtgctatagttagtgtatcaagtaaaggagttctttttatatacttaaaaaaaagttataaaaggtgttgactttataaaagaactatgctatactgaatatataataaaaacaaaggtggtaatattaatgaacaacaatcttaattactggacggatacactagcaatcaacttggcgggattaacattagtagaacgggaagaggtaactacacaggaagcactcatatcagagatgtatacaagagacttacaagctaacaaacctatgtttaataacttattagataacctagaagagttaaacgacaaagcacgagaagctcactcagaggctttctatcatgcagtagatgctattatatcgctgtgggacggaataagcaaaaacactttataaaggaggaatagttataatgacaaataaaaagaaaccaactgtacagatgacaggggaggacggaaatgcctttcttatcttaggaagagttactagtgcgctgaaacgggcgtgatacaccgaagaagaaagggaacagtactataccgaagccacatcaaaggactacgaccatctactggcggttacggctgaatgggtgaatgtagagtaagcatataaaccaatactattagaaaattattagataaaagggttgacaagttcaaccccttatggtatacttagtatatagtaaagagaggggacgagttacatgctaacactaaagaaactattgaaagctaacaaaggacaaaagaaagagattgagaaagtattatccgaaagagaacaagctatcgcaaacttagaaaagctagttaaactacaacaatctgcttaagcattcagttacgtcattagtggtaggcatatatacccaccccgtggttctgtgaccgtactttggggctatgctacacagggggctcctctcggggcacgtttttcgggaactttttttaaatttttttattcttaaagtcaacaaggaatggctctatagagggctttacaagaatctttataaaaagggcaccccgttgttttttcttaggtaattttaaggagaggcttctataaataaagtgaaaaaattccggtaaaaatttttaaaatagggaataaggaggtgttcctatagatatgaataaagataaagacaacgcattagagtttcgagcttggtgtaagataaaacagatgatggcagtggttatctctattaatttttataaaggagatatacaaggggaggatgtatttctctcttaccctgcacttgggtcatttaacttaagaggcttttccgaggttatccttatgaggtacacagggttacgagataagctagggacaaagatattcgaaggggatattgtagaggttcctttagaggcagaccatggatatgcaggctctgtgggggttgtcacgtatcgtaactatggtttaggggttatgacacctcaagggttcttttatttagaggaacctgagttgcttcttgttacagaagataaatatacgaataaagtagaggagggttaagaatgaaagcacctatttcagaatgtccttattgtggttctacagaagggtacacacttaggtcacaagcaagtggtactatctactggcggatgaaatttaatgaaaaagaggcagataattcagagtcacatgataatttacagtatagtccccataagtacgcttattgtaccgtatgtgagagtaaacttttcaaagcagaggaggtgaatgggtaagatggagcttcctaagcacattatagtaaaaattaaacgagcagggcaccataataaaaaagcaaatgacttacaaacggaagtacgcaagtggttagagagtagattaacagatggggaatctctccctgactatataggagaccaactgatagatgcgttggagcttggtgtagatggtagtgaggcgttacttaaattcttagctgaatatgaggagtgaatcttaatggaaaaattgaatgagaaccagcagattgtactaaagtggttaaagaattacttattagatgattctgatttttataacacggtatatgcttctaaacaattatacgtgaacggctttcctagtgataaaagtaatattgcttttatgaaactaacagacaaggagttcgctagcgtactactagcgttctctacatggtacttactagaattagaaaaggaggatttaacatgaaagagaaatgtagtatagagatttcttactataacggtgaagacaagtttacgggaaaagtagtgtgggttgtactagacaaagaagaaggtatccaatttgtacttactgagaaaccagaggacgaatactttgtttttgacagtgaggagtcggcaggtattgtaggtggtcttattagactaagactagcatgtgacaacatagaagatgactcccctgtaacaattaattaccttaaggaggaggagaataatgaatagctgggagtctattttagggctaggtagcacgcacaaaatggtaatagcacaagagttggtaagttttctttataaggaaaattgtgatactactacgctaggggagaatgagcttttcatactaaagtttctcctcgataacaaagtggagtttaacagaaaagaaacttattatggaacaacgttctacagaggttatactgtaaccgtaaatgttagggactctgtgttagcaactataggtaaggatatagcttccgtagataaagacgaagataaggctactccagaagcagtaagttggttagaagtggaatgttgtgatatactgtatgagttcctgtctactaaactaactgctgagaagggtagtggtagacttagtgacacgcttaaaataagtcgttcttatgaagaaaaggtaaaaggactagaaataactataacaaaaacaaaagtttttaacaaattaggaggagagtaataatgaaaaagagtttatggagtacaatactagaagacacagtactagaggataataacaactcaatagcatacgcaagtgttgttaacacacatcgcaagtttagacgattactagagaatgaggactatgaggctactattttacgtatgcttcttgaaaggaaacaagaaattgacggggtgtttcaaagcaacgttgagtttagtcccctaagtgtacgcccatcagcgccacctgtaaaagaacgagggtacgaaatagttattgatgtgaagagctttgtagccgaagcattagatatgcctaacgacctagatattaacctaggtattaatggctttgagcctgtctttgctaatatgctacaaaattatttaaatgaaaagatttttgaagaaaacgaggtaatatatgagtttgagggtagtacatctgtggagcaatctataaaagtagagggtgacggttgcatcgtagttgtgaaaaacataactcgtacttggtaaggaggaacttatgaatagaaaaacgtttagacctagttggcttggagaagcgtataaggggtatataccaccaaaaaatgattttcctgtgatacgtataaaaggagacatggtttcttggtttactatccttttagaacaaaatgaagaatttgtacgggagtggatgttaaagtataaaagaacaacacgtacaaaagaaacaggtgctcattatataggggatagtgactactccacttattgcgtgacaacctaccttgttaccacacgtacagcatcattcaatcccaaaaggcttgttatagctctatttactaaagaactagctcttttttctagggaatcgacatcagtaacactaacagaagaaaatctaaatttaaatgagcgtattactgaatatgttgaggcattgagtgagtatctagagtctgtaggtatttttccgttctctcttcctagttggtgtcatcccgactatcattacacatacgatggctgtcgttttactatctccaagagagaaagggaagtaatatctagcactaaaatgaacttagaagaatataaccaacatttactaaaaagcaaaggagagaaactaatgaaagtaactcaattagaaacatttaacgattttattgactacatggttaaaaagaatgaagaaggctggaagtgggaaaacaaagaccctctagtacctgtaagtttcgaaaggtggcaaatagatagagaccaaacacttatagctaaaaactttagaaataaggttatttatacagttagggtggaagaccttgaccaaacacctacaattgtatcatataagacagatgcggactgggtcggtagcttagacccaaaagaatatgaatgggagacgatgaaacctaaaaaagtacctattgtccacccgtgtacattaactgttgaagaactcgaaagggattggaagactaatacaaaagagtatgacgaagttaataatccttctcattatacgtctgggggcattgagcctattaaatatatccagagtcacaaaatgaactacgaaaaaggtaatgttattaagtatattactcgggcaggtaaaaagaaaggtcagagcgagattaaagaccttaagaaagcaatgaaatatattgggttactgattgaagatatagagaaatatgagaatagctaatttaataacaaaagtaatagggttgtggattctgtgtagactagtaagtgtctaagggttcacaaccctaatttaaaatgtgaaaacaaacgaagtataactgttgactttttacttatttcatggtatagtaagtatatattaaagaacaggagtgattacatgagtagagcaggctacaaaattcgttatgtaaaccagagaggtaagatggtttgggttaaaagtattgggtttggaacaaaggcattagaagtcgatactactaccaacaaagagaaggttaaagtatttttggataagaaatgggcacttcaagtatgtaaactcattgatggcgacctagatgttaattgtatgatagacgacattacttataagtattttatcttctatgaagcagaagataagaagtactttctaaagtcgctaactgtagttcaagataagattgagcctgaatggacagaaaacgagccagaacgagcggttattcataaatacaatgatttagatagaattttagggctagttaacgatagaattgataaggaagcaagagcacgtcacgatatgtttttactttaaacgaggaggtaataaaaatgattttatatggagtgtctctttatggggaggtaactgagtggactacagataaagctactgcaaataaatgggtagataatgcagtacaatctatagaggataatgatatagatgaggactgttatgttattttgttaagactagacttagaaaaacttctatatggcacattcgaccgtgaagtagatttaaacgaccaactgcatgatgaagctacagtactaaaagaattacgttggcaggtggacgaggaagggctatataatattgtggaggtggcggaatgaataataaaaagacagattataaagtatacaaaataacctacaagcaacgttttatgggggaggttcttattgactcgtatgaaagagtagtgagggatgataacgaactacagtctgcaattaacgctttatatgatgacccacatgtgttctcggttagtagtgaggaggtgacagaataatggagtttaaatcagtaaaagaaataaatacagagattagtttgttagctgacaaaggaggtatctcagacggctcacacacatttgaggagctttactttcatagaatggttttatttgcaacaatatgtaatgctaatcactctaaaagttggaagtctaagaaacatgaggacggttctatgtttgataactattttatcgttggtatttctacatcgaaaggtatgttttcttaccattatcacttagacaattgggactattttagtgttccagaactagaatttgcccctacatgggatggacatacagctaatgatgttactcgacttttagatatataagaaaatgtaagtaaacaaaggaggtactactatggatttaaataaatttttaactgaggtagataaagttacttctgcttacggggaggatgcaaactacgagatttcttttattcgtgagaaatatgcggacttagacgaaggtgccaaacaggactgtgaggaaacggacttctttgaggaggtagaatacatcagtcagatgcaaacaggagaagactgttttgagggctatatttttagaaaaataaaaaacacagattggtgtctttgtgtcacgtatgctatctgaggtcttggaattactactaactttttaggttggcttaaaatgtagtcaattagtaaacatattaaggaggtaaacataaatgatacaagcttatagagttcgttcacaagaagattttgttaacctaatggttaacttagaaaaaaaggattttaaatggtatagtgggttacctcctctagacggtgtatactacagacaaaacccagataaagacttagctagtgattctcttcttgttgttctaaactatgaacatcgtactattagatactatacagttgctaatttaagggaagcttacccactagcaaccatctatacgtacacccctagtagaacatctaaaacaaactcagtagacggttttactcctactcagttagtctatgacgaagaactttacctataaatgtactctacacaactaaaggcattatacatgtctttttttgttgactttatttatcttatatggtatactagtattataaataaaggaggtgtctttatgaggctatccctaagaaatatattggaaggttttcttctattctgcctagctttaccacctatcctagcagttatgggatttttattaggtattatatggattacattttactttcctatgtttggtctagctttttggagtattttaagtgcatgtttagtagagaacttgctagaaaaatgggataacagaaagactcttagaataaggaactttagtatgtggtatgttatagccttacttattgctcttactattactggtatagctatactagtgctgtttttattctagaacataaagagttatgtcttaagctagtttggtaataaaataaacataagaaggagaggttattttgaaagctttgttttttgtaataggcattctagtatgccttttaggtgttgctatcttagctacagcaatactgtggattactactagtcttcctgcaatcggtgtcccttttctaactattactttacttgtaggaatcctactagttctgcttatagaaggaaataagaaatactaaagaagtaatggaggttgttaaaatgagaaagatagacataaaccaagcaagtgtggatttacaactactaatcgaatttgagaatgaggactatacagtagcctatagaggagaagagagtaggcatacagaagaaatgaaggaagcaatcctttcagctcacaaagatacaatagaccgtttagaggtagctaacgataaactactagagacttatgaagacctgatagaattgaaaaaagaatatcacaccacgtataagaaaaatgagaagttagctgtaaaaaataaagaactaaaaaaagagttacgagatattacaaatgaaaaagaaaagttagaaaaagagctactccgcataaaagtgtctaagggggaactttaatgctagtatcccttacttttttagaggtacttttatgggtactgactttgttatactttggtatggtaatctacgtactcttatcccacctagttactagcgtagcttaccccgagtctgatggagttgctagttcttttatagactatagttatttatggggggaatctgatgagacgagcaaggctagtaactataaatgctacgaccatataaactacagaaaaacgagctattttaaccccacttataaaagcggtgaatttctaactagcacagaaaaggtgtctaagcataaaagaaaagtctttaaaaataaacaaaaagttaacttagatgaagaagaagagttataaaagacacataagaaaaggtacctgctaagggtaccttttacttttatactattttttgttttactggctctactatgttaaattcccctttatttgttatagtgatagcattaagttgcccttcaaaacagcaaccgccatctattcctataagtttttttctgtcgtgaacccacacgtcaaaattagaataattacggtctctaaacggtaagtaacaggtaggtgtgtgaccgaatacaattacttttcctgtgaggttctcttgcttataccagtccctaatccacataaactcgtggtctgttgtcttcttccagtcttccccgtataaagattgaatacctgcatgagtaaacagaacgtcaccaaactcgtaatacggcttagaagctctcaggagctctatatgatgtctatatttcttttgtatgcaaatacttgtaaagtgataaaaagacgatagcctatgcctagaacgctcctcctgtggcatattattgaagtagctagacaaggtgtttcctcctccactagcaagccatctgttaatagtgtactcattctgctcttcttctataaaatcaagcaacatttggtcgtggtttcctcttataacaaccacttggtcaccgtactccatctttaaatcacaaataatatctaaaacctctctagactcgtcccctctatctatatagtccccacacaagacaagcaattgtttatccctatcccaattgtttaacatttctaaaaactctaagctacaaccatggatgtcactaattgcaaaagcttctttgaatttagtcaccgaggcacctccttaatcaaaattaatttcttctttccaaccttttatataagatgttaggtctccttcgtaccccataagacgtatttccattaccaactgacctatcgtagcatttgaagtgctcataccctctaaaacctctataaacggtcttagtcttctgtccataatctcgtttgcagtttctatagcactctctttgtttctaccgttctctatgagtaaatttacgtaatcttcataagtcatgttaacatgggttacaccgtccaccacactagaaaacctacagtatctaccatttggttgttgtgagataaagctactcatccttgatattcatctcctctttaacagtactttttagccttttgttttccgcttttaccttttcgtataaccagagagctgtagacaagtcctttcgtgttacctcgctgttgtttcttacttttgctagtagctcctcatacgttttatctttaaagtccatagtagctccttcttctgcattgtcccaagcgacctcgtaagcaaccattcctgtcattccttgcatcacacccacaatagtacctatagatgtgttataacgatgttgtaccagttcaaactttttaaacttattcattcttatctatcccctagcaattatttgtaaaagtaattccaaaagattccatcctaccatatctaacagaaacacaagctgttaggtagtcacttactcgtatctttactttaacaatgtcaccatctataggagacttaaattttatttctatgtcaactgcaaaatgattgttgtgattaatccaagattttaaatggaatattccttcacgcatctttgttacagttgtgtatggatactctagtgaatgtttgtgtatatttcttggaaattctccaaacattgcaattacttcttttgggtttgttggtgctaacatgtgatgtctattttgatttgataataactctctgccttttttgtcaagctctaaaaagtcttctcctctatttttcagtaaatcaaataataatttttctgcaattacctttccttctttttgtctttcttttctctgctgtttatcatatttcttcttttctttaattatcctattccttgcatgactaaatttttccataattactgtcacctcacatctttatttacttataaaatctactaatattgccaaaataataagaaataatatcactatccaaacaaacaccactattactacccaaataattattggaataaacactgtccaccaaacaatattaataaaaccagttaattgaagtattaataaaactaggaaagctaccccgcatactgtacttaatctcatacttattattcaccttccacactggcttcttttaataattttattgtaacgttttcatcatctgttgctgttcccgcaagaacctgcttaaccatctcttcaaaattctctctttttggctcttcaaaatcttcttccgtatacacctcaacttctacttcctttttaggaaccaaaagtagtccaccaaattcatcattttcgactccataagtcatcttacctgcttctacatcttcgtttgttttaattgccattattgcaattacatctttaatagtatccaccatattcactctcctattagtttagtaagcttagccgggtaccacagtttttcattattattaatatcccattctattaaataggcaaaatcttcaccttgtactctagtgtatcattataaacttcctctactgtacctaactgttttttactctgtaaaatgaactctacttggtctcctactttaaacttcatgcttcttcctccatttctaatatttcgtgagggtctacctttacaaaatcagataaagagtacctaaatacaaccctgtctttttgagatacaagggcttcttgtttttcttttttatgacccctacaatgtttgcaaacaagaagtacatcacgagtgtaaatattcgtgttctctatagatttccacttagaagccgtaccctcccatatgtagacgtgactgcaagtaggcttacttctaactaaactagctagtttactagtgtatattttcatagtgtaaacacctccaactacattatatcataaaaaaagaagagtgtcaataaacactcttactttagatgttcccacccgtaggcactaaatatagcaattaccgctaatggtaaaaacgtaataaaggacacacgtaagataaatcttgagataggatttctgtagtaattccatagatatgttactttaacatcatggctatcatctttagactctaaataaataagtaacaggaacagtgggtatattgctacataaatttgaaggattagtaacaccatctatttagtatcctccttaagagacaaattatcgaggaaggctactaagtcttttttagctaagtttcctttgtgattgcctacaaagtcctttatagcagccaccagttcttcgtgaagtcttctgttctcttcgtaaacctcattctttaccgtccgctctaggttacttcttgcactctcttctcttttaagtaaaacgcaagcttccccctttagtctagtgttgtcttcctttagagtctctatctctttttccttttcttccatctgcccgacaaccctttgtaactctcttagttgccgttctttctcacccaaaataacttcaagagcgctcttttctcctacagatttccctagtttgctggttacctcttctagttcagtagtcatatttttaatagcttccttatctttcttttgttcttcataagcatctacaatatattcttttaaattcttatatgggatttctttctgttctttttcctttagtttgctattttttcttttagaactaattacctctaaaaacttcattcggattcctccttcattgcttcaatctctctatgcacttcttcatagtaactagctgtaaaagttactcttgaaaactcctcgtaagcctttagctcccgctcgtccctctcagttatctgctcccatgtaaaagttttgtcacactccttgcagtataaatcgtaatctaagtcataattacattgtaaacactctactgattcgtctccttcgtagtgcatatctatttaccacctttagagatactatcgaacttttccgctaaatctgcatacatatctgatagggagtcataccagactgttctagagaaagcaacagagtatacaatggctttttggtagtaaagtctgtcaactcttaccgcaagacgtctaaaactatctctactctcttctaggaaatatgtgttactagggtcaactgcttcttttatcacagggtaaatctctttaacaaaataagcttcttctttggagtcaattcccttttgagtcagtatacctaaaagaatacctgcacgttgttttacctcctctggtgtatctcctgatgctactcgtttaactaattctttaatatcttcttctgtacctacgcctataaggtgacaataaacttctaagtagaatttatttactcctaggtgatattcttcgtcttcaacatttttccagataaactctttaaggctttcagcaggtaagtctagcatttttaaaacacagctaacgttaagggctctgtacttgtctaataactcctcttctttttttctatcataagcaagggaagctgtatgtctgctaaatgtatctacgaaacattggtctttgaaaagatagttcagcttttcttcaccagattgttttacgcataccttaaaaagtttatacagacggtcataatcagcagaggataagtaagtaaaccaatcagctcctactttaggtgtgaaccctataaccttataactgttctcattacaaatacgaaactcttgtcttttaattatactagcgtaaacgaaaaatttacctttcttttccacaccaaaaatatttttgtctttaacaggttctgtagtgaccttaatatcccaacaggacttcaactccacccctaaaccacctaagatactgcccgctacattttctatcttaatactatcatttaatttgaactgactgtacttaagccccaattcctcagtaactacttctatcaccaaaggcattatatcctgtatacgttcctctgggttctcacttgacttgcattcaactataataagaggtagccatatagtcccattacccccttcacttgtactacccgcatctcttttttcataattatccttaatacgttttttatttagttcttctactatacctctgtagtaaatgttcatcattcgagtgttatctttgatgtctactaacggaatgttcttatactccttatgcttagccatactattcctcctcactacctattatttctttgtagaagtcctttacaaagtctgcttgctcctctttaggaacaatactattaatttcctcctcaaagcaaaatagaataaagcccactgttttgatagagccctcattatatagcttactgcttagaaccatgtagtccaacaattcttctttcttcgtattgtctctatgaagcttgtcaaaggtattaacagaaaaaccaaaaggactcagctcctttataactaacggctttaactcaccaaactctactttagggttagttttacttccgaaagaaagctccaactctttagtgtactttttaacaagttcttctacttctgtgtacgttcctacgctacagttgtttttctcctcctgtgagctatattttacgtaatactctttactcatactatcaactccttattctaattgtatcacaaaaactctattaagtcaacaaaaaaagaagatgttactcttcttttaatggcagtctagtttttagttttcgtgttcttcctttgtatctgagattgctagtatcagtgtgtactgaccttacttttacaatactctctttgacactatcatatagaaaatcccctacagctaccttacctaacaagctgtcatctaaactaaataaatatgttttagcataggtacagcttaggtcatcctgaacatagcttatgtaaacgtacttgttctcataatctgtcgctccccaagactcgtctacttcccatccaccaagtatagtttcaatattggcatttagtatctctgcaatgcttctagcttgcgttaagcttgtcagcggtcttacctgattaggttctcgaactttttcccagtttaatacaaaagtacctcttttgttcttaataaatgatgttgtgtagtatgcgtatggaaaccccttctggcgtagaacatacatatttcttttttttaccatataaatcaacctcctagtatttttggtaagtcctctgataagtttacagatacgtcttttcctagtaaattaagtaactctttgatagcttcttctagttctacaaagtactgtggtgacctatttcgactatcttgttgcaacttaatcacgcaatccatattcaaatcagggcatgaaccctctcttattcttataatattcagtttattatttaacgcatgttcgcacttctgtctatctttttcagcattttggtgccaacgacctccatcatattctaccactagtttcacagaaggtacgtagatgtcaaatctttttcctgctatatttttttctaatacaattcgagggaacgctttttttaaatagtaggcaattatcttatttggtttagaaacagtagaggaacacttagggcaagatttaaaccctacaaacttagacttcacactctgctgataagagccatgctccttacatttccaccaaacctttatgtcgcttcctcgacttacgttcttaggttttatacctatagcattgtttttttcatagtcccatagttttcttagaacagagctatgggactcactaataggtatacaccctctcttaagagctttagtagccatagttatctcctccgctctctactatatcttaggttgcaaaggtctacttctgcaaagaacatgtagtctacctccgcatgagacatccctaagtctcttaactcatatactttatcttttacagtttcccttacacgttccagttctttaataccttcggaagttaggttaccttccgcatctcttaatttaatagccatagaattttctcttattggagttagttgctctcctaaaattgtctgaatctgataatatagcggttgcctttaataactctccttttgtaagccagttgaataggtcatcaacagataccttcataagaacagctagttgctccataatgtcatcttggtctgtatgacatgacctgcttgcttggtctagtagtccgtaacaagcttttttaagtatcttcatgggtttatctgagatagtgctagaaggctcacgagaggataaagaaacaacaccagagcttgctctacaactagtgcacatgtagcacattccattaccgtactccttgccatataaatcagcattagatgtgtactttatattaccttgacataacgggcactttgtaggaatttccttcttcatacgtgccaacccccaatcgtcagtccatcgaataacataccagccgttattgctacagagacaatcaaaatcattaaaattattgacccccataaggtaagccctctggtgaagaactccttaaccgtaactaacagccaaataaatgaaataataaaaattatccaagtagttaatcccataactttgtctcctctcggtatttcttaatcttcaatcttgcatctgatataactgttgacaaaggtactagtagccttcctgccatgtaatcactatctacaccaatgttaatttggtctttacttggactaggtttactatgaatgtgtccatgaatgttgaatctacttttattacatccaagttccataggataatgggaaaaatgaaactctactgagtatgcgtgaataatcataccaatatcgtgtacttctactacactagggcataggtcactattgtataagtttactttttttcgtgtatcatggttccctcttataataatctttttacctcgtagagcattcataattttaaccgctttttcttcgtcacctaatgcaaagtctcccatgaagataacgacatcattatcattaacagtatcgttccactccattataaactgcgagttcatgttgtcaataaaaacgtatttatctcctctataatgttcaaacttaagtatgttatcgtgattaaaatgtgtatctgatgctaagaaaacattgttagaacttgaaatgttatatttaaccaaaaaatcagctagactaatcaattaaaaacacctgcctctagaggtacttctttataacctcctgtataaaaacttaagttgtactccgtcatagacttatctttactatatacttttactacacgattactggttactagaatactagctacctcttttttacctactacatgtttatgctttcttccgccaccatctgtgatttctacttctagtaattcacgaataccgaacataccaaacaactcctatctattctataataatattttttctgatttctaaccagtctacctctttgccaactgcttttaaagccttatcatcaatgtataagtcagcgccaatttttctagtatcatttttccatattgctatcttatctgggttgttttcatttacaaagtggtacggtatactgttattggcaagaaaatccaaacacaaatccataccaagctcttctctacatgtccaaatagtgataactgccccaagttcaacaagttcttctattgtctctttagcataaggtaggagctcccctactaaggggtagctctcctcctgctcactagcaatagttccatcaaagtctatagctatatacttatagcgtaaacttctcatctctacgcctttggtttgctatctttaaattttaactgctcttttgttgtagatggtaaacgataagactcatacaagtgcgggtggtctttctctaacttcttaccgttaaatcgtaaaccttccacctccgcttgataagttagttcaaaatctcctaaaatctctaatgattttacgtttgccttcaccatcataggcattaattcatctttaagtgcatctaactgtttttttaatatctcgtagttacgttttgttgttgcataatttttagctttgtcctgcaaggttttagtaatagacacccctgcaccctctttaagttctaccgcttgtaatgcaccttgttttgccatttataatctcctcctgtcttatttataaattaactataacacatattttattataagtcaacaaaaatgtatattttttatttactatcctctacttttaggattatttctctgagatgtactggaacactacgtccaagagactttttagctgtatcctcatcgtaaacaaaagctaagtcatctggtgattcctcactcataatatcatcatacacaattgagggtactaccttcattagttgggaaaagttagaaaattctaagttaggcaacttattttcctcttctagtctctgtagagctttcctaaccctagtctcagtgacgaattgtcgaatccactcggtactttcaggtaaatctttaacttccctttttctagattggactctttctgcgtgttttggggaaacacttttaaatagtttctggctaccatcgatgtctacttcgtctaaaaatttaacaataataccttctccttcattagtttccttagtaaatctggactgacctacaaattcgttaactgtatcaaagtcagtaaatacccctaagtaaaacatatctggcataggtagatttagtgtaaaagcaatactacgtacctcatctaaaggtaagtatcgcccttctacgttactgtacacgtcaaataagtagaagttatttctaaattgctccttatacggtgtatttaacttcttagaacctagccactcaccaaagaaagttaggtttactagacctgaatggacaacatcaggaatacttaccttgtctttaatgtaagtagtgaaacctgatagtgtgtttacctctcctacttcattaggaatgtctaactctatatttcttgagaatttacgcatagcatagccaccatcaggaagtttctctacaaaaaaactagcgttgctaccatctaacttctctagaatgataatttcacctgtatatgtttttgccatttgttgagctcttccaattctttgaatttttctatatctattcatattaactctcctcctaaatttttaagtctttgaacgaaatattatctagattgctgggttcaagctctcctgtacgaatataagtagtcatgagcttgctgtctaactcagtatcctcttttaataattcttcattaactttatttataagaatgccaatatcttttcctttagcgggaataggtatatttaatgatttagctatcttgtctacttctaacttaattgcctcagggagagtttcctgataaggcaagaactcatatagtaagccaatctccatacccaagtcatcagcttcactctctgttctagcaaaatcaatttgttgacctatttgttttacctcctgctttactgcttggcgtacctgctcatcagtaagttctgatacacttttaactttaacttctactcgcatgttatcaagtctagcaattaaattttggtatacattgatgaaatcacttttactttttctggcatgtataagactttctttcactctttctaccaaactagttcctttgtctttttcttctaacttcataaattttccctactttctctatattctctttttgtatcatcttttataagttttctctcatatctgtttcttagttttttgtctcttccttggcagtgtggaaacatcccatccttagtgaagagtagctcctcgtacactctattagccttattcttaaaatatttatttgtagattttttactcatttagttatctctcacacgttgtaaaattttacctaacaagtttgaacctctccataaatctttgtttaaaatacgagggtcactttctcgtagtcctacgccccatatagtgtcataagggcttgcctctactaaagtagttcccttagtagcttctagtttatcttttaattccttattttgagagaacttagccattaagataataaaaacaatagcctctttgttatcatcccacgtagcctcgttaaaacctctaacttttctacccagagatttaactgttttagggtctccactaggaacggatagaatttgtgaagccactaactcatcattgaatagacgtgccttctcatacatgaatgcctgttctgcattatgaaacataatagacttgttagcttctttcccttcttgcttgtactctaaagaaaaatttgatgggtgccaattactaaaggggctttcctttccccaaaaaaatgtgtatctatctctcattttataactcccctccttgacttagtaaataagtaggcgtttctgtgttagccattctgcttactcgggtagctaatgtctcaataaattttttattatcaccataaatattaatagtctgcgcttcttttttagttagtgtagtgaactttgtagttttgtcaaactgtacaaaacggtaataaaatttttgtgcttcttccacagaactagctaatacccaagttttttctttatttcgttctgaaatttctactactaattgtttcacgtgaaacaacctccttatttattttagtttgtgttctcttatttaattaatgcgccttaaaattatagattggtttaacaatgaagtcaatgtcaaccgtatctgtaatattctctatgatactctctttctttttataggcaaatggtgcctcatctagtgtagactccaacacagaggaagaccaaacatctttcattgtattcttaaaatcttccatttctagtgtttctttagctttagaacgggacagtaccctacctgccccatgaggtgctgaattgttccagtcttcgtttccctttcctgtagcaataatgctaccatcacgcatatttagtgggataactagttgttttcctttagaggcatctgtcgcacctttacgaataaccttattagggatgtcaatataattatgaatagagtcaaactcagaaactaccaagttttcccaacccattttagacataattactttatgaatagcttttcggttatcctccgcaaatttttgcgcaatagccatatcatttagatagtcctctactaatttaccgtctagataagcaaggtctttattaaaaggtgctgtacttagttttaagcgcaataactcttcctgtatgtctttttccctaccttgctcttttagagctctaatgagctcctgtggcttctcagtgttagaacaatacttaacagctagttcttggtgtgttttagctacttgaacgcctaagttacgggaacctgtgtgtgtcataaggaagattcgaccatcgtaggcacctagttcaatatagtggttccctcctcctagtgtaccttctgataaagcgattctattttttacctcttcttttaagtttccatagatagtcagattcttaagtgacttacttaccttctcctgatttagtgctactcctcttacggagtttcccgaaggaacaaactctttaataacactatctaatttttctaatgtagtattgtttacctctaaatctaattcggttactcgtattccgcaaccaatatctactccgataaggttaggtactactttgttagtataagtcatagtggttcctacaacacaccctttgcctgcatgaatatcaggcataaatctaacacgctctcctttaacaaattcctgacttaacaattctattacttgacccacagcggtttcttctacctcactagtaaaaaccttagctgtattataccttccaagtagttctatcattctgctcacgctccttttattatacttcttactaaaaaccattataccatatgtcagtttataagtcaacactaagttaaaaaaagaggaacacgtatgcccctctattttagttctttaaagttcttagagaagaactcctcataatattggtcttctgtagtaactttcctacctgagtctgatactactgttatatctcctactagaacaccgtcttctgtgatactatttacagtaaacgttctgttcttgttacctttttcatgaaatgtcttatttaaatatttttttccttttttagtcatacataacatcctcttttgttaattgtgctagtgctttacaatatttttctttttgtgaggcaaaagcaatagtgttatttgaaaaatagtctagaatctctgctagttttacttttttagctagattatcctgcttcaatctctggatatagcaagtatacgcctcattttgaatgcgtgttaggtgaactaaacttccaagtaaataatccttgtccactagagataaaaaagtttggacatcactaaatctgacggcggtatcctcaaatatatcatgcagtaaacttaaataataagcatcttcatctttgtctttcctaaagttttctgctactcggtacacgtgcgctagctgattttcctcataattagctaaaacagtctttaaatgattacttacgttgtctttaattgtaaacaatacgtagcctcctttaaaaaataattggtagaaccaatagtacacatattcctagagaaacaagtaataacataatagtatctcttactgaaagtaggaacttaagtttaggtacatatggcatagatacccctgctctgcctaaaagattagaagcaataggcacaaggttatactcaataagaaaagctaaatataaataagagaatattaaaataaaagtgattataatccccatagatgtctcctctctactcccctaaggagctattttcatgcttatctattttaaaaagctcactagaaggaacaccaaaatatttttctagtttcactcgatttctctctctaggaaacttcttacctgtctcccaataagaaattagggagtagttaacatctaaatcctgcgctaattgtcgaatagacaactgctgattctctctaattcttcttaaatttcttactttcccaatatcttctttatttgtcattttttttttcaccccttactttttatatatttattataacataaaagtcaacgtctgtcaagcgtacaaaaaaaagagcacgtattaggtgctctctaggttactatagaatgctgtcaataaagctgtaggcttcacttcttaaaacagataccataaaatcttttggtgtatttttttctaaagacattacatgtttaaagacagtaggtgtaattccagatactaaagcaactccgctttcagaaaaccgaactggtgagttatcattgcgggcatttacattccaaaaagttactgttggaagtaagtaacctgcttttgaaaacttacgtctcatactttcgtagaaagagctgtcgttctctttttgtgaactacactggtcgaactccatgtcagagataatgacaatatcagtaggtagctcactttgtggcagtttgctctttgttgcggtattcagaattaagttaaaaacagcctctaggttagtggacatgccccagtcgctactaatagcagtagaaacccgctcccgtaagcttaactcactaggaacatgaactaaagctggtttagaactgaatgtaataaagtggtctttgaacactccatttagtctctctgcgcagtatagccctaaagaaatagaaacgtccataggtgacacttcttttgccccaccatacatagaacctgatacgtcagaaactacaatagcatttctagtcatattttctacgtagttaggtagtgatttccaagttgcctctactgtgttgtctacagagtcgtcccagtcaccacttgcggtgtacttcttaactaactcataaggcataatgttgctggcatttagttttttatctgtggagtttactctgttaataaacatttcataggatgtgggcaggtgttttctaaaagcccctctatatttaaacagggcacgagaaggtaacttgtcaaaattgatagtatcgtaatccttgtcggctaagctagtctccactaagtttaactttgctcgtaaagcggaaagtaatttacgatactctcgactggtgtagcctagggaagcagacaagtctctagctgtatgtttcttctccttgtttggtgaagactccgaaggtaaccacttagctaataaagatatagattcatctgattttaagttctttttatctagttctagttgttcagaaattagttccactagcaccattgttaagactttgtttttagtttcacaactatagcgatatagagatacaacatcgtcccaacgaccaatttcactcataaactctgtaaacttaattagagtgtctaagtcaagctcagctagccccttaatggactcgttatacactgctcgttcacctaagccactaagtcggatattacgaagatgtactaagttcttgagtgctagctctcggtcttctatatatgccttcttaaataacagcttagcatcatttgttctatcacgcattgctcctgctcgtgagaaaaaatctaagttatagtctaaagtagtgctaaatgtgttagcccccttttctgttttacttaaagtagtctcttcttttaatttatttaaaaattctgacataataatctaccccttttcttttttataatcttatagctgacctaagctatattcaggtcagctatcgtatgtacaggaagcgtttttttctcgtacattttttttttacatggttagacaccatgtcgtactaactgctaatatgcgctgttttagtttttagtatttgctgtgtgcttcctaattaatttttacagagagcgtttttactttttcaaatagtataggttagtaattgctgttagctctctattttatattctgtagattatttttttatcacaggaatcatttttatattttatccaataatatagtagtaattgctgttagattcctaagtttattttatcttaaatttatgataacatattttgtctagtatgtcaacatctttttacattttgtttttgttgtcattctgccagttttcccaaaattgctctttttcactttttcggctctcgacaaaaccttgtgtcttcttacaatatgggcaatacatatgtttaatatgaccgtccttcttttgcttagctgaccttctgaatatagagactttttccccacactcagggcatgttaacgtggtttgtactgtctttaccatagttataaccagattgccccgttgggaatcgaacccaaaccgaggagttatgagctccttgctcgaccttctagctacgggacaatgagggaaacttctcttagttagagaaggctccaaaatgttttagttttccaccgagtgcctttgtcttgtctgatagttctatttttgatgttagacgagacttaccttctgccacgtacagaaaatctttctcaacagtaactaacttcatttcttttttgccagtctctttgttgtatcgttcctgtaattctttaacaggttcggaaattacgccgtaaaattttttcattgtaaaacaactcctaattttttattttttaactaactttgtaatttaataatacaataaaagaaatttggagagattccactctcaactacttcgtgcatcaatatattcagtcacggctaaactagattcttggtaaggagctaccttgtcctccatccagtctgtcaattaagactaagtttaaacacatcacgttggaagtgattaaaaatttaccgacaggactattttacctgtacatttcaccaagcatggaagagtatatttttattaataccaattttaaagatgtagattttggctactttgactcccatcatcccttatactcggctttcaccgaatcaactacataaatgacttttagtaagtagttttaaaagagtacttacattcccttctaaaacgcttcatcacgggcaggtaggatacttttatcttataacacacgtagcagtacctttgtctcacactaaccataactatggctcagtccccaacgcatactgatatttaccacatatactaatcgaggcggaaaatttaagtacattagaagagctaccttcttgttatgttacaaatttcctttattctattatcaatgttcttagtaacactataccatagatttcatatagtgtcaagtaataaatcaaggtggtaggattcgaacctacgacttcccgtctccaaagcgggtactctaaccaaactgagctacaccctgtcggttccgtacacaaaccttttaattgcacggcatatttttttgactagctacggacgtctagaacgccagccgacatctcctgtatcagagaagatgaagcagatatatagtattatctgtagtttaaggtatgaggatactagaaccagaaagaagcacctctattagttttcctgttcctatagtatcggaagcaaaaccgaagttataggtggatttcctatcttttttactcgttgtaactcactttagttaccgtttgcttttagcggtgtgagtggtaatctctttactgtacacctagtgtaccacactttgcgtttattgtcaacacttattttctaaaatttttcctgtaccaattttctatcatactatttatatagttattcatgtagtagtcagtaactataatacccttgtctgctgctaatgtatactcatgaacccgtaatatatagtaagtacttataggaagtacaacgttacctgaataaggtttagtagttctctctacatgttttataaatgactctatttctttcatttcttgttggaaagataggtattttccatgtctagtcctaaaaactagtttgcggtaaaacccaccatcagggatatgctcttctctagatattctagacctttgaagtttattcaaccttattttgtctagtctcctctcctttgaatgccagtccgctttactcttgggcgtcttcttataacttctgctcataggacacctcctaatgattatctacctagtatttctctctgatagtcattttaccattcttttcttctgctttacgaaaattctctctaactttagctactctagcttgctcttcttttttctcttcgggtgtcagctccattaaaccgagaactttcatagcatggtcatactcttcttgtgagttacacactactgtatgtttaaaattaatctctgtcaatattgaagcctcccttgttgataattatgtacctatggctgttaactcctattcatttaacttccagtctataagaagcaccacagtttgaaacagatttataaggttcctcaacctacatataagttaactttattatgtatttacaaagtcctcagcaggattcgaacccgcgctaagggagtagaaatcccttgtgcatccgctacaccataaggacactattctcctatacacacagccccgctcttaaccagatgagacgtaacggtcaccgaagcaaccgtaagggagtcgaaccctttccgagctgtgcaaaggaaagtaacataggtctcttattatgttacttagtctagtgtccgtccacataggttgagactacctcgggacacgtccgcattctgttaattcagatttatttagtattgtgttgcaacacaacgctgggatgactaatccctaaagcgggtagggggattcgaacccccactacatggttggaaaccttgggtactacccttatactatacccgcagaggcaacctacttagggcaagtaagttaccaatacaaaaggagtaaaaattaattaaaacaaaaaagcgtatcctgctggatttgaaccagcgacttccgacttaacaggacggcactctgacctctgagttaaggacacaaaatactcggcaaggattttcaccttgcaataaccttcatcattcgtttcagctatcaattcaagcatcggatatgctctcagcttacttctattataggttataccagtcctactgtgcgatattttttccgccacgagtataaaaaatagtaatttaagtacaataggtagcaagtttttgtaagtgctagcaagcgcaatactgtaccactcagtattaaaaaaggtgggatatacaattgtaatttcattttactgtgtttgtaattgttatcgtacatggtttcatacacattgctattgagttaaataacttgctaccgaggtaactgtactatttttgcctgctattcctaaagctcttacaattgacaggtaaggagtcgaaccttaataactaccactagcttgtctttaatacttatcggtgcaccttatagtagacttagggttactagacaaactaaagtatactacatacttgacttactacccgacatttgaagggaggttagaaattgattttagcctctttttatttgctgataatagttttttctactatagcagtaaggtgcaccgtttgtttgtctcaccacatagtaatcctaattttaaggaaggtaagggatttgaacccttgtaccctgtattccaagggcaaactagttttcaagactagcgtattaccaagctctaccaaccttccattttgttgctatggagtccgcaggtatcgaacctgcctctcctgcttttcaagcaagcgctaatccatctcagctaggactacgttaatggagccactaggattcgaacctagaataacggggtaaaaacccgcagtgatagccatttcaccataactcctttgtatagaggttagagggatttgaaccctccgtaactagctatatagtctagtatgtcgtcatttcatcttaacctctataataacattataacatactttaagatgtttgtcaactgttttacagggatagtaggacttgaacccacctcattggagttggaaaccaatattttaccagataaactatacccctaagctggtctagttggattcgaaccaacgtatcacggaatcaaaatccgttgccttaccgcttggctacagaccaatagtaccaagtaagctatgcttgtagtaggacttgaacccacgtctgaggcttacaaaaccactgttctaccaattgaactatacaagcttatttttttattaagtcagctctctaagtctccgctctttacgttagtaattattagctgactcgaagagctaacctattatgcgccatcaaggactcgaaccttggaaccccgaagggaggggagttacagtcccctgcaattgcctctatgcgaatggcacgttgtaaagctagagtacatacttcttgcttattaagataccctagctaatttttactacttgttatatacttcccagtcattagcaaatacatctaagttgctaatacaccacattacatgacttccagtagctgttctcatttgcaagtaaggttctactttaaatagtgcaccttcctctaactcataagcatctgctgtttgtttattagcagggataccctctggatagcctttctgagctactacaaacatgtctttaccattccaacctgttctgctagctttctttccttttttaattaaaggtaaaatctcttcgaatgtcataattttctcctccttctatgtcttattaataccgagagtgggattcgaacccacatatccttgggacagcagattctaagtctaccgtgtataccagttccaccatctcggcaagtgtttctactagtctttgattacattataatagttagcataatgagtagagataacaaacccacctcgaatgtttaagtatggagggattctttctaagtcctgtaaatcaataccatctactaagtaaccgtcattaggggcatctgttaatggggatacagcaagagagttgtctaccgaaatagttttcacgttaggatattttttatcaaaaatagttttctgaaactttgtgcttacaataacagagtatccaaattcctcagctatctccattatagcctgtgtttttccttttctcctatacttaacagtactgataaagaatagaggctccattcgatacatagccttcttaaatcgccttacttctttactctctttaaacttagacttttttactttcttatgagtgttcttttggttcagcatattcatcagtccctttcatttttattcaagcgcacgtaaggggaatcgaacccctatttctcgatagacagtcgagtataatagccattatatcatacgcacagtactgaggtacttctagctaaaaagttacctcagagtttaacttggcaatagactggtcggctgtgtttctttagctaaggaagccttctacgcactattagtaccaacacttggaattgaaccaaggactaccgattatcggtcggttgctttgcctcctaagctatgctggtatagtatactctctcgttgactactatacgggggaatgtattaataggaacgagagaatacctatttttaattataccacactctaacagctttctgagattttaatctcttgggttttcctttttcaaggtcaaggtttctggtttagtgtgtctactacaaaccttttttcgaaaggagagtaagggattcgaacccttggtactattactagtacaacgagttagcaacccgtatcattaagcctctctgacaactctccaagaaacaaaatttaaacttatatttatattataacataaattagtacgtttgtctactaataacttattttttttcataaagcgggtaatgggaattgaacccatatacgtagatttgcagtctactacctttccagttggttatacccgcaagttctagaagactaataagactcctagagtttttaaatatttagtgaaggtaaagatataaacctgatgataagaaaaacgtgttactaacaatcgtctaacttacctgtatatacaatatagcatacttactgataaaaagcaacagttttctataatatttttcatactgagctaagaaaatgtatagcaaccctaaaactatataattatctgacaactgtcctatttaattttattcgtcccctatattatattctaatatattatataattatttaaagatatgtaattagacttagaatataaaaggaatataatataggagaaccttaaaattaaataggacaattatctgaatattttataacgactactattacttgagtaacaatagaaacttgatactagtatcaaataaaaagtaatttaatattgatagtaacccacctcaggtacctaaatatattcagaaaatttatactactgtcctatttaattttatctacctcctatattatattctaatatattatataaagactataataaattattattattattattatttataatatagaagagtcctaaaattaaataggacaattatctgaatataaagtttaattaacataaaaagacacctgtaaaggcatctttattaaacatctaaaaatttcttttcgtaggatactacaagtgaggcatttacataaacttcttctccatctaatgctttagtagttctaaccttaataaatggtaactcagaacctcttacagcagagtttaacttagtactagggtcagtaacagtatcaaatacatcttcactgaaaagataaaatacatctcctgcaatagtctttagttctaaaacagtttgtcttctagccatttttattgttcctcctctgttatgtggtataattaaagtatatcacacattacttcttaactaaactgaaaacataggtaactaccgcacctagtataatcataaatactttttctactaagtcttttttaccttcatcttctttttggttcgtttgtcttagcgcttccacacctttctctaattgacctatctgatagaagatgtgtgactgcttttcagattgtagggcagaatctttatctagagccactacaatatctcgtaattccttgatagcttttgttagctcctcattgtcagtcttacgtgttagttcacccgtttgtactttatcttcaaggttttttattttatctagcagactttgaatatttgtatctttttccactcacatcctcctcctctcagttctttatttttggtagggtttacgaatgtgttctgatacatccgagctttcagggtagttatctaagaagttcctaattatatcttcataatcaggtgttacaattctcccgtaatgtgtaacaaagtttaggtgaaagtcaattctaccgtaggaacctaggtcaaggtacttaccaaccatgtcgtcttcatcaactttaaaataaccaaagtcaaaacctataagggttcccattgatagagccctactaataaagtagatatgatattcttcacgattttccgagaggaagttacgtaatatatctctaaattctacttttagcatatccctgtattctttcattgcagactcctttctttacatactccatgcaaccactactataaccataaaagatacaactattacaaaatatataataaaggtgtaaactaacgacataagtagtacaatgttagccacacctgctataaacgttgctataattagcgtaaagttttgcgtaataccttgtatatctcctagagaaacatggcgttgcaagtcacttataacgtggtatgaagtccaaaaaatacccacttgaaacaagacagaggctagtacagctataaacattatactcatagtaagacaacctccttaaagctagttctaatatagtatacgacatagaataatttaaagttag

SEQ ID NO: 4 - protein, 181 aa, Unknown
  Distal 181 amino acids of the receptor binding protein of bacteriophage P100
VTANTVAVERVVADRIITTLGVPSKWEYRAKASNFFFSASNSSRITLVSTQQNIITPNKFINNPGSLPLSAINPAITIPEDGMYQVMAALNINGILEKVYLVSELTLLINDVVHPATFGMVKTADNANGQYSLAGNGIYQLKKGDKLKLRSYCNNTGNNPYLDVDKLYISVGKIADISLFK

SEQ ID NO: 5 - protein, 181 aa, Unknown
  Distal 181 amino acids of the receptor binding protein of bacteriophage A511
VTANTVEVERVVADRIITTLGVPSKWEYRAKASNFFFSASNSSRITLVSTQQNIITPNKFINNPDSLPLSAINPAITIPEDGMYQVMVTLNINGILEKVYLVSELTLLVNDVVHPATFGMVKTVDNANGQYSLAGNGIYQLKKGDKLKLRSYCNNTGNNPYLDVDKLYISVGKIADISLFK

SEQ ID NO: 6 - protein, 181 aa, Unknown
  Distal 181 amino acids of the receptor binding protein of bacteriophage P100+
VTANTVEVERVVADRIITTLGVPSKWEYRAKASNFFFSASNSSRITLVSTQQNIITPNKFINNPGSLPLSAINPAITIPEDGMYQVMAALNINGILKKVYLVSDLTLLINDVVHPATFGMVKTADNANGQHSLAGNGIYQLKKGDKLKLRSYCNNTGNNPYLDVDKLYISVGKIADISLFK

SEQ ID NO: 7 - protein, 181 aa, Unknown
  Distal 181 amino acids of the receptor binding protein of bacteriophage P200
VTANTVAVERVVADRIITTLGVPSKWEYRAKASNFFFSASNSSRITLVSTQQNIITPNKFINNPGSLPLSAINPAITIPEDGMYQVMAALNINGILKKVYLVSELTLLINDVVHPATFGMVKTVDNANGQYSLAGNGIYQLKKGDKLKLRSYCNNTGNNPYLDVDKLYISVGKIADISLFK

SEQ ID NO: 8 - DNA, 988 bp, Unknown
  Recombinant construct encoding the distal part of the P200 receptor binding protein
gtgacagccaacactgtagcagtagagagggtagtcgcagataggattattactactctaggggtaccgagtaagtgggaatacagagctaaagcctctaatttcttctttagtgcttccaattcgtcacgtataacactagtatcgacccaacagaatattataacacctaataaatttattaataaccccggtagtttaccactatctgcgataaaccccgcaataactattccagaagatgggatgtaccaagtgatggcggccctaaacattaacggtattttaaaaaaagtttacttagtttctgaattaactctactaataaatgacgtagtacacccagctacatttggaatggttaaaactgttgataacgctaacggtcaatactctttagcagggaatggcatatatcaactaaaaaaaggggacaagttaaaactaagaagttactgtaacaatacagggaataacccttacttggatgtagacaaactttatatatctgttggtaagattgcagacatttcattatttaaataaacttagtatgctgagaaaggaagtcacaataatggcagaagaaaaattttcattggacggaaggcagtatgtaacagcagtcctatatatgtaccctaacctagaccctcagtacgactttggagttgtatcatccgaagggaaggtagaagcttttgaatggcacgtaaaaaatataacaaagccttctattgcacagttagaccaagcatataaagactatgtaaaatctatcggaagtccaagtaatccaccagccctagaaccagaggtatcaaaagaagcctcagtaggtaagattctaactgaaaaatttaacaccttagaagaaacaaatgctaatatgttattggaaaatctagagcttcaagctagcctagataaaacaaaacaacaaaattcagaactagtatttgagctagtagataggggagtcttatga

SEQ ID NO: 9 - DNA, 988 bp, Unknown
  Recombinant construct encoding the distal part of the P100+ receptor binding protein
gtgacagccaacactgtagaagtagagagggtagtcgcagataggattattactactctaggggtaccgagtaagtgggaatacagagctaaagcctctaatttcttctttagtgcttccaactcgtcacgtataacactagtatcgacccaacagaatattataacacctaataaatttattaataaccccgatagtttaccactatctgcgataaaccccgcaataactattccagaagatgggatgtaccaagtgatggcggccctaaacattaacggtattttaaaaaaagtttacttagtttctgatttaactctactaataaatgacgtagtacacccagctacatttggaatggttaaaactgctgataacgctaacggtcaacattctttagcagggaatggtatttatcaactaaaaaaaggggacaagttaaaactaagaagttattgtaacaatacaggaaataacccttacttggatgtagacaaactttatatatctgttggtaagattgcagacatttcattatttaaataaacttagtatgctgagaaaggaagtcacaataatggcagaagaaaaattttcattggacggaaggcagtatgtaacagcagtcctatatatgtaccctaacctagaccctcagtacgactttggagttgtatcatccgaagggaaggtagaagcttttgaatggcacgtaaaaaatataacaaagccttctattgcacagttagaccaagcatataaagactatgtaaaatctatcggaagtccaagtaatccaccagccctagaaccagaggtatcaaaagaagcctcagtaggtaagattctaactgaaaaatttaacaccttagaagaaacaaatgctaatatgttattggaaaatctagagcttcaagctagcctagataaaacaaaacaacaaaattcagaactagtatttgagctagtagataggggagtcttatga

(19) United States
(12) Patent Application Publication
(10) Pub. No.: US 2023/0380458 A1
(43) Pub. Date: Nov. 30, 2023

(54) A NOVEL PHAGE FOR LISTERIA, INCLUDING LISTERIA MONOCYTOGENES

(71) Applicant: Micreos Food Safety B.V., Wageningen (NL)

(72) Inventor: Steven HAGENS, Wageningen (NL)

(73) Assignee: Micreos Food Safety B.V., Wageningen (NL)

(21) Appl. No.: 18/246,681

(22) PCT Filed: Sep. 30, 2021
(86) PCT No.: PCT/EP2021/076894

§ 371 Date: Mar. 27, 2023

(30) Foreign Application Priority Data
Oct. 1, 2020 (EP) 20199513.1

(51) Int. Cl. A23L 3/3571; C12Q 1/04; C12N 7/00

(52) U.S. Cl. CPC A23L 3/3571; C12Q 1/04; C12N 7/00; C12N 2795/10031; C12N 2795/10021; C12N 2795/10022

(57) ABSTRACT

The invention relates to the field of microbiology, specifically to a bacteriophage or a combination of bacteriophages, to a composition comprising said bacteriophage or comprising said combination of bacteriophages for preventing, treating or controlling contamination with and/or growth of Listeria, including Listeria monocytogenes and Listeria serovar (SV) 1/2 mutants that are resistant to bacteriophage P100 and/or Listeria serovar 3. The invention further relates to the a method for controlling contamination with Listeria, including Listeria monocytogenes and Listeria serovar (SV) 1/2 mutants that are resistant to bacteriophage P100 and/or Listeria serovar 3, in a food product, on food processing equipment, or on food storage containers by applying the bacteriophage or the combination of bacteriophages, to the composition comprising said bacteriophage or said combination of bacteriophages to a food product or food processing equipment to reduce the amount of Listeria.

### FIELD OF THE INVENTION

The invention relates to the field of microbiology, specifically to a bacteriophage or a combination of bacteriophages, to a composition comprising said bacteriophage or comprising said combination of bacteriophages for preventing, treating or controlling contamination with and/or growth of Listeria, including Listeria monocytogenes and Listeria serovar (SV) 1/2 mutants that are resistant to bacteriophage P100 and/or Listeria serovar 3. The invention further relates to the a method for controlling contamination with Listeria, including Listeria monocytogenes, and Listeria serovar (SV) 1/2 mutants that are resistant to bacteriophage P100 and/or Listeria serovar 3, in a food product, on food processing equipment, or on food storage containers by applying the bacteriophage or the combination of bacteriophages, to the composition comprising said bacteriophage or said combination of bacteriophages to a food product or food processing equipment to reduce the amount of Listeria, including Listeria monocytogenes,.

### BACKGROUND OF THE INVENTION

Phages, as antibacterial agents, have the advantage of replicating within the bacterial target. Thus, when their progeny lyse the cell and escape into the extracellular milieu, they can infect and multiply in succeeding generations of bacteria, producing progeny levels far greater than that of the binary growth of the target bacteria, thereby increasing the phage population exponentially in numbers at the expense of the bacterial targets. Bacteriophages that are able to lyse several Listeria serovars are known to the person skilled in the art. Serovars are determined by variations in the sugar decoration of teichoic acids (cellular antigens) on one hand and the type of flagellum on the other. No flagellotropic Listeria phages are known to date and recognition of target bacteria appears to be determined by the type of teichoic acids. The cellular antigen appears in five forms, namely SV 1/2, 3, 4, 5 and 6. L. monocytogenes isolates are either SV 1/2,3 or 4, whereas L. seeligeri isolates are SV 1/2 or 3. L. welshimeri isolates are SV 6 as is L. innocua. SV 5 is only found in L. ivanovii. Bacteriophage P100 (genus Pecentumvirus) is such a bacteriophage and P100 has been used for quite some time to effectively control contamination of food products with Listeria, including Listeria monocytogenes (see e.g. WO2004/004495). Bacteriophage P100 is effective against several Listeria serovars, including serovars 1/2, 4, 5, and 6. Bacteriophage P100 was deposited at the American Type Culture Collection, 10801 University Blvd., Manassas Va. 20110-2209 on May 23, 2002, 2002, ATCC patent deposit designation number PTA-4383.

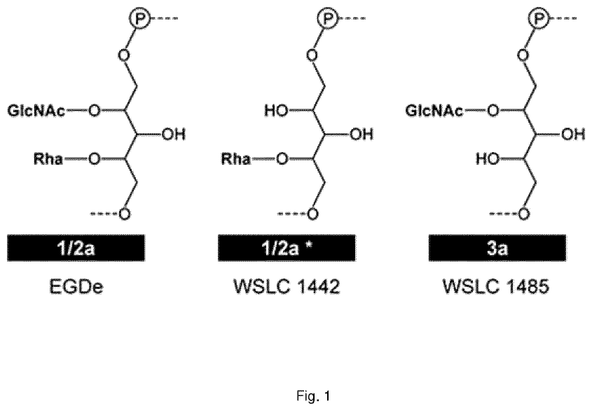

Several mutations in the cell-wall teichoic acids of Listeria serovar 1/, lead to bacteriophage resistance. The cell-wall teichoic acids of Listeria serovar 1/2 strain carry sugar decorations, namely an N-acetyglucosamine (GlucNac) residue and a rhamnose (Rha) molecule, strains that have lost the GlucNac are designated SV 1/2*, and those lacking Rha are designated SV 3 (see FIG. 1). These mutations are quite commonly found and none of the known members of the genus Pecentumviruses (such as bacteriophage P100) are able to recognize, infect and lyse these Listeria mutants, rendering the known bacteriophages ineffective against such strains. Since the mutants described here above (depicted serovar 1/2* and serovar 3) are quite common, there is thus a need for bacteriophages that are able to recognize, infect and lyse depicted Listeria serovar 1/2* and/or serovar 3.

TABLE 1
Overview of sequences

| SEQ ID NO: | Name construct | Remark |
|---|---|---|
| 1 | Genome of bacteriophage P100 | DNA |
| 2 | Genome of bacteriophage P100+ | DNA |
| 3 | Genome of bacteriophage P200 | DNA |
| 4 | Distal 181 amino acids of the receptor binding | Protein |
| | protein of bacteriophage P100 | |
| 5 | Distal 181 amino acids of the receptor binding | Protein |
| | protein of bacteriophage A511 | |
| 6 | Distal 181 amino acids of the receptor binding | Protein |
| | protein of bacteriophage P100+ | |
| 7 | Distal 181 amino acids of the receptor binding | Protein |
| | protein of bacteriophage P200 | |
| 8 | Recombinant construct encoding the distal part | DNA |
| | of the P200 receptor binding protein | |
| 9 | Recombinant construct encoding the distal part | DNA |
| | of the P100+ receptor binding protein | |

### BRIEF DESCRIPTION OF THE FIGS.

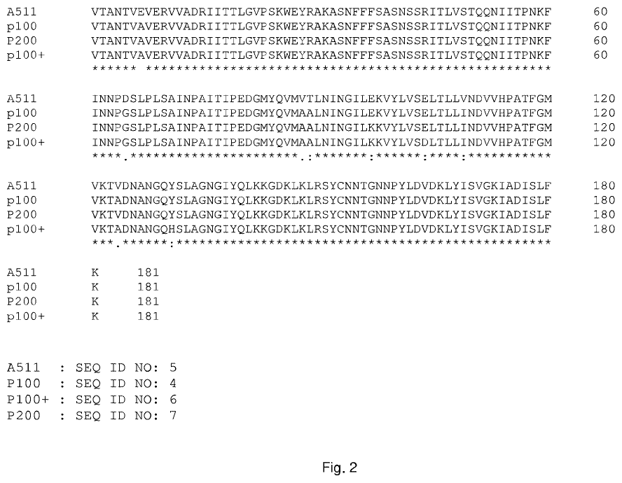

FIG. 1 depicts the sugar moieties of the cell-wall teichoic acids of various Listeria serovars. Serovar 1/2contains an N-acetyglucosamine (GlucNac) residue and a rhamnose (Rha) residue, strains that have lost the GlucNac are designated SV 1/2*, and those lacking Rha are designated SV 3. FIG. 2 depicts the distal 181 amino acid residues of the receptor binding proteins of phages A511, P100 and of the two phages with extended host ranges: P100+ and P200.

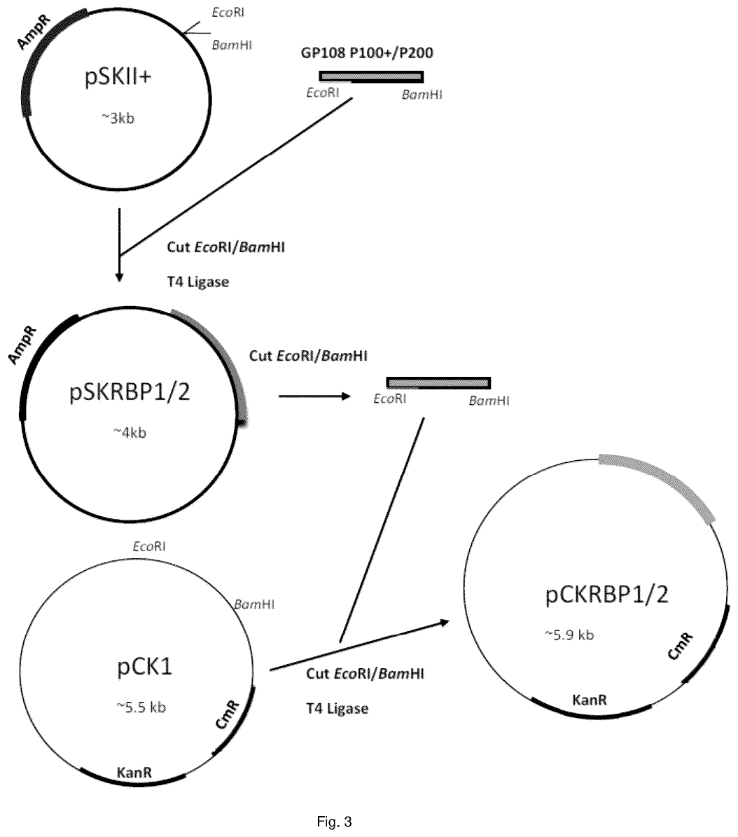

FIG. 3 depicts a schematic overview of the cloning steps in the preparation of bacteriophages A511::P200 and A511::P100+.

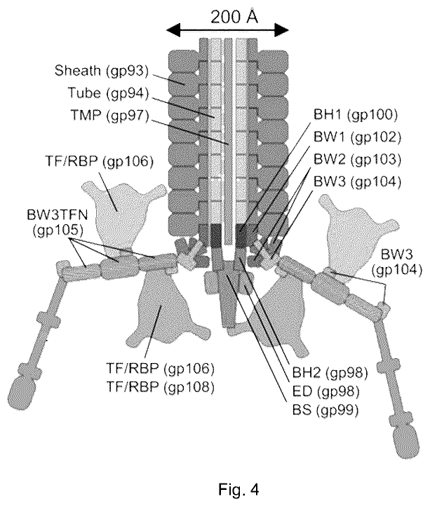

FIG. 4 depicts the location of the receptor binding protein at the end of the phage tail fiber.

### DESCRIPTION OF THE INVENTION

The inventors have established that specific variant bacteriophages have an extended host range in view of their counterparts known to date; they variants are able to recognize, infect and lyse depicted Listeria serovar 1/2* and/or serovar 3.

Accordingly, in a first aspect, there is provided a bacteriophage lytic for at least Listeria serovar 1/2* and/or serovar 3, wherein said bacteriophage has a genome that has at least 70% sequence identity with the genome of bacteriophage P100 as set forward in SEQ ID NO: 1 or with the genome of bacteriophage P100, ATCC patent deposit designation number PTA-4383, and wherein the distal part of the receptor binding protein as set forward in SEQ ID NO: 4 has at least one mutation selected from: G157R, E97K, E104D and Y131H. Said bacteriophage is herein referred to as a bacteriophage as disclosed herein. In an embodiment, the distal part of the receptor binding protein as set forward in SEQ ID NO: 4 has at least two mutations selected from:
G157R, E97K, E104D and Y131 H. In an embodiment, the distal part of the receptor binding protein as set forward in SEQ ID NO: 4 has the mutations selected from: E97K; E97K and E104D; E97K and Y131H; E97K, E104D and Y131H; E104D and Y131H; G157R and E97K; G157R, E97K and E104D; G157R, E97K and Y131 H; G157R, E97K, E104D and Y131H; G157R, E104D and Y131 H. In an embodiment, the bacteriophage as disclosed herein has a genome that has least 70, 75, 80, 81, 82, 83, 84, 85, 86, 87, 88, 89, 90, 91, 92, 93, 94, 95, 96, 97, 98, at least 99 or 100% sequence identity with the genome of bacteriophage P100 as set forward in SEQ ID NO: 1 or with the genome of bacteriophage P100, ATCC patent deposit designation number PTA-4383.

In an embodiment, the bacteriophage as disclosed herein is bacteriophage P100+as deposited under number CBS146750 at the Westerdijk Fungal Biodiversity Institute, Uppsalalaan 8, 3584 CT Utrecht, The Netherlands. In an embodiment, the bacteriophage as disclosed herein is bacteriophage P200 as deposited under number CBS146751 at the Westerdijk Fungal Biodiversity Institute. In an embodiment, the bacteriophage as disclosed herein is bacteriophage P100+having the genome according to GenBank accession nr. MT 178766.1 (corresponding to SEQ ID NO: 3). In an embodiment, the bacteriophage as disclosed herein is bacteriophage P200 having the genome according to GenBank accession nr. MT 178767.1 (corresponding to SEQ ID NO: 4).

In an embodiment, the bacteriophage as disclosed herein is also lytic for at least one of Listeria serovars 1/2, 4, 5 and 6, such as Listeria serovar 1/2, Listeria serovar 4, Listeria serovar 5, Listeria serovar 6, Listeria serovars 1/2 and 4, Listeria serovars 1/2 and 5, Listeria serovars 1/2 and 6, Listeria serovars 1/2, 4 and 5, Listeria serovars 1/2, 4 and 6, Listeria serovars 1/2, 5 and 6, Listeria serovars 1/2, 4, 5 and 6, Listeria serovars 4 and 5, Listeria serovars 5 and 6, and Listeria serovars 4 and 6.

In a second aspect, there is provided a composition comprising at least one bacteriophage according to the first aspect. The features of the second aspect are preferably those of the first aspect. The person skilled in the art will comprehend that such composition as disclosed herein may comprises two or more bacteriophages as disclosed herein, such as a P100+ bacteriophage and a P200 bacteriophage.

The person skilled in the art will comprehend that the composition as disclosed herein may comprise one or more further compounds, such other active agents, and excipients. In an embodiment, the composition as disclosed herein further comprises an agent selected from the group consisting of a bacteriophage endolysin, a surface disinfectant, an antibiotic, a surfactant, an antibacterial enzyme, a further bacteriophage lytic for Listeria, such as Listeria monocytogenes, and a bacteriophage specific for bacterial contaminants other than Listeria. A further bacteriophage lytic for Listeria, such as Listeria monocytogenes, may be, but is not limited, to bacteriophages phages B054, A118, A502, A006, A500, A511, PSA, P35 and related viruses). These bacteriophages are described in WO2004/004495 which is herein incorporated by reference; the person skilled in the art will comprehend that the subject matter of WO2004/004495 can be combined with the subject matter disclosed herein. A further bacteriophage may also be a virulent bacteriophage specific for Salmonella enterica (see e.g. Marti et al, 2013, Mol. Microbiol. 87(4), 818-834). Such bacteriophage may be bacteriophage S16 belonging to the order Caudovirales. Phage S16 has a contractile tail, which is the defining morphological feature of the Myoviridae family. Phage S16 is the first strictly virulent, non-toxic broad host range T-even like bacteriophage solely infecting Salmonella bacteria ever described. Phage S16 lacks any kind of virulence factors as is the case for other T-even phages described in the literature. Phage S16 is a new member of the genus of T4-like viruses, belonging to the T-even type of subgroup and is the first fully characterized member of the T4-like phages limited to infecting Salmonella. This bacteriophage and derivatives thereof are extensively described in WO2013/169102, which is herein incorporated by reference; the person skilled in the art will comprehend that the subject matter of WO2013/169102 can be combined with the subject matter disclosed herein. Phage S16 is deposited at the Westerdijk Fungal Biodiversity Centre under number CBS130493.

It has previously been demonstrated that Listeria bacteriophages when combined with an organic acid or a salt thereof have increased efficiency of lysing Listeria, such as Listeria monocytogenes, especially when applied on food for controlling Listeria, such as Listeria monocytogenes. Such combination does not have to be comprised in a single composition, the person skilled in the art will comprehend that the bacteriophage composition as disclosed herein and a composition comprising the organic acid may be administered separately, either consecutively of simultaneously. Accordingly, in an embodiment there is provided a composition as disclosed herein further comprising an organic acid, or a salt thereof, such as wherein the organic acid is selected from the group consisting of lactic acid, such as L-lactic acid, acetic acid, propionic acid and mixtures thereof, such as wherein the salt of the organic acid is selected from the group consisting of the sodium salt, potassium salt, ammonium salt and mixtures thereof, such as K-(L)lactate, Na-(L)lactate, K-acetate, Na-acetate, K-diacetate, Na-diacetate, K-propionate, Na-propionate and mixtures thereof. Such compositions comprising similar (e.g. P100 and A511) bacteriophages are extensively described in WO2018/141800, which is herein incorporated by reference; the person skilled in the art will comprehend that the subject matter of WO2018/141800 can be combined with the subject matter disclosed herein. In this embodiment, the organic acid may be selected from the group consisting of lactic acid, such as L-lactic acid, acetic acid, propionic acid and mixtures thereof. The salt of the organic acid may be selected from the group consisting of the sodium salt, potassium salt, ammonium salt and mixtures thereof, such as K-(L)lactate, Na-(L)lactate, K-acetate, Na-acetate, K-diacetate, Na-diacetate, K-propionate, Na-propionate and mixtures thereof. The organic acid may be acetic acid in a buffered aqueous solution comprising 2% to 30% acetate, such as comprising 5% to 20% acetate, such as comprising 10% to 20% acetate, such as comprising 15% to 20% acetate, such as comprising 15, 16, 17, 18, 19 or 20% acetate, such as comprising 17% acetate. The pH of the acetic acid in a buffered aqueous solution may be 2 to 7, such as pH 5 to 6.5, such as pH 5.7 to 6.3. Buffering may be performed using sodium acetate, acetic acid, sodium hydroxide, sodium carbonate and/or sodium bicarbonate.

The salt of the organic acid may be in an aqueous solution comprising a mixture of K-(L)lactate and Na-diacetate, such as comprising 50% to 80% K-(L)lactate and 2% to 10% Na-diacetate, such as comprising 60% to 80% K-(L)lactate and 3% to 10% Na-diacetate, such as comprising 70% to 75% K-(L)lactate and 4% to 6% Na-diacetate, such as comprising 72.8% K-(L)lactate and 5.2% Na-diacetate. The aqueous solution may be buffered as described previously herein.

In an embodiment, in the composition as disclosed herein, the bacteriophage as disclosed herein is present in an aqueous liquid and comprises 1 ×10⁷ PFU/ml to 1×10¹¹ PFU/ml, such as 1×10⁸ PFU/ml 1×10¹⁰ PFU/ml, such as 1×10⁹ PFU/ml to 1×10¹¹ PFU/ml, such as 1×10⁹ PFU/ml. PFU herein is Plaque Forming Unit. The person skilled in the art knows how to calculate and assay PFU's.

A bacteriophage and composition disclosed herein can conveniently be used for controlling Listeria, including Listeria monocytogenes, contamination in a food product. Accordingly, in a third aspect, there is provided, a method for controlling Listeria, including Listeria monocytogenes, contamination in a food product, on food processing equipment or on food storage containers, comprising contacting a bacteriophage or a combination of bacteriophages as disclosed herein, or a composition comprising said bacteriophage or comprising said combination of bacteriophages as disclosed herein, to a food product or to food processing equipment to reduce the amount of Listeria, including Listeria monocytogenes. Said method is herein referred to as a method as disclosed herein. The features of said method as disclosed herein are preferably those of the first and second aspect. The food product may be any food product that is known in the art and is susceptible to bacterial contamination and/or spoilage, the food product may be selected from the group consisting of poultry, such as chicken and turkey, beef, pig, horse, donkey, rabbit, goat, sheep, salmon, trout, lobster, clamps, shrimps, crawfish, cheese, ice cream, sausage, such as frankfurter, bologna, meatloaf, roast beef, ham, sliced meat and mixtures thereof, as known in the art.

In an embodiment, the food product is a processed, non-processed, cured or uncured food product selected from the group consisting of meat, fish, shellfish, pastry, dairy product, vegetables, fruit and mixtures thereof. In an embodiment, the food product is selected from the group consisting of beef, pork, lamb, fruit, vegetables, including but not limited to lettuce, leafy greens, baby leafy greens, sprouts. The processing or curing of the food product may be a process selected from the group consisting of cooking, salting, baking, steaming, smoking, grilling, roasting, drying and brining (e.g. injecting with brine). In an embodiment, 1×10⁵ PFU/ml to 1×10⁹ PFU/ml of a bacteriophage as disclosed herein is administered per cm² of food product or food processing equipment, such as 1×10⁶ PFU/ml to 1×10⁸ PFU/ml, such as 1×10⁷ PFU/ml to 1×10⁸ PFU/ml, such as 1×10⁷ PFU/ml, 2×10⁷ PFU/ml, 3 ×10⁷ PFU/ml, 4×10⁷ PFU/ml, 5×10⁷ PFU/ml, 6×10⁷ PFU/ml, 7×10⁷ PFU/ml, 8×10⁷ PFU/ml, 9 ×10⁷ PFU/ml, or 1×10⁸ PFU/ml.

The food product may be packaged, usually immediately after performing a method as disclosed herein or as a step within a method as disclosed herein. The food product may be packaged such that it can be stored and transported from the manufacturer via the distributor, wholesale, retail to the customer. The package may be a package under vacuum or under protective atmosphere using an inert gas. The packaging material may contain oxygen scavengers or the like. The packaged food product may be stored at temperatures below room temperature, such as 4 degrees Celsius/29 Fahrenheit.

In a method as disclosed herein, a bacteriophage or a composition as disclosed herein may be administered to the food product in any way known to the person skilled in the art, such as by mixing with the food product or dipping the food product in a composition comprising a bacteriophage as disclosed herein. In an embodiment, a method for administering is wherein the administration of a bacteriophage as disclosed herein is performed by spraying or misting said bacteriophage or composition. Spraying of bacteriophage compositions is known in the art and any suitable method known may be used; the person skilled in the art knows to select a proper method.

In an embodiment, the food storage container is a salad bar.

In an embodiment, the food processing equipment is selected from the group consisting of a tube through which milk is being pumped, a high-salt content tank for processing cheese, a container from which cultures are applied to a surface of a cheese, a storage shelve for a food product, a shelve on which a food product is dried and cured, and a floor drain.

In an embodiment, a bacteriophage as disclosed herein or a composition as disclosed herein are sprayed onto a surface selected from the group consisting of food products, food processing equipment and food storage containers.

When the method as disclosed herein comprises administration of a bacteriophage as disclosed herein and of an organic acid or a salt thereof, said bacteriophage and organic acid, or a salt thereof, may be present in different solutions and the spraying may be performed from different nozzles. The spraying of the at least one bacteriophage and of the organic acid, or a salt thereof, is may be performed consecutively or simultaneously. Simultaneously herein means that the spraying of the two compounds is performed at the same time or shortly after each other, e.g. at most 1, 2, 3, 4, 5, 20, 30, 40, 50 or 60 seconds after each other.

Further provided is a food product obtainable of obtained by a method as disclosed herein. Such food product may be packaged as defined previously herein.

Further provided is a food product that comprises at least 1×10³ PFU, or at least 1×10³ PFU equivalents, per average gram of food product of a bacteriophage as disclosed herein. At least 1×10³ PFU may be at least 1×10³ PFU or 1×10⁴ PFU or 1×10⁵ PFU or 1×10⁶ PFU or 1×10⁷ PFU or 1×10⁸ PFU or at least 1×10⁹ PFU. At least 1×10³ PFU equivalents may be at least 1×10³ PFU equivalents or 1×10⁴ PFU equivalents or 1×10⁵ PFU equivalents or 1×10⁶ PFU equivalents or 1×10⁷ PFU equivalents or 1×10⁸ PFU equivalents or at least 1×10⁹ PFU equivalents. PFU equivalent means that said amount of bacteriophage as disclosed herein has been administered by treatment of the food product. The food product maybe any food product, such as the food products described elsewhere herein.

The bacteriophages as disclosed herein can conveniently be used for the detection of Listeria, including Listeria monocytogenes. Accordingly, in a fourth aspect, there is provided a method for detection of Listeria, including Listeria monocytogenes, such as serovar 1/2* or serovar 3, comprising obtaining a sample suspected to contain Listeria, including Listeria monocytogenes, incubating said sample with a bacteriophage as disclosed herein or a bacteriophage as disclosed herein further comprising a detectable label, and detecting a change in said sample caused by said bacteriophage, as an indication of the presence of Listeria, including Listeria monocytogenes. The features of this aspect are preferably those of the first, second and third aspect.

In an embodiment, said change in the sample is due to lysis of said Listeria, including Listeria monocytogenes, by said bacteriophage or said change in the sample is due to the detectable label.

### Definitions

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In an embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds.,

Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. The amino acid change may be conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence. A “nucleic acid construct” is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for a polypeptide as disclosed herein such that the control sequence directs the production/expression of a polypeptide as disclosed herein in a cell and/or in a subject. “Operably linked” may also be used for defining a configuration in which a sequence is appropriately placed at a position relative to another sequence coding for a functional domain such that a chimeric polypeptide is encoded in a cell and/or in a subject. “Expression” is construed as to include any step involved in the production of the peptide or polypeptide including, but not limited to, transcription, post-transcriptional modification, translation, post-translational modification and secretion.

A “control sequence” is defined herein to include all components which are necessary or advantageous for the expression of a polypeptide. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended herein, the term usually refers to an extrachromosomal, self-replicating vector which harbors a selectable antibiotic resistance.

An “expression vector” may be any vector which can be conveniently subjected to recombinant DNA procedures and can bring about the expression of a nucleotide sequence encoding a polypeptide as disclosed herein in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context herein, a promoter typically ends at nucleotide −1 of the transcription start site (TSS).

A “polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) typically means that the value may be the given value (of 10) more or less 10% of the value.

All patent and literature references cited in the present specification are hereby incorporated by reference in their entirety.

The following examples are offered for illustrative purposes only, and are not intended to limit the scope of the present invention in any way.

### Further Embodiments

Further embodiments of the invention are listed here below.
1. A bacteriophage lytic for at least Listeria serovar 1/2* and/or serovar 3, wherein said bacteriophage has a genome that has at least 70% sequence identity with the genome of bacteriophage P100 as set forward in SEQ ID NO: 1, and wherein the distal part of the receptor binding protein as set forward in SEQ ID NO: 4 has at least one mutation selected from: G157R, E97K, E104D and Y131H.
2. A bacteriophage according to embodiment 1, wherein the distal part of the receptor binding protein as set forward in SEQ ID NO: 4 has at least two mutations selected from: G157R, E97K, E104D and Y131H.
3.A bacteriophage according to embodiment 1 or 2, wherein the distal part of the receptor binding protein as set forward in SEQ ID NO: 4 has the mutations selected from: E97K; E97K and E104D; E97K and Y131H; E97K, E104D and Y131H; E104D and Y131H; G157R and E97K; G157R, E97K and E104D; G157R, E97K and Y131H; G157R, E97K, E104D and Y131H; G157R, E104D and Y131H.
4. A bacteriophage according to any one of embodiments 1 to 3, wherein said bacteriophage is bacteriophage P100+ as deposited under number CBS146750 or is bacteriophage P200 as deposited under number CBS146751.
5. A bacteriophage according to any one of embodiments 1 to 4, wherein said bacteriophage is bacteriophage P100+ having the genome according to GenBank accession nr. MT 178766.1 (SEQ ID NO: 3) or is bacteriophage P200 having the genome according to GenBank accession nr. MT 178767.1 (SEQ ID NO: 4).
6. A bacteriophage according to any one of embodiments 1 to 5, wherein said bacteriophage is also lytic for at least one of Listeria serovars 1/2, 4, 5 and 6.
7. A composition comprising at least one bacteriophage according to any one of embodiments 1 to 6.
8. A composition according to embodiment 7, further comprising an agent selected from the group consisting of a bacteriophage endolysin, a surface disinfectant, an antibiotic, a surfactant, an antibacterial enzyme, a further bacteriophage lytic for Listeria and a bacteriophage specific for bacterial contaminants other than Listeria.
9. A composition according to embodiment 7 or 8, further comprising an organic acid, or a salt thereof, such as wherein the organic acid is selected from the group consisting of lactic acid, such as L-lactic acid, acetic acid, propionic acid and mixtures thereof, such as wherein the salt of the organic acid is selected from the group consisting of the sodium salt, potassium salt, ammonium salt and mixtures thereof, such as K-(L)lactate, Na-(L)lactate, K-acetate, Na-acetate, K-diacetate, Na-diacetate, K-propionate, Na-propionate and mixtures thereof.
10. A method for controlling Listeria contamination in a food product, on food processing equipment or on food storage containers, comprising contacting a bacteriophage or a combination of bacteriophages according to any one of embodiments 1 to 6, or a composition comprising said bacteriophage or comprising said combination of bacteriophages according to any one of embodiments 6 to 9, to a food product or to food processing equipment to reduce the amount of Listeria.
11. A method according to embodiment 10, wherein the food product is a processed, non-processed, cured or uncured food product selected from the group consisting of meat, fish, shellfish, pastry, dairy product, vegetables, fruit and mixtures thereof.
12. A method according to embodiment 11, wherein the food product is selected from the group consisting of beef, pork, lamb, fruit, vegetables, including but not limited to lettuce, leafy greens, baby leafy greens, sprouts.
13. A method according to any one of embodiments 10 to 12, wherein said bacteriophage or composition is administered by spraying or misting said bacteriophage or composition to the food product or by dipping or soaking the food product into said composition.
14. A method according to embodiment 10, wherein said food storage container is a salad bar.

method according to embodiment 10, wherein said food processing equipment is selected from the group consisting of a tube through which milk is being pumped, a high-salt content tank for processing cheese, a container from which cultures are applied to a surface of a cheese, a storage shelve for a food product, a shelve on which a food product is dried and cured, and a floor drain.
16. A method according to any one of embodiments 10 to 15, wherein said bacteriophage or composition are sprayed onto a surface selected from the group consisting of food products, food processing equipment and food storage containers.
17. A method for detection of Listeria, including serovar 1/2* or serovar 3, comprising obtaining a sample suspected to contain Listeria, incubating said sample with a bacteriophage as defined in any one of embodiments 1 to 6 or a bacteriophage as defined in any one of embodiments 1 to 6 further comprising a detectable label, and detecting a change in said sample caused by said bacteriophage, as an indication of the presence of Listeria.
18. A method according to embodiment 17, wherein said change in the sample is due to lysis of said Listeria by said bacteriophage or wherein said change in the sample is due to the detectable label.

### EXAMPLES

This example relates to mutations in the receptor binding protein (tail fiber protein) in view of phage P100 that allow infection of Listeria serovar (SV) 1/2 and (SV) 3 mutants that are resistant to P100 and all relatives of P100 (genus Pecentumvirus) reported to date.

The two mutant viruses, designated P100+(P100plus) and P200, have been deposited under the regulations of the Budapest Treaty in the restricted CBS collection of Westerdijk Fungal Biodiversity Institute (Uppsalalaan 8, 3584 CT Utrecht, The Netherlands) and their sequences have been deposited in GenBank.

CBS 146750 Listeria virus P100+deposited at the GenBank accession nr. MT 178766.1; CBS 146751 Listeria virus P200 GenBank accession nr. MT 178767.1.

### Introduction

The cell-wall teichoic acids of Listeria serovar (SV) 1/2 strain carry sugar decorations, namely an N-acetyglucosamine (GlucNac) residue and a rhamnose (Rha) molecule, strains that have lost the GlucNac are designated SV 1/2*, and those lacking Rha are designated SV 3 (see FIG. 1) By including the newly identified bacteriophages in a product against Listeria, its usefulness would be greatly expanded.

We sequenced newly identified bacteriophages P100+and P200 and observed changes in a receptor binding protein (CDS 39722-41014 in the GenBank deposits).

FIG. 2 depicts the distal 181 residues of the receptor binding protein (the full-length protein has 430 residues) of four phage isolates. Bacteriophage A511 (deposited at ATCC with deposited number PTA-4608; see also Loessner et. al., Applied and Environmental Microbiology, June 1990, p. 1912-1918, 1990) has 94% sequence identity with bacteriophage P100 and has almost identical structural proteins and identical host-range compared to P100. Phage A511 was used for recombination experiments to allow quick differentiation between recombinants and contamination since we work with P200 and P100+ in the same lab). P100, P100+ and P200 are closely similar phages, but the latter two have mutations that broaden their host range. The distal portion of the receptor binding protein is depicted in FIG. 2; this is the region of the protein that interacts with the Listeria receptor molecule.

In order to demonstrate that the changes observed in this area are responsible for the altered host range we conducted recombination experiments that change this portion of the receptor binding protein in phage A511, resulting in the tail fiber protein being identical to that of either P200 or P100+. The methodology is described here below.

### Materials and Methods

The regions of interest of P100+ and P200 were introduced into phage A511 by homologous recombination of the phage and plasmid pCK1 harboring the P100+ or P200 region including upstream and downstream sequences. Infection by the phage of Listeria ivanovii WSLC 3009 strain harboring this plasmid allows for recombination to occur.

The regions of interest of P100+ and P200 were first synthetically generated and inserted into plasmid pCK1 (Gasson MJ, Anderson PH. High copy number plasmid vectors for use in lactic Streptococci. FEMS Microbiol Lett 1985; 30:193-6). The two synthetic sequences included restriction sited allowing insertion into the plasmid (see FIG. 3). The resulting plasmids were transformed into Listeria ivanovii WSLC 3009.

Cultures of both strains (containing the plasmid with either the P100+ or the P200 regions of interest) were subsequently infected with phage A511. After phage propagation dilutions of the progeny phage were plated on strains L. monocytogenes WSLC 1442 (serovar 1/2*) or L. monocytogenes WSLC1031 (serovar 3) respectively. Only P100+ is able to generate plaques on WSLC1442 while P100 and A511 do not, and P200 is able to generate plaques on WSLC1031 while P100 and A511 do not. The resulting phages were designated A511::P200 and A511::P100+.

Sequencing confirmed that indeed the regions of interest had recombined with A511 in resulting phages. The results are depicted here below.

>A511_P200_recombinant;
SEQ ID NO: 8
GTGACAGCCAACACTGTAGCAGTAGAGAGGGTAGTCGCAGATAGGATTAT
TACTACTCTAGGGGTACCGAGTAAGTGGGAATACAGAGCTAAAGCCTCTA
ATTTCTTCTTTAGTGCTTCCAATTCGTCACGTATAACACTAGTATCGACC
CAACAGAATATTATAACACCTAATAAATTTATTAATAACCCCGGTAGTTT
ACCACTATCTGCGATAAACCCCGCAATAACTATTCCAGAAGATGGGATGT
ACCAAGTGATGGCGGCCCTAAACATTAACGGTATTTTAAAAAAAGTTTAC
TTAGTTTCTGAATTAACTCTACTAATAAATGACGTAGTACACCCAGCTAC
ATTTGGAATGGTTAAAACTGTTGATAACGCTAACGGTCAATACTCTTTAG
CAGGGAATGGCATATATCAACTAAAAAAAGGGGACAAGTTAAAACTAAGA
AGTTACTGTAACAATACAGGGAATAACCCTTACTTGGATGTAGACAAACT
TTATATATCTGTTGGTAAGATTGCAGACATTTCATTATTTAAATAAACTT
AGTATGCTGAGAAAGGAAGTCACAATAATGGCAGAAGAAAAATTTTCATT
GGACGGAAGGCAGTATGTAACAGCAGTCCTATATATGTACCCTAACCTAG
ACCCTCAGTACGACTTTGGAGTTGTATCATCCGAAGGGAAGGTAGAAGCT
TTTGAATGGCACGTAAAAAATATAACAAAGCCTTCTATTGCACAGTTAGA
CCAAGCATATAAAGACTATGTAAAATCTATCGGAAGTCCAAGTAATCCAC
CAGCCCTAGAACCAGAGGTATCAAAAGAAGCCTCAGTAGGTAAGATTCTA
ACTGAAAAATTTAACACCTTAGAAGAAACAAATGCTAATATGTTATTGGA
AAATCTAGAGCTTCAAGCTAGCCTAGATAAAACAAAACAACAAAATTCAG
AACTAGTATTTGAGCTAGTAGATAGGGGAGTCTTATGA
>A511_P100+_recombinant;
SEQ ID NO: 9
GTGACAGCCAACACTGTAGAAGTAGAGAGGGTAGTCGCAGATAGGATTAT
TACTACTCTAGGGGTACCGAGTAAGTGGGAATACAGAGCTAAAGCCTCTA
ATTTCTTCTTTAGTGCTTCCAACTCGTCACGTATAACACTAGTATCGACC
CAACAGAATATTATAACACCTAATAAATTTATTAATAACCCCGATAGTTT
ACCACTATCTGCGATAAACCCCGCAATAACTATTCCAGAAGATGGGATGT
ACCAAGTGATGGCGGCCCTAAACATTAACGGTATTTTAAAAAAAGTTTAC
TTAGTTTCTGATTTAACTCTACTAATAAATGACGTAGTACACCCAGCTAC
ATTTGGAATGGTTAAAACTGCTGATAACGCTAACGGTCAACATTCTTTAG
CAGGGAATGGTATTTATCAACTAAAAAAAGGGGACAAGTTAAAACTAAGA
AGTTATTGTAACAATACAGGAAATAACCCTTACTTGGATGTAGACAAACT
TTATATATCTGTTGGTAAGATTGCAGACATTTCATTATTTAAATAAACTT
AGTATGCTGAGAAAGGAAGTCACAATAATGGCAGAAGAAAAATTTTCATT
GGACGGAAGGCAGTATGTAACAGCAGTCCTATATATGTACCCTAACCTAG
ACCCTCAGTACGACTTTGGAGTTGTATCATCCGAAGGGAAGGTAGAAGCT
TTTGAATGGCACGTAAAAAATATAACAAAGCCTTCTATTGCACAGTTAGA
CCAAGCATATAAAGACTATGTAAAATCTATCGGAAGTCCAAGTAATCCAC
CAGCCCTAGAACCAGAGGTATCAAAAGAAGCCTCAGTAGGTAAGATTCTA
ACTGAAAAATTTAACACCTTAGAAGAAACAAATGCTAATATGTTATTGGA
AAATCTAGAGCTTCAAGCTAGCCTAGATAAAACAAAACAACAAAATTCAG
AACTAGTATTTGAGCTAGTAGATAGGGGAGTCTTATGA
>Native_P100;
SEQ ID NO: 4
VTANTVAVERVVADRIITTLGVPSKWEYRAKASNFFFSASNSSRITLVST
QQNIITPNKFINNPGSLPLSAINPAITIPEDGMYQVMAALNINGILEKVY
LVSELTLLINDVVHPATFGMVKTADNANGQYSLAGNGIYQLKKGDKLKLR
SYCNNTGNNPYLDVDKLYISVGKIADISLFK
>Native_A511;
SEQ ID NO: 5
VTANTVEVERVVADRIITTLGVPSKWEYRAKASNFFFSASNSSRITLVST
QQNIITPNKFINNPDSLPLSAINPAITIPEDGMYQVMVTLNINGILEKVY
LVSELTLLVNDVVHPATFGMVKTVDNANGQYSLAGNGIYQLKKGDKLKLR
SYCNNTGNNPYLDVDKLYISVGKIADISLFK
>A511_P100+_recombinant;
SEQ ID NO: 6
VTANTVEVERVVADRIITTLGVPSKWEYRAKASNFFFSASNSSRITLVST
QQNIITPNKFINNPGSLPLSAINPAITIPEDGMYQVMAALNINGILKKVY
LVSDLTLLINDVVHPATFGMVKTADNANGQHSLAGNGIYQLKKGDKLKLR
SYCNNTGNNPYLDVDKLYISVGKIADISLFK
>A511_P200_recombinant;
SEQ ID NO: 7
VTANTVAVERVVADRIITTLGVPSKWEYRAKASNFFFSASNSSRITLVST
QQNIITPNKFINNPGSLPLSAINPAITIPEDGMYQVMAALNINGILKKVY
LVSELTLLINDVVHPATFGMVKTVDNANGQYSLAGNGIYQLKKGDKLKLR
SYCNNTGNNPYLDVDKLYISVGKIADISLFK

As can be observed here above, changes in both A511 recombinants resulted in a receptor binding protein identical to that of either P200 or P100+.

Isolation of these phages from either WSLC 1442 or WSLC 1031 already demonstrated that a host range change had occurred, but we conducted further experiments to corroborate that host ranges of the recombinants overlap with their P100 mutant counterparts, and that these changes do not lead to a loss of efficacy on other serovars. To this end we carried out host range experiments on several other strains, including further SV 1/2*, 3, 4, 5 and 6 strains which also include species other than Listeria monocytogenes.

Here below, the result of pull-down experiments are depicted. These experiments involved mixing of a small number of phages (˜3000 PFU) with 1 mL of overnight bacterial cultures, incubating for 5 minutes followed by centrifugation. Phages left in the supernatant were enumerated. This type of experiment is far more sensitive to indicate binding of the phage to the host than classical plaque experiments.

| | P100+ | A511::P100+ | P200 | A511::P200 | A511 | P100 |
|---|---|---|---|---|---|---|
| 2627 (L. in SV 6) | + | + | + | + | + | + |
| 1442 (L. mo SV 1/2*) | + | + | − | − | − | − |
| 40137 (L. se SV 3) | + | + | + | + | − | − |
| EGDe WT (L. mo SV 1/2) | + | + | + | + | + | + |
| EGDeΔ2550 (L. mo SV 1/2*) | + | + | − | − | − | − |
| 1031 (L. mo SV 3) | + | + | + | + | − | − |
| 1032 (L. mo SV 3) | + | + | + | + | − | − |
| 1042 (L. mo SV 4) | + | + | + | + | + | + |
| Arl1 (L. mo SV 1/2*) | + | + | − | − | − | − |
| C33R (L. mo SV 1/2*) | + | + | − | − | − | − |
| 1001 (L. mo SV 1/2) | + | + | + | + | + | + |
| 20100 (L. mo SV 6) | + | + | + | + | + | + |
| 40140 (L. se SV 1/2) | + | + | + | + | + | + |
| 3009 (L. iv SV 5) | + | + | + | + | + | + |
| 5008 (L. we SV 1/2) | + | + | + | + | + | + |

L. mo = L. monocytogenes, L. in = L. innocua, L. se = L. seeligeri, L. iv = L. ivanovii, L. we = L. welshimeri
“+” designates >80% binding of the phages; “−” represents less than 10% binding.

### Results and Discussion

Phages P100+ and P200 are superior in host range to P100 and other described Pecentumviruses. As such, their inclusion in anti-Listerial products for the food industry is highly desirable. Such an extended host-range would also be of interest phage-based bacteria detection.

The ability to recognize serovar 3 strains is the result of a single amino acid change at position 97 of the distal portion of the receptor binding protein from a glutamic acid to a lysine. This change is observed both in P200 and P100+. The ability to recognize SV 1/2* as observed in P100+ requires two (additional) changes. A change from glutamic acid to aspartic acid at position 104 and from tyrosine to histidine at position 131 of the distal part of the receptor binding protein. These positions correspond to residues 276 (for the change in both P100+ and P200), 283 and 310 in the full-length receptor binding protein. FIG. 4 indicates the location of the receptor binding protein on the phage tail.

### REFERENCES

Loessner et. al., Applied and Environmental Microbiology, June 1990, p. 1912-1918, 1990. Gasson MJ, Anderson PH. High copy number plasmid vectors for use in lactic Streptococci. FEMS Microbiol Lett 1985; 30:193-6

1. A bacteriophage lytic for at least Listeria serovar 1/2* and/or serovar 3, wherein said bacteriophage has a genome that has at least 70% sequence identity with the genome of bacteriophage P100 as set forward in SEQ ID NO: 1, and wherein the distal part of the receptor binding protein as set forward in SEQ ID NO: 4 has at least one mutation selected from: G157R, E97K, E104D and Y131H.

2. The bacteriophage according to claim 1, wherein the distal part of the receptor binding protein as set forward in SEQ ID NO: 4 has at least two mutations selected from:
G157R, E97K, E104D and Y131H.

3. The bacteriophage according to claim 1, wherein the distal part of the receptor binding protein as set forward in SEQ ID NO: 4 has the mutations selected from:
E97K; E97K and E104D; E97K and Y131H; E97K, E104D and Y131H; E104D and Y131H; G157R and E97K; G157R, E97K and E104D; G157R, E97K and Y131H; G157R, E97K, E104D and Y131H; G157R, E104D and Y131H.

4. The bacteriophage according to any one of claim 1, wherein said bacteriophage is bacteriophage P100+ as deposited under number CBS146750 or is bacteriophage P200 as deposited under number CBS146751.

5. The bacteriophage according to claim 1, wherein said bacteriophage is bacteriophage P100+ having the genome according to GenBank accession nr. MT 178766.1 (SEQ ID NO: 3) or is bacteriophage P200 having the genome according to GenBank accession nr. MT 178767.1 (SEQ ID NO: 4).

6. The bacteriophage according to claim 1, wherein said bacteriophage is also lytic for at least one of Listeria serovars 1/2, 4, 5 and 6.

7. The composition comprising at least one bacteriophage according to claim 1, preferably further comprising an agent selected from the group consisting of a bacteriophage endolysin, a surface disinfectant, an antibiotic, a surfactant, an antibacterial enzyme, a further bacteriophage lytic for Listeria and a bacteriophage specific for bacterial contaminants other than Listeria.

8. The composition according to claim 1, further comprising an organic acid, or a salt thereof, such as wherein the organic acid is selected from the group consisting of lactic acid, such as L-lactic acid, acetic acid, propionic acid and mixtures thereof, such as wherein the salt of the organic acid is selected from the group consisting of the sodium salt, potassium salt, ammonium salt and mixtures thereof, such as K-(L)lactate, Na-(L)lactate, K-acetate, Na-acetate, K-diacetate, Na-diacetate, K-propionate, Na-propionate and mixtures thereof.

9. A method for controlling Listeria contamination in a food product, on food processing equipment or on food storage containers, comprising contacting a bacteriophage or a combination of bacteriophages according to claim 1, or a composition comprising said bacteriophage or comprising said combination of bacteriophages, to a food product or to food processing equipment to reduce the amount of Listeria.

10. The method according to claim 9, wherein the food product is a processed, non-processed, cured or uncured food product selected from the group consisting of meat, fish, shellfish, pastry, dairy product, vegetables, fruit and mixtures thereof.

11. The method according to claim 10, wherein the food product is selected from the group consisting of beef, pork, lamb, fruit, vegetables, including but not limited to lettuce, leafy greens, baby leafy greens, sprouts.

12. The method according to claim 10, wherein said bacteriophage or composition is administered by spraying or misting said bacteriophage or composition to the food product or by dipping or soaking the food product into said composition, wherein said food storage container is preferably a salad bar.

13. The method according to claim 9, wherein said food processing equipment is selected from the group consisting of a tube through which milk is being pumped, a high-salt content tank for processing cheese, a container from which cultures are applied to a surface of a cheese, a storage shelve for a food product, a shelve on which a food product is dried and cured, and a floor drain.

14. The method according to claim 9, wherein said bacteriophage or composition are sprayed onto a surface selected from the group consisting of food products, food processing equipment and food storage containers.

15. A method for detection of Listeria, including serovar 1/2* or serovar 3, comprising obtaining a sample suspected to contain Listeria, incubating said sample with a bacteriophage as defined in claim 1 further comprising a detectable label, and detecting a change in said sample caused by said bacteriophage, as an indication of the presence of Listeria, wherein said change in the sample is preferably due to lysis of said Listeria by said bacteriophage or wherein said change in the sample is due to the detectable label.
